(19) United States
(12) Patent Application Publication
(10) Pub. No.: US 2024/0041914 A1
(43) Pub. Date: Feb. 8, 2024

(54) RNAi APPROACH FOR CROP PEST PROTECTION

(71) Applicant: Donald Danforth Plant Science Center, St. Louis, MO (US)

(72) Inventor: Bala P. Venkata, St. Louis, MO (US)

(73) Assignee: Donald Danforth Plant Science Center, St. Louis, MO (US)

(21) Appl. No.: 18/450,977

(22) Filed: Aug. 16, 2023

Related U.S. Application Data
(62) Division of application No. 16/610,267, filed on Nov. 1, 2019, now abandoned.
(60) Provisional application No. 62/492,556, filed on May 1, 2017.

(51) Int. Cl. A61K 31/713; C12N 15/113; C12N 15/82; A01N 63/60

(52) U.S. Cl. CPC A61K 31/713; C12N 15/113; C12N 15/8286; A01N 63/60; C12N 2310/14

(57) ABSTRACT

Provided herein is the identification of insect RNAi target genes (IRTG) involved in gut microbial clearance and containment and examples of a novel biotechnology for devising pesticidal RNAi approaches.

### CROSS-REFERENCE TO RELATED APPLICATIONS

The present application is a divisional of the U.S. Non-Provisional patent application Ser. No. 16/610,267 filed on Nov. 1, 2019, which claims priority to PCT Application No. PCT/US2018/030506, filed May 1, 2018, which, in turn claims the benefit of U.S. Provisional Application No. 62/492,556, filed May 1, 2017. The disclosures of each of the aforementioned applications are incorporated herein by reference in its entirety.

### REFERENCE TO A SEQUENCE LISTING

This application contains a Sequence Listing that has been submitted in .XML format via PatentCenter and is hereby incorporated by reference in its entirety. The .XML is named Sequence_Listing.xml, and is 255 kilobytes in size.

### BACKGROUND

Insect pests are detrimental to crop production and human health throughout the world and insect control can in some instances consume between 10-25% of a country's gross national product (GNP). (http World Wide Web internet site “fao.org/3/a-av013e.pdf”). In the U.S., annual loss due to crop pests is estimated to exceed $120 billion USD/year. (Polaszek A. (1998) Wallingford, UK: CABI. 530 pp.).

Within crop pests, Lepidoptera are the most detrimental insect pests of cereal crop cultivation. Chemical control is often expensive, inefficient, and can be associated with negative environmental consequences. Host plant resistance is an attractive option but impeded by lack of robust Lepidoptera resistant germplasm (http World Wide Web internet site “cnbc.com/2015/05/08/insects-feast-on-plants-endangering-crops-and-costing-billions.html”).

Since 1996, commercialization of crop plants genetically engineered to produce Bacillus thuringiensis (Bt) insecticidal proteins have resulted in efficient pest control, increased yield, reduced insecticidal use, and enhanced farmer profits. (Khan Z R, et al. (2014). Philos. Trans. R Soc. Lond Biol Sci. 369: 1639).

Consequently, the cumulative area planted with Bt crops worldwide reached greater than 1 billion acres during 2011. Within the U.S., Bt Corn, Bt Soybean, and Bt Cotton accounted for 90% of all the total corn, soybean and cotton acres during 2013 (Tabashnik B E, et al. (2013). Nat. Biotech. 31: 510-521). However, evolution of field resistance against Bt in lepidopteran pests raises potential concerns about the sustainability of this approach. (Campagne P., et al. (2013) PLoS ONE 8(7): e69675. Doi:10.1371). That is further exacerbated by loss of resistance against pyramided Bt traits as well (https World Wide Web internet site dt npf.com/agriculture/web/ag/news/article/2016/08/10/rootworm-resistance-pyramided-bt.html).

### SUMMARY

Provided herein is an isolated double stranded RNA (dsRNA) molecule comprising a nucleic acid sequence complementary to about 200 to 1000 contiguous nucleotides of a target gene sequence—wherein the target gene is a MIGGS-IRTG as defined herein-involved in gut microbe clearance and/or containment induced by microbes ingested during feeding and/or active feeding. In certain aspects, the target gene is critical for insect immune responses and certain aspects provide that it is abundantly expressed in the midgut. In certain aspects, the target gene sequence includes at least one of the protein coding region, the 5′ UTR region, the 3′ UTR region, and any combination thereof, of a target gene. Further, certain aspects provide that the dsRNA molecule silences the target gene when ingested by an insect. In certain aspects, the target gene is a type 1 MIGGS RNAi target or a type 2 MIGGS RNAi target as defined elsewhere herein. In certain aspects, the target gene is a pattern recognition receptor (PRR) class gene or an insect midgut structural component gene. In certain aspects, the target gene is expressed abundantly in a midgut specific manner during active feeding.

In certain aspects, a dsRNA molecule disclosed anywhere herein comprises two annealed complementary RNA strands. In certain aspects, said dsRNA molecule comprises a single RNA strand comprising an inversely repeated sequence with a spacer in between, wherein the single RNA strand can anneal to itself to form a hairpin loop structure.

In certain aspects, a dsRNA molecule disclosed anywhere herein comprises a nucleic acid sequence complementary to about 200 to 1000 contiguous nucleotides of the protein coding region of the target gene sequence. In certain aspects, said dsRNA molecule comprises a nucleic acid sequence complementary to about 200 to 1000 contiguous nucleotides of the 5′ UTR region or the 3′ UTR region of the target gene sequence. In certain aspects, said dsRNA molecule comprises a nucleic acid sequence complementary to a contiguous region comprising at least about 50%, 60%, 70%, 80%, 90%, 95%, 98%, 99%, or 100% of the length of the target gene sequence protein coding region, 5′ UTR region, or 3′ UTR region. In certain aspects, said dsRNA molecule comprises a nucleic acid sequence complementary to about 200 to 650 contiguous nucleotides of a target gene sequence.

Certain aspects of this disclosure are drawn to a target gene selected from the group consisting of M. sexta-Hemolin (MsHEM), M. sexta-Serine proteinase homolog 3 (MsSPH-3), M. sexta-Peptidoglycan recognition protein 2 (MsPGRP2), M. sexta-Beta-1, 3-glycan-recognition protein 2 (MsβGRP2), M. sexta-Relish family protein 2A (MsREL2A), M. sexta-Dorsal (MsDor), M. sexta-Spatzle (MsSPZ1A), M. sexta-Toll receptor (MsTOLL), M. sexta-Scolexin A (MsSCA1), M. sexta-Hemolymph proteinase 18 (MsHP18), M. sexta-Transferrin (MsTRN), M. sexta-Arylphorin beta subunit (MsARP), M. sexta-Chymotrypsinogen-like protein 1 (MsCTL1), M. sexta-Valine Rich Midgut Protein (MsVMP1), M. sexta-Imd (MsImd), M. sexta-FADD (MsFADD), M. sexta-Dredd (MsDRD), M. sexta-Relish F (MsReIF), M. sexta-Cdc42 (MsCdc42), M. sexta-Dsor1 (MsDsor1), M. sexta-Fos (MsFos), M. sexta-Jra (MsJra), M. sexta-Caudal (MsCAD1), M. sexta-Atg8 (MsAtg8), M. sexta-Atg13 (MsAtg13), M. sexta-IAP1 (MsIAP1), M. sexta-Chitin synthase 2 (MsChs2), M. sexta-Beta-1 tubulin (MspTub), M. sexta-Beta fructofuranosidase 1 (MsSuc1), and orthologs thereof. In certain aspects, the target gene is selected from the group consisting of M. sexta-Hemolin (MsHEM), M. sexta-Serine proteinase homolog 3 (MsSPH-3), M. sexta-Peptidoglycan recognition protein 2 (MsPGRP2), M. sexta-Beta-1, 3-glycan-recognition protein 2 (MsβGRP2), M. sexta-Relish family protein 2A (MsREL2A), M. sexta-Dorsal (MsDor), M. sexta-Spatzle (MsSPZ1A), M. sexta-Toll receptor (MsTOLL), M. sexta-Scolexin A (MsSCA1), M. sexta-Hemolymph proteinase 18 (MsHP18), M. sexta-Transferrin (MsTRN), M. sexta-Arylphorin beta subunit (MsARP), M. sexta-Chymotrypsinogen-like protein 1 (MsCTL1), M. sexta-Valine Rich Midgut Protein (MsVMP1), M. sexta-Imd (MsImd), M. sexta-FADD (MsFADD), M. sexta-Dredd (MsDRD), M. sexta-Relish F (MsReIF), M. sexta-Cdc42 (MsCdc42), M. sexta-Dsor1 (MsDsor1), M. sexta-Fos (MsFos), M. sexta-Jra (MsJra), M. sexta-Caudal (MsCAD1), M. sexta-Atg8 (MsAtg8), M. sexta-Atg13 (MsAtg13), M. sexta-IAP1 (MsIAP1), M. sexta-Chitin synthase 2 (MsChs2), M. sexta-Beta-1 tubulin (MspTub) and M. sexta-Beta fructofuranosidase 1 (MsSuc1).

Also provided herein is an isolated double stranded RNA (dsRNA) molecule comprising a nucleic acid sequence complementary to about 200 to 1000 contiguous nucleotides of a target gene sequence, wherein the target gene comprises a nucleic acid sequence selected from the group consisting of SEQ ID NOs: 1-14, 16-29, 31-70, 71-75, 76-88, 89-105, and 106-110. In certain aspects, the target gene sequence includes at least one of the protein coding region, the 5′ UTR region, the 3′ UTR region, and any combination thereof, of a target gene. Further, certain aspects provide that the dsRNA molecule silences the target gene when ingested by an insect.

In certain aspects disclosed herein, the target gene is sequence selected from the group consisting of: i) SEQ ID NOs: 1-9, 11, 14, 31, 39, 43, 44, and 71-75.

In certain aspects disclosed herein, the target gene is sequence selected from the group consisting of: ii) SEQ ID NOs: 3, 4, and 43. In certain aspects, the dsRNA molecule causes impeded growth, developmental progression, and/or mortality and the like of TH, DMB, and FAW in an orthologous manner.

In certain aspects disclosed herein, the target gene is sequence selected from the group consisting of: iii) SEQ ID NOs: 76, 77, 80, 81, 85, 87, and 88. In certain aspects, the dsRNA molecule causes impeded growth, developmental progression, and/or mortality and the like of DBM. Further, in certain aspects, the DBM is a Bt resistant strain.

In certain aspects disclosed herein, the target gene is sequence selected from the group consisting of: iv) SEQ ID NOs: 89, 92, 96, 101, 103, and 105. In certain aspects, the dsRNA molecule causes impeded growth, developmental progression, and/or mortality and the like of FAW.

In certain aspects disclosed herein, the target gene is sequence selected from the group consisting of: v) SEQ ID NOs: 107-110. In certain aspects, the dsRNA molecule caused impeded growth, developmental progression, and/or mortality and the like of RFB.

In certain aspects of the dsRNA molecule disclosed above, the dsRNA molecule comprises two annealed complementary RNA stands. In certain aspects, the dsRNA molecule comprises a single RNA strand comprising an inversely repeated sequence with a spacer in between and where the single RNA strand can anneal to itself to form a hairpin loop structure.

In certain of the dsRNA molecule disclosed above, the dsRNA molecule comprises a nucleic acid sequence complementary to about 200 to 1000 contiguous nucleotides of the protein coding region of the target gene sequence. In certain aspects, the dsRNA molecule comprises a nucleic acid sequence complementary to a contiguous region comprising at least about 50%, 60%, 70%, 80%, 90%, 95%, 98%, 99%, or 100% of the length of a sequence selected from the group consisting of SEQ ID NOs: 1-14, 16-29, 31-70, 71-75, 76-88, 89-105, and 106-110. In certain aspects, the dsRNA molecule comprises a nucleic acid sequence complementary to a contiguous region comprising at least about 50%, 60%, 70%, 80%, 90%, 95%, 98%, 99%, or 100% of the length of the target gene sequence protein coding region, 5′ UTR region, or 3′ UTR region.

In certain aspects disclosed herein the dsRNA molecule comprises a nucleic acid sequence selected from the group consisting of SEQ ID NOs: 111-119, 120-126, 127-135, and 136-139. In certain aspects, the dsRNA is a fragment of at least about 200 nucleotides thereof. In certain aspects, i) the isolated dsRNA molecule comprising a nucleic acid sequence selected from the group consisting of SEQ ID NOs: 110-119, or the fragment thereof, causes impeded growth, developmental progression, and/or mortality and the like of TH; ii) the isolated dsRNA molecule comprising a nucleic acid sequence selected from the group consisting of SEQ ID NOs: 120-126, or the fragment thereof, causes impeded growth, developmental progression, and/or mortality and the like of DBM; iii) the isolated dsRNA molecule comprising a nucleic acid sequence selected from the group consisting of SEQ ID NOs: 127-135, or the fragment thereof, causes impeded growth, developmental progression, and/or mortality and the like of FAW; or iv) the isolated dsRNA molecule comprising a nucleic acid sequence selected from the group consisting of SEQ ID NOs: 136-139, or the fragment thereof, causes impeded growth, developmental progression, and/or mortality and the like of RFB.

In certain of any of the above aspects, the dsRNA molecule can form siRNA. Certain aspects provide for an isolated siRNA molecule derived from the processing of said dsRNA molecule.

Certain further aspects provide of an insecticidal composition comprising an isolated dsRNA molecule or an siRNA molecule disclosed anywhere herein, and a synthetic carrier or microbial conduit. In certain aspects, a microorganism has a natural capacity or is engineered to produce and/or deliver dsRNA to increase its bioavailability and/or biostability for causing RNA interference including but not restricted to plant growth promoting organisms, normal commensal and/or symbiotic microorganisms associated with the target insect pest or parasites and/or natural enemies of the target pest or pest target host or host cultivation range etc. from an insect or parasite and/or natural enemies of the target pest engineered or identified from natural populations containing microbial conduit to produce and/or deliver dsRNA and/or drive the transmission of such microbial conduits into natural populations of insect pests as a control option. In certain aspects of an insecticidal composition disclosed herein, the dsRNA molecule is conjugated with the synthetic carrier.

Certain aspects are also drawn to a recombinant DNA construct encoding a dsRNA molecule disclose anywhere herein. In certain aspects, the recombinant DNA construct comprising a gene silencing sequence comprising about 200 to 1000 contiguous nucleotides of a target gene sequence. In certain aspects, the target gene is a MIGGS-IRTG, as defined herein, involved in gut microbe clearance and/or containment induced by microbes ingested during feeding and/or active feeding. In certain aspects, the target gene is critical for insect immune responses. In certain aspects, the target gene is abundantly expressed in the midgut. In certain aspects, said target gene sequence includes at least one of the protein coding region, the 5′ UTR region, the 3′ UTR region, and any combination thereof, of a target gene. In certain aspects, the target gene is a type I MIGGS RNAi target or a type 2 MIGGS RNAi target as described elsewhere herein. In certain aspects, the target gene is a pattern recognition receptor (PRR) class gene or an insect midgut structural component gene. In certain aspects, the target gene is expressed abundantly in a midgut specific manner during active feeding.

In any of the above aspects of a recombinant DNA construct, the gene silencing sequence comprises about 200 to 1000 contiguous nucleotides of the protein coding region of the target gene sequence. In certain aspects, the gene silencing sequence comprises about 200 to 1000 contiguous nucleotides of the 5′ UTR region or the 3′ UTR region of the target gene sequence. In certain aspects, the gene silencing sequence comprises at least 50%, 60%, 70%, 80%, 90%, 95%, 98%, 99%, or 100% contiguously of the length of target gene sequence protein coding region, 5′ UTR region, or 3′ UTR region. In certain aspects, the gene silencing sequence comprises about 200 to 650 contiguous nucleotides of the target gene sequence.

In any of the above aspects of a recombinant DNA construct, and as noted throughout this disclosure, in certain aspects, a target gene can be selected from the group consisting of M. sexta-Hemolin (MsHEM), M. sexta-Serine proteinase homolog 3 (MsSPH-3), M. sexta-Peptidoglycan recognition protein 2 (MsPGRP2), M. sexta-Beta-1, 3-glycan-recognition protein 2 (MsβGRP2), M. sexta-Relish family protein 2A (MsREL2A), M. sexta-Dorsal (MsDor), M. sexta-Spatzle (MsSPZ1A), M. sexta-Toll receptor (MsTOLL), M. sexta-Scolexin A (MsSCA1), M. sexta-Hemolymph proteinase 18 (MsHP18), M. sexta-Transferrin (MsTRN), M. sexta-Arylphorin beta subunit (MsARP), M. sexta-Chymotrypsinogen-like protein 1 (MsCTL1), M. sexta-Valine Rich Midgut Protein (MsVMP1), M. sexta-Imd (MsImd), M. sexta-FADD (MsFADD), M. sexta-Dredd (MsDRD), M. sexta-Relish F (MsReIF), M. sexta-Cdc42 (MsCdc42), M. sexta-Dsor1 (MsDsor1), M. sexta-Fos (MsFos), M. sexta-Jra (MsJra), M. sexta-Caudal (MsCAD1), M. sexta-Atg8 (MsAtg8), M. sexta-Atg13 (MsAtg13), M. sexta-IAP1 (MsIAP1), M. sexta-Chitin synthase 2 (MsChs2), M. sexta-Beta-1 tubulin (MspTub), M. sexta-Beta fructofuranosidase 1 (MsSuc1), and orthologs thereof. In certain aspects a target gene can be selected from the group consisting of M. sexta-Hemolin (MsHEM), M. sexta-Serine proteinase homolog 3 (MsSPH-3), M. sexta-Peptidoglycan recognition protein 2 (MsPGRP2), M. sexta-Beta-1, 3-glycan-recognition protein 2 (MsβGRP2), M. sexta-Relish family protein 2A (MsREL2A), M. sexta-Dorsal (MsDor), M. sexta-Spatzle (MsSPZ1A), M. sexta-Toll receptor (MsTOLL), M. sexta-Scolexin A (MsSCA1), M. sexta-Hemolymph proteinase 18 (MsHP18), M. sexta-Transferrin (MsTRN), M. sexta-Arylphorin beta subunit (MsARP), M. sexta-Chymotrypsinogen-like protein 1 (MsCTL1), M. sexta-Valine Rich Midgut Protein (MsVMP1), M. sexta-Imd (MsImd), M. sexta-FADD (MsFADD), M. sexta-Dredd (MsDRD), M. sexta-Relish F (MsReIF), M. sexta-Cdc42 (MsCdc42), M. sexta-Dsor1 (MsDsor1), M. sexta-Fos (MsFos), M. sexta-Jra (MsJra), M. sexta-Caudal (MsCAD1), M. sexta-Atg8 (MsAtg8), M. sexta-Atg13 (MsAtg13), M. sexta-IAP1 (MsIAP1), M. sexta-Chitin synthase 2 (MsChs2), M. sexta-Beta-1 tubulin (MspTub) and M. sexta-Beta fructofuranosidase 1 (MsSuc1).

Further aspects provide for a recombinant DNA construct comprising a gene silencing sequence comprising about 200 to 1000 contiguous nucleotides of a target gene sequence, wherein the target gene comprises a nucleic acid sequence selected from the group consisting of SEQ ID NOs: 1-14, 16-29, 31-70, 71-75, 76-88, 89-105, and 106-110. In certain aspects, the target gene sequence includes at least one of the protein coding region, the 5′ UTR region, the 3′ UTR region, and any combination thereof, of a target gene.

In any of the above aspects of a recombinant DNA construct, the target gene sequence is selected from the group consisting of: i) SEQ ID NOs: 1-9, 11, 14, 31, 39, 43, 44, and 71-75; ii) SEQ ID NOs: 3, 4, and 43; iii) SEQ ID NOs: 76, 77, 80, 81, 85, 87, and 88; iv) SEQ ID NOs: 89, 92, 96, 101, 103, and 105; and v) SEQ ID NOs: 107-109, and 110. In certain aspects, the gene silencing sequence comprises about 200 to 1000 contiguous nucleotides of a sequence selected from the group consisting of SEQ ID NOs: 1-14, 16-29, 31-70, 71-75, 76-88, 89-105, and 106-110. In certain aspects, the gene silencing sequence comprises at least 50%, 60%, 70%, 80%, 90%, 95%, 98%, 99%, or 100% contiguously of a sequence selected from the group consisting of SEQ ID Nos: 1-14, 16-29, 31-70, 71-75, 76-88, 89-105, and 106-110. In certain aspects, the gene silencing sequence comprises about 200 to 650 contiguous nucleotides of a sequence selected from the group consisting of SEQ ID NOs: 1-14, 16-29, 31-70, 71-75, 76-88, 89-105, and 106-110. In certain aspects, the gene silencing sequence is operably linked to one or more promoters for the expression of a dsRNA molecule that silences the target gene when ingested by an insect. In certain aspects, the construct is an expression vector. And, in certain aspects, the expression vector can target single or multiple insect RNAi target genes or chimeric RNAi target genes.

Certain aspects of the disclosure also provide for a host cell comprising the dsRNA molecule, the siRNA molecule, a polynucleotide encoding a dsRNA molecule, and/or the construct or a dsRNA encoding segment thereof disclose anywhere herein. In certain aspects, the host cell is a bacterial or plant cell or organelle. In certain aspects, the organelle is a plastid. In certain aspects, the host cell is a transgenic and/or transplastomic plant cell. In certain aspects, the hose cell expresses a dsRNA and/or produces an siRNA disclosed anywhere herein.

Certain aspects also provide for a transgenic and/or transplastomic plant comprising a dsRNA molecule, an siRNA molecule, a polynucleotide encoding the dsRNA, and/or a construct or a dsRNA encoding segment disclosed anywhere herein. In certain aspects, at least one cell of the plant expresses the dsRNA molecule and/or produces the siRNA molecule. Further, certain aspects provide for a seed, part, tissue, cell, or organelle of the above transgenic and/or transplastomic plant. In certain aspects, the seed, part, tissue, cell, or organelle comprises the dsRNA molecule and/or the siRNA molecule. In certain aspects, the organelle is a plastid.

Certain aspects provide for a method of silencing: (i) an insect immune response gene and/or (ii) an insect gene encoding for structural components of the insect midgut. In certain aspects, the method comprises providing for ingesting an isolated dsRNA molecule, an siRNA molecule, an insecticidal composition, a host cell, a transgenic and/or transplastomic plant, transplastomic plant and/or a seed, part, tissue, cell, or organelle as disclosed anywhere herein, to an insect.

Certain aspects provide for a method of protecting a plant from an insect pest of the plant. In certain aspects, the method comprises topically applying to a plant an isolated dsRNA molecule, an siRNA molecule, and/or an insecticidal composition disclosed anywhere herein, and providing the plant in the diet of the insect pest. In certain aspects, the dsRNA is topically applied by expressing the dsRNA in a microbe and topically applying the microbe onto the plant.

Certain aspects provide for a method of producing a plant resistance to a pest insect of said plant. In certain aspects, the method comprises transforming a plant with a polynucleotide encoding the dsRNA molecule and/or a construct or a dsRNA encoding segment thereof as disclosed anywhere herein, wherein the plant expresses a dsRNA molecule and/or produces an siRNA disclose anywhere herein.

Certain aspects provide for a method of improving crop yield. In certain aspects, the method comprises growing a population of crop plants transformed with a polynucleotide encoding a dsRNA molecule and/or a construct or a dsRNA encoding segment thereof wherein the plant expresses a dsRNA molecule and/or produces an siRNA molecule as discloses anywhere herein. In certain aspects, the population of transformed plants produces higher yields in the presence of pest insect infestation than a control population of untransformed plants.

Certain aspects provide for a method for producing a plant resistant against a pest insect of said plant. In certain aspects, the method comprises: a) transforming a plant cell and/or organelle with a polynucleotide encoding a dsRNA molecule and/or a construct or a dsRNA encoding segment thereof as disclosed anywhere herein; b) regenerating a plant from the transformed plant cell and/or organelle; and c) growing the transformed plant under conditions suitable for the expression of said double stranded RNA molecule, wherein said transformed plant of (c) is resistant to the plant pest insect compared to an untransformed plant.

In certain of any of the aforementioned aspects, the method the dsRNA is ingested by an actively feeding stage of the insect. In certain aspects, the ingestion of the dsRNA induces a melanotic response in the insect larvae. In certain aspects, the ingestion of the dsRNA results in perturbation of gut microbial homeostasis. In certain aspects, the ingestion of the dsRNA results in defective clearance of opportunistic microbes. In certain aspects, the ingestion of the dsRNA results in defective containment of gut microbes.

In certain of any of the aforementioned aspects, the silencing of the target gene results in reduced appetite and/or developmental defects resulting in incomplete development and/or mortality and/or decrease the reproductive success of the insect. In certain aspects, the reduced appetite and/or developmental defects and/or mortality and/or reduced reproductive fitness of the insect is observed after sustained feeding for at least 72 hours.

In certain of any of the aforementioned aspects, the insect is of the order Lepidoptera, Coleoptera, Hemiptera, Blattodea, or Diptera. In certain aspects, the insect is Manduca sexta (M. sexta) (tobacco hornworm), Spodoptera frugiperda (fall armyworm), Ostrinia nubilalis (European corn borer), Plutella xylostella (Diamondback moth), Leptinotarsa decemlineata Say (Colorado potato beetle), Diabrotica spp. (Corn rootworm complex), Tribolium castaneum (Red flour beetle), Popillia japonica (Japanese beetle), Agrilus planipennis (Emerald ash borer), Diaphorina citri (Asian citrus psyllid), Cimex lectularius (Bed bug), a cockroach or termite, or insect pests such as mosquitoes and flies.

In certain of any of the aforementioned aspects, the plant host is selected from the group consisting of Zea mays L (corn), Sorghum bicolor (sorghum), Setaria italica (fox tail millet), Pennisetum glaucum (Pearl millet), Solanum tuberosum (potato), Oryza sativa (rice), Lycopersicon esculentum (tomato), Solanum melongena (eggplant), cultivars of the Brassica oleracea family, Citrus sinensis (Orange), trees of the Oleaceae family, and crops of Rosaceae.

One aspect of the instant disclosure encompasses a method of silencing an insect immune response gene, an insect gene encoding structural components of an insect midgut, or both. The method comprising providing for ingestion (a) an isolated double stranded RNA (dsRNA) molecule, or a dsRNA molecule in a host cell, in a transgenic or transplastomic plant or cell, organelle, or part thereof, in a microbial conduit, or in an insecticidal composition, wherein the dsRNA molecule comprises a nucleic acid sequence complementary to about 21 to 2000 contiguous nucleotides of a target gene sequence comprising a nucleic acid sequence of SEQ ID NO: 76, wherein the dsRNA molecule silences the target gene when ingested by an insect; (b) an siRNA molecule derived from the processing of the dsRNA molecule; (c) a polynucleotide, a construct, or a dsRNA encoding segment encoding the dsRNA molecule; or (d) a combination of (a)-(d).

The microbial conduit can comprise plant growth promoting organisms, normal commensal and/or symbiotic microorganisms associated with a target insect pest or parasite, and/or natural enemies of the target pest or pest target host or host cultivation range etc. from an insect or parasite, and/or natural enemies of the target pest engineered or identified from natural populations containing microbial conduit to produce and/or deliver dsRNA and/or drive transmission of such microbial conduits into natural populations of insect pests as a control option.

In some aspects, the dsRNA molecule is bound to a synthetic carrier. In some aspects, the synthetic carrier comprises chitosan, liposomes, carbon quantum dots, biodegradable particles of plant, or soil.

Ingestion of the dsRNA, the siRNA molecule, the polynucleotide, construct, or dsRNA encoding segment encoding the dsRNA molecule, or any combination thereof can silence the target gene to thereby induce (a) a melanotic response; (b) results in perturbation of gut microbial homeostasis; (c) results in defective clearance of opportunistic microbes; (d) results in defective containment of gut microbes, or any combination of (a) to (d).

The host cell can be a bacterial cell, a yeast cell, or a fungal cell. The target gene sequence can include at least one of a protein coding region, a 5′ untranslated region (UTR), a 3′ UTR, or any combination thereof.

The dsRNA molecule can comprise a single RNA strand comprising an inversely repeated sequence with a spacer in between and where the single RNA strand can anneal to itself to form a hairpin loop structure; or wherein the dsRNA molecule comprises two separate complementary RNA stands annealed together.

The target gene can be selected from the group consisting of Manduca sexta-Peptidoglycan recognition protein 2 (MsPGRP2), Plutella xylostella PGRP2, and Tribolium castaneum PGRP2.

In some aspects, the dsRNA comprises a nucleic acid sequence of SEQ ID NO: 76, or a fragment of at least about 21 nucleotides thereof, optionally.

In some aspects, the construct encoding the dsRNA comprises a gene silencing sequence operably linked to one or more promoters for expression of a dsRNA molecule that silences the target gene when ingested by an insect, optionally wherein the construct further comprises an additional transcription regulatory region or an additional transcriptional regulatory element. The construct can be an expression vector, and the expression vector can target single or multiple insect RNAi target genes or chimeric RNAi target genes.

The dsRNA molecule can cause impeded growth, developmental progression, and/or mortality and the like of DBM, optionally wherein the DBM is a Bt resistant strain. In some aspects, the silencing of the target gene results in reduced appetite and/or developmental defects resulting in incomplete development and/or mortality and/or decreased reproductive success of the insect, optionally wherein the reduced appetite and/or developmental defects and/or mortality and/or reduced reproductive fitness of the insect is observed after sustained feeding for at least 24 hours.

In some aspects, the insect is of the order Lepidoptera, Coleoptera, Hemiptera, Blattodea, or Diptera. In some aspects, the insect is Manduca sexta (M. sexta) (tobacco hornworm), Spodoptera frugiperda (fall armyworm), Ostrinia nubilalis (European corn borer), Plutella xylostella (Diamondback moth), Leptinotarsa decemlineata Say (Colorado potato beetle), Diabrotica spp. (Corn rootworm complex), Tribolium castaneum (Red flour beetle), Popillia japonica (Japanese beetle), Agrilus planipennis (Emerald ash borer), Diaphorina citri (Asian citrus psyllid), Cimex lectularius (Bed bug), a cockroach or termite, or insect pests such as mosquitoes and flies. In some aspects, the plant is selected from the group consisting of Zea mays L (corn), Sorghum bicolor (sorghum), Setaria italica (fox tail millet), Pennisetum glaucum (Pearl millet), Solanum tuberosum (potato), Oryza sativa (rice), Lycopersicon esculentum (tomato), Solanum melongena (eggplant), cultivars of the Brassica oleracea family, Citrus sinensis (Orange), trees of the Oleaceae family, and crops of Rosaceae.

Another aspect of the instant disclosure encompasses a method of protecting a plant from an insect pest of the plant. The method comprises topically applying to the plant (a) an isolated double stranded RNA (dsRNA) molecule, or a dsRNA in a host cell, in a transgenic or transplastomic plant or cell, organelle, or part thereof, in a microbial conduit, or in an insecticidal composition, and providing the plant in the diet of the insect pest, wherein the dsRNA molecule comprises a nucleic acid sequence complementary to about 21 to 2000 contiguous nucleotides of a target gene sequence comprising a nucleic acid sequence of SEQ ID NO: 76, and wherein the double stranded RNA molecule silences the target gene when ingested by an insect; (b) an siRNA molecule derived from processing of the dsRNA molecule; (c) a polynucleotide, a construct, or a dsRNA encoding segment encoding the dsRNA molecule; or a combination of (a)-(c).

Yet another aspect of the instant disclosure encompasses a method of producing a transgenic or transplastomic plant. The method comprises (a) transforming the plant with a polynucleotide encoding a dsRNA, a construct or a dsRNA encoding segment encoding the dsRNA, or both to generate a transformed plant cell; (b) regenerating a plant from the transformed plant cell and/or organelle to generate a transformed plant; and (c) growing the transformed plant under conditions suitable for expression of said dsRNA. The transformed plant of (c) is resistant to a plant pest insect compared to an untransformed plant and wherein the dsRNA molecule comprises a nucleic acid sequence complementary to about 21 to 2000 contiguous nucleotides of a target gene sequence comprising a nucleic acid sequence of SEQ ID NO: 76.

One aspect of the instant disclosure encompasses a method of improving crop yield. The method comprises growing a population of transgenic or transplastomic plants comprising a polynucleotide encoding a dsRNA, a construct or a dsRNA encoding segment encoding the dsRNA molecule, wherein the dsRNA comprises a nucleic acid sequence complementary to about 21 to 2000 contiguous nucleotides of a target gene sequence comprising a nucleic acid sequence of SEQ ID NO: 76 and wherein the population of transformed plants produces higher yields in the presence of pest insect infestation than a control population of untransformed plants.

### BRIEF DESCRIPTION OF THE DRAWINGS

The application file contains at least one photograph executed in color. Copies of this patent application publication with color photographs will be provided by the Office upon request and payment of the necessary fee.

FIG. 1 shows schematic representation of bacterially ingestible dsRNA assay. (Kamath R S, et al. (2000) Genome Biol. 2: 1-10; Newmark et al. (2003) Proc. Natl. Acad. Sci. USA 100: 11861-11865).

FIG. 2A-C shows representative phenotypes of TH larvae. TH larvae exposed to bacterially (HT115 (DE3)) expressed dsRNA against MIGGS RNAi targets MsPGRP2 (A); MsVMP1(B); and negative control dsRNA against Cassava plant specific gene MeCAT1 (C).

FIG. 3A,B shows feeding activity of TH larvae exposed to dsRNA against negative control MeCAT1 (A) and MIGGS RNAi target MsPGRP2 (B) containing bacterial (HT115 (DE3)) plates.

FIG. 4 shows survival rates of healthy conventionally-reared (CR) and germ-free (GF) first instar TH larvae exposed to bacterially (HT115 (DE3)) expressed dsRNA against MIGGS RNAi targets MsPGRP2; MsVMP1 and negative control dsRNA against Cassava plant specific gene MeCAT1. The differences observed using 4 replicates/treatment were statistically significant across all time points at a P value between P<0.001 to P<0.05.

FIG. 5 shows percentage melanotic reaction of healthy conventionally-reared (CR) and germ-free (GF) first instar TH larvae exposed to bacterially (HT115 (DE3)) expressed dsRNA against MIGGS RNAi targets MsPGRP2; MsVMP1 and negative control dsRNA against Cassava plant specific gene MeCAT1. The differences observed using 4 replicates/treatment were statistically significant across all time points at a P value between P<0.001 to P<0.05.

FIG. 6 shows schematic representation of dsRNA producing L4440 vector containing coding sequence of Cassava CAT1 (MeCAT1) and TH MIGGS RNAi targets MsPGRP2 and MsVMP1.

FIG. 7 shows midgut-preferred expression of two TH MIGGS RNAi target genes MsHEM and MsSPH3 in comparison to the control gene RPS3. The control cDNA libraries were derived from conventionally reared larvae and treatment cDNA libraries were derived from TH larvae injected with 75 CFU of E. coli. The control and treatment larvae were used to isolate hemolymph fraction (HL), dissect midgut (MDG) to obtain rest of the body (RB). The HL, MDG and RB were used for RNA isolation and cDNA synthesis.

FIG. 8 shows schematic representation of oral induction procedure for MIGGS RNAi target genes. The insect larvae were reared on induction media containing live gram-negative bacteria E. coli and lyophilized cell wall signatures from gram-positive bacteria and fungi, following a previously published protocol (Wang et al. (2006). J Biol. Chem. 281(14): 9271-9278).

FIG. 9A,B shows expression of MIGGS RNAi target genes in TH larvae in response to feeding on induction media. Genes with immunity function (A,B) are induced (I) between 24-48 hours post larval exposure to induction media and mostly not detected in the absence of induction (UI).

FIG. 9C,D also shows expression of MIGGS RNAi target genes in TH larvae in response to feeding on induction media. Genes with immunity function (C) are induced (I) between 24-48 hours post larval exposure to induction media and mostly not detected in the absence of induction (UI). While, the genes essential for midgut structural integrity (D) are expressed under both conditions.

FIG. 10A-F shows representative phenotypes of TH larvae (initial size shown in A) exposed to bacterially expressed dsRNA against insecticidal MIGGS-RNAi target genes MsToll2, MsSuc1, and MspTub in comparison to negative (B) and positive (C) control treatment. The insecticidal activity is manifested as stunted growth and development, loss of appetite and melanotic reaction (D-F) in comparison to regular growth and development observed with negative control treatment (B).

FIG. 11 shows percentage mortality of TH larvae feeding on bacterially expressed dsRNA against insecticidal MIGGS RNAi targets MsToll2, MsSuc1, and MspTub. The insecticidal MIGGS candidates confer statistically significant mortality that is comparable to positive control treatment (MsVATPaseE). Data is average of 3-replicates/treatment±SEM at p≤0.001(***).

FIG. 12A-C shows the induction of MIGGS-IRTGS in DBM larvae feeding on induction media. The genes with immunity function (A-B) are induced (I) between 24-48 hours post larval exposure to induction media and mostly not detected in the absence of induction (UI). While, the genes essential for midgut structural integrity (C) are expressed under both conditions.

FIG. 13A-H shows representative phenotypes of Bt resistant DBM larvae (initial size shown in A) exposed to bacterially expressed dsRNA against insecticidal DBM MIGGS RNAi targets PxPGRP2 (D), PxIMD (E), PxβGRP2 (F), PxCAC (G), and PxCHS1 (H) in comparison to positive (C) and negative control (B) treatment. The insecticidal activity is manifested as stunted growth and development, loss of appetite and melanotic reaction (D-H) in comparison to regular growth and development observed with negative control treatment (B).

FIG. 14 shows percentage mortality of DBM larvae feeding on bacterially expressed dsRNA against insecticidal MIGGS RNAi targets PxPGRP2, PxIMD, PxβGRP2, PxCAC, and PxCHS1. The insecticidal MIGGS candidates confer statistically significant mortality that is comparable to positive control treatment (MsVATPaseE). Data is average of 3-replicates/treatment±SEM at p≤0.001(***) p≤0.01(**).

FIG. 15A,B illustrates a sprayable RNAi set up using dsRNA against TH MIGGS targets MsPGRP2, Ms βGRP2, MsCHS2, and MsVMP1. One cm² leaf discs from field soil grown tobacco plants (A) were drop inoculated with various concentrations of purified dsRNA against TH MIGGS RNAi targets (B). The bioassays were carried out for 5 days with 3 first instar larvae per well and leaf discs changed at the end of every 24 hours.

FIG. 16 shows percentage mortality of TH larvae feeding on various concentrations of dsRNA against the core set of TH MIGGS RNAi targets MsPGPRP2, MsβGRP2, MsCHS2, and MsVMP1. The leaf disc coated dsRNA causes significant mortality at 8 and 16 μg of dsRNA concentration. The leaf disc coated dsRNA against three core MIGGS targets confers statistically significant mortality that is comparable to positive control treatment (MsVATPaseE). Data is average of 3-replicates/treatment±SEM at p≤0.001(***); p≤0.01(**) and p≤0.05(***).

FIG. 17A-H shows representative phenotypes of TH larvae (initial size shown in A and E) exposed to 8 and 16 μg of dsRNA against the core set of MIGGS RNAi targets MsPGPRP2 (D), MsβGRP2 (F), MsCHS2 (G), and MsVMP1 (H). The insecticidal activity is comparable to the positive control treatment (C) and manifested as stunted growth and development, loss of appetite and melanotic reaction (D,F-G) in contrast to the regular growth and development observed with negative control treatment (B).

FIG. 18A-D shows representative phenotypes of TH larvae feeding on leaves from transplastomic plants expressing dsRNA against core MIGGS targets MsPGRP2 (PTS-28-10 and PTS-28-7-B), MsβGRP2 (PTS-26-19 and PTS-26-4-C), and dsCHS2 (PTS-27-3-1 and PTS-27-13-1-D). The TH larvae feeding on leaves expressing dsRNA against MIGGS targets MsPGRP2 (B), MsβGRP (C), and MsCHS2 (D) display stunted growth, development, loss of appetite and melanotic reaction in comparison to negative control PTS-27-13-2 (A).

FIG. 18E shows that the insecticidal activity of transplastomic lines is manifested as significant reduction in mean weights in comparison to negative control.

FIG. 18F shows that the transplastomic events confer significant mortality in comparison to negative control. The mortality rate was scored on a 0-3 score were 0, 1, 2 and 3 indicated ≤0, 25, 50 or ≥50 mortality respectively. Data is average of 6 replicates/treatment (N=24)±SEM at p≤0.001(***); p≤0.01(**) and p≤0.05(*).

FIG. 19 shows percentage mortality of Bt resistant DBM larvae feeding on various concentrations of dsRNA against the DBM orthologs of TH core MIGGS RNAi targets PxPGPRP2, PxβGRP2, PxβTUB, and PxCHS1. The leaf disc coated dsRNA causes significant mortality at 0.5-1 μg of dsRNA concentration. The leaf disc coated dsRNA against all core MIGGS targets confers statistically significant mortality between 0.5 μg and 1 μg dsRNA dosage that is comparable to positive control treatment (MsVATPaseE). Data is average of 3-replicates/treatment±SEM at p≤0.01(**) and p≤0.05(*).

FIG. 20A-H shows representative phenotypes of Bt resistant DBM larvae (initial size shown in A and E) exposed to 0.5-1 μg of dsRNA against the DBM orthologs of TH core MIGGS RNAi targets PxPGPRP2(D), PxβGRP2(F), PxβTUB(G), and PxCHS1(H). The insecticidal activity is comparable to the positive control treatment (C) and manifested as stunted growth and development, loss of appetite and melanotic reaction (D,F-H) in contrast to the regular growth and development observed with negative control treatment (B).

FIG. 21A-D illustrates MIGGS-IRTG induction procedure in FAW feeding on wheat plants grown microbe rich field soil (A) and microbe depleted sterile surface (B). Ten first instar FAW larvae (C) were infested into each pot containing five wheat seedlings and contained using porous netting material (D). The larval samples were collected at various time points after infestation and used for pooled RNA-Seq approach to identify differential expressed transcripts in response to induction by the microbes in the filed soil.

FIG. 22A,B shows bi-clustering comparison of differential gene expression in FAW larvae feeding on microbe-depleted plants (A) in comparison to larvae feeding on microbe rich plants (B). RNA-Seq data indicated that plants growing on field soil caused preferential up-regulation of MIGGS pathway genes in FAW. In total, 100 differential expressed genes were identified, 30 of which were associated with MIGGS pathways. Notably, FAW orthologs of TH insecticidal targets including PGRP2, βGRP2 and IMD were captured in the data set, indicating clearly that MIGGS pathway genes are up regulated in response to insect feeding on plants exposed to soil microbiome.

FIG. 23A-F shows representative phenotypes of FAW larvae exposed to pure dsRNA against FAW orthologs of TH core MIGGS RNAi targets SFCHS2 (B), SFβGRP2 (C), SFPGRP2 (D), and two novel MIGGS RNAi targets from RNA-Seq study SFRC (un-annotated-E) and C-type lectin-6 (SFCTL-F). Leaf discs coated with dsRNA causes reduced growth, development and loss of appetite in comparison to negative control treatment (A) when supplied at 8 and 16 μg of dsRNA concentration per leaf disc.

FIG. 23G,H illustrates from FIG. 23A-F significant weight reduction (G) and mortality (H). The rates of mortality was scored on a 0-3 scale where 0, 1, 2 and 3 indicated ≤0, 25, 50 or ≥50 mortality respectively. The dsRNA treatments imposed caused statistically significant reduction in mean weights (G) that translated into significant rates of mortality (H) in comparison to negative control. Data is average of 3 replicates/treatment±SEM at p≤0.001(***); p≤0.01(**) and p≤0.05(*).

FIG. 24 shows mean weights (A) of FAW larvae exposed to 16 μg of pure dsRNA against newly discovered MIGGS RNAi targets from the RNA-Seq data. The rates of mortality was scored on a 0-3 score were 0, 1, 2 and 3 indicated ≤0, 25, 50 or ≥50 mortality respectively. The dsRNA treatments imposed caused statistically significant reduction in mean weights (A) that also translated into significant rates of mortality (B) in comparison to negative control. Data is average of 3 replicates/treatment±SEM at p≤0.001(***); p≤0.01(**) and p≤0.05(*).

FIG. 25 shows representative phenotypes of RFB beetles feeding on rice flour mixed with 1 μg of pure dsRNA against core MIGGS targets in RFB TcPGRP2, MsβGRP2, dsCHS2, and a previously discovered target MDGP. The RFB beetles feeding on dsRNA against all MIGGS targets displayed significant mortalities at the end of 72 hours of feeding in comparison to negative control treatment (TE). The rates of mortality were significantly higher than negative control treatment and were comparable to the positive control treatment TcvATPaseE. Mortality rate was scored on a 0-3 scale were 0, 1, 2 and 3 indicated ≤0, 25, 50 or ≥50 mortality respectively. Data is average of 6 replicates/treatment (N=18)±SEM at p≤0.001(***); p≤0.01(**) and p≤0.05(*).

FIG. 26A,B shows imageJ analyzed RT-PCR data correlating TH larval phenotypes observed when exposed to 8-16 μg pure dsRNA against a positive control (A) and a core insecticidal MIGGS RNAi target MsβGRP2 (B). The transcript down regulation is correlated with the larval phenotypes observed in FIG. 17. For each pair of vertical bars, 8 μg pure dsRNA is the left bar and 16 μg pure dsRNA is the right bar.

FIG. 26C,D shows imageJ analyzed RT-PCR data correlating TH larval phenotypes observed when exposed to 8-16 μg pure dsRNA against core set of insecticidal MIGGS RNAi targets MsPGPRP2 (C) and MsCHS2 (D). The transcript down regulation is correlated with the larval phenotypes observed in FIG. 17. For each pair of vertical bars, 8 μg pure dsRNA is the left bar and 16 μg pure dsRNA is the right bar.

### DETAILED DESCRIPTION

### Overview

RNAi-mediated gene-silencing offers a sustainable alternative approach to insect control. Most of the successful RNAi-based pest control strategies thus far employ homology dependent silencing of essential gene functions. Despite this, effective RNAi-based crop protection is lacking for Lepidopteran pests, due to their variable sensitivity to ingested double stranded RNA (dsRNA). (Terenius O, et al. (2011). J Insect Physiol. 57(2): 231-245).

Plant pests are in constant contact with, and ingest significant amount of microbes during herbivory. (Gayatri Priya N. et al. (2012). 7(1), PLos ONE. E30768; Peñuelas and Terradas (2014). 19(5): Trends Plant Sci. 278-280; Engel and Moran (2013). FEMS microbiol. rev. 37(5): 699-735). This interaction between ingested microbes and insect midgut is often considered passive. Recent studies suggest, however, an active role of midgut specific immune responses in reducing variation of core microbial communities during insect herbivory through the activation of pattern recognition receptors (PRR). (Casanova-Torres and Goodrich-Blair (2013). Insects. 4: 320-338; Tang X, et al. (2012) 7(7) PLoS ONE:e36978; Ryu J H. et al. (2008). Science. 37(5): 777-82; Shrestha S. et al. (2009). J Asia Pac. Entomol. 12: 277-283; Buchon, N. et al. (2013). Front. Cell. Infect. Microbiol. 11: 615-626). Further, maintenance of core gut microbial communities via active immune responses and/or their containment in the midgut is key to successful herbivory.

Although, the core innate immune response pathways are conserved, their specific components are under strong selection for diversification. (Casanova-Torres and Goodrich-Blair (2013). Insects. 4: 320-338). Therefore, it is contemplated herein that these pathways provide novel and specific targets for devising sustainable pesticidal RNAi biotechnologies against insect pests. Although gut immune responses have been studied from an immunological perspective, their active manipulation via genetic engineering for pest protection is currently lacking.

Provided herein is the identification of “insect RNAi target genes” (IRTGs) involved in gut microbial clearance and/or containment induced by microbes ingested during feeding and/or active feeding (referred to herein as microbe-induced gut specific genes (MIGGS)) and examples of a novel biotechnology for insect protection via inter-specific silencing of MIGGS-IRTGs. In certain aspects, the MIGGS-IRTGs are Lepidoptera-specific. For example, in certain aspects detailed below, the insect is Manduca sexta (M. sexta; Lepidoptera) (tobacco hornworm (TH)). For example, in certain aspects detailed below, the insect is Spodoptera frugiperda (fall armyworm (FAW)). For example, in certain aspects detailed below, the insect is Plutella xylostella (Diamondback moth (DBM). For example, in certain aspects detailed below, the insect is Ostrinia nubilalis (European corn borer). Still, it is also considered understood that successful, feeding-induced loss of appetite, developmental defects, and/or lethality has the potential to provide protection beyond the order Lepidoptera in an orthologous manner. For example, protection against coleopteran pests such as Leptinotarsa decemlineata (Say) (Colorado potato beetle), Diabrotica spp. (Corn rootworm complex), and Tribolium castaneum (Red Flour Beetle (RFB)). Additionally, this MIGGS-RNAi technique may allow containment of disease transmitting insect vectors and/or enable further manipulation of the plant-microbe-insect interactions for devising pesticidal RNAi for crop protection.

In certain aspects detailed below, silencing of a target gene can result in reduced appetite and/or developmental defects and/or mortality and/or reduced fitness of the insect. In certain aspects these effects are observed after sustained feeding for at least about 24, 36, 48, or 72 hours, or any time inbetween.

### Definitions

To the extent necessary to provide descriptive support, the subject matter and/or text of the appended claims is incorporated herein by reference in their entirety.

It will be understood by all readers of this written description that the exemplary embodiments described and claimed herein may be suitably practiced in the absence of any recited feature, element or step that is, or is not, specifically disclosed herein.

It is to be noted that the term “a” or “an” entity refers to one or more of that entity; for example, “a dsRNA molecule,” is understood to represent one or more dsRNA molecules. As such, the terms “a” (or “an”), “one or more,” and “at least one” can be used interchangeably herein.

Furthermore, “and/or” where used herein is to be taken as specific disclosure of each of the specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, technical and scientific terms used herein have the same meaning as commonly understood by one of ordinary skill in the art to which this disclosure is related. Numeric ranges are inclusive of the numbers defining the range. Even when not explicitly identified by “and any range in between,” or the like, where a list of values is recited, e.g., 1, 2, 3, or 4, unless otherwise stated, the disclosure specifically includes any range in between the values, e.g., 1 to 3, 1 to 4, 2 to 4, etc.

The headings provided herein are solely for ease of reference and are not limitations of the various aspects or aspects of the disclosure, which can be had by reference to the specification as a whole.

As used herein, the term “non-naturally occurring” condition, substance, polypeptide, polynucleotide, composition, entity, plant, organism, individual, and/or any combination thereof, or any grammatical variants thereof and the like, is a conditional term that explicitly excludes, but only excludes, those forms that are well-understood by persons of ordinary skill in the art as being “naturally-occurring,” or that are, or might be at any time, determined or interpreted by a judge or an administrative or judicial body to be, “naturally-occurring.”

As used herein, the term “identity,” e.g., “percent identity” to an amino acid sequence or to a nucleotide sequence disclosed herein refers to a relationship between two or more amino acid sequences or between two or more nucleotide sequences. When a position in one sequence is occupied by the same nucleic acid base or amino acid in the corresponding position of the comparator sequence, the sequences are said to be “identical” at that position. The percentage of “sequence identity” is calculated by determining the number of positions at which the identical nucleic acid base or amino acid occurs in both sequences to yield the number of “identical” positions. The number of “identical” positions is then divided by the total number of positions in the comparison window and multiplied by 100 to yield the percentage of “sequence identity.” Percentage of “sequence identity” is determined by comparing two optimally aligned sequences over a comparison window. In order to optimally align sequences for comparison, the portion of a nucleotide or amino acid sequence in the comparison window can comprise additions or deletions termed gaps while the reference sequence is kept constant. An optimal alignment is that alignment which, even with gaps, produces the greatest possible number of “identical” positions between the reference and comparator sequences. Percentage “sequence identity” between two sequences can be determined using, e.g., the program “BLAST” which is available from the National Center for Biotechnology Information, and which program incorporates the programs BLASTN (for nucleotide sequence comparison) and BLASTP (for amino acid sequence comparison), which programs are based on the algorithm of Karlin and Altschul ((1993). Proc. Nat. Acad. Sci. USA. 90(12): 5873-5877).

As used herein, the term “complementary” refers to the ability of polynucleotides to form base pairs with one another. Base pairs are typically formed by hydrogen bonds between nucleotide units in antiparallel polynucleotide strands. Complementary polynucleotide strands can base pair in the Watson-Crick manner (e.g., A to T, A to U, C to G), or in any other manner that allows for the formation of duplexes. When using RNA as opposed to DNA, uracil (U) rather than thymine (T) is the base that is considered to be complementary to adenosine. However; when a U is denoted in the context of the present invention, the ability to substitute a T is implied, unless otherwise stated.

As used herein, the term “polypeptide” is intended to encompass a singular “polypeptide” as well as plural “polypeptides,” and refers to a molecule composed of monomers (amino acids) linearly linked by peptide bonds (also known as amide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and unless specifically stated otherwise the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-standard amino acids. A polypeptide can be derived from a natural biological source or produced by recombinant technology, but is not necessarily translated from a designated nucleic acid sequence. Thus, it can be generated in any manner, including by chemical synthesis.

As used herein, the term “protein” refers to a single polypeptide, i.e., a single amino acid chain as defined above, but can also refer to two or more polypeptides that are associated, e.g., by disulfide bonds, hydrogen bonds, or hydrophobic interactions, to produce a multimeric protein.

As used herein, the term “nucleotide” refers to a ribonucleotide or a deoxyribonucleotide or modified form thereof, as well as an analog thereof. Nucleotides include species that comprise purines, e.g., adenine, hypoxanthine, guanine, and their derivatives and analogs, as well as pyrimidines, e.g., cytosine, uracil, thymine, and their derivatives and analogs. Further, the term nucleotide also includes those species that have a detectable label, such as for example a radioactive or fluorescent moiety, or mass label attached to the nucleotide.

As used herein, the term “polynucleotide” refers to polymers of nucleotides, and includes but is not limited to DNA, RNA, DNA/RNA hybrids including polynucleotide chains of regularly and/or irregularly alternating deoxyribosyl moieties and ribosyl moieties (i.e., wherein alternate nucleotide units have an —OH, then and —H, then an —OH, then an —H, and so on at the 2′ position of a sugar moiety), and modifications of these kinds of polynucleotides, wherein the attachment of various entities or moieties to the nucleotide units at any position are included. The term “polynucleotide” is also intended to encompass a singular nucleic acid as well as plural nucleic acids, and refers to an isolated nucleic acid molecule or construct, e.g., messenger RNA (mRNA) or plasmid DNA (pDNA). A polynucleotide can comprise a conventional phosphodiester bond or a non-conventional bond (e.g., an amide bond, such as found in peptide nucleic acids (PNA)). A polynucleotide can be single stranded or double stranded.

As used herein, the term “nucleic acid” refers to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding a polypeptide subunit contained in a vector is considered isolated as disclosed herein. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides. Isolated polynucleotides or nucleic acids further include such molecules produced synthetically. In addition, polynucleotide or a nucleic acid can be or can include a regulatory element such as a promoter, ribosome binding site, or a transcription terminator.

As used herein, a “coding region” is a portion of nucleic acid comprising codons translatable into amino acids. Although a “stop codon” (TAG, TGA, or TAA) is not translated into an amino acid, it can be considered to be part of a coding region, but any flanking sequences, for example 5′ untranslated regions (5′ UTRs; also known as a leader sequence), 3′ untranslated regions (3′ UTRs; also known as a trailer sequence), promoters, ribosome binding sites, transcriptional terminators, introns, and the like, are not part of a coding region. Two or more coding regions can be present in a single polynucleotide construct, e.g., on a single vector, or in separate polynucleotide constructs, e.g., on separate (different) vectors. Furthermore, any vector can contain a single coding region, or can comprise two or more coding regions, e.g., a single vector can separately encode a selection marker gene and a gene of interest. In addition, a vector, polynucleotide, or nucleic acid can encode heterologous coding regions, either fused or unfused to a nucleic acid encoding a polypeptide subunit or fusion protein as provided herein. Heterologous coding regions include without limitation specialized elements or motifs, such as a secretory signal peptide or a heterologous functional domain.

A variety of transcription regulatory regions are known to those skilled in the art. These include, without limitation, transcription regulatory regions that function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription regulatory regions include those derived from vertebrate genes such as actin, heat shock protein, bovine growth hormone and rabbit 0-globin, as well as other sequences capable of controlling gene expression in eukaryotic cells. Additional suitable transcription regulatory regions include tissue-specific promoters and enhancers.

Similarly, a variety of translation regulatory elements are known to those of ordinary skill in the art. These include, but are not limited to ribosome binding sites, translation initiation and termination codons, and elements derived from picornaviruses (particularly an internal ribosome entry site, or IRES).

As used herein, the term “vector” is nucleic acid molecule as introduced into a host cell or organelle, thereby producing a transformed host cell or organelle. A vector can include nucleic acid sequences that permit it to replicate in a host cell, such as an origin of replication. A vector can also include one or more selectable marker gene and other genetic elements known in the art. Illustrative types of vectors include plasmids, phages, viruses and retroviruses.

As used herein, the term “transformed” cell or organelle, or a “host” cell organelle, is a cell or organelle into which a nucleic acid molecule has been introduced by molecular biology techniques. As used herein, the term transformation encompasses those techniques by which a nucleic acid molecule can be introduced into such a cell or organelle, including transfection with viral vectors, transformation with plasmid vectors, and introduction of naked DNA by electroporation, lipofection, and particle gun acceleration. A transformed cell or a host cell can be a bacterial cell or a eukaryotic cell.

As used herein, the term “expression” refers to a process by which a gene produces a biochemical, for example, a polynucleotide or a polypeptide. The process includes any manifestation of the functional presence of the gene within the cell including, without limitation, gene knockdown as well as both transient expression and stable expression. It includes without limitation transcription of the gene into messenger RNA (mRNA), and the translation of such mRNA into polypeptide(s). It also includes without limitation transcription of the gene into an RNA molecule that is not translated into a polypeptide but is capable of being processed by cellular RNAi mechanisms. If the final desired product is a biochemical, expression includes the creation of that biochemical and any precursors. Expression of a gene produces a “gene product.” As used herein, a gene product can be either a nucleic acid, e.g., an RNA produced by transcription of a gene or a polypeptide that is translated from a mRNA transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

As used herein the term “engineered” includes manipulation of nucleic acid or polypeptide molecules by synthetic means (e.g. by recombinant techniques, in vitro peptide synthesis, by enzymatic or chemical coupling of peptides or some combination of these techniques).

As used herein, the term “hpRNA” refers to hairpin RNA comprising a single-stranded loop region and a base-paired stem of an inversely repeated sequence. hpRNA can be generated from an hpRNA construct (or vector) and/or an hpRNA transgene comprising an inversely-repeated sequence of the RNAi target gene with a spacer region between the repeats. The RNA transcribed from such a sequence self-hybridizes to form a hairpin structure. The stem can be used as a substrate for the generation of siRNAs, but few or none are generated from the loop. Since a spacer region is needed for the stability of the transgene construct, but is not involved in siRNA production, an intron sequence is often used in this position. (Watson J M, et al. (2005). FEBS Letters. 579: 5982-8987).

As used herein, the term “siRNA” refers to small (or short) interfering RNA (or alternatively, silencing RNA) duplexes that are capable of inducing the RNA interference (RNAi) pathway. These molecules can vary in length (generally between 18-30 base pairs) and contain varying degrees of complementarity to their target mRNA in the antisense strand. Some, but not all, siRNA have unpaired overhanging bases on the 5′ or 3′ end of the sense strand and/or the antisense strand. The term “siRNA” includes duplexes of two separate strands, as well as single strands that can form hairpin structures comprising a duplex region.

As used herein, the phrase “duplex region” refers to the region in two complementary or substantially complementary polynucleotides that form base pairs with one another, either by Watson-Crick base pairing or any other manner that allows for a stabilized duplex between polynucleotide strands that are complementary or substantially complementary. For example, a polynucleotide strand having 21 nucleotide units can base pair with another polynucleotide of 21 nucleotide units, yet only 19 bases on each strand are complementary or substantially complementary, such that the “duplex region” has 19 base pairs. The remaining bases may, for example, exist as 5′ and 3′ overhangs. Further, within the duplex region, 100% complementarity is not required; substantial complementarity is allowable within a duplex region. Substantial complementarity as used herein refers to 79% or greater complementarity. For example, a mismatch in a duplex region consisting of 19 base pairs results in 94.7% complementarity, rendering the duplex region substantially complementary.

As used herein, the phrase “gene silencing” refers to a process by which the expression of a specific gene product is lessened or attenuated. Silencing of a gene does not require that the expression or presence of the gene product is completely absent, but that in the context (e.g., comparing expression of a target gene in a plant expressing a gene silencing nucleic acid compared to a control plant or the health of an insect feeding on a gene silencing nucleic acid compared to a control insect), an observable effect in comparison to a control is observed. While gene silencing can take place by a variety of pathways, unless specified otherwise, as used herein, gene silencing refers to decreases in gene product expression that results from RNA interference (RNAi) as understood by one of ordinary skill in the art. The level of gene silencing can be measured by a variety of means, including, but not limited to, measurement of transcript levels by Reverse transcription polymerase chain reaction (PCR), Northern Blot Analysis, B-DNA techniques, transcription-sensitive reporter constructs, expression profiling (e.g. DNA chips), and related technologies. Alternatively, the level of silencing can be measured by assessing the level of the protein encoded by a specific gene. This can be accomplished by performing a number of studies including Western Analysis, measuring the levels of expression of a reporter protein that has e.g. fluorescent properties (e.g. GFP) or enzymatic activity (e.g. alkaline phosphatases), or several other well-known procedures. Further, gene silencing can be assessed by its effect on a pest insect such as resulting in reduced appetite and/or developmental defects and/or mortality of an insect.

As used herein, the term “control” is consistent with its well-established scientific use that refers to a standard of comparison recognized by one of ordinary skill in the art as having a representative level of expression, phenotype, resistance, feeding, mortality, development, etc. Further, one of ordinary skill in the art will recognize, for example, that a statistical outlier and/or non-representative result produced by chance, abnormal environmental condition, manipulation, or other reason, that varies from a standard representation, would not be an appropriate control.

As used herein, “microbe-induced gut specific genes (MIGGS)” refers to a gene or group of genes expressed in the insect midgut in response to microbes ingested during normal process of insect feeding and primarily functioning to clear or respond to the ingested microbes and/or contain the microbes to insect gut via maintenance of midgut structural integrity.

As used herein, “actively feeding stage of the insect” refers to all feeding stages of insects with both complete and incomplete metamorphosis.

### Nucleic Acids for Gene Silencing

Provided herein are nucleic acid molecules for use in, among other things, crop protection from insect pests. In certain aspects disclosed herein, the nucleic acid molecules are isolated. The nucleic acid molecules specifically target certain insect genes (referred to herein interchangeably as “target genes,” “RNAi target genes,” “insect RNAi target genes,” and “IRTGs”), in insects for gene silencing. For example, in certain aspects, the nucleic acid molecules target certain insect microbe-induced gut gene (MIGGS) RNAi targets. In certain aspects, the silencing of a target gene occurs when a nucleic acid molecule of this disclosure is ingested by an insect. In certain aspects, the target gene is an insect gene that is implicated in insect immune responses (type 1 MIGGS RNAi target). A critical immune response gene is a genetically tractable nuclear or cytoplasmic loci that is important for providing cellular and/or humoral defense in insects against internal microorganisms, external microorganisms, and/or other insect parasites. In certain aspects, the immune response genes (type 1 MIGGS RNAi target) can also be a pattern recognition receptor (PRR) gene (Casanova-Torres and Goodrich-Blair (2013). Insects. 4:320-338). A PPR gene is a genetically tractable loci of an insect that encodes soluble or membrane bound proteins that recognize signatures associated with and/or released by microorganisms. PRR genes can activate or be activated by the immune response pathways to minimize microbial infection and can be co-regulated by the immune deficiency (IMD) pathway (Tang X, et al. (2012) 7(7) PLoS ONE:e36978; Ryu J H. et al. (2008). Science. 37(5): 777-82; Shrestha S. et al. (2009). In certain aspects the PRR type genes are co-regulated by the immune deficiency (IMD) pathway in TH were identified, these genes having been recently summarized. (Casanova-Torres and Goodrich-Blair (2013). Insects (4): 320-338; Zhong X, et al. (2012). Insect Biochem. Mol. Biol. 42(7): 514-524); Zhang X, et al. (2015). Insect Biochem. Mol. Biol. 62:38-50; Cao X, et al. (2015). Insect Biochem. Mol. Biol. 62:64-74; Kanost M R, et al. (2016). Insect Biochem. Mol. Biol 76:118-147). In certain aspects, the target gene is an insect gene that is necessary for structural integrity of insect organs including the mid-gut and also facilitates the containment of the ingested microbes to the insect gut. (type 2 MIGGS RNAi target). In certain aspects, the target gene is an insect midgut structural component gene (type 2) (Odman-Naresh et al. (2013). PLoS ONE 8:e82015. 10.1371/journal.pone.0082015). A midgut structural component gene is a genetically tractable loci in an insect that encodes chitin fibrils, proteins, or glycoproteins that form a protective sac-like structure called peritrophic matrix enveloping the insect food bolus/midgut also functioning to contain the ingested microbes in the gut (Engel and Moran (2013). FEMS Microbiol Rev. 37 699-735). In certain aspects, the target genes (type 1 and 2 MIGGS RNAi targets) are predominantly expressed in the insect midgut, for example, abundantly and/or exclusively expressed in the larval and/or adult insect midgut in response to active feeding and/or microbial infection and/or responding to microbes ingested during feeding. In certain aspects, the target gene is induced predominantly in a midgut specific manner during active feeding (type 1 and type 2 MIGGS RNAi targets). The midgut abundance of both type 1 and 2 MIGGS RNAi target genes may mitigate problems associated with reduced amounts of bioavailability.

Representative examples of insect MIGGS RNAi target genes and their nucleic acid sequences identified from published literature are provided herein. In certain aspects, the target gene is one or more of M. sexta-Hemolin (MsHEM), M. sexta-Serine proteinase homolog 3 (MsSPH-3), M. sexta-Peptidoglycan recognition protein 2 (MsPGRP2), M. sexta-Beta-1, 3-glycan-recognition protein 2 (MsβGRP2), M. sexta-Relish family protein 2A (MsREL2A), M. sexta-Dorsal (MsDor), M. sexta-Spatzle (MsSPZ1A), M. sexta-Toll receptor (MsTOLL), M. sexta-Scolexin A (MsSCA1), M. sexta-Hemolymph proteinase 18 (MsHP18), M. sexta-Transferrin (MsTRN), M. sexta-Arylphorin beta subunit (MsARP), M. sexta-Chymotrypsinogen-like protein 1 (MsCTL1), M. sexta-Valine Rich Midgut Protein (MsVMP1), M. sexta-Imd (MsImd), M. sexta-FADD (MsFADD), M. sexta-Dredd (MsDRD), M. sexta-Relish F (MsReIF), M. sexta-Cdc42 (MsCdc42), M. sexta-Dsor1 (MsDsor1), M. sexta-Fos (MsFos), M. sexta-Jra (MsJra), M. sexta-Caudal (MsCAD1), M. sexta-Atg8 (MsAtg8), M. sexta-Atg13 (MsAtg13), M. sexta-IAP1 (MsIAP1), M. sexta-Chitin synthase 2 (MsChs2), M. sexta-Beta fructofuranosidase 1 (MsSuc1), and orthologs thereof.

In certain aspects, the target gene is one or more of M. sexta-Hemolin (MsHEM), M. sexta-Serine proteinase homolog 3 (MsSPH-3), M. sexta-Peptidoglycan recognition protein 2 (MsPGRP2), M. sexta-Beta-1, 3-glycan-recognition protein 2 (MsβGRP2), M. sexta-Relish family protein 2A (MsREL2A), M. sexta-Dorsal (MsDor), M. sexta-Spatzle (MsSPZ1A), M. sexta-Toll receptor (MsTOLL), M. sexta-Scolexin A (MsSCA1), M. sexta-Hemolymph proteinase 18 (MsHP18), M. sexta-Transferrin (MsTRN), M. sexta-Arylphorin beta subunit (MsARP), M. sexta-Chymotrypsinogen-like protein 1 (MsCTL1), M. sexta-Valine Rich Midgut Protein (MsVMP1), M. sexta-Imd (MsImd), M. sexta-FADD (MsFADD), M. sexta-Dredd (MsDRD), M. sexta-Relish F (MsReIF), M. sexta-Cdc42 (MsCdc42), M. sexta-Dsor1 (MsDsor1), M. sexta-Fos (MsFos), M. sexta-Jra (MsJra), M. sexta-Caudal (MsCAD1), M. sexta-Atg8 (MsAtg8), M. sexta-Atg13 (MsAtg13), M. sexta-IAP1 (MsIAP1), M. sexta-Chitin synthase 2 (MsChs2), and M. sexta-Beta fructofuranosidase 1 (MsSuc1), M. sexta-Sickie (MsSck), M. sexta-Akirin (MsAki), M. sexta-Cactus (MsCac), M. sexta-Gloverin (MsGlv) and M. sexta-Beta-1-tubulin (MspTub).

In certain aspects, the target gene is an ortholog of one or more of M. sexta-Hemolin (MsHEM), M. sexta-Serine proteinase homolog 3 (MsSPH-3), M. sexta-Peptidoglycan recognition protein 2 (MsPGRP2), M. sexta-Beta-1, 3-glycan-recognition protein 2 (MsβGRP2), M. sexta-Relish family protein 2A (MsREL2A), M. sexta-Dorsal (MsDor), M. sexta-Spatzle (MsSPZ1A), M. sexta-Toll receptor (MsTOLL), M. sexta-Scolexin A (MsSCA1), M. sexta-Hemolymph proteinase 18 (MsHP18), M. sexta-Transferrin (MsTRN), M. sexta-Arylphorin beta subunit (MsARP), M. sexta-Chymotrypsinogen-like protein 1 (MsCTL1), M. sexta-Valine Rich Midgut Protein (MsVMP1), M. sexta-Imd (MsImd), M. sexta-FADD (MsFADD), M. sexta-Dredd (MsDRD), M. sexta-Relish F (MsReIF), M. sexta-Cdc42 (MsCdc42), M. sexta-Dsor1 (MsDsor1), M. sexta-Fos (MsFos), M. sexta-Jra (MsJra), M. sexta-Caudal (MsCAD1), M. sexta-Atg8 (MsAtg8), M. sexta-Atg13 (MsAtg13), M. sexta-IAP1 (MsIAP1), M. sexta-Chitin synthase 2 (MsChs2), M. sexta-Beta fructofuranosidase 1 (MsSuc1), and other IMD pathway or structural integrity genes.

One of ordinary skill in the art would understand that nucleic acid molecules can be, for example, deoxyribonucleic acid (DNA) or ribonucleic acid (RNA). In certain aspects of any target gene silencing nucleic acid molecule described anywhere herein, the nucleic acid molecule is a DNA molecule. In certain aspects of any target gene silencing nucleic acid molecule described anywhere herein, the nucleic acid molecule is a RNA molecule. In certain aspects of any target gene silencing nucleic acid molecule described anywhere herein, the RNA molecule is a double stranded molecule (dsRNA), for example, for use in the RNA interference (RNAi) process. As used herein, a dsRNA molecule is a RNA molecule comprising at least one annealed, double stranded region. In certain aspects, the double stranded region comprises two separate RNA strands annealed together. In certain aspects, the double stranded region comprises one RNA strand annealed to itself, for example, as can be formed when a single RNA strand contains an inversely repeated sequences with a spacer in between. One of ordinary skill in the art will understand that complementary nucleic acid sequences are able to anneal to each other but that two sequences need not be 100% complementary to anneal. The amount of complementarity needed for annealing can be influenced by the annealing conditions such as temperature, pH, and ionic condition. In certain aspects, the annealed RNA sequences are 100% complementary across the annealed region. In certain aspects, the annealed RNA sequences are less than 100% complementary across the annealed region but have enough complementarity to anneal within their environment, such as in a host cell or the gut of an insect. In certain aspects, the annealed RNA sequences are substantial complementarity as defined elsewhere herein.

It is contemplated that the nucleic acid molecules disclosed anywhere herein for the silencing of target genes derive their specificity from comprising a nucleic acid sequence that is complementary or substantially complementary to at least a portion of a target gene sequence. Substantially complementary sequences, however, may be more likely to have reduced specificity and produce off-target effects. As referred to anywhere herein, a target gene sequence can include at least the target gene protein coding region, the 5′ untranslated region (5′ UTR), and/or the 3′ untranslated region (3′ UTR) and any portion or combination thereof. For example, predicted UTR regions can be identified using previously established criteria (Siepel, et al. (2005). Genome Res. 15: 1034-1050) when corresponding genomic sequences are available.

In certain aspects, an isolated double stranded RNA (dsRNA) molecule comprises a nucleic acid sequence complementary to about 21 to 2000 contiguous nucleotides of a target gene sequence discloses anywhere herein. For example, in certain aspects, an isolated double stranded RNA (dsRNA) molecule comprises a nucleic acid sequence complementary to about any of 21, 22, 23, 24, 25, 30, 40, 50, 60, 100, 120, 200, 240, 300, 400, 500, 600, 650, 750, 1000 to about any of 23, 24, 25, 30, 40, 50, 100, 200, 300, 400, 500, 600, 650, 750, 1000, or 2000 contiguous nucleotides of a target gene sequence. For example, in certain aspects, an isolated dsRNA molecule comprises a nucleic acid sequence complementary to about 100 to 1000 or about 200 to 1000 contiguous nucleotides of a target gene sequence. For example, in certain aspects, an isolated dsRNA molecule comprises a nucleic acid sequence complementary to about 100 to 1000 or about 200 to 1000 contiguous nucleotides of the protein coding region of a target gene sequence. For example, in certain aspects, an isolated dsRNA molecule comprises a nucleic acid sequence complementary to about 100 to 1000 or about 200 to 1000 contiguous nucleotides of the 5′ UTR region or the 3′ UTR region of a target gene sequence. In certain aspects, the isolated dsRNA molecule comprises a nucleic acid sequence complementary to a contiguous region comprising at least about 50%, 60%, 70%, 80%, 90%, 95%, 98%, 99%, or 100% of the length of the target gene sequence protein coding region, the target gene sequence 5′ UTR region, the target gene 3′ UTR region, and/or any combination thereof. For example, if a target gene sequence protein coding region is determined to be 200 nucleotides long, then an isolated dsRNA molecule comprising a nucleic acid sequence complementary to a contiguous region comprising 95% of the length of the target gene sequence protein coding region would be complementary to a contiguous region 190 nucleotides long.

In certain aspects of any target gene silencing nucleic acid molecule described anywhere herein, including dsRNA molecules for RNAi, the target gene comprises one or more of the nucleic acid sequence of SEQ ID NO: 1-14, 16-29, 31-69, 70-75, 76-88, 89-105, and 106-110. In certain aspects, the target gene comprises the nucleic acid sequence of SEQ ID NO: 3 or 14. Thus, in certain aspects, the isolated dsRNA molecule comprises a nucleic acid sequence complementary to about any of 21, 22, 23, 24, 25, 30, 40, 50, 100, 200, 300, 400, 500, 600, 650, 750, 1000 to about any of 23, 24, 25, 30, 40, 50, 100, 200, 300, 400, 500, 600, 650, 750, 1000, or 2000 contiguous nucleotides of a target gene sequence comprising a nucleic acid sequence selected from the group consisting of SEQ ID NOs: 1-14, 16-29, 31-69, 70-75, 76-88, 89-105, and 106-110. For example, in certain aspects, the isolated dsRNA molecule comprises a nucleic acid sequence complementary to about 100 to 1000 or about 200 to 1000 contiguous nucleotides of a target gene sequence comprising a nucleic acid sequence selected from the group consisting of SEQ ID NOs: 1-14, 16-29, 31-69, 70-75, 76-88, 89-105, and 106-110. In certain aspects, the isolated dsRNA comprises a nucleic acid sequence complementary to about 200 to 1000 contiguous nucleotides of the protein coding region of a target gene sequence comprising a nucleic acid sequence selected from the group consisting of SEQ ID NOs: 1-14, 16-29, 31-69, 70-75, 76-88, 89-105, and 106-110. In certain aspects, the isolated dsRNA molecule comprises a nucleic acid sequence complementary to a contiguous region comprising at least about 50%, 60%, 70%, 80%, 90%, 95%, 98%, 99%, or 100% of the length of the target gene sequence protein coding region, the target gene 5′ UTR region, and/or the target gene 3′ UTR region. In certain aspects, the isolated dsRNA molecule comprises a nucleic acid sequence complementary to a contiguous region comprising at least about 50%, 60%, 70%, 80%, 90%, 95%, 98%, 99%, or 100% of the length of a sequence selected from the group consisting of SEQ ID NOs: 1-14, 16-29, 31-69, 70-75, 76-88, 89-105, and 106-110.

In certain aspects of any target gene silencing nucleic acid molecule described anywhere herein, the nucleic acid molecule can form siRNA. Thus, certain aspects provide for an siRNA molecule derived from the processing of a dsRNA molecule for silencing a target gene disclosed herein.

### Insecticidal Compositions

Certain aspects of the disclosure provide for an insecticidal composition comprising a nucleic acid molecule disclosed anywhere herein for silencing a target gene, including long dsRNA, hpRNA, and siRNA. In certain aspects, the insecticidal composition also comprises a synthetic carrier or a microbial conduit. For example, a microbial conduit can be a microorganism that has a natural capacity or is engineered to produce and/or deliver dsRNA to increase its bioavailability and/or biostability for causing RNA interference. Representative examples include plant growth promoting organisms, normal commensal and/or symbiotic microorganisms associated with the target insect pest or pest target host or host cultivation range etc. from an insect engineered or identified from natural populations to produce and/or deliver dsRNA. In certain aspects a microbial conduit can be used as a direct topical application on a whole plant or coated onto a seed or mixed with growth media or transmitted through fertilizer or irrigation, etc. In certain aspects, the nucleic acid molecule of the insecticidal composition is conjugated to the synthetic carrier. For example, a synthetic carrier can be an inert chemical compound with a natural or engineered affinity to bind (conjugate) a dsRNA molecule to increase its biostability and/or bioavailability for causing RNA interference. In certain aspects, a synthetic carrier comprises a combination of inert chemicals or nanoparticles that upon combining and/or individually have a net positive charge or general affinity to bind to negatively charged dsRNA. Representative examples include chitosan, liposomes, carbon quantum dots, biodegradable particles of plant (e.g. coconut coir or grain flour, etc.) or soil (e.g. calcified clay) origin etc. In certain aspects, the dsRNA conjugated with a synthetic carrier can be used as a direct topical application directly and/or after aerosolization on a whole plant or coated onto a seed or mixed with growth media or transmitted through fertilizer or irrigation, etc. In certain aspects, dsRNA or a composition comprising dsRNA can be used as a direct topical spray on application to whole plant, coated onto a seed or mixed with growth media or transmitted through fertilizer or irrigation or combined with plant growth promoting microbes etc.

### Recombinant Constructs

Certain aspects of this disclosure provide for a recombinant nucleic acid construct, such as a DNA vector, comprising and/or encoding a nucleic acid molecule disclosed anywhere herein for silencing a target gene, including long dsRNA, hpRNA, and siRNA. Certain aspects provide for recombinant nucleic acid constructs comprising and/or encoding an RNAi precursor of a nucleic acid molecule disclosed anywhere herein for silencing a target gene, including long dsRNA, hpRNA, and siRNA.

Certain aspects of this disclosure provide for a recombinant nucleic acid construct, such as a DNA vector, comprising a target gene silencing sequence for silencing a target gene described anywhere herein. In certain aspects, a recombinant DNA construct comprises a gene silencing sequence comprising about any of 21, 22, 23, 24, 25, 30, 40, 50, 60, 100, 120, 200, 240, 300, 400, 500, 600, 650, 750, 1000 to about any of 23, 24, 25, 30, 40, 50, 100, 200, 300, 400, 500, 600, 650, 750, 1000, or 2000 contiguous nucleotides of a target gene sequence disclosed anywhere herein. In certain aspects, a recombinant DNA construct comprises a gene silencing sequence comprising about 100 to 1000 or about 200 to 1000 contiguous nucleotides of a target gene sequence. In certain aspects, the gene silencing sequence comprises about 100 to 1000 or about 200 to 1000 contiguous nucleotides of the protein coding region of the target gene sequence. In certain aspects, the gene silencing sequence comprises about 100 to 1000 or about 200 to 1000 contiguous nucleotides of the 5′ UTR region or the 3′ UTR region of the target gene sequence. In certain aspects, the gene silencing sequence comprises at least 50%, 60%, 70%, 80%, 90%, 95%, 98%, 99%, or 100% contiguously of the length of target gene sequence protein coding region, the target gene sequence 5′ UTR region, target gene sequence 3′ UTR region and/or any combination thereof.

In certain aspects, the target gene comprises a nucleic acid sequence selected from the group consisting of SEQ ID NOs: 1-14, 16-29, 31-69, 70-75, 76-88, 89-105, and 106-110. In certain aspects, the gene silencing sequence comprises about 100 to 1000 or about 200 to 1000 contiguous nucleotides of a sequence selected from the group consisting of SEQ ID NOs: 1-14, 16-29, 31-69, 70-75, 76-88, 89-105, and 106-110. In certain aspects, the gene silencing sequence comprises at least 50%, 60%, 70%, 80%, 90%, 95%, 98%, 99%, or 100% contiguously of the length of a sequence selected from the group consisting of SEQ ID NOs: 1-14, 16-29, 31-69, 70-75, 76-88, 89-105, and 106-110.

In certain aspects, the recombinant DNA construct has a gene silencing sequence operably linked to one or more promoters for the expression of a dsRNA molecule that silences the target gene when ingested by an insect. Thus, in certain aspects, the construct is an expression vector. Representative promoters for use in expressing a dsRNA molecule include, but are not limited to, CaMV35S or ZmUbil promoters etc. In certain aspects, the expression vector can target single or multiple insect RNAi target genes, for example, the vector could comprise one or more gene silencing sequences or could employ multiple vectors to target multiple insect RNAi target genes or chimeric dsRNA molecules.

### Host Cells and Plants

Provided herein are host cells, plants, and plants parts comprising, expressing, processing, and the like a dsRNA as described anywhere herein for inducing RNAi in an insect. In certain aspects, a host cell comprises a dsRNA molecule, siRNA molecule, a polynucleotide encoding a dsRNA molecule, and/or a construct or a dsRNA encoding segment thereof described anywhere herein. Representative examples of host cells include bacterial cells, fungal cells, yeast cells, plant cells, plant organelles (e.g., including plastids), and mammalian cells. In certain aspects, the host cell is a bacterial or plant cell. In certain aspects, the host cell is a transgenic and/or transplastomic plant cell. One of ordinary skill will understand that there are many well-known methods for introducing a nucleic acid, such as a vector, into a host cell including well-known methods for generating transgenic and/or transplastomic plant cells. In certain aspects, the hose cell expresses a dsRNA molecules and/or produces siRNA to silence a target gene. In certain aspects, a transgenic and/or transplastomic plant can comprise a dsRNA molecule, siRNA, a polynucleotide encoding a dsRNA, and/or a construct or a dsRNA encoding segment thereof. In certain aspects, at least one cell of a transgenic and/or transplastomic plant expresses a dsRNA molecule and/or produces a siRNA for silencing a target gene. Certain aspects provide for a seed, part, tissue, cell, or organelle of a plant described herein, wherein said seed, part, tissue, cell, or organelle comprises a dsRNA molecule and/or the siRNA for silencing a target gene.

### Methods of Insect Control

Also provided for herein are various methods of using a dsRNA molecule or vector encoding such dsRNA described anywhere herein for inducing RNAi in an insect and/or silencing a target gene. In certain aspects, this provides for control of insect pests.

Certain aspects provide for a method of silencing an insect immune response gene and/or an insect gene encoding for structural components of the insect midgut. In certain aspects, a method provides for the silencing an insect MIGGS-IRTG. Such method comprises providing for ingestion through spray, drenches, granules, seed coating or plant-incorporated protectant, or the like, to an insect an isolated dsRNA (pure or crude extract), siRNA, insecticidal composition, host cell, transgenic and/or transplastomic plant, and/or the seed, part, tissue, cell, or organelle thereof described anywhere herein.

Certain aspects provide for a method of silencing an insect immune response gene and/or an insect gene encoding for structural components of the insect midgut. In certain aspects, a method provides for the silencing an insect MIGGS-IRTG. Such method comprises providing for ingestion through spray, drenches, granules, seed coating or plant-incorporated protectant, or the like, to an insect an isolated dsRNA, siRNA, insecticidal composition, host cell, transgenic and/or transplastomic plant, and/or the seed, part, tissue, cell, or organelle thereof described anywhere herein.

Certain aspects provide for protecting a plant, such as a crop plant, from an insect pest including but not limited to pests of the order Lepidoptera like Manduca sexta (tobacco hornworm), Spodoptera frugiperda (fall armyworm), Ostrinia nubilalis (European corn borer), Plutella xylostella (Diamondback moth) or pests of the order Coleoptera like Leptinotarsa decemlineata Say (Colorado potato beetle), Diabrotica spp. (Corn rootworm complex), Tribolium castaneum (Red flour beetle), Popillia japonica (Japanese beetle), Agrilus planipennis (Emerald ash borer) or pests of the order Hemiptera like Diaphorina citri (Asian citrus psyllid), Cimex lectularius (Bed bug) or pests of the order Blattodea like all species of cockroaches and termites or insect pests of the order Diptera like all species of Mosquitoes and flies etc. Representative examples of plant hosts include, but are not restricted to, Zea mays L (corn), Sorghum bicolor (sorghum), Setaria italica (fox tail millet), Pennisetum glaucum (Pearl millet), Solanum tuberosum (potato), Oryza sativa (rice), Lycopersicon esculentum (tomato), Solanum melongena (eggplant), all cultivars of Brassica oleracea family, Citrus sinensis (Orange), trees of Oleaceae family and crops of Rosaceae etc. Such methods comprise, for example, topically applying to the plant the isolated dsRNA (pure or crude extract), the siRNA, and/or the insecticidal composition described anywhere herein, and providing the plant in the diet of the insect pest. In certain aspects the dsRNA molecule is topically applied by expressing the dsRNA in a microbe followed by topically applying the microbe onto the plant and/or seed.

Certain aspects provide for producing a plant resistance to a pest insect of said plant. Such methods comprises transforming the plant with a polynucleotide encoding a dsRNA and/or a construct or a dsRNA encoding segment describe anywhere herein, wherein the plant expresses a dsRNA and/or siRNA and/or the plant comprises a dsRNA and/or siRNA containing insecticidal compositions described anywhere herein, for silencing a target gene. In certain aspects, the transformed plant is more resistant to a pest insect of said plant than untransformed plants.

Certain aspects provide for improving crop yield. Such methods comprise growing a population of crop plants transformed with a polynucleotide encoding a dsRNA and/or the construct or a dsRNA encoding segment thereof described anywhere herein, wherein the plant expresses a dsRNA and/or siRNA and/or the plant comprises a dsRNA and/or siRNA containing insecticidal compositions described anywhere herein, for silencing a target gene. In certain aspects, a population of transformed plants produces higher yields in the presence of pest insect infestation than a control population of untransformed plants.

Certain aspects of the disclosure provide for an insecticidal composition comprising a nucleic acid molecule disclosed anywhere herein for silencing a target gene, including long dsRNA, hpRNA, and siRNA. In certain aspects, the insecticidal composition also comprises a synthetic carrier or a microbial conduit. For example, a microbial conduit can be a microorganism that has a natural capacity or is engineered to produce and/or deliver dsRNA to increase its bioavailability and/or biostability for causing RNA interference. Representative examples include plant growth promoting organisms, normal commensal and/or symbiotic microorganisms associated with the target insect pest or parasites and/or natural enemies of the target pest or pest target host or host cultivation range etc. from an insect or parasite and/or natural enemies of the target pest engineered or identified from natural populations containing microbial conduit to produce and/or deliver dsRNA and/or drive the transmission of such microbial conduits into natural populations of insect pests as a control option. In certain aspects a microbial conduit can be used as a direct topical application on a whole plant or coated onto a seed or mixed with growth media or transmitted through fertilizer or irrigation, etc. In certain aspects, the nucleic acid molecule of the insecticidal composition is conjugated to the synthetic carrier. For example, a synthetic carrier can be an inert chemical compound with a natural or engineered affinity to bind (conjugate) a dsRNA molecule to increase its biostability and/or bioavailability for causing RNA interference. In certain aspects, a synthetic carrier comprises a combination of inert chemicals or nanoparticles that upon combining and/or individually have a net positive charge or general affinity to bind to negatively charged dsRNA. Representative examples include chitosan, liposomes, carbon quantum dots, biodegradable particles of plant (e.g. coconut coir or grain flour, etc.) or soil (e.g. calcified clay) origin etc. In certain aspects, the dsRNA conjugated with a synthetic carrier can be used as a direct topical application directly and/or after aerosolization on a whole plant or coated onto a seed or mixed with growth media or transmitted through fertilizer or irrigation, etc. In certain aspects dsRNA or composition comprising the dsRNA can be used as a direct topical spray on application to whole plant, coated onto a seed or mixed with growth media or transmitted through fertilizer or irrigation or combined with plant growth promoting microbes etc.

Certain aspects provide for producing a plant resistant against a pest insect of said plant. Such methods comprise first transforming a plant cell with a polynucleotide encoding the dsRNA and/or the construct or a dsRNA encoding segment described anywhere herein. Next, a plant is regenerated from the transformed plant cell. The plant is then grown under conditions suitable for the expression of the dsRNA. In certain aspects, the transformed plant confers genetically tractable (maternal and/or paternal inherited) gain of function phenotypically manifested as an ability to impair the normal feeding and/or growth and/or development and/or reproductive success of the target plant pest and is consequently resistant to the plant pest insect compared to a control untransformed plant.

In certain aspects of any of the aforementioned methods, the insect larvae ingest the dsRNA. In certain aspects of any of the aforementioned methods, ingestion of the dsRNA induces a melanotic response in the insect larvae. In certain aspects of any of the aforementioned methods, ingestion of the dsRNA results in perturbation of gut microbial homeostasis. In certain aspects of any of the aforementioned methods, ingestion of the dsRNA results in defective clearance of opportunistic microbes. In certain aspects of any of the aforementioned methods, ingestion of the dsRNA results in defective containment of gut microbes.

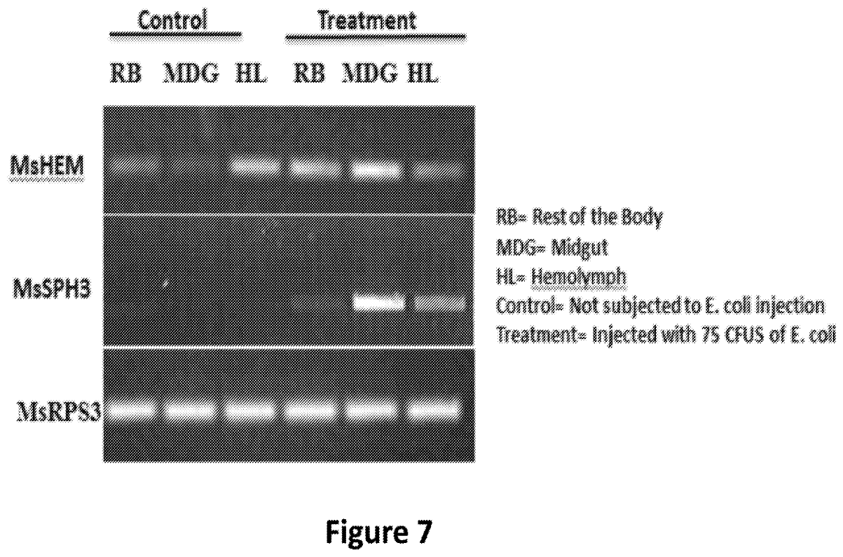

One of ordinary skill in the art will recognize that, the inventors have demonstrated midgut specific expression of a representative number of MIGGS-IRTGS in TH in response to their feeding on a lab strain of Escherichia coli (E. coli) bacteria. For example, a set of 20 MIGGS-IRTGS (SEQ ID NOs: 1-9, 11, 14, 31, 39, 43, 44, 71-75) that are induced in TH larvae feeding on an induction medium (Wang et al. (2006). J. Biol. Chem. 281(14): 9271-9278) is disclosed herein. It was previously reported that these twenty genes are expressed abundantly in the gut (Table 1). The gut specific expression of 2/20 genes were tested and validated the same (FIG. 7).

Orthologs of the representative TH MIGGS-IRTG (SEQ ID NOs: 76-88) set were identified from transcriptomic resources of an economically important Bt. resistant lepidopteran pest DBM using a combination of reciprocal best Blast analysis (Ward et al. (2014). PLoS ONE 9(7): e101850) and literature curation. Most of these MIGGS-IRTGS were induced in response to the feeding of DBM larvae on induction medium (Wang et al. (2006). J. Biol. Chem. 281(14): 9271-9278), similar to observations with TH larvae.

It was also demonstrated that most of the MIGGS-IRTGS were induced in another economically important lepidopteran pest, FAW, feeding on plants grown on representative field soil, using a RNA-Seq approach.

The RNA-Seq approach also identified additional RNAi candidates (SEQ ID NOs: 89-105) belonging to the MIGGS category. Further, orthologs of the expanded representative TH MIGGS-IRTG set (SEQ ID NO: 106-110) were identified from transcriptomic resources of an economically important Coleopteran pest RFB. It was demonstrated that most of the MIGGS-IRTGS were induced in response to the feeding of RFB beetles on induction medium (Wang et al. (2006). J. Biol. Chem. 281(14): 9271-9278), similar to observations with the order lepidoptera.

It was demonstrated that the targeted silencing of 9 out of 20 MIGGS-IRTGS employing bacterially expressed dsRNA protocol (Timmons L. et al. (2001). Gene. 263:103-112.) is insecticidal to TH larvae. Insecticidal activity against TH larvae correlated with the down regulation of target transcripts. It was demonstrated that the targeted silencing of 7 out of 14 MIGGS-IRTGS using bacterially expressed dsRNA protocol (Timmons L. et al. (2001). Gene. 263:103-112.) is insecticidal to DBM larvae. It was demonstrated that targeted silencing of 9 MIGGS-IRTGS are insecticidal to FAW larvae using bacterially expressed dsRNA protocol (Timmons L. et al. (2001). Gene. 263:103-112.). A core set of three MIGGS-IRTGS (SEQ ID NO: 3, 4, and 43) was identified that are efficacious against all three lepidopteran pests TH, DBM and FAW in an orthologous manner and that leaf discs coated with dsRNA against the core MIGGS-IRTGS are insecticidal against TH, DBM and FAW larvae. Further, plastidal expressed dsRNA against the core MIGGS-IRTGS impacted larval growth and survival.

The following examples are included to demonstrate certain embodiments of the disclosure. It should be appreciated by those of skill in the art that the techniques disclosed in the examples which follow represent techniques discovered by the inventor to function well in the practice of the disclosure. However, those of skill in the art should, in light of the present disclosure, appreciate that many changes can be made in the specific embodiments which are disclosed and still obtain a like or similar result without departing from the spirit and scope of the disclosure.

### EXAMPLES

In certain aspects, work was performed towards the identification, induction, isolation and cloning of the selected M. sexta (tobacco hornworm (TH)) MIGGS-IRTGS into a bacterial expression system capable of enabling the cloned genes to produce dsRNA (Timmons L. et al. (2001). Gene. 263:103-112.). Upon ingestion by M. sexta larvae, the bacterially expressed dsRNA is intended to silence, or at least knock-down, reduce, and the like the corresponding MIGGS-IRTG in order to curtail the feeding behavior and/or cause lethal effects in the insect pests. Based upon these results, additional testing was done on other representative insect species to demonstrate and establish for purposes of support wide applicability of the compositions and approaches of the disclosure.

For MIGGS-IRTG selection, PRR type genes co-regulated by the immune deficiency (IMD) pathway in TH were identified, these genes having been recently summarized (Casanova-Torres and Goodrich-Blair (2013). Insects (4): 320-338; Zhong X, et al. (2012). Insect Biochem. Mol. Biol. 42(7): 514-524); Zhang X, et al. (2015). Insect Biochem. Mol. Biol. 62:38-50; Cao X, et al. (2015). Insect Biochem. Mol. Biol. 62:64-74; Kanost M R, et al. (2016). Insect Biochem. Mol. Biol 76:118-147). Notably, most of the PRR genes selected for this study are abundantly induced or predicted to express in a midgut specific manner (Pauchet Y, et al. (2010). Insect. Mol. Biol. 19: 61-75; Kim and Lee (2014). Front. Cell. Infect. Microbiol. 3: 116; Lee and Hase (2014). Nat. Chem. Biol., 10: 416-424).

The TH-Transferrin, Arylphorin β subunit, chymotrypsinogen-like protein 1 and few other immunity related genes do not belong to the conventional PRR type immune responsive genes. However, these genes were included as they potentially contribute to the midgut microbial homeostasis through IMD co-regulation (Pauchet Y, et al. (2010). Insect. Mol. Biol. 19: 61-75). Additionally identified were a TH gene indicated to be a valine rich midgut protein critical for the formation of midgut peritrophic matrix and related genes critical for maintaining the structural integrity of the midgut, which are possibly involved, in the gut microbial containment. (Odman-Naresh et al. (2013). PLoS ONE 8:e82015. 10.1371/journal.pone.0082015; Engel and Moran (2013). FEMS Microbiol Rev. 37 699-735). A non-insect gene that encodes for catalase 1 from cassava (Manihot esculanta) was used as a control.

### Materials and Methods

TH larvae required for isolation of IRTG genes and subsequent bioassays were reared as follows: Eggs were procured from Carolina Biological Sciences (Burlington, NC, USA). The eggs were not surface sterilized and used directly for conventional rearing (CR). The larval colony establishment and maintenance was performed employing a Phytatray II (Sigma, MO, USA) unit containing Gypsy Moth diet. (Gunaratna R T and Jiang H (2013). Dev. Comp. Immunol. 39: 388-398).

Bt.-resistant DBM eggs were procured from Benzon Research (Carlisle, PA, USA) and reared conventionally, as described above.

FAW eggs were procured from Benzon Research (Carlisle, PA, USA) and reared conventionally, as described above.

For germ free (GF) rearing procedure, all eggs were surface sterilized with a solution of Tween-80 (polyoxyethylene sorbitan monooleate), bleach, and distilled water as described previously. (Broderick N A, et al. (2009). Environ. Entomol. 29:101-107). The surface sterilized eggs were transferred to Phytatray II (Sigma, MO, USA) unit containing Gypsy Moth diet augmented with antibiotics (500 mg/l each of penicillin, gentamicin, rifampicin, streptomycin). (Broderick N A, et al. (2009). Environ. Entomol. 29:101-107; Gregory R. Richards (2008). Journal of Bacteriology. 190, 4870-4879).

Both CR and GF larvae were reared in an environmental chamber with a 16:8 hours (light:dark) photoperiod at 25° C., until use. For the induction of a representative set of MIGGS-IRTGS of PRR category, 75 colony forming units (CFU) of DH5α competent cells (Invitrogen, CA, USA) re-suspended in PBS buffer were injected into healthy 2-3 instar M. sexta larvae. The larvae were snap frozen in liquid nitrogen and processed for RNA isolation and cDNA synthesis described herein.

For testing the up-regulation of MIGGS-IRTGS by oral feeding on induction media, first instar larvae were reared on Luria broth agar media plated with a mixture of live E. coli (3×107 cells), M. luteus (30 μg), and curdlan (30 μg) in 50 μl of H2O (Wang et al. (2006). J. Biol. Chem. 281(14): 9271-9278).

Total RNA was isolated using RNeasy Mini Kit reagent (QIAGEN, NY, USA) and treated with TURBO DNase (Ambion-Life Technologies, NY, USA) using manufacturer's protocols. One μg of DNase treated RNA was used for cDNA synthesis using iScript cDNA synthesis kit (Bio-Rad, CA, USA). The cDNA was used as a template for amplifying near full-length transcripts of the IRTG. Similarly, a control gene from cassava was also amplified. For tissue specific cDNA synthesis the control and treatment larvae were squeezed to isolate hemolymph fraction (HL), dissect midgut (MDG) to obtain rest of the body as described in Pauchet et al. (2010). Insect. Mol. Biol. 19: 61-75). The cDNA template was used for RT-PCR reactions were appropriate using the SuperScript III One-Step RT-PCR system following manufacturers protocol (Thermo Scientific; USA).

Transcripts of TH, DBM, and FAW MIGGS-IRTGS and non-insect control genes were PCR amplified using PrimeSTAR GXL DNA Polymerase (Clontech Laboratories, CA, USA). The PCR reactions were conducted using the following conditions: denaturation at 98° C. for 30 s, annealing at 55/60° C. for 30 s and elongation at 72° C. for 45 s, for 35 cycles. The PCR products were resolved by agarose gel electrophoresis and stained with ethidium bromide. The transcripts were gel eluted using QIAquick gel extraction kit (QIAGEN, NY, USA).

Sequence confirmed transcripts were cloned into pCR8/GW vector (Invitrogen, CA, USA) using manufacturer's protocol. The sequence confirmed recombinant pCR8 clones were cloned into L4440gtwy using LR clonase enzyme (Inivtrogen, CA, USA). The L4440gtwy is a modified version of Timmons and Fire feeding Vector and was a kind gift from Guy Caldwell (Addgene plasmid #11344). (Timmons & Fire (1998). Nature, 395: 854).

For ingestible RNAi bioassays, sequence confirmed MIGGS-IRTG were cloned into an L4440 feeding vector between two T7 promoters in inverted orientation and transformed into an E. coli bacterial strain carrying IPTG-inducible expression of T7 polymerase, HT115 (DE3). (Timmons & Fire (1998). Nature, 395: 854). Modification of IRTG in this manner was previously demonstrated to induce the expression of dsRNA. (Timmons L, et al. (2001). Gene, 263, 103-112; Kamath R S, et al. (2000). Genome Biol. 2: 1-10.).

The HT115 (DE3) strain is an RNase III-deficient E. coli strain whose T7 polymerase activity is IPTG-inducible. The HT115 (DE3) genotype is as follows: F—, mcrA, mcrB, IN (rinD-rrnE) 1, lambda -, mc14::Tn10 (DE3 lysogen: lavUV5 promoter-T7 polymerase) (IPTG-inducible T7 polymerase) (RNase III minus), with tetracycline as a selectable marker. (Kamath R S, et al. (2000). Genome Biol. 2:1-10). The standard heat shock protocol for transformation of L4440::IRTG and control construct was used.

Single colonies of HT115 bacteria containing cloned L4440 plasmids were picked and grown in a 5 mL LB culture with 50 mg/ml ampicillin (Amp). One mL of the liquid culture was saved for plasmid isolation followed by sequence confirmation of the L4440::IRTG clone. The recombinant bacterial clones were grown for 8 hours in liquid culture and were seeded directly on LB plates containing 1 mM IPTG and 50 mg/ml Amp for inducing the dsRNA. (Kamath R S, et al. (2000). Genome Biol. 2: 1-10). Seeded plates were allowed to dry under laminar airflow chamber and incubated at 37° C. temperature overnight.

For larval bioassay three to five 1-2-instar TH, DBM, and FAW larvae were placed on induced plates containing HT115 (DE3) cells containing the desired L4440::IRTG. Bioassays were conducted testing the TH larvae against MIGGS-IRTGS and controls listed in Table 1.

TABLE 1
List of total MIGGS-IRTGS from TH tested.

| Manduca sexta MIGGS | NCBI | Insect Biological | Midgut | Insecticidal |
|---|---|---|---|---|
| RNAI Target Genes | Acession Number | Function | Expression | Activity |
| Ms_PGRP2 (SEQ ID NO: 3) | GQ293365.1 | Immunity | Yes* | Yes |
| Ms_IMD (SEQ ID NO: 31) | Msex2.05477-RA | Immunity | Yes\hat{ } | No |
| Ms_Toll2 (SEQ ID NO: 8) | EF442782.1 | Immunity | Yes\hat{ } | Yes |
| Ms_Sck (SEQ ID NO: 71) | Msex2.03324-RA | Immunity | Yes\hat{ } | No |
| Ms_Akl (SEQ ID NO: 72) | Msex2.12479-RA | Immunity | Yes\hat{ }\hat{ } | No |
| Ms_Rel2A (SEQ ID NO: 5) | HM363513.1 | Immunity | Yes\hat{ } | Yes |
| Ms_Spz1A (SEQ ID NO: 47) | GQ249944.1 | Immunity | Yes\hat{ } | Yes |
| Ms_Cac (SEQ ID NO: 73) | Msex2.02793-RA | Immunity | Yes\hat{ } | No |
| Ms_Dorsal (SEQ ID NO: 6) | HM363515.1 | Immunity | Yes\hat{ }\hat{ } | No |
| Ms_Cad1 (SEQ ID NO: 39) | Msex2.04570-RA | Immunity | Yes\hat{ } | No |
| Ms_Hemolin1 (SEQ ID NO: 1) | M64346.1 | Immunity | Yes++ | No |
| Ms_SPH3 (SEQ ID NO: 2) | AF413067.1 | Immunity | DNA | No |
| Ms_Transferrin1 (SEQ ID NO: 11) | M62802.1 | Immunity | Yes+++ | No |
| Ms_Gloverin1 (SEQ ID NO: 74) | Gl110649240 | Immunity | Yes\hat{ }\hat{ }\hat{ } | No |
| Ms_HPA18 (SEQ ID NO: 9) | AY672794.1 | Immunity | No | No |
| Ms_βGRP2 (SEQ ID NO: 4) | AY135522.1 | Immunity | Yes* | Yes |
| Ms_CHS2 (SEQ ID NO: 43) | AY821560.1 | Structural Integrity | Yes* | Yes |
| Ms_βTub (SEQ ID NO: 75) | AF030547 | Structural Integrity | Yes+ | Yes |
| Ms_Suc1 (SEQ ID N: 44) | GQ293363.1 | Structural Integrity | Yes* | Yes |
| Ms_VMP1 (SEQ ID NO: 14) | NA | Structural Integrity | Yes+ | Yes |
| Ms_vATPaseE (Positive Control) | X67131 | ATP Hydrolysis | Yes | Yes |
| Me_Catalase 1 (Negative Control) | AF170272 | None | No | No |

Phenotypic differences in the larval development on L4440::IRTG containing HT115 (DE3) plates were documented and compared with the larval growth on negative and positive controls containing HT115 (DE3) plates. The larval phenotypes for a given treatment with appropriate controls were only considered true and documented if they were reproducibly observed in ⅔ or ⅘ larvae, in at least two independent feeding experiments.

For sprayable RNAi, a 24-well plate-based bioassay system was developed using a modified cetyl trimethylammonium bromide method of MEGAscript RNAi kit following manufacturer's protocol (Thermo Scientific, USA) for large-scale purification of dsRNA against a given MIGGS-IRTG. Integrity of the dsRNA was determined by electrophoresis on 1% agarose gel and its concentration determined using NanoDrop UV-VIS spectrometer. Leaf discs of 1 cm² diameter were detached from Nicotiana benthamiana (for TH) or Arabidopsis Col-WT (for DBM) or wheat cultivar Bobwhite (for FAW) plants grown on field soil were drop inoculated with 0, 4, 8 or 16 μg of purified dsRNA in TE buffer. Air-dried dsRNA coated leaf discs were placed in the bioassay plate containing 1 mL of 1% Murashige and Skoog agar medium per well. Each well contained one leaf disc and was infested with conventionally reared three first instar TH or DBM or FAW larvae. dsRNA coated leaf discs were replaced once every 24 hours and insecticidal activity of each dsRNA measured as a function of larval mortality after five days continuous feeding. The RFB bioassays were conducted using a previously published flour disc assay protocol (Cao et al. (2018). Int. J. Mol. Sci. 19, 1079) with adult beetles.

For RNA-Seq analysis to test if the MIGGS pathway genes are induced by soil microbiome in FAW, wheat (Triticum aestivum) seeds were surface sterilized and planted in 4.5″ pots containing field soil or filled with 4:1 sterile turface:sand mix. Seedlings were grown a growth chamber at for 20 days and infested with ten first-instar FAW larvae per pot. Vigorous larval feeding activity was confirmed and larval samples collected for RNA-Seq analysis. A “pooled RNA-Seq” approach (Rajkumar et al. (2015). BMC Genomics. 16(1): 548) was used to obtain a snap shot of differential FAW gene expression in response to feeding on plants grown on microbe rich (field soil) and microbe depleted (sterile surface) substrate.

In order to demonstrate additional dsRNA delivery methods a plastidal dsRNA expression system was employed. Since high concentrations of long dsRNAs can be stably produced in plastids (Zhang et al. (2015). Science. 347(6225): 991-994), three of the most potent insecticidal TH MIGGS-IRTGS (SEQ ID NO: 3, 4, and 43) dsRNAs (dsMsPGRP2; dsMsβGRP2 and dsMsCHS2) were expressed by plastid transformation in tobacco plants in collaboration with Plastomics Inc. following a previously published protocol (Zhang et al. (2015). Science. 347(6225): 991-994). Detached leaves of stable transplastomic lines expressing dsRNA against MIGGS targets were fed to TH larvae and assessed for insecticidal activity.

### Results

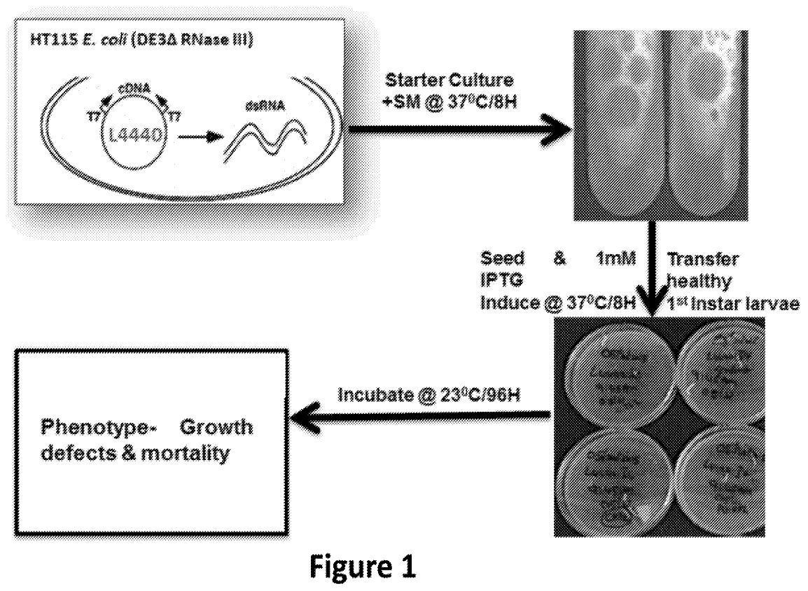

Bioassays (FIG. 1) indicated that the oral feeding activity of TH larvae on L4440::MsPGRP2 and L4440::MsVMP1 containing HT115 (DE3) plates resulted in growth impediment and/or mortality. Contrawise, the larvae growing on L4440::MeCAT1 containing HT115 (DE3) plates developed normally without any aberrant phenotypes.

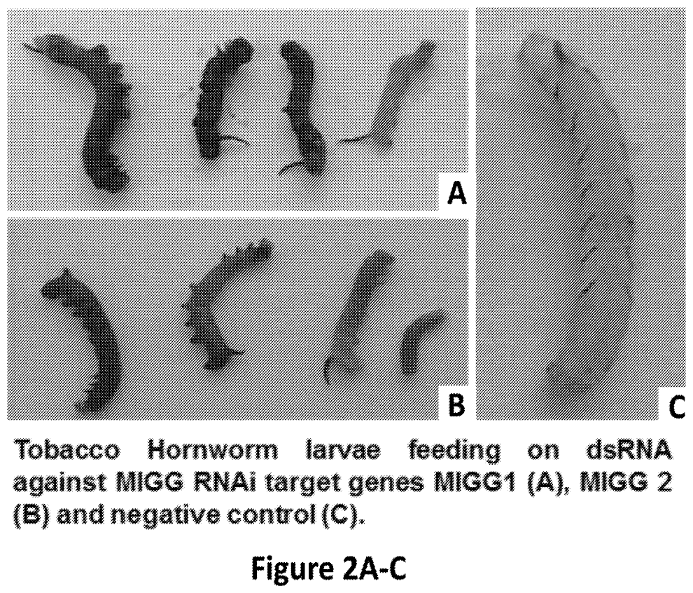

The representative phenotypes of TH larvae at 192 hours post exposure (HPE) to larvae exposed to bacterially (HT115 (DE3)) expressed dsRNA against MIGGS RNAi targets MsPGRP2 (FIG. 2A); MsVMP1 (FIG. 2B) and negative control dsRNA against Cassava plant specific gene MeCAT1 (FIG. 2C). The phenotypic effects leading to larval mortality were usually associated with the development of melanotic reaction, reduced appetite, growth and development (FIGS. 2A and B).

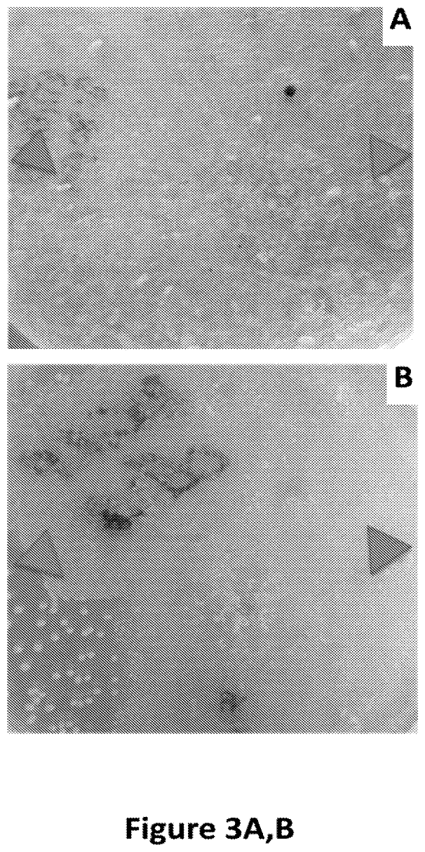

Consistent with above, the TH larvae exposed to bacterially expressed negative control MeCAT1 dsRNA displayed vigorous feeding (area between the arrowheads FIG. 3A). In contrast, the TH larvae exposed to bacterially expressed dsRNA against the MIGGS RNAi target MsPGRP2 displayed a curtailed feeding (representative picture, area between arrowheads FIG. 3B).

In general, melanization is a highly conserved immune response and is often associated with microbial infection of insects. (Kim S R, et al. (2005). Insect molecular biology, 14(2): 185-194. doi:10.1111/j.1365-2583.2004.00547). The intensification of melanotic response in TH larvae upon continued exposure to bacterially expressed dsRNA against the MIGGS RNAi targets MsPGRP2 and MsVMP1 containing HT115 (DE3) plates strongly indicates an infection, possibly due to the defective clearance of opportunistic microbes ingested during feeding. Such defective clearance has been previously associated with the perturbation of gut microbial homeostasis. (Packey and Sartor (2009). Curr. Opin. Infect. Dis. 22(3): 292-301).

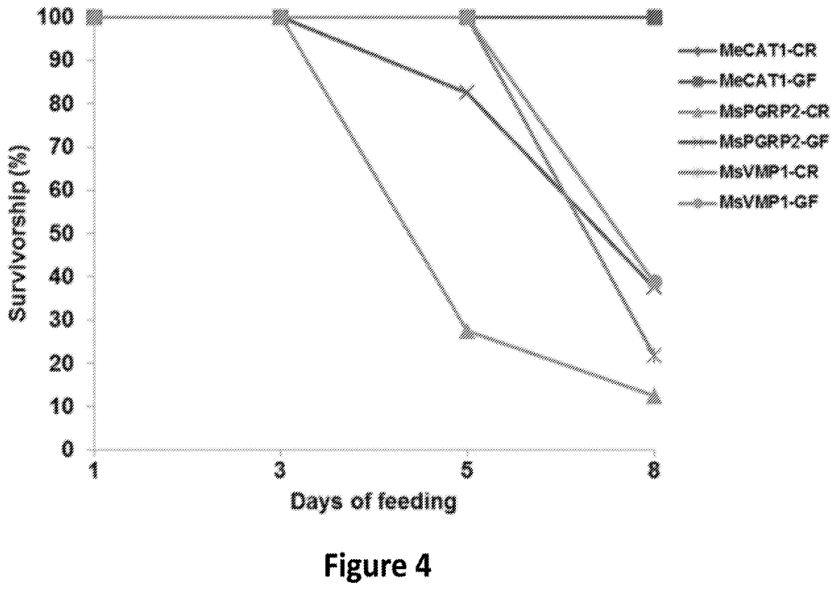

Closer observation indicated that the CR larvae feeding on bacterially expressed dsRNA against the MIGGS RNAi targets MsPGRP2 and MsVMP1 displayed discernable mortality starting at day 5, reaching up to 100% and 80% mortality respectively, by day 8 (FIG. 4).

To test if both the incidence and intensity of the observed phenotype could be delayed by clearing gut microbiotas, a GF set of TH larvae were also subjected to the above treatment with appropriate controls, in parallel. Interestingly, that the incidence of larval mortality was not only delayed, but also lower in GF larvae in comparison to CR larvae (FIG. 4) was observed.

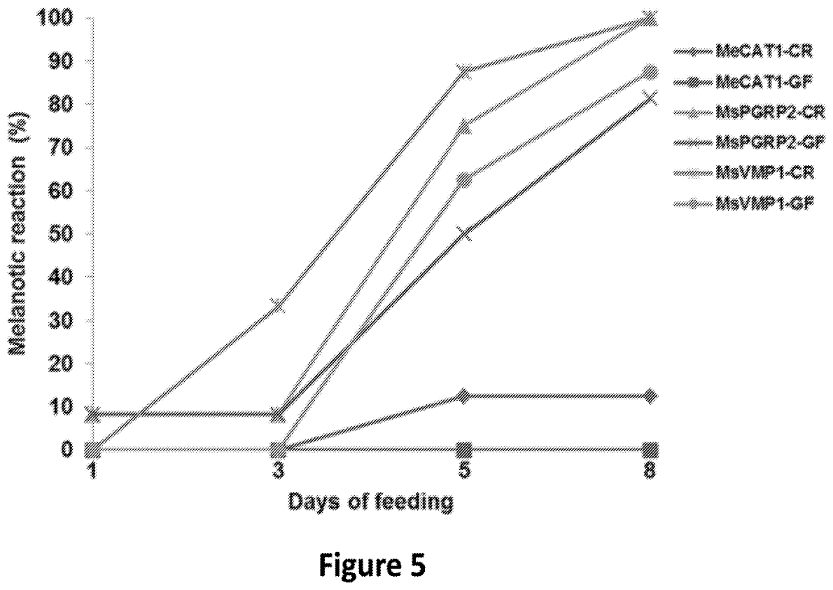

The incidence of larval mortality on bacterially expressed dsRNA against MIGGS RNAi targets MsPGRP2 and MsVMP1 plates also correlated with the development of melanotic reaction (FIG. 5). Most notably, there was a concomitant delay in the development of melanotic reaction in GF larvae in comparison to CR larvae (FIG. 5). All the phenotypes observed were statistically significant at a p-value between 0.001 and 0.05. No significant development of mortality or melanotic reaction was observed in larvae exposed to the bacterially expressed negative control MeCAT1 dsRNA (FIGS. 4 and 5).

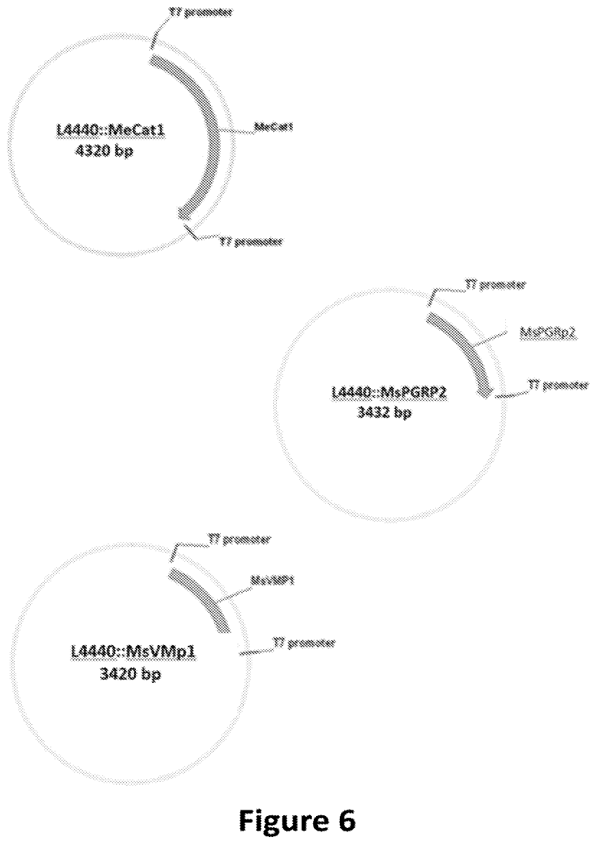

Although MsVMP1 is not directly involved in immune responses, down regulation may abrogate microbial containment, resulting in an infectious phenotype (FIG. 2B). Alternatively or in addition, down regulation of MsVMP1 may have resulted in wounding of the peritrophic matrix (the protective lining of the larval midgut) that also may have contributed to a sepsis mediated infectious phenotype (FIG. 2B); this is further substantiated by delayed onset of infectious symptoms in the larvae (CR or GF) exposed to dsRNA against MsVMP1 in comparison with larvae exposed to dsRNA against MsPGRP2 (FIGS. 4 and 5). Both defective clearance and defective containment of opportunistic microbes have been previously associated with lethal phenotypes. (Packey and Sartor (2009). Curr. Opin. Infect. Dis. 22(3): 292-301). The schematic representation of dsRNA delivery vectors used for above study is indicated in FIG. 6.

Most of the TH MIGGS RNAi targets disclosed herein (e.g., Table 1) are inducible and have been identified from open access midgut specific immunotranscriptome and/or other datasets (Pauchet Y, et al. (2010) Insect. Mol. Biol. 19:61-75; Odman-Naresh et al. (2013) PLoS ONE 8:e82015; Kanost M R, et al. (2016) Insect Biochem. Mol. Biol 76:118-147; Brummett et al. (2017) Insect Biochem Mol Biol. 81: 1-9; Cao X, et al. (2015) Insect Biochem. Mol. Biol. 62:64-74); Zhong X, et al. (2012) Insect Biochem. Mol. Biol. 42(7): 514-524; Xia Xu et al (2012). Dev Comp Immunol. 38(2): 275-284). A validation study targeting two MIGGS RNAi targets MsHEM and MsSPH3 confirmed their preferential midgut specific manner (FIG. 7) when injected with 75 CFU of gram negative E. coli. Further, literature curation confirmed the abundant expression of these MIGGS targets in the insects gut (Table 1).

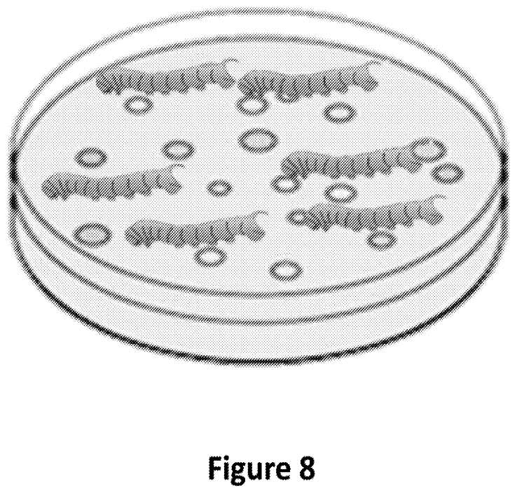

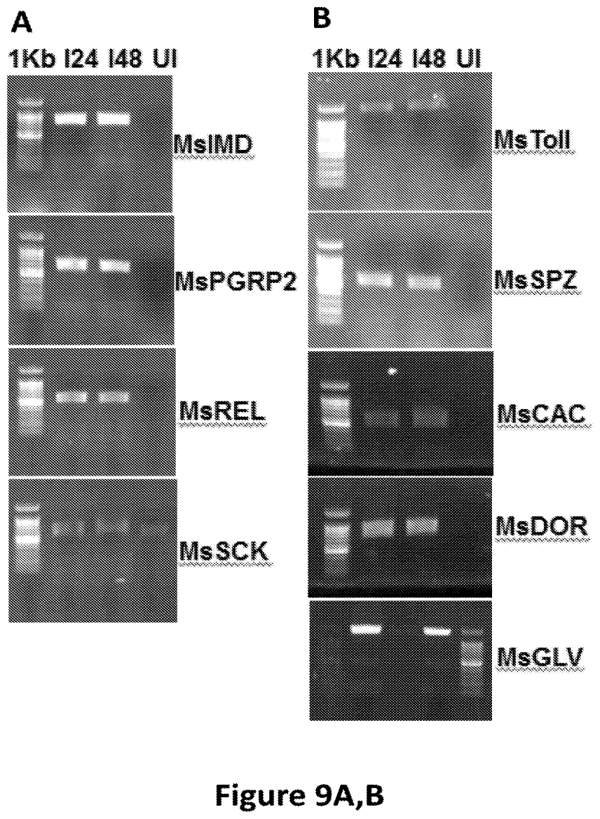

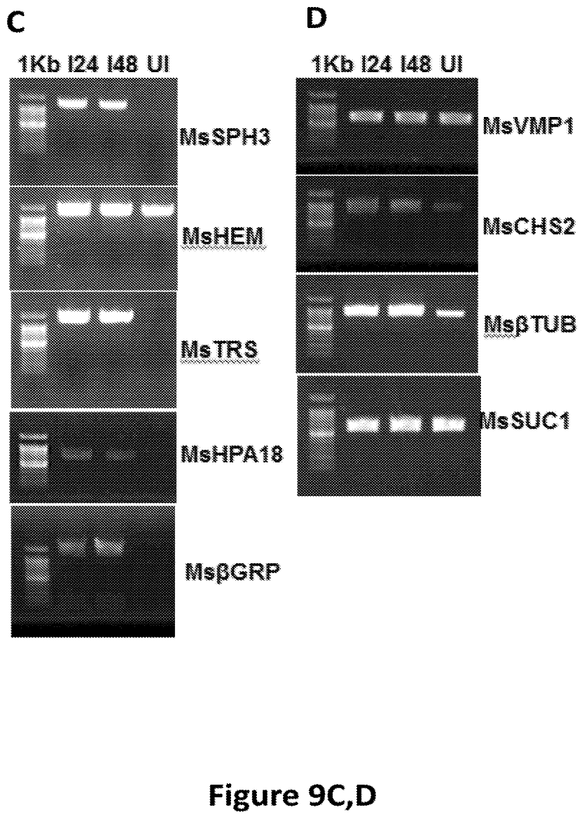

Oral feeding of TH larvae on induction media containing a mixture of live E. coli and lyophilized cell wall signatures from gram positive bacteria and fungi (FIG. 8) successfully induced (FIG. 9) the MIGGS RNAi target genes listed in Table 1. The genes with immunity related function were induced between 24-48 hours post larval exposure to induction media and mostly not detected in the absence of induction (FIG. 9A-C). While, the genes essential for midgut structural integrity are expressed under both conditions (FIG. 9D). This, clearly suggests that the MIGGS RNAi targets are induced in response to microbes ingested during feeding.

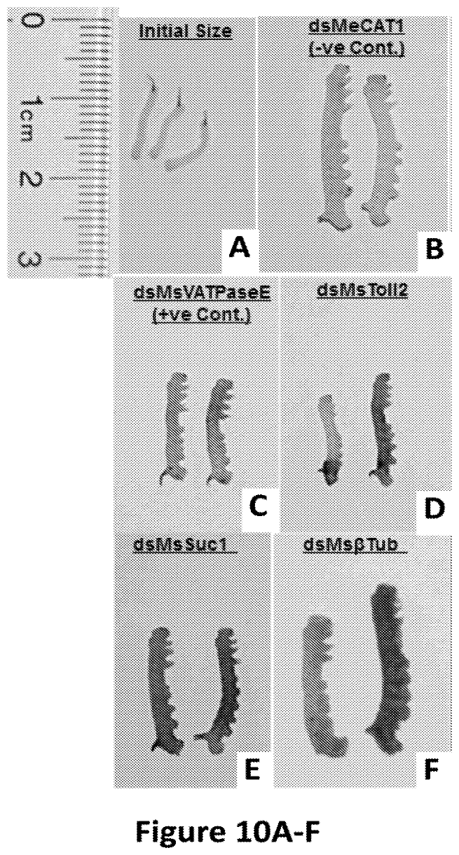

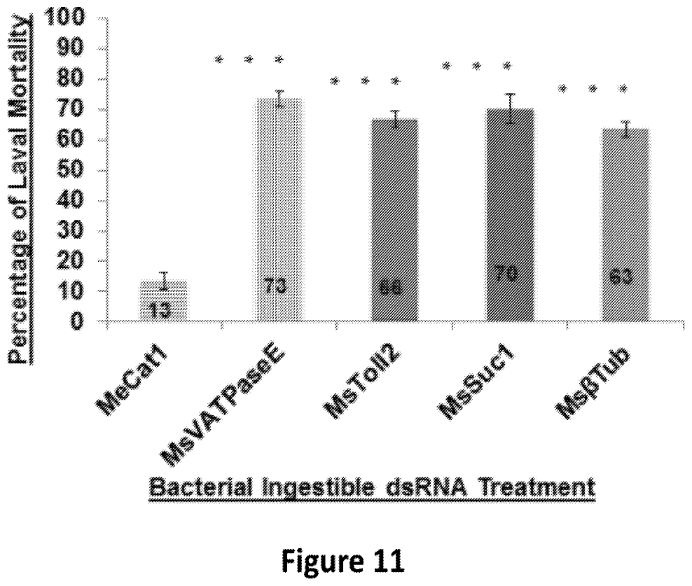

High-throughput screening of microbe induced MIGGS RNAi targets (FIG. 9) using bacterially delivered dsRNA screen identified 3 insecticidal candidates (FIG. 10). These MIGGS RNAi targets include Toll receptor (MsToll2), Beta fructofuranosidase 1 (MsSuc1) and Beta tubulin (MspTub) genes (FIG. 10D-F). The insecticidal activity was manifested as stunted growth and development, loss of appetite and melanotic reaction (FIG. 10D-F) as observed with the positive control MsVATPaseE treatment (FIG. 10C) in comparison to the negative control treatment (FIG. 10B). The developmental defects due to the bacterially expressed dsRNA exposure resulted in lethality ranging from 60-70% (FIG. 11), with MsSuc1 being the most effective target for killing TH larvae. Mortality rates were statistically significant and comparable to the MsVATPaseE positive control.

Additionally, a combination of reciprocal best BLAST analysis and literature curation was used to identify the orthologs of TH MIGGS RNAi targets from the DBM transcriptomic resources (Table 2).

TABLE 2
List of MIGGS-IRTGS identified from DBM transcriptome resources.

| | | | Midgut | Insecticidal |
|---|---|---|---|---|
| | | | Expression | Activity in |
| Manduca sexta MIGGS RNAi | Insect Biological | Putative Plutella | in Plutella | Plutella |
| Target Genes | Function | Xylostella Orthologs | Xylostella | Xylostella |
| Ms_PGRP2 | Immunity | Px_PGRP2 (SEQ ID NO: 76) | Yes* | Yes |
| Ms_IMD | Immunity | Px_IMD (SEQ ID NO: 77) | Yes* | Yes |
| Ms_Rel2A | Immunity | Px_Rel (SEQ ID NO: 78) | Yes* | No |
| Ms_Tol2 | Immunity | Px_Toll2 (SEQ ID NO: 79) | Yes* | No |
| Ms_Cac | Immunity | Px_Cac (SEQ ID NO: 80) | Yes* | Yes |
| Ms_Dorsal | Immunity | Px_Dorsal (SEQ ID NO: 81) | Yes* | Yes |
| Ms_Hemolin1 | Immunity | Px_Hemolin1 (SEQ ID NO: 82) | Yes* | No |
| Ms_SPH3 | Immunity | Px_SPH3 (SEQ ID NO: 83) | DNA* | No |
| Ms_Transferrin1 | Immunity | Px_Transferrin1 (SEQ ID NO: 84) | Yes* | No |
| Ms_βGRP2 | Immunity | Px_βGRP2 (SEQ ID NO: 85) | Yes* | Yes |
| Ms_Gloverin1 | Immunity | Px_Geoverin (SEQ ID NO: 86) | Yes* | No |
| Ms_CHS2 | Structural Integrity | Px_CHS1 (SEQ ID NO: 87) | Yes* | Yes |
| Ms_βTub | Structural Integrity | Px_βTUB (SEQ ID NO: 88) | Yes* | Yes |
| Ms_vATPaseE (Positive Control) | ATP Hydrolysis | Px_vATPaseE | Yes* | Yes |
| Me_Catalase 1 (Negative Control) | None | Ne_Catalase 1 | No* | No |

| Manduca sexta MIGGS RNAi | NCBI Accession | | NCBI Acession |
|---|---|---|---|
| Target Genes | Number | Putative Plutella Xylostella Orthologs | Number |
| Ms_PGRP2 | GQ293365.1 | Px_PGRP2 (SEQ ID NO: 76) | AFV15800.1 |
| Ms_IMD | Msex2.05477-RA | Px_IMD (SEQ ID NO: 77) | Px103008 |
| Ms_Rel2A | HM363511.1 | Px_Rel (SEQ ID NO: 78) | Px102858 |
| Ms_Tol2 | EF442782.1 | Px_Toll2 (SEQ ID NO: 79) | Px106338 |
| Ms_Cac | Msex2.02793-RA | Px_Cac (SEQ ID NO: 80) | Px116565 |
| Ms_Dorsal | HM363515.1 | Px_Dorsal (SEQ ID NO: 81) | Px100110 |
| Ms_Hemolin1 | M64346.1 | Px_Hemolin1 (SEQ ID NO: 82) | ACN53154.1 |
| Ms_SPH3 | AF413067.1 | Px_ SPH3 (SEQ ID NO: 83) | XP_004322155.1 |
| Ms_Transferrin1 | M62802.1 | Px_Transferrin1 (SEQ ID NO: 84) | BAF36818.1 |
| Ms_βGRP2 | AY135522.1 | Px_βGRP2 (SEQ ID NO: 85) | QβVIJ95.1 |
| Ms_Gloverin1 | G1110649240 | Px_Geoverin (SEQ ID NO: 86) | ACN69342.1 |
| Ms_CHS2 | AY821560.1 | Px_CHS1 (SEQ ID NO: 87) | KX420588.1 |
| Ms_βTub | AF030547 | Px_βTUB (SEQ ID NO: 88) | EU1Z7912.2 |
| Ms_vATPaseE (Positive Control) | X67131 | Px_vATPaseE | AB189032 |
| Me_Catalase 1 (Negative Control) | AF170272 | Ne_Catalase 1 | AF170272 |

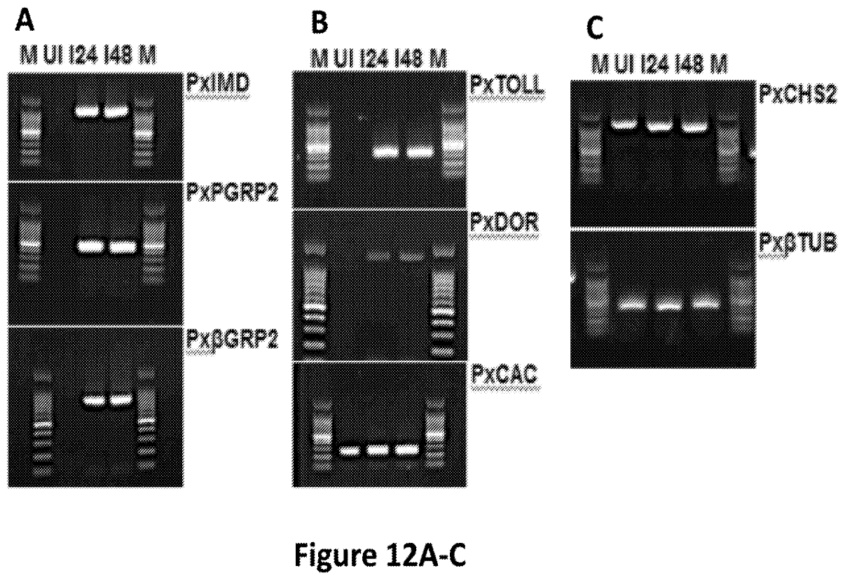

It was demonstrated that 14 MIGGS RNAi target genes identified could also be induced in Bt resistant strain of DBM feeding on the induction media (FIG. 12), thus suggesting that oral induction of microbe associated cell wall signatures could induce the MIGGS RNAi target genes in economically important DBM.

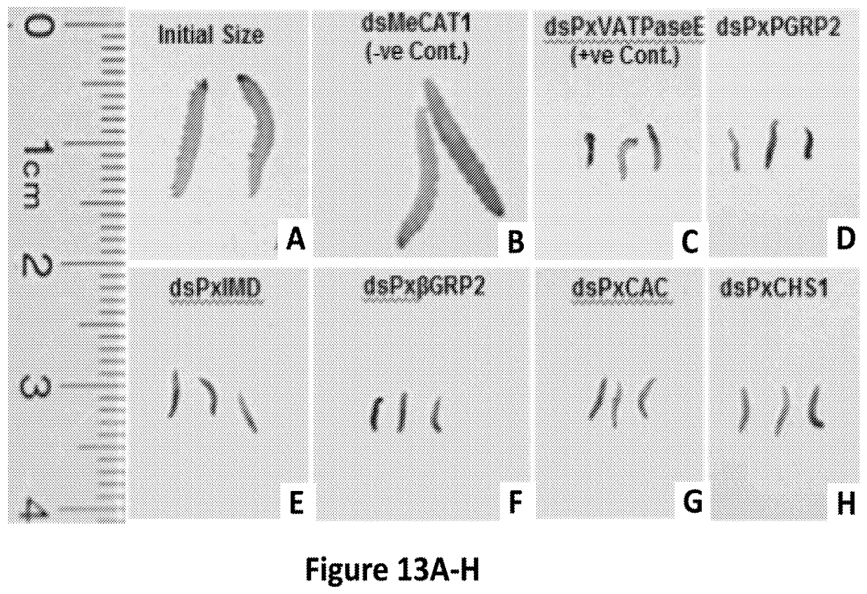

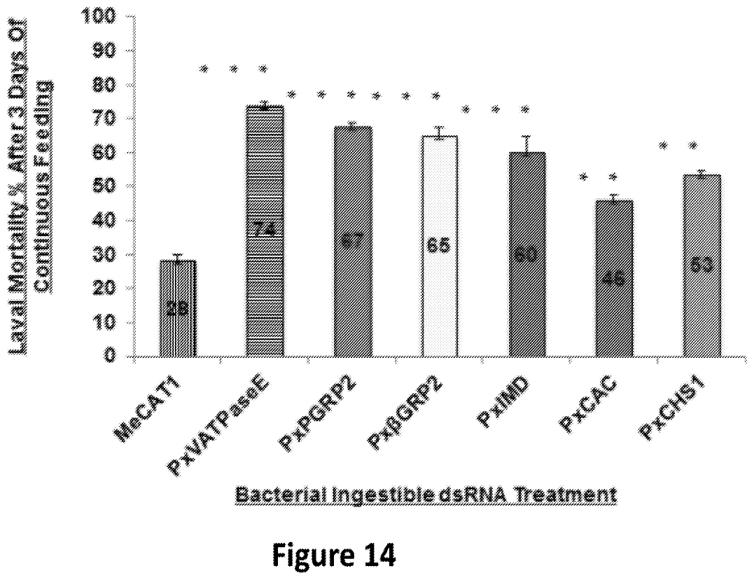

High throughput screening of microbe induced DBM MIGGS RNAi target genes (FIG. 12) using bacterially delivered dsRNA screen indicated that 7/15 MIGGS RNAi targets tested showed insecticidal activity against a Bt resistant strain of DBM (FIG. 13). The DBM insecticidal targets included PxPGRP2 (SEQ ID NO. 76), PxβGRP2 (SEQ ID NO. 85), PxCHS2 (SEQ ID NO. 87), PxCAC (SEQ ID No. 80), PxIMD1 (SEQ ID No. 77), PxDor (SEQ ID No. 81) and PxβTub (SEQ ID NO. 88). The insecticidal activity was manifested as stunted growth and development, loss of appetite and melanotic reaction (FIG. 13D-H) as observed with the positive control MsVATPaseE treatment (FIG. 13C) in comparison to the negative control treatment (FIG. 13B). The developmental defects due to the bacterially expressed dsRNA exposure resulted in lethality ranging from 53-70% (FIG. 14), with PxPGRP2 the most effective target for killing DBM larvae (FIG. 14). Mortality rates were statistically significant and comparable to the PxVATPaseE positive control (FIG. 14).

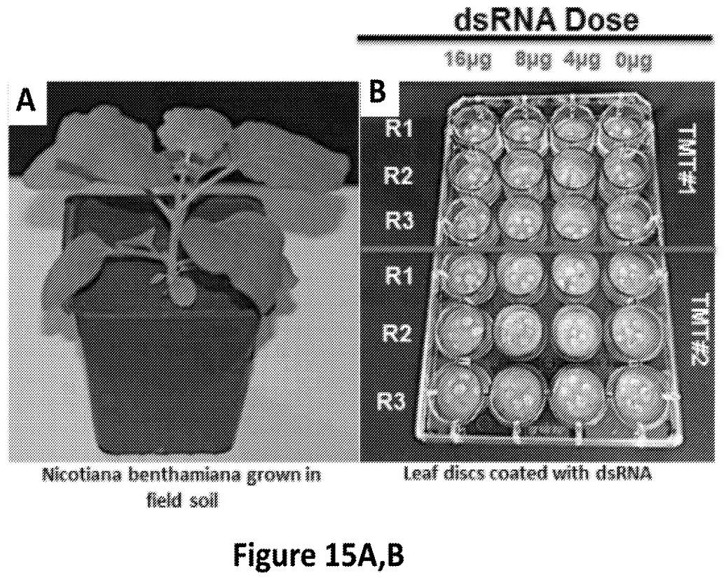

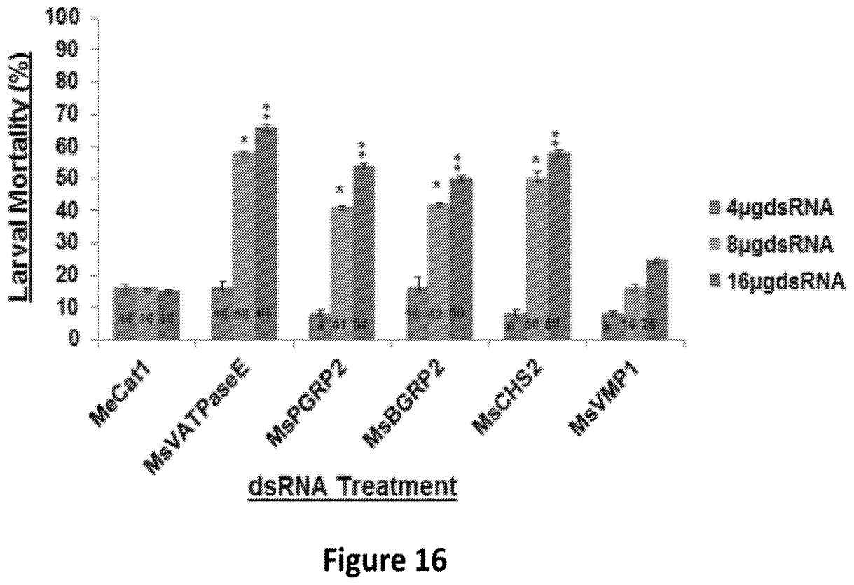

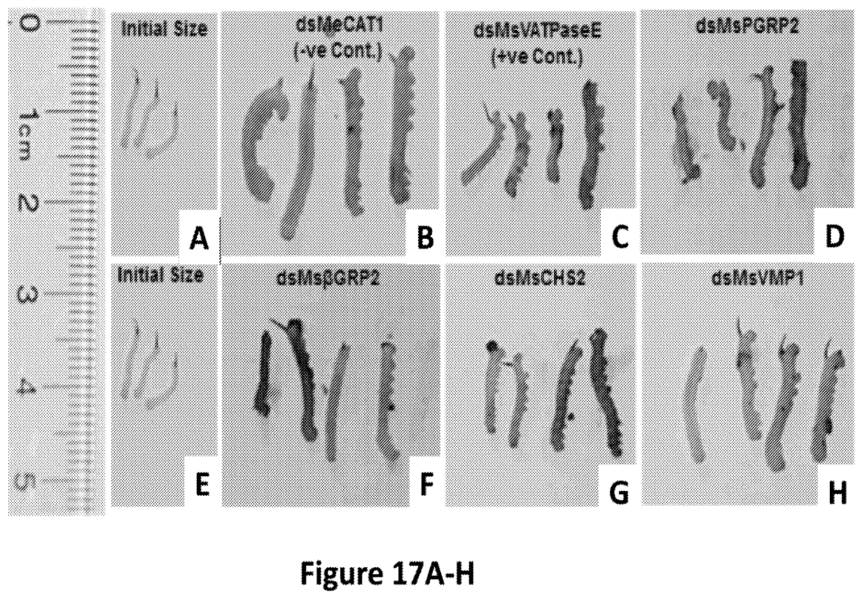

In order to test if our MIGGS RNAi technology could work using multiple dsRNA delivery platforms we tested if dsRNA against the four TH MIGGS insecticidal targets could work in a sprayable format. We used dsRNA against MsPGRP2, MsβGRP2, MsCHS2 and MsVMP1 for sprayable RNAi assays (FIG. 15) at a concentration of 0, 4, 8 or 16 μg of purified dsRNA in TE buffer. Most efficacious leaf disc coated dsRNA was 16 μg of dsRNA against MsCHS2 that caused 58% mortality. Similar, but slightly reduced, mortality was observed when insects were fed 8 μg of leaf disc coated dsRNA against the same MIGGS targets. Observed mortality rates were statistically significant and comparable to those observed in TH larvae exposed to dsRNA against a known positive control target MsVATPaseE (FIG. 16). No statistically significant larvae death was observed when feeding on 4 μg dsRNA (FIG. 16), indicating that 8-16 μg dsRNA per leaf disc was required to cause mortality in this assay. In addition to death, TH larvae feeding on dsRNA were characterized by developmental defects, loss of appetite, melanotic reaction and reduced growth compared to negative controls, indicating significant detrimental impact of MIGGs targeting on insect health (FIG. 17). The insecticidal TH MIGGS targets MsPGRP2, MsβGRP2 and MsCHS2 will be henceforth referred to as core set.

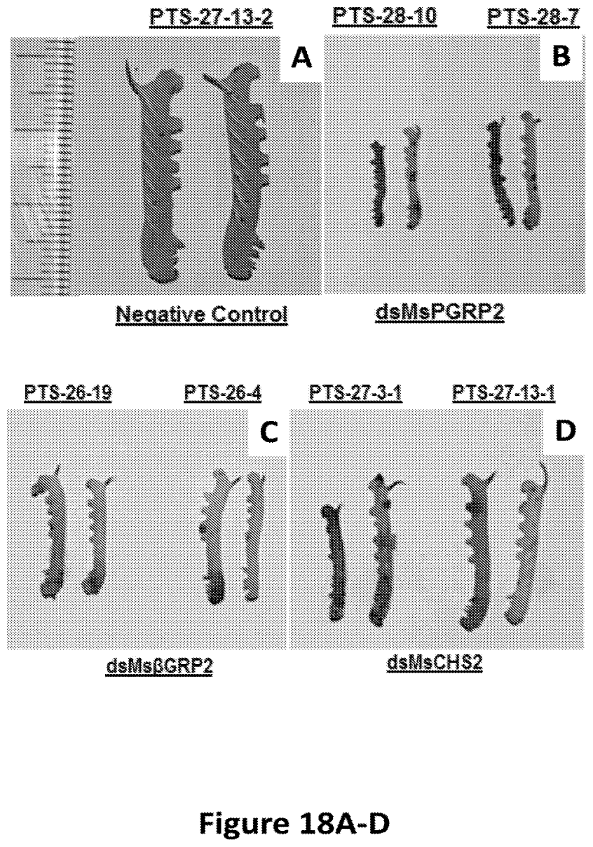

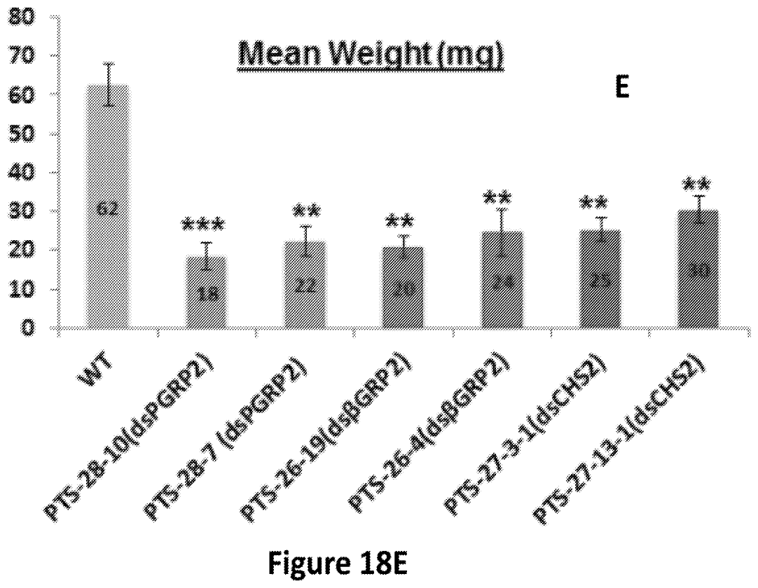

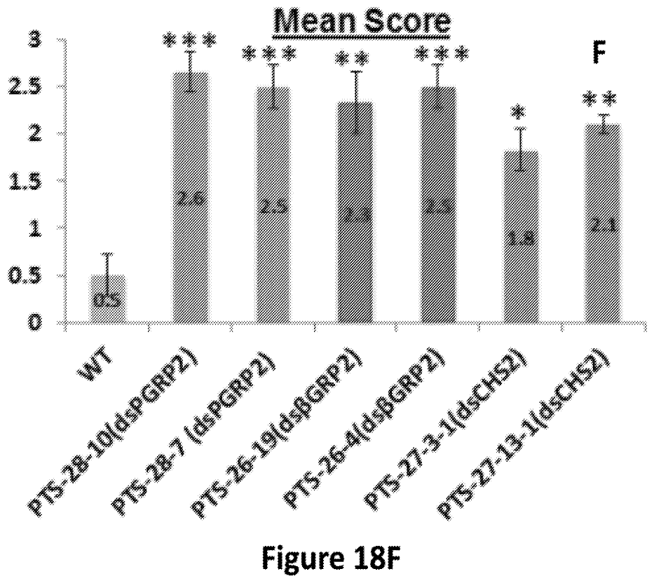

Similarly, in order to test an additional delivery platform, the core insecticidal MIGGS RNAi targets MsPGRP2; MsβGRP2 and MsCHS2 were expressed by plastid transformation in tobacco plants in collaboration with Plastomics Inc. Detached leaves of stable transplastomic lines (FIG. 18) expressing dsRNA against MIGGS targets indicated that the TH larvae feeding on leaves expressing dsRNA against MIGGS targets MsPGRP2 (B); MsβGRP (C) and MsCHS2 (D) display stunted growth, development, loss of appetite and melanotic reaction in comparison to negative control (A). The insecticidal activity is manifested as significant reduction in mean weights in comparison to negative control (E). The mortality rate was scored on a 0-3 score were 0, 1, 2 and 3 indicated ≤0, 25, 50 or ≥50 mortality respectively. The transplastomic events confer significant mortality in comparison to negative control (F). Data is average of 6 replicates/treatment (N=24)±SEM at p≤0.001(***); p≤0.01(**) and p≤0.05(*). Clearly suggesting that the stably expressed dsRNA against the core MIGGS RNAi targets also impacts larval growth and development.

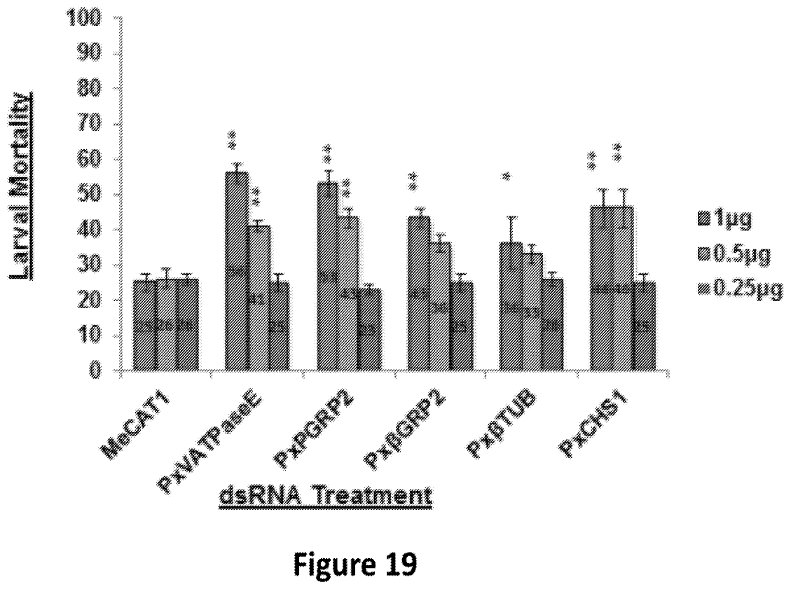

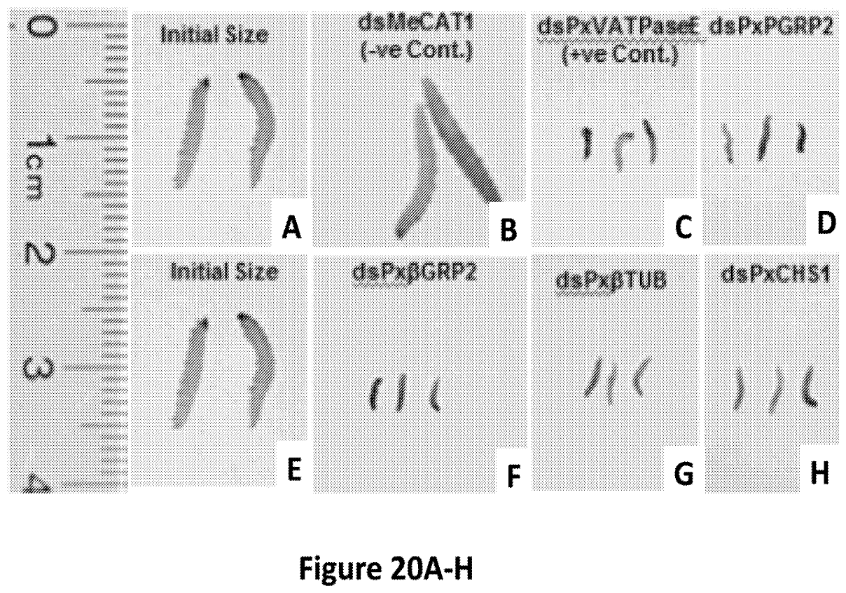

Next, we tested if our MIGGS RNAi technology can also work in a sprayable format against Bt resistant strain of DBM. We used the DBM orthologs of insecticidal TH MIGGS core set (PxPGRP2, PxβGRP2 and PxCHS2) and two newly discovered insecticidal DBM MIGGS targets (PxCAC and PxIMD1) for sprayable RNAi assays at a concentration of 0.1, 0.5 and 0.25 μg. We observed lethality ranging from 53-67%, with PxPGRP2 being the most effective target for killing DBM larvae (FIG. 19). Mortality rates were statistically significant and comparable to the PxVATPaseE positive control (FIG. 19). In a manner similar to that seen for sprayable dsRNA against TH, in addition to death, DBM larvae exposed dsRNA treatments were characterized by developmental defects, loss of appetite, melanotic reaction and arrested growth (FIG. 20). Clearly suggesting that dsRNA against the insecticidal MIGGS RNAi targets identified herein when used in sprayable format is also effective against Bt resistant strain of DBM.

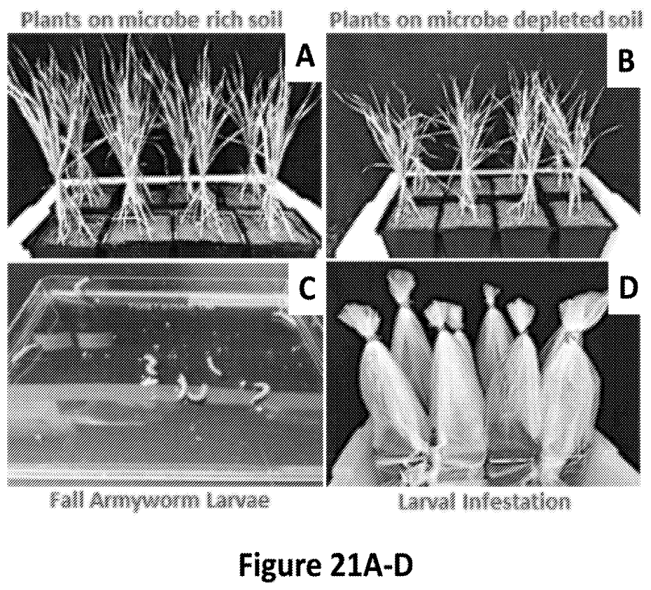

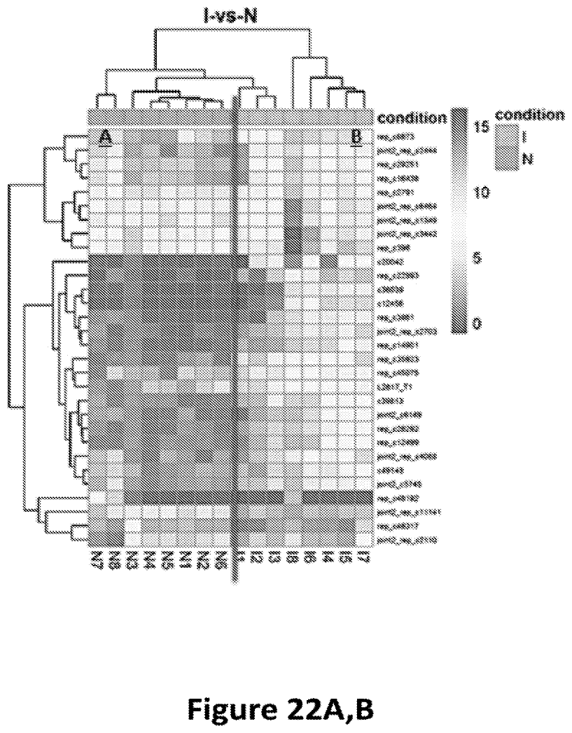

Most of our MIGGS RNAi target gene induction procedures thus far relied upon either direct injection or oral feeding of extraneously supplied microbial signatures. To determine if our MIGGS RNAI target genes are induced under field conditions, we exposed the larvae of economically important lepidopteran pest FAW to wheat seedlings grown on microbe rich and microbe depleted plants (FIG. 21). Pooled RNA-Seq analysis indicated that plants growing on field soil caused preferential up-regulation of MIGGS pathway genes in FAW. In total, 100 differential expressed genes were identified, thirty of which were MIGGS pathway related genes (FIG. 22).

TABLE 3
List of MIGGS-IRTGS identified from using RNA-Seq approach in FAW.

| Spodoptera fruglperda MIGGS RNAI | | Putative Insect | Midgut | Insecticidal |
|---|---|---|---|---|
| Target Genes | Query_Contigs_ID* | Biological Function | Expression* | Activity |
| Sf_PGRP1 (SEQ ID NO: 89) | rep_c7951 | Immunity | Yes | Yes |
| Sf Attacin (SEQ ID NO: 90) | rep_c9395 | Immunity | Yes | Yes |
| Sf_Ctypelectin15 (SEQ ID NO: 91) | joint2_rep_c488 | Immunity | Yes | No |
| Sf_Galectin4 (SEQ ID NO: 92) | rep_c25253 | Immunity | Yes | No |
| Sf_Lysozyme (SEQ ID NO: 93) | rep_c18992 | Immunity | Yes | No |
| Sf_Hemolymph proteinase 10 | c12881 | Immunity | No | Yes |
| (SEQ ID NO: 94) | | | | |
| Sf_Trypsin like Serine protease | rep_c48453 | Immunity | Yes | Yes |
| (SEQ ID NO: 95) | | | | |
| Sf-C type Lectin 6 (SEQ ID NO: 96) | joint2_rep_c448 | Immunity | Yes | Yes |
| Sf_Serine protease 13 (SEQ ID NO: 97) | rep_c1904 | Immunity | Yes | No |
| Sf_Cecropin (SEQ ID NO: 98) | rep_c42380 | Immunity | Yes | Yes |
| Sf_Relish (SEQ ID NO: 99) | c13122 | Immunity | Yes | No |
| Sf_Toll2 (SEQ ID NO: 100) | joint2_c3284 | Immunity | Yes | No |
| Ms_βBGRP2 (SEQ ID NO: 101) | EF641300 | Immunity | Yes | Yes |
| Sf_c20042 (SEQ ID NO: 102) | c20042 | Immunity | Yes | No |
| Sf_rc16438 (SEQ ID NO: 103) | rc16438 | Immunity | Yes | Yes |
| Sf_L2rC2367 (SEQ ID NO: 104) | joint2_rep_C2367 | Immunity | Yes | No |
| Sf_CHSB (SEQ ID NO: 105) | AY525599 | Structural Integrity | Yes | Yes |

Most notably, FAW orthologs of TH insecticidal targets including PGRP2, PGRP2 and IMD were captured in the data set. This discovery indicated that MIGGS pathway genes are up regulated in response to insect feeding on plants exposed to microbes in the field soil.

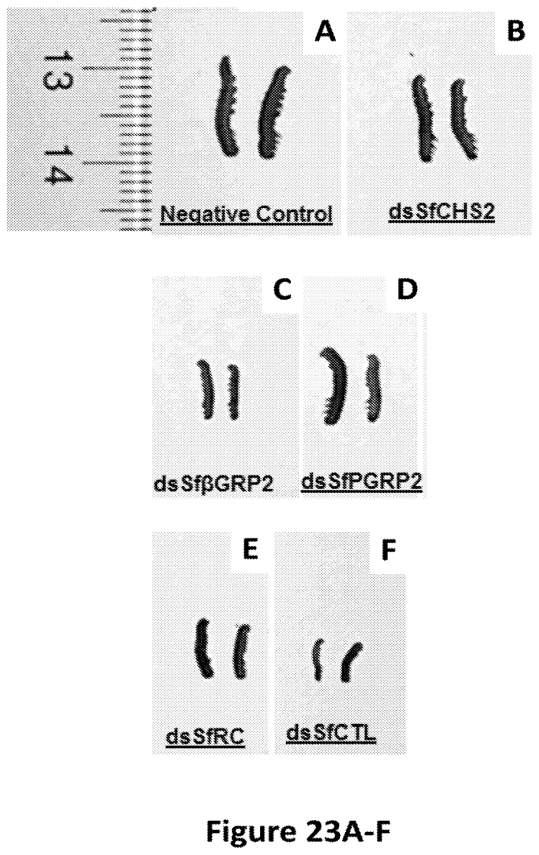

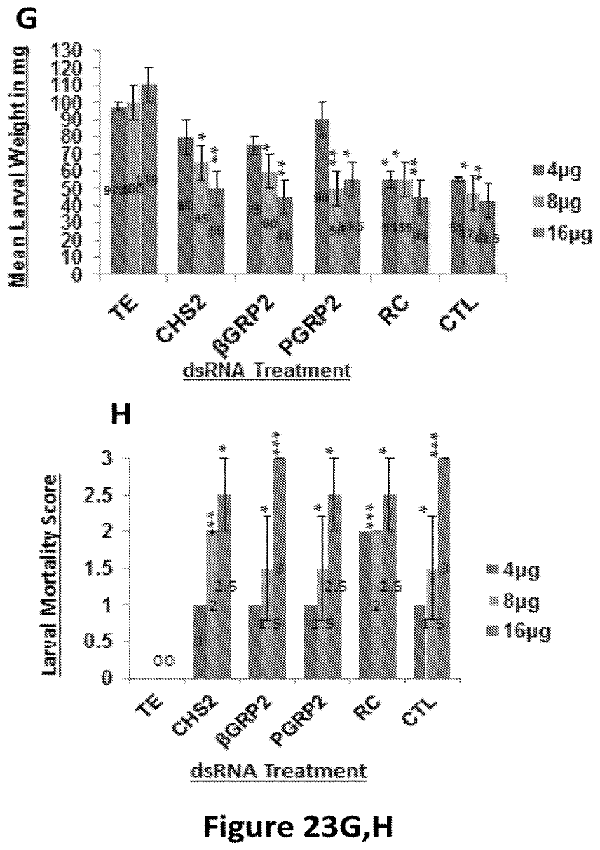

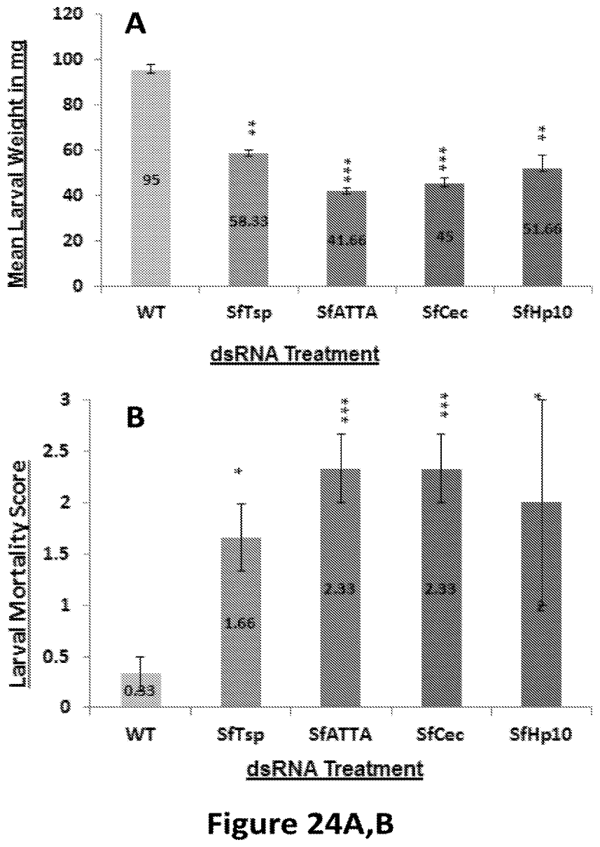

Preliminary experiments were performed to screen the insecticidal activity of 17 FAW MIGGS RNAi targets discovered during RNA-Seq (Table 3, above) in which dsRNA against the FAW orthologs of insecticidal TH MIGGS core set (SfPGRP2 (SEQ ID NO. 89), SfβGRP2 (SEQ ID NO. 101) and SfCHS2 (SEQ ID NO. 105) and three newly discovered MIGGS targets SfCTL (SEQ ID NO. 96), SfRC (SEQ ID NO. 103) and SfGAL (SEQ ID NO. 92) from RNA-Seq were fed to FAW larvae at 0, 4, 8 or 16 μg-purified dsRNA in TE buffer. FAW 1st instar larvae were allowed to feed on dsRNA coated leaves following the bioassay described for TH above. Data indicated that FAW larvae (FIG. 23) exposed to pure dsRNA against SFCHS2 (B); SFβGRP2 (C); SFPGRP2 (D), SFRC (E), and SFCTL (F) causes reduced growth, development and loss of appetite in comparison to negative control treatment (A) resulting in significant weight reduction (G) and mortality (H) at 8 and 16 μg of dsRNA concentration. The rates of mortality was scored on a 0-3 score were 0, 1, 2 and 3 indicated ≤0, 25, 50 or ≥50 mortality respectively. The dsRNA treatments imposed caused statistically significant reduction in mean weights (G) that also translated into significant rates of mortality (H) in comparison to negative control (FIG. 23). Screening of insecticidal activity of additional MIGGS RNAi target genes identified from the RNA-Seq dataset indicated that 16 μg of leaf disc coated dsRNA against MIGGS RNAi targets SfTSP (SEQ ID NO. 95), SfAtta (SEQ ID NO. 90), SfCec (SEQ ID NO. 98) and SfHp10 (SEQ ID NO. 94) caused statistically significant reduction (FIG. 24) in mean weights (A) that also translated into significant rates of mortality (B) in comparison to negative control. Data is average of 3 replicates/treatment±SEM at p≤0.001(***); p≤0.01(**) and p≤0.05(*).

Experiments with an economically important coleopteran pest RFB were also conducted to test if MIGGS-IRTGS targets identified by a combination of reciprocal best BLAST analysis and literature curation (Table 4) are insecticidal against the order coleoptera.

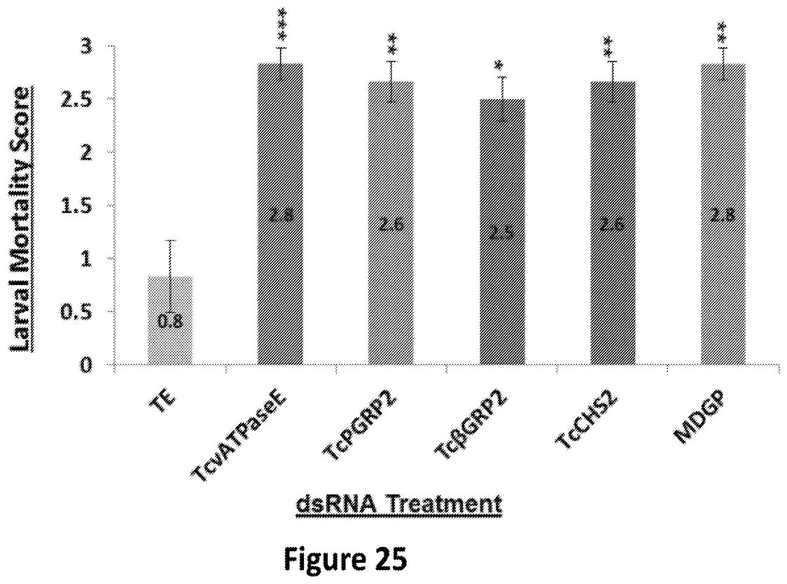

Preliminary feeding trails with 1 μg of purified indicated that dsRNA against RFB MIGG RNAi targets TcPGRP2 (SEQ ID NO. 107), TcβGRP2 (SEQ ID NO. 108), TcMDGP (SEQ ID NO. 109) and TcCHS2 (SEQ ID NO. 110) is insecticidal to the adult RFB beetles. Significant rates of RFB mortalities were observed (Figure. 25) when scored on a 0-3 scale were 0, 1, 2 and 3 indicated ≤0, 25, 50 or ≥50 mortality respectively in comparison to the negative control treatment. Data is average of 3 replicates/treatment±SEM at p≤0.001(***); p≤0.01(**) and p≤0.05(*).

TABLE 4
List of MIGGS-IRTGS identified from RFB transcriptome resources.

| Spodoptera frugiperda | | | | |
|---|---|---|---|---|
| MIGG RNAI Target Genes | Query_Contigs_ | Putative Insect | Midgut | Insecticidal |
| (SEQ ID NO) | ID | Biological Function | Expression | Activity |
| TcPGRPL0 | DT786101.1 | Immunity | Yes* | No |
| (SEQ ID NOL: 106) | | | | |
| TcPGRP2 | XM_965754.3 | Immunity | Yes* | Yes |
| (SEQ ID NO: 107) | | | | |
| TcβGRP2 | XM_966587.4 | Immunity | DNA | Yes |
| (SEQ ID NO: 108) | | | | |
| TcMDGP | XM_971351 | Structural Integrity | Yes** | Yes |
| (SEQ ID NO: 109) | | | | |
| TcCHS2 | EFA 10719.1 | Structural Integrity | Yes*** | Yes |
| (SEQ ID NO: 110) | | | | |

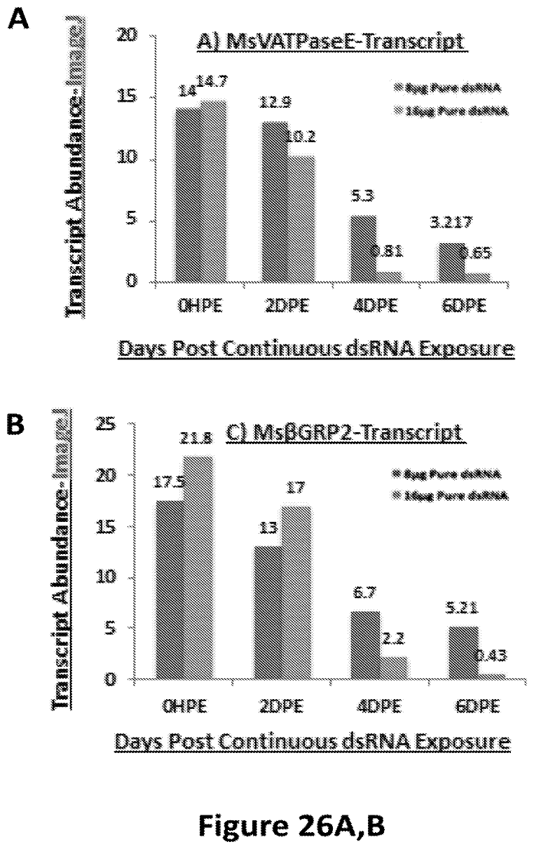

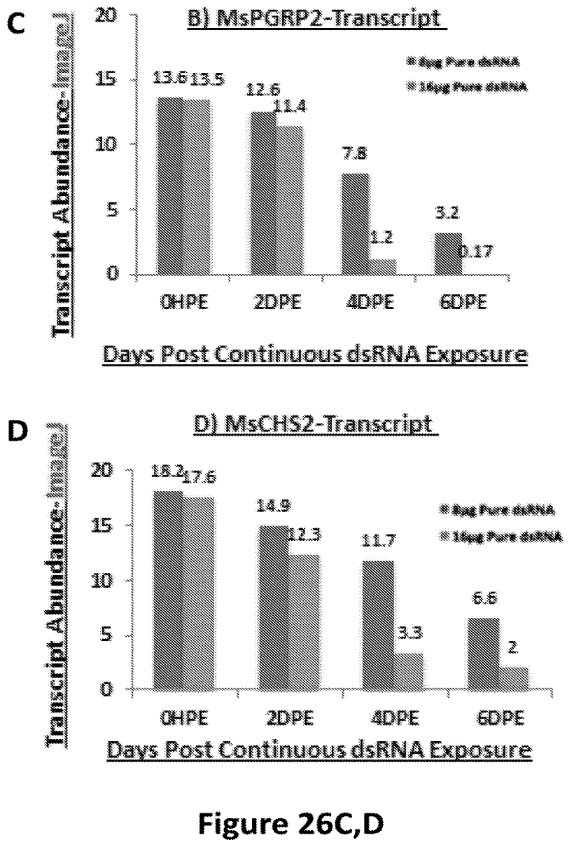

One possible reason for larval mortality could involve down regulation of MIGGS-IRTGS transcripts upon feeding on exogenously supplied dsRNA against the target genes. Preliminary RT-PCR data indicated that the larval phenotypes (FIG. 17) also correlated with down regulation of target transcripts (FIG. 26).

Importantly RNA-Seq analysis indicated that the MIGGS-IRTG pathway targets are induced by soil microbiome indicating that our novel RNAi approach could be effective even under field conditions.

Given the ease of identification, high specificity, and applicability to diverse pests and delivery platforms, RNAi silencing of the MIGGS-IRTG pathway genes identified, this approach offers an unprecedented potential as a novel pesticidal strategy.

The translation of these preliminary findings into a pesticidal RNAi technology against economically important pests might lead to sustainable alternatives including but not restricted to the methods described anywhere herein.

Additionally, the proposed approach will shed more light into understanding the tri-trophic interaction between plants-microbe-insect interactions as it pertains to sustainable insect pest protection.

### Sequences

SEQ ID NOs: 1-14 and 31-44 are representative examples of M. sexta-RNAi target gene sequences.

SEQ ID NO: 15 is non-insect gene sequence that encodes for catalase 1 from cassava (Manihot esculanta).

SEQ ID NOs: 16-29 are coding region sequences of representative M. sexta-RNAi target genes.

SEQ ID NO: 30 is the coding region sequence of catalase 1 from cassava (Manihot esculanta).

SEQ ID NOs: 45-70 are 5′UTR and 3′UTR region sequences of representative M. sexta-RNAi target genes.

SEQ ID NOs: 71-75 are the coding region sequences of additional representative M. sexta-RNAi target genes.

SEQ ID NOs: 76-88 are the coding region sequences of representative P. xylostella-RNAi target genes.

SEQ ID NOs: 89-105 are the coding region sequences of representative S. frugiperda-RNAi target genes.

SEQ ID NOs: 106-110 are the coding region sequences of representative T. castaneum-RNAi target genes.

SEQ ID NOs: 111-119 are representative examples of Manduca sexta insecticidal dsRNA sequences.

SEQ ID NOs: 120-126 are representative examples of Plutella xylostella insecticidal dsRNA sequences.

SEQ ID NOs: 127-135 are representative examples of Spodoptera frugiperda insecticidal dsRNA sequences.

SEQ ID NOs: 136-139 are representative examples of Tribolium castaneum insecticidal dsRNA sequences.

>M. sexta-Hemolin (MsHEM); M64346.1
(SEQ ID NO: 1)
ATGGTTTCAAAAAGTATCGTCGCTTTGGCTGCGTGCGTCGCAATGTGCGTAGCCCAGCCA
GTGGAGAAGATGCCTGTGCTGAAGGACCAACCCGCTGAAGTCCTCTTCCGGGAGTCTCAG
GCCACCGTTCTCGAATGTGTTACCGAGAATGGCGATAAAGATGTCAAATATTCTTGGCAA
AAAGACGGCAAAGAATTCAAATGGCAGGAACACAATATCGCCCAGCGCAAAGACGAAGGC
AGCCTGGTCTTCCTCAAGCCCGAGGCTAAAGATGAAGGCCAATACAGATGTTTCGCTGAG
TCGGCCGCCGGAGTCGCCACCTCCCACATCATCTCCTTTAGAAGGACCTACATGGTCGTA
CCTACTACTTTTAAGACTGTAGAAAAGAAACCGGTAGAAGGGTCATGGCTCAAACTTGAG
TGCAGCATCCCCGAAGGTTATCCTAAACCTACTATTGTATGGAGAAAGCAGCTTGGTGAA
GACGAAAGTATAGCAGATTCTATACTGGCACGTCGTATTACACAATCTCCAGAGGGAGAC
CTGTACTTCACGAGCGTCGAGAAAGAAGACGTAAGCGAAAGCTATAAATACGTTTGCGCT
GCTAAGTCACCGGCTATTGATGGGGATGTCCCTCTTGTTGGATACACTATTAAAAGCTTA
GAAAAGAATACAAATCAGAAAAACGGTGAGCTGGTCCCGATGTACGTCAGTAATGATATG
ATAGCTAAGGCCGGAGACGTTACTATGATCTACTGCATGTATGGTGGAGTCCCAATGGCT
TACCCCAACTGGTTCAAAGACGGTAAGGACGTGAACGGCAAACCGAGCGACCGCATCACC
CGCCACAACAGAACCTCCGGCAAAAGACTGTTCATCAAGGAGACGCTGCTCGAAGATCAG
GGCACTTTTACTTGCGACGTGAACAACGAAGTCGGCAAGCCACAGAAACATTCCGTCAAA
CTTACCGTAGTCAGTGGACCCAGATTTACGAAGAAACCAGAAAAGCAAGTCATCGCTAAG
CAGGGCCAGGACTTTGTAATCCCCTGTGAAGTATCCGCCTTACCGGCCGCCCCTGTCTCC
TGGACGTTCAACGCCAAGCCCATCAGCGGCAGCCGCGTGGTAGCCAGCCCGAGCGGACTG
ACCATCAAGGGCATCCAGAAGTCTGACAAGGGTTATTATGGCTGCCAGGCCCACAACGAG
CACGGAGATGCCTACGCTGAGACGCTCGTGATTGTTGCTTAA
>M. sexta-Serine proteinase homolog 3 (MsSPH-3); AF413067.1
(SEQ ID NO: 2)
ATGTTGTTGCTTCTGTATTGTCTTGTGGCGGCCTCCGCGCCGTTCTTTATTGCAGCGGAC
CAAGGCAGCCCTGACCTGCCTTTAGCTACCGAACCACCAACAGAATGCGGAACAATAGCA
CCTGATGATAGCTTAGTATTAGATGGGTCCGTTGGTAAAAGTGACAAATTACCTTGGTAT
GCTATTATCTACACAACCACCACCCGGCCATACAAGCAGATCGGTGGAGGAACCCTCATC
ACTCCTTCAGTAGTAATCTCAGCCGCTCACTGTTTCTGGCGCAATGGTGAGGTTCCATCT
AAGGATAATTACGCCGTGGCGCTCGGCAAGACCCATAGTGCTTGGAATAGCCATGCCGAT
GTAAACGCTCACAAGTCTGATGTAAAAGAAATACACATACCACCGCAGTTTAAGGGAAGG
AACACTAATTATCGGAATGATATAGCAATCGTGGTCATGTCAGACCCTGTGACCTACAAA
GTGGACATCCGCCCTATCTGTTTGAACTTCGATGTACAATTTGAAAGACTGCAATTAAAA
GACGGCATTATGGGGAAGATCGGCACATGGAATGTAAGTCGTGAGACACTGAAACTATCG
AAAACATTAAAAGTGGTGGAGAATCCATACATTGACGCAGCGACTTGTATTAGTGAGTCT
CCGGCAAGCTTCAGAAATTCCATCACTGCGGACAAAATATGCATCGGATACGTTAACGGC
ACCGGGCTATGTAGAGGTGATGGCGGCGCTGGGGTGGCTTTCCCTAGCCAGGAACAAGGA
GTGCAACGTTACTACCTCAGAGGTGTTATATCTACAGCCCATACCAGCGATGATGGCAAC
TTATGTGCAGATGGATTTGTAACTGCCACTGCTATAGGCCATCACGAACATTTTATCAAA
CAGTTTATAAGCGTTTAG
>M. sexta-Peptidoglycan recognition protein 2 (MsPGRP2); GQ293365.1
(SEQ ID NO: 3)
ATGGCGAGCTTCGCTTTAATAGTTATCCTTAGCGTAATTGGCTTTATATCGGCCTATCCT
AGTCCTGAAGGTTACAGTTCTGCCTTCAACTTTCCATTCGTAACCAAGGAGCAGTGGGGC
GGCAGGGAGGCACGCACGTCGACGCCACTCAACCACCCAGTGCAGTTCGTGGTGATCCAC
CACAGTTACATTCCCGGCGTGTGCCTCAGCCGGGACGAGTGCGCGCGCAGCATGCGCTCC
ATGCAGAACTTCCACATGAACAGTAACGGGTGGAGTGATATTGGATACAACTTCGCTGTC
GGCGGTGAAGGGTCGGTGTACGAGGGCCGCGGCTGGGACGCGGTCGGCGCACACGCAGCT
GGCTATAACAGTAACAGTATCGGCATCGTGCTCATCGGCGATTTTGTTTCAAACCTCCCG
CCGGCGGTGCAAATGCAAACCACACAAGAATTGATCGCAGCGGGCGTGCGACTCGGTTAC
ATCAGGCCCAACTACATGCTCATCGGGCATCGTCAGGTCTCCGCCACTGAGTGCCCAGGA
ACCAGACTCTTCAACGAAATCACCAACTGGAACAACTTCGTGAGGATATGA
>M. sexta-Beta-1, 3-glucan-recognition protein 2 (MsßGRP2); AY135522.1
(SEQ ID NO: 4)
ATGTGGATCAAGAGCGTCTGTTTGTTCGCAACCATTGCGGGCTGCTTGGGCCAGCGAGGG
GGTCCATACAAGGTGCCTGATGCGAAACTCGAAGCTATCTACCCCAAAGGCTTGAGAGTC
TCTGTGCCAGATGATGGCTACTCCCTATTTGCCTTCCACGGCAAGCTCAATGAGGAGATG
GAAGGTTTAGAGGCTGGCCATTGGTCCAGAGACATCACCAAAGCGAAGCAGGGCAGATGG
ATATTCAGAGATAGGAATGCTGAGCTGAAGCTTGGAGACAAAATTTACTTCTGGACTTAC
GTTATTAAGGATGGATTGGGATACAGGCAGGACAATGGAGAATGGACTGTTACAGAATTC
GTCAATGAGAACGGTACAGTGGTGGACACTAGTACAGCGCCGCCACCAGTAGCACCCGCC
GTTTCAGAGGAAGATCAATCGCCAGGTCCTCAGTGGAGACCTTGCGAAAGATCCCTGACT
GAGTCCTTGGCCCGCGAACGCGTTTGCAAAGGCAGCCTTGTCTTTAGCGAGGACTTTGAT
GGTTCCAGTTTGGCCGACTTGGGCAATTGGACCGCTGAAGTCAGATTCCCTGGCGAACCG
GACTACCCGTACAACTTGTACACTACGGACGGCACTGTGGGATTCGAAAGTGGGTCTCTG
GTGGTGAGACCCGTCATGACCGAGTCCAAATACCACGAGGGCATCATATACGACCGCCTC
GACCTTGAGAGATGTACAGGACAGCTGGGTACGCTGGAATGCAGGCGAGAGAGCAGCGGC
GGTCAGATTGTACCACCTGTGATGACAGCTAAACTGGCCACTCGACGCAGCTTCGCGTTC
AAGTTCGGCAGGATCGATATAAAGGCGAAGATGCCGCGCGGGGACTGGTTGATACCAGAA
CTCAACCTCGAACCTTTAGATAACATATACGGCAACCAGCGATACGCTTCGGGTCTCATG
CGGGTCGCGTTCGTGAGAGGAAACGATGTATACGCCAAGAAGCTCTACGGAGGTCCGATA
ATGTCCGACGCGGACCCGTTCAGGTCCATGCTGTTGAAGGACAAGCAAGGGTTGGCCAAC
TGGAATAATGATTACCACGTCTACTCGCTGCTGTGGAAGCCTAACGGTTTAGAGCTGATG
GTGGACGGTGAAGTGTACGGCACCATCGACGCTGGCGATGGCTTCTACCAGATTGCGAAG
AACAACCTCGTGAGCCACGCCTCGCAGTGGCTCAAGGGCACCGTCATGGCGCCGTTTGAT
GAAAAGTTCTTCATCACTCTGGGTCTTCGCGTGGCGGGTATCCACGACTTCACGGACGGT
CCGGGCAAACCTTGGGAGAACAAGGGCACCAAGGCCATGATCAACTTCTGGAACAATCGG
TTCCGCTGGTTCCCCACGTGGCACGACACCAGTCTTAAAGTCGACTACGTCAGAGTCTAT
GCTCTTTAG
>M. sexta-Relish family protein 2A (MsREL2A); HM363513.1
(SEQ ID NO: 5)
ATGTCCTCTTGTCCAAGCGACTATGATCCCAGTGAATCGTCCAAATCTCCACAAAGTATT
TGGGAGTCAGGAGGATACAGTTCTCCGTCGCAACAAGTTCCTCAATTGACTTCTAACTTA
ACAGAATTGTCTGTTGATCACAGCTATAGATACAATGGAAATGGACCATATCTACAGATC
ACAGAGCAACCACAGAAATACTTTCGGTTCCGTTATGTTAGCGAGATGGTGGGAACACAT
GGATGTTTGCTTGGCAAATCTTATACAACAAACAAAGTTAAAACTCATCCGACAGTTGAA
CTCGTGAATTACACCGGTCGAGCCCTGATAAAGTGCCAACTATCGCAAAACAAGAGCGAA
GACGAACACCCGCACAAACTGCTCGATGAACAAGACAGAGACATGAGCCACCACGTTCCC
GAGCACGGCAGTTATAGAGTGGTATTTGCTGGTATGGGTATAATTCATGCTGCCAAAAAG
GAAGTTGCGGGGTGGCTCTATAGAAAATATATACAGCAGAACAAGAATGAAAAGTTTAAT
AAGAAAGAGCTCGAAGCGCATTGTGAGAGGATGTCCAAAGAGATCGATTTAAATATAGTT
AGACTGAAGTTTAGCGCTCACGATATTGACACTGGCATTGAAATTTGCCGGCCAGTGTTC
TCTGAACCCATTTATAATTTGAAGTGTGCGTCTACGAATGATTTGAAAATATGCCGCATA
AGCCGTTGTTACGGTAGACCGAGAGGCGGCGAAGATATCTTCATATTTGTCGAAAAGGTC
AACAAGAAAAACATCCAAGTTCGGTTCTTTAGACTGGAAAACGGGGAGCGCACCTGGTCA
GCGATGGCGAACTTTCTGCTAAGCGATGTTCACCACCAATACGCTATCGCTTTTAGAACG
CCACCGTACGTCAATCACCAAATTTCTGAAGACGTGCAAGTTTTTATAGAACTCGTACGC
CCTTCAGACGGTAGGACGAGCGCTCCCATGGAGTTCACATACAAGGCTGAGCAAATCTAT
AAACAGAACAAGAAACGTAAAACTACTTCGTCGTACTCGTCGCTCGACAGCTCCTCAGGT
TCGGCCGGTTCAATTAAAAGCATCAGCGAACTGCCCGCGCCCGTTGTTTTTGCTGAAAAC
GTAAGTTTTTTCTATGACACATTACTCATTCTTCAACCCATGACGAATCTATAA
>M. sexta-Dorsal (MsDor); HM363515.1
(SEQ ID NO: 6)
ATGCTTGTGACGTTATGCGGCGGGAACTATAGTGGATTGTCGTTAACAAAAACTAATCAT
TATATGTCACCAAAATCATATGTGCCAGGAAATGGTTATGACGCCGCCGTAATCCTAGGT
ACCACGGAGCAGAATGACAGCGAACCCTCAAACTTGAATATTAGTGATGTTTTTGAAGCC
ATCACGCTCGCTGATCCGTCGTTCGGCGCGGGCGTGCCGTCGGTAGAGGAGACGATGGCG
CACACGCAGCCCCAGCCGCTGCAAATGCCGTACGTGGTCGTCGTGCAGCAGCCCGCCAGC
AAAGCGCTCAGATTTCGATATGAGTGCGAGGGCAGATCAGCCGGTTCGATTCCCGGCGCG
TCGAGCACGCCCGAGAACCGAACCTTCCCCGCCATCAAGATAATCGGCTACACCGGCACC
GTCTCCATCGTAGTGTCGTGTGTCACCAAAGATGAGCCTTGCAGGCCGCACCCACACAAC
CTGGTCGGGCGCGACCACTGCGACCGCGGCGTGTTCTCCGTCCGCATCGAGATCACCGAC
GAGAATAACGAATACCAGTTTCGGAACCTGGGCATACAGTGCGTCAAGCGGCGCGACATC
GGCGAGGCGCTGCGGATCCGAGAGGACCTGCGCGTCGATCCGTTCAAAACCGGCTTCACC
CACCGGAACCACCCGCAAGGCATCGATCTGAATGCAGTGCGGCTCGCGTTCCAAGTGTTC
CTGCCGCACTCCAGCGGCAAGATGCGGCGCACGCTCGCGCCCGTCGTGTCCGACGTCATC
TACGACAAGAAGGCCATGAGCGACCTGCTCATCGTGCGCGCGAGCCACTGCGCCGGCCCG
GCGCGCGGCGGCACGCAGGTCGTACTGCTCTGTGAAAAGGTGACTCGCGAGGACACCGTG
GTGGTGTTCTACCAGGAGGACAACAACCGCGTGCTGTGGGAGGAGATGGCGATCATCATC
GTGGTGCACAAACAGTATGCCATAGCGTTCGAGACGCCGCCATACAAGAACCCAAACATT
ACTGATAATGTCAATGTACGATTCCAGCTGAGAAGGCTCTCCGACAAGATGACGAGCAAC
TCGCTGCCGTTCGAGTACATTCCCGAATACCAAGATTACCCTAGTTACAGGCAGGATAAC
TCAGAAAGAAATCCCCAATCGCAGCCAATTACGCACAAGGTAACGGTGGAGGACTTTGAA
TCGACAACTAAAAGATATTTTACGCGAAGCACTGACAACAGTAATTACGGTTGGGATGCG
GTTCCGGTCACGTACAATGGAAGAAAGAAGGTTTGCTATTGCCCCAAGAGGAGCTAA
>M. sexta-Spatzle (MsSPZ1A); GQ249944.1
(SEQ ID NO: 7)
ATGGCCTGGATCCAGCATTTACTCGTTTGGCTCTTCGTTATGTCAACATCAGCATACAAA
TGCAAAGACTGCTTCAGTTTCGCATCACAATATCCGTCGTACGATAGTCAAGTATACGAA
CAACCTGACAGACGGATAGCGGGACGGTCAGCACAATACGAACATTTAAGAACAAACGAG
AGGTCTCTCCCGGTCTACAGCGAGACCCAGAGGATACAAGCAGAAGAGAGAAGAAGACAC
AGTTCGAGACTAGAAGAACCGAGACAACGTGCTGAGAATGGTTCATATAAGATATTGAAT
AACCCTCCGAAACCCTGTATTACTAATAGGAGAAGTCAAATTGATTCGTCGAATGATAGG
GTAGTGTTCCCCGGTCCGACTTCAGAAAGGTCGTACGTACCCGAAGTGCCAGAGGAATGC
AAGAAAATCGGCATATGCGACAGTATACCGAATTACCCAGAAGAACACGTAGCTAATATT
ATATCTCGACTTGGAGACAAAGGAAAAGTATTACAAATAGACGAACTGGACGTATCAGAC
ACTCCAGATATCGCCCAGAGGTTGGGTCCGCAGGAGGACAACATGGAACTATGTAGCTTT
AGAGAAAAGATTTTTTACCCCAAGGCAGCGCCAGACAAAGATGGAAATTGGTTCTTCGTT
GTGAATTCAAAAGAAAACCCAGTACAGGGTTATAAAGTTGAAATTTGCGACCGTCAGCAA
TTACCATGCGCGGAGTTCGCGAGCTTCCAACAGGGATATGAAGCGAGGTGCATCCAGAAA
TACGTTCGCCGGACCATGTTGGCGTTGGATCCCAAGGGTCAGATGACCGACATGCCCCTT
AAAGTGCCCAGCTGTTGCTCATGCGTGGCCAAATTGACAATCATATGA
>M. sexta-Toll receptor (MsTOLL); EF442782.1
(SEQ ID NO: 8)
ATGCAGGCTCGGCGGTGGTGCGCGGCACTGCTATTAATGCAGATGCTGAGCTGGCTCGGA
GTCAGTGGACACTTACCGCGTCCCGAGTGCGCGCCAGCCGCAGATTGCCAACTTATACGA
GACAACATAATCGATGGATATGCACAATTCTACTTCAACGTATCAGGACATGAAGTGAAA
TTTGAACATTACATCGGAAACGACTTCGATGTCGAATTGTCATGCAATTACATCGCCATG
GACAACGCAATGCTGCCGCGGTTCTCAACGACCTTTTCAGTCAACGTAATAGTGGTTAAA
GAATGTGCTTTGCCAAGAAGTGGGTCAATCGATGCCGCTGTCGCTGCACTTAATATCAAC
GTTTTGACGGAGCTGACTCTGGACAAATTCCTAGAGCCGGCGGTGATCACGCGCGCACAT
CTTACCAGTTTACAACGACTAGAGAGGCTGGAGCTACACGGTAACTCAAACACAAGCCTC
GCCCCCGGCGCACTGGCCGCGCTCTCCGCCGCGTCCGCACTGAAATGTCTTGTATTGCAT
GCAGTACGCGTGCCCGCCGCTGACCTGGCGCGCTTGCCGTCGTCACTGCAAGAACTAGCG
TTGTTGGATGTGGGCGCTGCGAGTATGCATTTAGATTCATCGGTTAATTTGACGTCACTC
TTCGTAATCGATACACATTATCCTGTCGTCGTGAATGTGAGCAACGCCGTTGCGCTCAGA
GACTTGCACATAAATACCCCAAGTACTGTGTTGACCGAAGACGTGCTCCCGTCGTCACTC
AACTCACTTGAACTAGAGGGGTGGAACGAAACGCATCCGGTGCCTAAGACACGTTGTGTA
CTACTTAAGGAACTTAATGTAATCGGCACCGACAATGATGCCTATCCGGTGACTCTCCCG
GACGAGTGGCTGTCCAACTGCGGACAGCTGAGGGATCTTGAAATGATCTCCGTGCCGATT
AGCGCCGTACTTCCGGCGCGGATGCTGGCTAACGCAATTAGGCTTGAAACGATTACTATC
TGGAACTGTAACCTGACCGCGTTGCCGTCGGGCCTGTTAGACGACACGCTGAACCTCGCC
ACACTCGACTTGTCCAACAATCAACTTGCATCGTTGCCCAGAAAATTATTTGAGCACACG
AAGTTACTACGCAGTCTCATACTATCGAACAATCAGCTGACGAGCGAGGTAGTGTGGACG
CTGTCGACCGTCACCTCGCTTGTTGAACTAAAGCTCAGCAATAATAACCGCATAGGCGAC
TTATGTTCCCACGACTCAGTGTCAGCAGGTCCCTCACCACTGAGTTCACTGACGGGGCTA
AACTATCTCCATCTGAGCAACACAGGAGTATCACACGTGTGCTCCGACTGGCGAGAAAAG
CTAACCTACCTCACGAATCTCAACTTAAGGGACAACCCCATCACTCTCTACAATTTGGCG
GATCTACAGTTTCGTCGAATCTGGGTGGACGCTCGTGTGTATTTAGGACATTTCAAGCAG
CAGTTTACGCGCACAGATTACGAACTCGCCAGTAATAATAACACTGAGGCGGTCGTAACT
TTATCCGGTTCGTTAGAATGCGACTGCAATTCATACTGGGCAGCACAGGTGTTCCGTATG
AAAGCTTGGCAGGCATCCAGCTCCATGATATATTGTGAAAAAAAACCGGTCATTGAGGTG
GATCCCGACACCTTTACCTGCCTTGAGCCAGCGAAGTGTGCTGCGCTGGCGGATAGTTGT
ACGTGCCGTATCCGCGACGACATTCAGTACAAACAAGTGGTCGTTGTGCACTGCACCGGA
CTCGCCGAGTTCCCGCGCCTGCCACTAACCACGGACAAGTGGATCCTGCACCTGCCCCAC
AACAACATATCATACCTCGCCGCGGCCGACGTATCGCCGAACATCGTGGAACTCGATCTA
AGAAACAATTCAATCAAGAATATCGACGTACAGGCATCAGCAAAACTAGCCTTCGTCCGG
CTGCAATTGGGTGGTAACCCGATCGAGTGTGACTGCGAGGCGTTGAAGCTGCTGGCGCCG
CTGCTCAAACCTGACTCAAAGCTGCTTGACAGAAAGGACGTGAAATGTGAGAACGACGCG
CAGATTACCTTGGCGATGCTGAAATTATGTACTAAGTCATCCAATGGGCTGATGTACTTG
CTGTTCCTGTTGCTGTTACTCGCATTTGTCGTAACCGGACTGCTCGCACGAACCGCAATT
CGCCTGCGCATCAAAATGATCCTCATGAGACTGGGTTGGATGTCGAGACTACTGGAGCCC
GCGGACGACGATCGCCCGTACGACGCGTTTGTGTCTTTCGCACACGAGGATGAGGAGCTG
GTGATGGAGCAGCTGGCGGCACGGCTCGAGAGCGGCTCGCGGCCGTACCGACTGTGTCTG
CACTACCGCGATTGGGCGCCAGGCGAGTGGATCCCGGCGCAAATAGCGGCTTCGGTGCGG
GCCTCTCGGCGCACGGTGGCGGTTGTGTCGGCGCACTACTTACAGTCGGGCTGGGCGCTT
GCCGAGATCCGGGAGGCGACCGCAGCCTCGCTGCAGGAAGGCATGCCACGTCTCATCATC
GTGCTGCTCGACGAGACCGACCGGTTGATGCTCGATATAGACCCTGAGTTGCACGCCTAT
GTGCGCAACAATACCTACGTGCGCTGGCATGATCCATGGTTTTGGGAGAAGCTGAAGCAG
GCGCTGCCTCCACCGCGGGAACAACGGTCGCCAATAGCTCCGTCGCTACCAGCGCTGGCG
CTGTCCCATGACAGCCTAACTCTGCGCACGTACTCTCCCAGAGAAAGTGACCCAGCGCCC
GCTGCTAAGCCGGCGCACACTCCGCACGAGACGGACGAGCCAGCGCCGGGCGCGAGTCCT
TGCTACAAATGA
>M. sexta-Scolexin A (MsSCA1); AF087004.1
(SEQ ID NO: 9)
CGGCAGTCGGTTGTGTTGGCAGTGGCGGCGGTGCTCTTCGGGTGCGCGTGCGCAGCGCCC
AATCCTGGCGCCAACGACATACAACTTAATCAAAAATTAAGTATCGAAGCTAAGGGGGCA
AAGCAGCCAATTGATACGAGGGCAGTGAAGGAACGGTATCCATACGCAGTTCGGAGTTTC
GGAGGCTTCTGCGGAGGAACCATTATCAGTCCCACCTGGATCCTGACCGCCGGCCACTGC
TCGATACTCTATGCGGGGAGCGGCCTACCGGCCGGCACCAACATTACCGAGGTATCTAGC
TTGTACCGCTTCCCCAAGCGGCTCGTCATACACCCGCTCTTCTCCATAGGACCCGTCTGG
CTCAACGCTACGGAGTTCAACCTCAAACAGGCGGCTGCACGATGGGACTTCTTGTTGATA
GAACTGGAGGAACCGCTGCCGTTGGACGGCAAGATCCTGGCGGCTGCGAAGCTCGACGAC
CAGCCCGACCTCCCCGCAGGCCTCGACGTGGGCTATCCGAGCTACAGCACCGACACCTAC
GAGGCTAAGATACAAAGCGAGATGCACGGAAAGAAGCTTTCGGTTCAATCTAACGAGGTG
TGCTCGAAGCTAGAGCAGTTCAAGGCGGAGGACATGTTGTGCGCCAAGGGACGTCCACCG
CGATACGACTTCGTCTGCTTCAGCGACAGTGGCAGTGGGCTAGTAGACAACAATGGTCGC
CTAGTCGGCGTGGTGTCGTGGGCCGAGAACAACGCTTTCGAGTGCCGCAACGGCAACCTG
GCGGTCTTCTCGCGAGTGTCCAGCGTACGCGAGTGGATCCGACAAGTCACCAACATATAA
>M. sexta-Hemolymph proteinase 18 (MsHP18); AY672794.1
(SEQ ID NO: 10)
ATGGTTTATATTTTAATAATTTTAGTGATTTGCAATTTTAGTTGTATTAGTTGTCAGTCC
GGGACAGTGGAAAGCAGGATTCATTTTAAAGATGAAGGGCCGGAATGTTATGATGCAAAT
AAAAAGGGCACCTGTGTTAGTGCTCACAGATGCCTTGATGTAGTTAGAAAACTTAAAGAC
GGAGAGAAACCCACGATATGTGGCTACCAAGGCACGGAACCAATGGTGTGTTGCACAGAC
TGTACTCTGGTTGATAATATTAGTAATTTGGTCGTAAGTTCCATATCCGGGTACCTGTGG
AAGGATGGTCAGAAAGCGTGGGACAAATGTCTGGAATACGTTGACAAGCTGTCGTACCCA
TGCGCTTCAACCTACTCCCACTACCTCAGCTCCGTTTGGGAGAAAGATAAGGAGTGCAGT
ATGGTTCAGTTTGTTGGCGTGAGGCGATTCGCCTCGTATAACGGACAACCGGCGAAACGG
AACGAGTACCCTCACATGGCTCTGCTCGGCTACGGCGACGACCAGGAGACGGCGCAGTGG
CTCTGCGGCGGCTCAGTGATCAGTGATCAATTCATCCTCACGGCTGCACACTGCATCTTT
ACAAATCTATTGGGTCCAGTACGTTTCGCAGCGCTAGGAATACTGCAGCGATCGGATCCA
GTAGAGTTATGGCAAGTTTACAAGATCGGCGGCATAGTTCCCCATCCGCAGTATAAGTCA
CCTATCAAGTACCACGACATTGCTCTCCTGAAGACTGAAAACAAAATAAAGTTTAATGAG
AACGTGCTGCCAGCGTGTTTGTTCATAGAGGGCAGAGTGGGTGGGAGTGAGCAGGCTAAA
GCGACCGGTTGGGGCGCGCTTGGACATAAACAGACGGCAGCTGACGTACTGCAAGTGGTT
GACCTTCAAAAGTTCAGTGACGAAGAGTGCGGAAGTACCTACCGTCCTTACCGGCATTTG
CCTCAAGGCTACGACAGCGCCACGCAGATGTGCTACGGCGACAAGGGAAAACTGAATATG
GACACCTGTGAGGGCGACAGCGGCGGTCCTCTACAGTTCCAAAACTCCTCGCTCCTCTGC
ATACACATAGTAGCGGGAGTGACGTCATTCGGCGACGCGTGCGGGTTTGCGGGCGGCGCC
GGGATGTACACACGAGTGTCGTACTATATTCCCTGGATCGAGAGCGTTGTATGGCCGTGA
>M. sexta-Transferrin (MsTRN); M62802.1
(SEQ ID NO: 11)
ATGGCTTTGAAACTTTTAACTTTGATAGCCCTGACTTGTGCGGCTGCGAATGCAGCTAAA
TCTTCATACAAACTATGCGTGCCAGCAGCATACATGAAGGACTGCGAGCAGATGCTTGAA
GTACCCACGAAGTCTAAAGTGGCCTTGGAATGTGTACCGGCTAGAGACAGGGTGGAATGC
CTCAGCTTTGTTCAGCAGCGACAGGCGGACTTCGTCCCCGTCGACCCTGAGGACATGTAC
GTGGCCTCCAAGATCCCCAACCAGGACTTCGTCGTCTTCCAGGAGTACAGGACTGATGAA
GAGCCTGATGCGCCATTCCGTTATGAAGCCGTTATTGTGGTTCACAAAGACCTACCCATC
AACAACTTGGATCAGCTGAAGGGACTGAGGTCTTGCCACACCGGAGTCAATCGTAACGTC
GGGTACAAGATCCCACTAACGATGTTGATGAAACGTGCCGTGTTCCCGAAAATGAACGAC
CACAGCATTTCGCCGAAAGAGAACGAACTGAAAGCGCTATCGACGTTCTTCGCAAAGTCG
TGCATCGTCGGCAAATGGTCGCCTGACCCCAAAACCAACTCGGCTTGGAAATCACAATAC
AGCCATTTGTGTTCAATGTGCGAACACCCGGAGCGTTGTGACTATCCCGACAATTACAGC
GGGTACGAGGGCGCGTTGAGATGCCTCGCCCACAACAACGGGGAGGTCGCGTTCACCAAA
GTCATATTCACACGTAAATTCTTTGGGCTTCCAGTAGGTACCACTCCAGCGAGTCCATCA
AACGAAAATCCCGAAGAGTTCAGATATCTCTGCGTGGACGGATCTAAAGCCCCCATCACT
GGCAAGGCTTGTTCATGGGCTGCCAGACCTTGGCAAGGACTGATCGGTCACAATGACGTA
CTTGCCAAACTCGCTCCGCTCAGAGAGAAGGTTAAGCAACTTGCTGATTCTGGTGCAGCT
GACAAACCGGAGTGGTTCACCAAAGTCCTTGGTCTATCAGAGAAGATCCACCATGTCGCT
GACAATATCCCAATCAAGCCCATCGACTACCTGAACAAGGCTAACTACACGGAGGTCATT
GAAAGAGGACATGGAGCTCCCGAGCTGGTCGTCAGGCTATGTGTGACGTCAAACGTGGCA
TTATCTAAGTGCCGGGCTATGTCCGTGTTCGCATTCAGTAGAGACATCAGGCCGATCCTA
GACTGTGTTCAAGAAAACAGCGAAGATGCCTGTCTTAAGAGCGTCCAAGACAACGGTTCA
GATCTTGCCTCAGTAGACGATATGAGAGTAGCTGCAGCGGCTAAGAAGTACAACTTACAT
CCAGTTTTCCACGAAGTGTATGGAGAGCTAAAGACGCCCAACTACGCAGTGGCTGTTGTC
AAGAAGGGCACTGCCTACAACAAGATCGACGACTTAAGGGGAAAGAAATCTTGCCACAGC
TCTTACAGTACTTTCAGCGGTCTGCACGCGCCTCTCTTCTACCTTATTAACAAGAGGGCC
ATTCAATCTGACCACTGCGTGAAGAACTTGGGAGAATTCTTCTCAGGCGGATCTTGCTTG
CCTGGTGTCGACAAACCCGAAAACAACCCAAGCGGTGATGATGTGTCTAAATTGAAGAAG
CAATGTGGATCCGACAGCAGCGCTTGGAAGTGCTTGGAAGAGGACAGAGGAGACGTCGCA
TTTGTTTCAAGTGCCGATCTGTCCCACTTCGACGCCAACCAATACGAGCTGCTCTGCCTG
AACCGCGACGCTGGCGGTAGAGATGTTCTCTCCAGTTTCGCCACTTGCAACGTCGCCATG
GCCCCGTCCAGGACCTGGGTGGCTGCGAAGGACTTCCTGTCTGACGTATCTATCGCCCAC
ACACCATTGAGCCTCGCCCAAATGCTCGCTACGAGACCTGACCTCTTCAACATTTACGGA
GAGTTCTTGAAGAACAACAATGTTATTTTCAATAATGCCGCTAAAGGCTTAGCAACAACT
GAGAAACTTGACTTCGAGAAGTTCAAGACCATCCACGACGTCATCTCTTCATGTGGTCTC
GCCTAA
>M. sexta-Arylphorin ß subunit (MsARP); M28397.1
(SEQ ID NO: 12)
ATGAAGACTGTCATAATCCTAGCGGGGTTGGTGGCCCTGGCCCTCGGCAGCGAAGTGCCT
GTCAAGCACTCCTTCAAAGTTAAGGATGTTGATGCGGCTTTCGTCGAACGTCAAAAGAAG
GTCTTAGATCTTTTCCAAGATGTCGACCAAGTAAATCCTAACGATGAGTACTACAAGATT
GGCAAGGAATACAACATCGAGGCTAACATCGACAATTACTCGAACAAGAAGGCCGTCGAA
GAATTCTTGCAGTTATACAGGACAGGTTTCTTGCCTAAGTACTATGAATTTTCACCCTTC
TATGACAGACTAAGGGACGAGGCCATTGGTGTTTTCCACCTCTTTTACTACGCTAAAGAT
TTTGATACGTTCTACAAATCTGCCGCATGGGCGCGTGTGTACCTCAACGAAGGACAGTTC
TTATACGCCTACTACATTGCTGTGATTCAGCGTAAAGATACTCAGGGCTTCGTTGTACCA
GCACCGTATGAAGTCTACCCTCAATTCTTCGCAAACTTGAACACTATGCTCAAAGTCTAC
CGTACCAAAATGCAGGATGGAGTTGTTAGTGCCGATTTAGCTGCACAACACGGCATCGTA
AAGGAGAAAAACTACTACGTATACTATGCCAATTACTCCAACTCATTAGTGTACAACAAC
GAGGAACAGAGACTGTCGTACTTCACTGAGGACATCGGCTTGAATTCGTACTACTACTAC
TTCCACTCTCACTTGCCTTTCTGGTGGAATTCTGAGAGATACGGAGCACTAAAATCGCGC
CGTGGTGAAATCTACTATTACTTCTATCAGCAATTAATTGCACGTTATTACTTTGAACGT
CTCTCGAACGGCCTGGGTGACATTCCCGAATTCTCATGGTACTCACCAGTCAAGTCTGGC
TACTATCCACTGATGTCTTCTTATTACTACCCCTTCGCTCAAAGGCCCAACTACTGGAAC
GTGCACAGCGAAGAAAACTACGAGAAAGTACGATTCTTGGACACGTATGAAATGTCATTC
CTTCAGTTCCTCCAAAACGGACACTTCAAAGCGTTTGACCAGAAGATTGACTTCCACGAT
TTCAAAGCTATCAACTTTGTTGGAAACTACTGGCAAGATAATGCTGACCTGTACGGTGAG
GAAGTTACTAAGGACTACCAACGTTCATATGAAATTATAGCCCGCCAAGTGCTTGGTGCT
GCACCTAAACCATTCGACAAGTACACATTCATGCCCAGCGCTTTAGACTTCTACCAGACG
TCTCTGCGTGACCCAATGTTCTACCAACTTTACAACAGAATTCTGAAGTACATATATGAG
TACAAGCAGTACCTGCAACCGTACTCTTCAGAAAAACTGGCATTCAAGGGTGTCAAGGTG
GTCGATGTTGTAGTAGACAAACTGGTTACCTTCTTCGAGTACTACGACTTTGATGCGTCC
AACAGCGTTTTCTGGAGCAAAGAGGAGGTTAAATCTAGCTACCCCCATGATTTCAAGATC
CGTCAGCCACGCCTTAACCACAAGCCATTCTCTGTCTCTATCGACATCAAATCTGAAGCT
GCCGTTGATGCCGTTGTCAAGATATTCATGGCACCTAAATACGACGATAATGGATTCCCT
CTGAAATTAGAAAACAACTGGAACAAATTCTTCGAGCTGGACTGGTTCACATACAAATTT
GTTGCTGGTGACAACAAAATCGTGAGGAACTCAAACGACTTCTTGATCTTCAAGGACGAC
TCTGTTCCCATGACTGAGTTGTACAAATTATTAGAACAAAATAAGGTTCCACACGACATG
TCCGAGGATTACGGCTACCTGCCTAAAAGACTGATGCTGCCAAGAGGTACTGAGGGTGGT
TTCCCATTCCAGTTCTTCGTTTTCGTATATCCATTCAACGCTGACAGCAAAGATCTTGCA
CCGTTCGAGGCCTTCATCCAGGACAACAAACCTTTGGGCTATCCATTCGACCGTCCCGTT
GTTGACGCTTACTTCAAGCAACACAACATGTTCTTCAAGGACGTCTTCGTATACCATGAC
GGCGAGTACTTCCCGTACAAGTTCAATGTTCCTTCCCATGTGATGCACTCAAACGTTGTT
CCTAAACACTGA
>M. sexta-Chymotrypsinogen-like protein 1 (MsCTL1); AM419170.1
(SEQ ID NO: 13)
ATGTACGTGAAAGTAGCACTTCTGTTGGTAGCCCTCATTGCTGGGAGCTGGGCCTTCCCA
AAGCTCGAAGATGAGCAGGACATGTCCATCTTCTTCACGCAGCTCGATTCGAGCGCGCGT
ATCGTGGGTGGTACCCAGGCCCCCAGCGGAAGTCACCCTCACATGGTGGCGATGACCACC
GGTACCTTCATCAGGAGCTTCAGCTGTGGAGGCTCAGTTGTCGGTAGACGTTCCGTTCTG
ACTGCGGCTCATTGCATCGCTGCTGTTTTCAGTTTCGGTTCCCTCGCCAGTACCCTCCGC
TTGACGGTCGGCACCAACTTCTGGAACCAGGGAGGCACCATGTACACCGTCGCTCGCAAC
ATAACCCACCCCCACTACGTCTCTGCGACCATCAAGAACGACATCGGTCTGTTCATCACT
CACAACAACATCATCGACACGACTGTCGTCCGCAGCATCCCTCTTAACTTTGACTATGTG
CCCGGTGGTGTTCTCACTAGAGTCGCCGGATGGGGCAGGATCAGGACCGGCGGTGCCATC
TCTCCCTCTCTGCTGGAGATCATTGTGCCTACTATCAGTGGAAGCGCATGCGTAGCCAGT
GCAATCCAAGCTGGCATCGATCTGAACATGAGACCACCTCCCGTCGAGCCTCACATCGAG
CTGTGCACCTTCCACGGTCCTAACGTAGGCACTTGTAATGGTGACTCCGGCAGCGCTCTT
GCCCGCCTAGACAACGGCCAGCAGATCGGTGTGGTATCGTGGGGCTTCCCGTGCGCACGC
GGCGGTCCCGACATGTTCGTCAGGGTCAGCGCCTACCAATCCTGGCTGCAGCAGAGCATC
GTATAA
>M. sexta-Valine Rich Midgut Protein (MsVMP1); NCBI accession number not assigned
as yet
(SEQ ID NO: 14)
ATCATTGACGGACCTTCCGTTGGACCNGCCATCATCGGCGCTGGAGACATCGCTGTCGGC
CCTGCTATCGTCGACTTCCCTTTCCCCGACGGCGGTGCCGTGTCTGCCCCCGTTGAGCCT
TCCCCCATCGCCATCGGACCCGCTATCGTCGGTGAATCCCCTATCTCCGTCGGACCTGCC
ATCGTTGAGGCCGGAGACATCGCTGTTGGACCCGCTATCATCGACTTCCCCCTTCCCGAC
GGTGGCGCCGTGTCCGCCCCCGTTGAGGTTTCTCCCGTCGACTCCGTCGTCGTCGGCCCT
GCCGCCGGCTCTCAGAGCTCTCCCCTCGTCCAGATCATCATCAACGTTAAGGCCCCCGCT
GGTGCCGGCCCCGTTGTCGATGCCGTCGCTGACAAGCCCATGGACATCATTGATGTTATG
CCCGTCGTCGACCCTGCTGATTTCGTGGACCTCACCCCCGTTGTAGAGCCTGTAGAAGTC
GTCGACATTGTCGATGTCATGCCCGTGGTTGACCCCATCAACATCATCGATGTTATGCCT
GTTGTTAAGCCCGTAAACCCCCTTGCCCGTTCTTAAGGG
>M. esculanta-Catalase 1 (MeCAT1); AF170272
(SEQ ID NO: 15)
ATGGATCCTTGCAAGTTCCGTCCATCAAGCTCAAACAATACCCCCTTCTGGACCACCGAT
GCTGGTGCTCCAGTATGGAACAACAATTCCTCCATGACTGTTGGAACCAGAGGTCCAATC
CTTTTGGAGGACTATCATATGATAGAGAAACTTGCCAACTTTACCAGAGAGAGGATTCCA
GAGCGTGTCGTCCATGCTAGGGGAATGAGTGCAAAGGGCTTCTTTGAAGTCACCCACGAT
GTCTCTCACCTTACTTGTGCTGATTTCCTTCGAGCCCCTGGAGTTCAAACCCCTGTCATC
GTCCGTTTCTCCACTGTTATCCACGAGCGTGGCAGCCCTGAAACACTCAGGGATCCTCGA
GGTTTTGCGACTAAGTTCTACACCAGAGAGGGCAACTTTGATATTGTGGGAAACAACTTC
CCTGTCTTCTTCATCCGTGATGGAATAAAATTCCCAGATGTGATACACGCTTTTAAGCCC
AATCCCAAGTCTCACATCCAAGAATACTGGAGGATCTTTGACTTCTTATCACACCATCCT
GAGAGCTTGAGCACCTTCGCCTGGTTCTTCGATGATGTTGGAATTCCCCAAGATTACAGA
CACATGGAAGGTTTCGGTGTTCACACCTTTACTTTCATCAACAAGGCTGGAAAAGTAACC
TACGTGAAATTTCACTGGAAACCCACTTGCGGGGTCAAGTGTTTGATGGATGATGAGGCA
CTTAAGATCGGAGGTGCCAACCACAGCCATGCTACGCAGGATTTATACGACTCCATTGCC
GCTGGCAACTATCCTGAGTGGAGACTCTTCATCCAGACAATGGATCCAGCTGATGAAGAC
AAATTCGACTTTGATCCACTTGATATGACCAAGATCTGGCCTGAGGATATTTTTCCTCTA
CAGCAAATTGGCCGTTTGGTCTTGAACAGGAACATCGATAACTGGTTTGCTGAGAATGAA
ATGCTCGCATTCGACCCTGGTCATATTGTTCCTGGCATTCACTATTCAAACGACAAGTTG
TTTCAGCTCAGAACCTTTGCATATGCTGACACTCAGAGGCACCGTCTCGGACCCAACTAT
AAGATGCTCCCTGTTAATGCTCCCAAGTGTGCTTATCACAACAATCATTACGATGGTTTC
ATGAATTTCATGCACAGGGATGAGGAGGTGGATTACTTCCCATCCAGGTATGATCCAGTT
CGCCATGCTGAGAGAAGCCCCATTCCTAACGCTATCTGTAGTGGAAGGCGTGAAAAGTGC
GTCATTGAAAAGGAGAACAATTTCAAGCAACCTGGAGAGAGATATCGATCCTGGGCACCT
GATAGACAAGAAAGATTCCTGTGCAGATTGGTTAACGCCTTATCAGAGCCACGTATCACC
TTTGAGATTCGCAGTATCTGGGTCTCTTACTGGTCTAAGTGCGACGCGTCTCTGGGTCAA
AAGCTGGCTTCTCGTCTCAACGTGAGGCCAAATATATGA
>M. sexta-Hemolin (MsHEM); M64346.1
(SEQ ID NO: 16)
GGCAAAGAATTCAAATGGCAGGAACACAATATCGCCCAGCGCAAAGACGAAGGCAGCCTG
GTCTTCCTCAAGCCCGAGGCTAAAGATGAAGGCCAATACAGATGTTTCGCTGAGTCGGCC
GCCGGAGTCGCCACCTCCCACATCATCTCCTTTAGAAGGACCTACATGGTCGTACCTACT
ACTTTTAAGACTGTAGAAAAGAAACCGGTAGAAGGGTCATGGCTCAAACTTGAGTGCAGC
ATCCCCGAAGGTTATCCTAAACCTACTATTGTATGGAGAAAGCAGCTTGGTGAAGACGAA
AGTATAGCAGATTCTATACTGGCACGTCGTATTACACAATCTCCAGAGGGAGACCTGTAC
TTCACGAGCGTCGAGAAAGAAGACGTAAGCGAAAGCTATAAATACGTTTGCGCTGCTAAG
TCACCGGCTATTGATGGGGATGTCCCTCTTGTTGGATACACTATTAAAAGCTTAGAAAAG
AATACAAATCAGAAAAACGGTGAGCTGGTCCCGATGTACGTCAGTAATGATATGATAGCT
AAGGCCGGAGACGTTACTATGATCTACTGCATGTATGGTGGAGTCCCAATGGCTTACCCC
AACTGGTTCAA
>M. sexta-Serine Proteinase homolog 3 (MsSPH-3); AF413067.1
(SEQ ID NO: 17)
ATGTTGTTGCTTCTGTATTGTCTTGTGGCGGCCTCCGCGCCGTTCTTTATTGCAGCGGAC
CAAGGCAGCCCTGACCTGCCTTTAGCTACCGAACCACCAACAGAATGCGGAACAATAGCA
CCTGATGATAGCTTAGTATTAGATGGGTCCGTTGGTAAAAGTGACAAATTACCTTGGTAT
GCTATTATCTACACAACCACCACCCGGCCATACAAGCAGATCGGTGGAGGAACCCTCATC
ACTCCTTCAGTAGTAATCTCAGCCGCTCACTGTTTCTGGCGCAATGGTGAGGTTCCATCT
AAGGATAATTACGCCGTGGCGCTCGGCAAGACCCATAGTGCTTGGAATAGCCATGCCGAT
GTAAACGCTCACAAGTCTGATGTAAAAGAAATACACATACCACCGCAGTTTAAGGGAAGG
AACACTAATTATCGGAATGATATAGCAATCGTGGTCATGTCAGACCCTGTGACCTACAAA
GTGGACATCCGCCCTATCTGTTTGAACTTCGATGTACAATTTGAAAGACTGCAATTAAAA
GACGGCATTATGGGGAAGATCGGCACATGGAATGTAAGTCGTGAGACACTGAAACTATCG
AAAACATTAAAAGTGGTGGAGAATCCATACATTGACGCAGCGACT
>M. sexta-Peptidoglycan recognition protein 2 (MsPGRP2); GQ293365.1
(SEQ ID NO: 18)
ATGGCGAGCTTCGCTTTAATAGTTATCCTTAGCGTAATTGGCTTTATATCGGCCTATCCT
AGTCCTGAAGGTTACAGTTCTGCCTTCAACTTTCCATTCGTAACCAAGGAGCAGTGGGGC
GGCAGGGAGGCACGCACGTCGACGCCACTCAACCACCCAGTGCAGTTCGTGGTGATCCAC
CACAGTTACATTCCCGGCGTGTGCCTCAGCCGGGACGAGTGCGCGCGCAGCATGCGCTCC
ATGCAGAACTTCCACATGAACAGTAACGGGTGGAGTGATATTGGATACAACTTCGCTGTC
GGCGGTGAAGGGTCGGTGTACGAGGGCCGCGGCTGGGACGCGGTCGGCGCACACGCAGCT
GGCTATAACAGTAACAGTATCGGCATCGTGCTCATCGGCGATTTTGTTTCAAACCTCCCG
CCGGCGGTGCAAATGCAAACCACACAAGAATTGATCGCAGCGGGCGTGCGACTCGGTTAC
ATCAGGCCCAACTACATGCTCATCGGGCATCGTCAGGTCTCCGCCACTGAGTGCCCAGGA
ACCAGACTCTTCAACGAAATCACCAACTGGAACAACTTCGTGAG
>M. sexta-Beta-1, 3-glucan-recognition protein 2 (MsßGRP2); AY135522.1
(SEQ ID NO: 19)
GCGTCTGTTTGTTCGCAACCATTGCGGGCTGCTTGGGCCAGCGAGGGGGTCCATACAAGG
TGCCTGATGCGAAACTCGAAGCTATCTACCCCAAAGGCTTGAGAGTCTCTGTGCCAGATG
ATGGCTACTCCCTATTTGCCTTCCACGGCAAGCTCAATGAGGAGATGGAAGGTTTAGAGG
CTGGCCATTGGTCCAGAGACATCACCAAAGCGAAGCAGGGCAGATGGATATTCAGAGATA
GGAATGCTGAGCTGAAGCTTGGAGACAAAATTTACTTCTGGACTTACGTTATTAAGGATG
GATTGGGATACAGGCAGGACAATGGAGAATGGACTGTTACAGAATTCGTCAATGAGAACG
GTACAGTGGTGGACACTAGTACAGCGCCGCCACCAGTAGCACCCGCCGTTTCAGAGGAAG
ATCAATCGCCAGGTCCTCAGTGGAGACCTTGCGAAAGATCCCTGACTGAGTCCTTGGCCC
GCGAACGCGTTTGCAAAGGCAGCCTTGTCTTTAGCGAGGACTTTGATGGTTCCAGTTTGG
CCGACTTGGGCAATTGGACCGCTGAAGTCAGATTCCCTGGCGAACCGGACTACCCGTACA
ACTTGTACACTACGGACGGCACTGTGGGATTCGAAAGTGGGTCTCTGGTGGTGAGACCCG
TCATGACCGAGTCCAAATACCACGAGGGCATCATATACGACCGCCTCGACCTTGAGAGAT
GTACAGGACAGCTGGGTACGCTGGAATGCAGGCGAGAGAGCAGCGGCGGTCAGATTGTAC
CACCTGTGATGACAGCTAAACTGGCCACTCGACGCAGCTTCGCGTTCAAGTTCGGCAGGA
TCGATATAAAGGCGAAGATGCCGCGCGGGGACTGGTTGATACCAGAACTCAACCTCGAAC
CTTTAGATAACATATACGGCAACCAGCGATACGCTTCGGGTCTCATGCGGGTCGCGTTCG
TGAGAGGAAACGATGTATACGCCAAGAAGCTCTACGGAGGTCCGATAATGTCCGACGCGG
ACCCGTTCAGGTCCATGCTGTTGAAGGACAAGCAAGGGTTGGCCAACTGGAATAATGATT
ACCACGTCTACTCGCTGCTGTGGAAGCCTAACGGTTTAGAGCTGATGGTGGACGGTGAAG
TGTACGGCACCATCGACGCTGGCGATGGCTTCTACCAGATTGCGAAGAACAACCTCGTGA
GCCACGCCTCGCAGTGGCTCAAGGGCACCGTCATGGCGCCGTTTGATGAAAAGTTCTTCA
TCACTCTGGGTCTTCGCGTGGCGGGTATCCACGACTTCACGGACGGTCCGGGCAAACCTT
GGGAGAACAAGGGC
>M. sexta-Relish family protein 2A (MsREL2A); HM363513.1
(SEQ ID NO: 20)
AGTGTGCGTCTACGAATGATTTGAAAATATGCCGCATAAGCCGTTGTTACGGTAGACCGA
GAGGCGGCGAAGATATCTTCATATTTGTCGAAAAGGTCAACAAGAAAAACATCCAAGTTC
GGTTCTTTAGACTGGAAAACGGGGAGCGCACCTGGTCAGCGATGGCGAACTTTCTGCTAA
GCGATGTTCACCACCAATACGCTATCGCTTTTAGAACGCCACCGTACGTCAATCACCAAA
TTTCTGAAGACGTGCAAGTTTTTATAGAACTCGTACGCCCTTCAGACGGTAGGACGAGCG
CTCCCATGGAGTTCACATACAAGGCTGAGCAAATCTATAAACAGAACAAGAAACGTAAAA
CTACTTCGTCGTACTCGTCGCTCGACAGCTCCTCAGGTTCGGCCGGTTCAATTAAAAGCA
TCAGCGAACTGCCCGCGCCCGTTGTTTTTGCTGAAAACGTAAGTTTTTTCTATGACACAT
TACTCATTCTTCAACCCATGACGAATCTATAA
>M. sexta-Dorsal (MsDor); HM363515.1
(SEQ ID NO: 21)
ATGCTTGTGACGTTATGCGGCGGGAACTATAGTGGATTGTCGTTAACAAAAACTAATCAT
TATATGTCACCAAAATCATATGTGCCAGGAAATGGTTATGACGCCGCCGTAATCCTAGGT
ACCACGGAGCAGAATGACAGCGAACCCTCAAACTTGAATATTAGTGATGTTTTTGAAGCC
ATCACGCTCGCTGATCCGTCGTTCGGCGCGGGCGTGCCGTCGGTAGAGGAGACGATGGCG
CACACGCAGCCCCAGCCGCTGCAAATGCCGTACGTGGTCGTCGTGCAGCAGCCCGCCAGC
AAAGCGCTCAGATTTCGATATGAGTGCGAGGGCAGATCAGCCGGTTCGATTCCCGGCGCG
TCGAGCACGCCCGAGAACCGAACCTTCCCCGCCATCAAGATAATCGGCTACACCGGCACC
GTCTCCATCGTAGTGTCGTGTGTCACCAAAGATGAGCCTTGCAGGCCGCACCCACACAAC
CTGGTCGGGCGCGACCACTGCGACCGCGGCGTGTTCTCCGTCCGCATCGAGATCACCGAC
GAGAATAACGAATACCAGTTTCGGAACCTGGGCATACAGTGCGTCAAGCGGCGCGACATC
GGCGAGGCGCTGCGGATCCGAGAGGACCTGCGCGTCGATCCGTTCAAAACCGGCTTCACC
CACCGGAACCACCCGCAAGGCATCGATCTGAATGCAGTGCGGCTCGCGTTCCAAGTGTTC
CTGCCGCACTCCAGCGGCAAGATGCGGCGCACGCTCGCGCCCGTCGTGTCCGACGTCATC
TACGACAAG
>M. sexta-Spatzle (MsSPZ1A); GQ249944.1
(SEQ ID NO: 22)
CGAACAACCTGACAGACGGATAGCGGGACGGTCAGCACAATACGAACATTTAAGAACAAA
CGAGAGGTCTCTCCCGGTCTACAGCGAGACCCAGAGGATACAAGCAGAAGAGAGAAGAAG
ACACAGTTCGAGACTAGAAGAACCGAGACAACGTGCTGAGAATGGTTCATATAAGATATT
GAATAACCCTCCGAAACCCTGTATTACTAATAGGAGAAGTCAAATTGATTCGTCGAATGA
TAGGGTAGTGTTCCCCGGTCCGACTTCAGAAAGGTCGTACGTACCCGAAGTGCCAGAGGA
ATGCAAGAAAATCGGCATATGCGACAGTATACCGAATTACCCAGAAGAACACGTAGCTAA
TATTATATCTCGACTTGGAGACAAAGGAAAAGTATTACAAATAGACGAACTGGACGTATC
AGACACTCCAGATATCGCCCAGAGGTTGGGTCCGCAGGAGGACAACATGGAACTATGTAG
CTTTAGAGAAAAGATTTTTTACCCCAAGGCAGCGCCAGACAAAGATGGAAATTGGTTCTT
CGTTGTGAATTCAAAAGAAAACCCAGTACAGGGTTATAAAGTTGAAATTTGCGACCGTCA
GCAATTACCATGCGCGGAGTTCGCGAGCTTCCAACAGGGATATGAAGCGAGGTGCATCCA
GAAATACGTTCGCCGGACCATGTTGGCGTTGGATCCCAAGGGTCAGATGACCGACATGCC
CCTTAAAGTGCCCAGCTGTTGCT
>M. sexta-Toll receptor (MsTOLL); EF442782.1
(SEQ ID NO: 23)
CAAGTGGTCGTTGTGCACTGCACCGGACTCGCCGAGTTCCCGCGCCTGCCACTAACCACG
GACAAGTGGATCCTGCACCTGCCCCACAACAACATATCATACCTCGCCGCGGCCGACGTA
TCGCCGAACATCGTGGAACTCGATCTAAGAAACAATTCAATCAAGAATATCGACGTACAG
GCATCAGCAAAACTAGCCTTCGTCCGGCTGCAATTGGGTGGTAACCCGATCGAGTGTGAC
TGCGAGGCGTTGAAGCTGCTGGCGCCGCTGCTCAAACCTGACTCAAAGCTGCTTGACAGA
AAGGACGTGAAATGTGAGAACGACGCGCAGATTACCTTGGCGATGCTGAAATTATGTACT
AAGTCATCCAATGGGCTGATGTACTTGCTGTTCCTGTTGCTGTTACTCGCATTTGTCGTA
ACCGGACTGCTCGCACGAACCGCAATTCGCCTGCGCATCAAAATGATCCTCATGAGACTG
GGTTGGATGTCGAGACTACTGGAGCCCGCGGACGACGATCGCCCGTACGACGCGTTTGTG
TCTTTCGCACACGAGGATGAGGAGCTGGTGATGGAGCAGCTGGCGGCACGGCTCGAGAGC
GGCTCGCGGCCGTACCGACTGTGTCTGCACTACCGCGATTGGGCGCCAGGCGAGTGGATC
CCGGCGCAAATAGCGGCTTCGGTGCGGGCCTCTCGGCGCACGGTGGCGGTTGTGTCGGCG
CACTACTTACAGTCGGGCTGGGCGCTTGCCGAGATCCGGGAGGCGACCGCAGCCTCGCTG
CAGGAAGGCATGCCACGTCTCATCATCGTGCTGCTCGACGAGACCGACCGGTTGATGCTC
GATATAGACCCTGAGTTGCACGCCTATGTGCGCAACAATACCTACGTGCG
>M. sexta-Scolexin A (MsSCA1); AF087004.1
(SEQ ID NO: 24)
TCGAAGCTAAGGGGGCAAAGCAGCCAATTGATACGAGGGCAGTGAAGGAACGGTATCCAT
ACGCAGTTCGGAGTTTCGGAGGCTTCTGCGGAGGAACCATTATCAGTCCCACCTGGATCC
TGACCGCCGGCCACTGCTCGATACTCTATGCGGGGAGCGGCCTACCGGCCGGCACCAACA
TTACCGAGGTATCTAGCTTGTACCGCTTCCCCAAGCGGCTCGTCATACACCCGCTCTTCT
CCATAGGACCCGTCTGGCTCAACGCTACGGAGTTCAACCTCAAACAGGCGGCTGCACGAT
GGGACTTCTTGTTGATAGAACTGGAGGAACCGCTGCCGTTGGACGGCAAGATCCTGGCGG
CTGCGAAGCTCGACGACCAGCCCGACCTCCCCGCAGGCCTCGACGTGGGCTATCCGAGCT
ACAGCACCGACACCTACGAGGCTAAGATACAAAGCGAGATGCACGGAAAGAAGCTTTCGG
TTCAATCTAACGAGGTGTGCTCGAAGCTAGAGCAGTTCAAGGCGGAGGACATGTTGTGCG
CCAAGGGACGTCCACCGCGATACGACTTCGTCTGCTTCAGCGACAGTGGCAGTGGGCTAG
TAGACAACAATGGTCGCCTAGTCGGCGTGGTGTCGTGGGCCGAGAACAACGCTTTCGAGT
GCCGCAACGGCAACCTGGCGGTCTTCTCGCGAGTGTCCAGCGTACGCGAGTGGATCCGAC
AAGTC
>M. sexta-Hemolymph proteinase 18 (MsHP18); AY672794.1
(SEQ ID NO: 25)
GATGGTCAGAAAGCGTGGGACAAATGTCTGGAATACGTTGACAAGCTGTCGTACCCATGC
GCTTCAACCTACTCCCACTACCTCAGCTCCGTTTGGGAGAAAGATAAGGAGTGCAGTATG
GTTCAGTTTGTTGGCGTGAGGCGATTCGCCTCGTATAACGGACAACCGGCGAAACGGAAC
GAGTACCCTCACATGGCTCTGCTCGGCTACGGCGACGACCAGGAGACGGCGCAGTGGCTC
TGCGGCGGCTCAGTGATCAGTGATCAATTCATCCTCACGGCTGCACACTGCATCTTTACA
AATCTATTGGGTCCAGTACGTTTCGCAGCGCTAGGAATACTGCAGCGATCGGATCCAGTA
GAGTTATGGCAAGTTTACAAGATCGGCGGCATAGTTCCCCATCCGCAGTATAAGTCACCT
ATCAAGTACCACGACATTGCTCTCCTGAAGACTGAAAACAAAATAAAGTTTAATGAGAAC
GTGCTGCCAGCGTGTTTGTTCATAGAGGGCAGAGTGGGTGGGAGTGAGCAGGCTAAAGCG
ACCGGTTGGGGCGCGCTTGGACATAAACAGACGGCAGCTGACGTACTGCAAGTGGTTGAC
CTTCAAAAGTTCAGTGACGAAGAGTGCGGAAGTACCTACCGTCCTTACCGGCATTTGCCT
CAAGGCTACGACAGCGCCACGCAGATGTGCTACGGCGACAAGGGAAAACTGAATATGGAC
ACCTGTGAGGGCGACAGCGGCGGTCCTCTACAGTTCCAAAACTCCTCGCTCCTCTGC
>M. sexta-Transferrin (MsTRN); M62802.1
(SEQ ID NO: 26)
ATGGCTTTGAAACTTTTAACTTTGATAGCCCTGACTTGTGCGGCTGCGAATGCAGCTAAA
TCTTCATACAAACTATGCGTGCCAGCAGCATACATGAAGGACTGCGAGCAGATGCTTGAA
GTACCCACGAAGTCTAAAGTGGCCTTGGAATGTGTACCGGCTAGAGACAGGGTGGAATGC
CTCAGCTTTGTTCAGCAGCGACAGGCGGACTTCGTCCCCGTCGACCCTGAGGACATGTAC
GTGGCCTCCAAGATCCCCAACCAGGACTTCGTCGTCTTCCAGGAGTACAGGACTGATGAA
GAGCCTGATGCGCCATTCCGTTATGAAGCCGTTATTGTGGTTCACAAAGACCTACCCATC
AACAACTTGGATCAGCTGAAGGGACTGAGGTCTTGCCACACCGGAGTCAATCGTAACGTC
GGGTACAAGATCCCACTAACGATGTTGATGAAACGTGCCGTGTTCCCGAAAATGAACGAC
CACAGCATTTCGCCGAAAGAGAACGAACTGAAAGCGCTATCGACGTTCTTCGCAAAGTCG
TGCATCGTCGGCAAATGGTCGCCTGACCCCAAAACCAACTCGGCTTGGAAATCACAATAC
AGCCATTTGTGTTCAATGTGCGAACACCCGGAGCGTTGTGACTATCCCGACAATTACAGC
GGGTACGAGGGCGCGTTGAGATGCCTCGCCCACAACAACGGGGAGGTCGCGTTCACCAAA
GTCATATTCACACGTAAATTCTTTGGGCTTCCAGTAGGTACCACTCCAGCGAGTCCATCA
AACGAAAATCCCGAAGAGTTCAGATATCTCTGCGTGGACGGATCTAAAGCCCCCATCACT
GGCAAGGCTTGTTCATGGGCTGCCAGACCTTGGCAAGGACTGATCGGTCACAATGACGTA
CTTGCCAAACTCGCTCCGCTCAGAGAGAAGGTTAAGCAACTTGCTGATTCTGGTGCAGCT
GACAAACCGGAGTGGTTCACCAAAGTCCTTGGTCTATCAGAGAAGATCCACCATGTCGCT
GACAATATCCCAATCAAGCCCATCGACTACCTGAACAAGGCTAACTACACGGAGGTCATT
GAAAGAGGACATGGAGCTCCCGAGCTGGTCGTCAGGCTATGTGTGACGTCAAACGTGGCA
TTATCTAAGTGCCGGGCTATGTCCGTGTTCGCATTCAGTAGAGACATCAGGCCGATCCTA
GACTGTGTTCAAGAAAACAGCGAAGATGCCTGTCTTAAGAGCGTCCAAGACAACGGTTCA
GATCTTGCCTCAGTAGACGATATGAGAGTAGCTGCAGC
>M. sexta-Arylphorin ß subunit (MsARP); M28397.1
(SEQ ID NO: 27)
CTGTCATAATCCTAGCGGGGTTGGTGGCCCTGGCCCTCGGCAGCGAAGTGCCTGTCAAGC
ACTCCTTCAAAGTTAAGGATGTTGATGCGGCTTTCGTCGAACGTCAAAAGAAGGTCTTAG
ATCTTTTCCAAGATGTCGACCAAGTAAATCCTAACGATGAGTACTACAAGATTGGCAAGG
AATACAACATCGAGGCTAACATCGACAATTACTCGAACAAGAAGGCCGTCGAAGAATTCT
TGCAGTTATACAGGACAGGTTTCTTGCCTAAGTACTATGAATTTTCACCCTTCTATGACA
GACTAAGGGACGAGGCCATTGGTGTTTTCCACCTCTTTTACTACGCTAAAGATTTTGATA
CGTTCTACAAATCTGCCGCATGGGCGCGTGTGTACCTCAACGAAGGACAGTTCTTATACG
CCTACTACATTGCTGTGATTCAGCGTAAAGATACTCAGGGCTTCGTTGTACCAGCACCGT
ATGAAGTCTACCCTCAATTCTTCGCAAACTTGAACACTATGCTCAAAGTCTACCGTACCA
AAATGCAGGATGGAGTTGTTAGTGCCGATTTAGCTGCACAACACGGCATCGTAAAGGAGA
AAAACTACTACGTATACTATGCCAATTACTCCAACTCATTAGTGTACAACAACGAGGAAC
AGAGACTGTCGTACTTCACTGAGGACATCGGCTTGAATTCGTACTACTACTACTTCCACT
CTCACTTGCCTTTCTGGTGGAATTCTGAGAGATACGGAGCACTAAAATCGCGCCGTGGTG
AAATCTACTATTACTTCTATCAGCAATTAATTGCACGTTATTACTTTGAACGTCTCTCGA
ACGGCCTGGGTGACATTCCCGAATTCTCATGGTACTCACCAGTCAAGTCTGGCTACTATC
CACTGATGTCTTCTTATTACTACCCCTTCGCTCAAAGGCCCAACTACTGGAACGTGCACA
GCGAAGAAAACTACGAGAAAGTACGATTCTTGGACACGTATGAAATGTCATTCCTTCAGT
TCCTCCAAAACGGACACTTCAAAGCGTTTGACCAGAAGATTGACTTCCACGATTTCAAAG
CTATCAACTTTGTTGGAAACTACTGGCAAGATAATGCTGACCTGTACGGTGAGGAAGTTA
CTAAGGACTACCAACGTTCATATGAAATTATAGCCCGCCAAGTGCTTGGTGCTGCACCTA
AACCATTCGACAAGTACACATTCATGCCCAGCGCTTTAGACTTCTACCAGACGTCTCTGC
GTGACCCAATGTTCTACCAACTTTACAACAGAATTCTGAAGTACATATATGAGTACAAGC
AGTACCTGCAACCGTACTCTTCAGAAAAACTGGCATTCAAGGGTGTCAAGG
>M. sexta-Chymotrypsinogen-like protein 1 (MsCTL1); AM419170.1
(SEQ ID NO: 28)
ATGTACGTGAAAGTAGCACTTCTGTTGGTAGCCCTCATTGCTGGGAGCTGGGCCTTCCCA
AAGCTCGAAGATGAGCAGGACATGTCCATCTTCTTCACGCAGCTCGATTCGAGCGCGCGT
ATCGTGGGTGGTACCCAGGCCCCCAGCGGAAGTCACCCTCACATGGTGGCGATGACCACC
GGTACCTTCATCAGGAGCTTCAGCTGTGGAGGCTCAGTTGTCGGTAGACGTTCCGTTCTG
ACTGCGGCTCATTGCATCGCTGCTGTTTTCAGTTTCGGTTCCCTCGCCAGTACCCTCCGC
TTGACGGTCGGCACCAACTTCTGGAACCAGGGAGGCACCATGTACACCGTCGCTCGCAAC
ATAACCCACCCCCACTACGTCTCTGCGACCATCAAGAACGACATCGGTCTGTTCATCACT
CACAACAACATCATCGACACGACTGTCGTCCGCAGCATCCCTCTTAACTTTGACTATGTG
CCCGGTGGTGTTCTCACTAGAGTCGCCGGATGGGGCAGGATCAGGACCGGCGGTGCCATC
TCTCCCTCTCTGCTGGAGATCATTGTGCCTACTATCAGTGGAAGCGCATGCGTAGCCAGT
GCAATCCAAGCTGGCATCGATCTGAACATGAGACCACCTCCCGTCGAGCCTCACATCGAG
CTGTGCACCTTCCACGGTCCTAACGTAGGCACTTGTAATGGTGACTCCGGCAGCGCTCTT
GCCCGCCTAGACAACGGCCAGCAGATCGGTGTGGTATCGTGGGGCTTCCCGTGCGCACGC
GGCGGTCCCGACATGTTCGTCAGGGTCAGCGCCTACCAATCCTGGCTGCAG
>M. sexta-Valine Rich Midgut Protein (MsVMP1); NCBI accession number not assigned
as yet
(SEQ ID NO: 29)
ATCATTGACGGACCTTCCGTTGGACCNGCCATCATCGGCGCTGGAGACATCGCTGTCGGC
CCTGCTATCGTCGACTTCCCTTTCCCCGACGGCGGTGCCGTGTCTGCCCCCGTTGAGCCT
TCCCCCATCGCCATCGGACCCGCTATCGTCGGTGAATCCCCTATCTCCGTCGGACCTGCC
ATCGTTGAGGCCGGAGACATCGCTGTTGGACCCGCTATCATCGACTTCCCCCTTCCCGAC
GGTGGCGCCGTGTCCGCCCCCGTTGAGGTTTCTCCCGTCGACTCCGTCGTCGTCGGCCCT
GCCGCCGGCTCTCAGAGCTCTCCCCTCGTCCAGATCATCATCAACGTTAAGGCCCCCGCT
GGTGCCGGCCCCGTTGTCGATGCCGTCGCTGACAAGCCCATGGACATCATTGATGTTATG
CCCGTCGTCGACCCTGCTGATTTCGTGGACCTCACCCCCGTTGTAGAGCCTGTAGAAGTC
GTCGACATTGTCGATGTCATGCCCGTGGTTGACCCCATCAACATCATCGATGTTATGCCT
GTTGTTAAGCCCGTAAACCCCCTTGCCCGTT
>M. esculanta-Catalase 1 (MsCAT1) AF170272
(SEQ ID NO: 30)
ATGGATCCTTGCAAGTTCCGTCCATCAAGCTCAAACAATACCCCCTTCTGGACCACCGAT
GCTGGTGCTCCAGTATGGAACAACAATTCCTCCATGACTGTTGGAACCAGAGGTCCAATC
CTTTTGGAGGACTATCATATGATAGAGAAACTTGCCAACTTTACCAGAGAGAGGATTCCA
GAGCGTGTCGTCCATGCTAGGGGAATGAGTGCAAAGGGCTTCTTTGAAGTCACCCACGAT
GTCTCTCACCTTACTTGTGCTGATTTCCTTCGAGCCCCTGGAGTTCAAACCCCTGTCATC
GTCCGTTTCTCCACTGTTATCCACGAGCGTGGCAGCCCTGAAACACTCAGGGATCCTCGA
GGTTTTGCGACTAAGTTCTACACCAGAGAGGGCAACTTTGATATTGTGGGAAACAACTTC
CCTGTCTTCTTCATCCGTGATGGAATAAAATTCCCAGATGTGATACACGCTTTTAAGCCC
AATCCCAAGTCTCACATCCAAGAATACTGGAGGATCTTTGACTTCTTATCACACCATCCT
GAGAGCTTGAGCACCTTCGCCTGGTTCTTCGATGATGTTGGAATTCCCCAAGATTACAGA
CACATGGAAGGTTTCGGTGTTCACACCTTTACTTTCATCAACAAGGCTGGAAAAGTAACC
TACGTGAAATTTCACTGGAAACCCACTTGCGGGGTCAAGTGTTTGATGGA
>M. sexta-IMD (MsIMD); Msex2.05477-RA
(SEQ ID NO: 31)
ATGACTTCTTTGAAAAGCAAATTAGCAGAATTCTTGAAGGGGTTAAAATCAGATGCAACC
CCAAGCCCCGAGGCCATCGACAGAACCCAGGGTAAATCCACAAACTGAGCAATGAAAATG
ACGCACCCTCGGATAGTGAACCTGAAGAAATCATTATAGAAGATATGACGATACGAAGAA
AAAGAAAAAGGCAAGTTACAAGAAACCGTTCTTTAGCTCCAAACCTGGTGCATTCCCCGA
AAAGAAAAAAGACAAGAATACCAAAGACGATTACAGCAACTTTGTGAATACTCAAGCCAC
TGGTGATGTCATCAATATTGTAGGCTGTAACAATTTCCGCTGGGGTAATAATTATTATTT
GGGAAATACCAAGAAACAGGCTCCTCCTAAGAAATATTTCCAAGAAGAGGAAGACAGTGA
ACCAGAAGATGATATTCAGAAATGCAACTTAATCAAACTGCTATTTGAAGCTGAAAATAA
GCCAGAACATGAGTACCTGGACTACATTTCCCAGAACATGAATGAAAAGTGGCACAGATT
CTTTGTGAAACTAGGTTTTACACCAGGAAGGATCAAAACATCCATCATAGATAATGCCAG
TTATGGTATTTCAGAGGCTCGATACGCATTGCTACTTGAGTGGGTGAATAAAAAACGGGA
CTCTAATCTTGGACAGCTATCGAATTTATTGTGGAAACACGGCGAGAGGCGAATTGTCAA
AGAATTAGCCATTATGTACTCTGCAAGCAAGGCCAAATCTGATGATGAATAG
>M. sexta-FADD (MsFADD); Msex2.03129-RB
(SEQ ID NO: 32)
ATGACTCTATCGGAATCAAAATTTAAACAATTAAAAGAACAAATTATTTTACATGCAAGT
GCAACTGAAAGACATGCTCAAATTTTGAACTCATTAAAGGATTTGTTTAAAGAAGATATT
AATTCTGTTCGAAGATTTGAACAAATCAGCAATATAGCACAATTATTAAAAGTTTTAGAA
ATAAGAGATGTGTTGTCAGAGGATGATGTTGCTCCTTTGAAAGATGTGGCACGCCAACTA
CCAAATAGCTCAGAAATGCTGCGGAAAATTGCTGAATATGAAGAAAATCACAAGTGTGGA
GAGTTTATTTCTGTTCCTAAGTCACCTCCAAAACAAAAAGAACATAGTCATTCTGGATGG
GATATTAACAGCATACAGAATTCTGAATATTCTGTAAAAAAGGAAAGGATATTTGAAGTT
ATTTCTGAAGAAATTGGCACCCATTGGAGAAATCTAGCGAGATATTTGAAAACAAGGGAA
TGTACAATAATTGAAATTGACAGTAAAAACATACCACTTTCAAGTAAAGCTATGGAGATT
TTGAAATTGCATTGTAAAAAAGCAAATCCACAAAAGTGGTTTTTTGACTTGTGTAATGCA
TTGGACAAAGTGCATAGGACTGACATAGTGTTATCACTGCAAGAAATTATGTCTATGAAT
ATTTAG
>M. sexta-Dredd (MsDRD); Msex2.04297-RC
(SEQ ID NO: 33)
ATGTTGTCTTCCGATGCCAGAAGTTCCCTAAATTCCAAGAGTGATAATGAAACGTTTATA
TTAAACTTGGATTCGATTTCAAAGATTGAAAAACAATTACAAGATAATCCTTATGATATG
ATCTCTTTAGTGTTCCTGTTGTACGACGTGCCGGATACGGCGCTGCAGAGATTGATGGTG
TATCAACGAGTGACGAGTGACGTCAGTGGAACAAATATTAATTTGTTGCAAGAATGGTAC
TGTCACGCCAGCAGTCGCCCTGACTGGCAACATGAATTATTAGAAGCATTAATGATATGC
CAATTGAACTCCATTGTGAAGAGCCTTGGTTTCCATATACCCACTATGAGAGTATTTTAC
CAATCAAATGATCCCTTCAGCAGCAAGTACATAAATCCCGTAAAGAAAGTCCTTTATCAT
GCCTGTGAAAATATAAACTCAACTAATCTTTTGAAATTAAAAAAATCACTTCTTTCATAT
GATATAAATGTAATGGAATATACAACTTGTGAGCTTATATTTTTAGAGCTCCTGTCCAAT
AAATTTATTACTATTAATAATATTGGCCAAAAGATTAAAACAAACCAGAACTGCATATGT
AATGTAGAAAACCTTGTGAAGATCTTAGACAACTTAAATGGATTGAAAAAAGTGGCTATG
AATTTGAGATATTTTCAAAGTAAATTCAATGATGAAGAGGTGGATTCATTCGCGTCTGTC
AATGGAAGCAGCTCACCATCACTTCCAGTGCCTCCTCTTGATGGTACAAAATTAAAGCAA
GATGACAAGAATTATGGAGCTGAAGATTTCTCAGAGCTATTCGACATAATAAATAATATG
CCAGAACCGCTTGAAAACAATTTTAAATCTGATACAATGTCAACTAAACAGAATAGATAT
GAGATAAAAAATCCGGAACAACTAGGTGTTTGCATAGTTATAAACCAAGAGAATTTTTAT
CCATCCAAAAATAGTATTGAAGACCACCAAATTGTTCCATTAGAAGAGAGAAAAGGATCT
AGTGTGGATAAAAGGACTTTGGAAAGAACCATGACATCATTAAAATTTCAAGTTCACAGT
TGCTCTGATTTAGATCATGATGAAATGATAGAATTCATCAAAAAGAAAATTAATAAACAT
GTTACATCAAATGACAGTATTTTTATGTTGTGTATACTGTCACATGGTGTAAGGGACCAT
GTGTATGCTGCTGACTCTGTGAAAATTAAAGTAGAGTCTATACAAAATCTGTTGGATTCA
GATGAAATGAGCCACTTGAGAGGCATACCGAAGGTGTTTATTATACAAGCTTGCCAAGTT
GAGGATACACCTCATCCTACATTTGCTGCTGACAATGTCCAAACAAATTACTACTTGGGA
AAATTAGACTTCCTTATTTACTGGGCCACTGCACCTGAATATGAAGCCTTCAGACATGAG
CAGACGGGGTCATTATTCATTCAAGCTCTTTGTAAACTGTTGCGTCAAAGAGCTAAACAT
GATCACTTACATGAAATCTTTACACAAGTAAATAACAATGTTACCAATCTGTGCACTAAG
TTGCAGCGTGCACAAGTACCTCTCTTCAAAAGTACTCTGAGGAAAAATTTATATTTACAA
GTGCCAGAATAA
>M. sexta-Relish F (MsRelF); Msex2.08004-RE
(SEQ ID NO: 34)
ATGTCCTCTTGTCCAAGCGACTATGATCCCAGTGAATCGTCCAAATCTCCACAAAGTATT
TGGGAGTCAGGAGGATACAGTTCTCCGTCGCAACAAGTTCCTCAATTGACTTCTAACTTA
ACAGAATTGTCTGTTGATCACAGCTATAGATACAATGGAAATGGACCATATCTACAGATC
ACAGAGCAACCACAGAAATACTTTCGGTTCCGTTATGTTAGCGAGATGGTGGGAACACAT
GGATGTTTGCTTGGCAAATCTTATACAACAAACAAAGTTAAAACTCATCCGACAGTTGAA
CTCGTGAATTACACCGGTCGAGCCCTGATAAAGTGCCAACTGTCGCAAAACAAGAGCGAA
GACGAACACCCGCACAAACTGCTCGATGAACAAGACAGAGACATGAGCCACCACGTTCCC
GAGCACGGCAGTTATAGAGTGGTATTTGCCGGTATGGGTATAATTCATACTGCCAAAAAG
GAAGTTGCAGGGTGGCTCTATAGAAAATATATACAGCAGAACAAGAATGAAAAGTTTAAT
AAGAAAGAGCTCGAAGCGCATTGTGAGAGGATGTCCAAAGAGATCGATTTAAACATAGTT
AGACTGAAGTTTAGCGCTCACGATATTGACACTGGCATTGAAATTTGCCGGCCAGTGTTC
TCTGAACCCATTTATAATTTGAAGTGTGCGTCTACGAATGATTTGAAAATATGCCGCATA
AGCCGTTGTTACGGTAGGCCGAGAGGCGGCGAAGATATCTTCATATTTGTCGAAAAGGTC
AACAAGAAAAACATCCAAGTTCGGTTCTTTAGACTGGAAAACGGGGAGCGCACCTGGTCA
GCGATGGCGAACTTTCTGCTAAGCGATGTTCACCACCAATACGCTATCGCTTTTAGAACG
CCACCGTACGTCAATCACCAAATTTCTGAAGACGTGCAAGTTTTTATAGAACTCGTACGC
CCTTCAGACGGTAGGACGAGCGCTCCCATGGAGTTCACATACAAGGCTGAGCAAATCTAT
AAACAGAACAAGAAACGTAAAACTACTTCGTCGTACTCGTCGCTCGACAGCTCCTCAGGT
TCGGCCGGTTCAATTAAAAGCATCAGCGAACTGCCCGCACCCGTTGTTTTTGCTGAAAAC
TTACCTGAAAACAATGAGCGTATTATAGATATACCCGTACAACAGAATATGCTGTACCAG
GTTCTGCCAAGTCAATGTGATTTAGCCGACGCGTTCATGGAGGTGGACAGCAAGGGAAGT
CCGGCCAGCGTCGTGGACCCGATGTGGGCCGGCGCCGACGTCGGGCTGCAGATGGCGTCG
AGCCTGCCCGCCATCAAACTCGGCTCCACCGAACTAGAGAGCTTGGCGCACCGCAGCCGG
GACGGAGTGCCCATGGACAAAGAATTCCTTGACAATTATCTCAGCTCTCTCAACTCGCTT
GGTGAACTTAACGACGATGATGAGATCAATCATATGCAATATGTGCGCTCGTTACAGATG
GTCCACGCCGACTCGGCACGACGAAAGGCTGAACCGCAACCCAAAGACACTGTAACCAAA
CCGACTCAATCTTCCCCGAAGGACACAGACTATGGCGCCCAGTCGGGACGCCCCACTGAA
TATTCTGCTTATTATAAAATGGAAGACGGCGTGGAAGTCAAAAAACTGGTGAAAGAATTG
TGTAGTATCATACAGAACAAAGCGGGATATAAAAAACAAGAAGTGAGAAACAAATTGGAG
AGGCTGTTCACCTACCGTCTGTCCAACGGAGATACATTCCTTCATATGACATTGTGCAGC
AATCAAAGTAGTTTTGAATACATCGTCAAAATCATACATAGCGTGAAAATGACACATCTA
TTGGACTATTGTAATAATAAACAGCAAACCATATTACACATGGCTATTGTAAACGACCTG
CCTCGAATGGTCTCTTTACTTATTGCAAAAGGTTGCAATCCAATGAATAAAGACAGCGAG
GGCGACAATGCGGTGCACTACGCTGTGAGGAGCGAATGTTGTTTAGAAGCATTATTGGAC
GCCATCAAAAATAACAATGTTCGGTGCGATTTGAACGACTGCAATAACGAGAAGCAGACG
GCGCTGCACCTGTCGACGAGCGGCGCGAGCGCGCGGCAGCTGGCGGCGCGCGGCGCCGAC
CCGCGCGTGCGCGACGCGCAGGGCCGCACGCCGCTGCACCTCGCCGCCTACGACGACAAC
TGCGACGTCGTCAGGGCGCTGCTCGAGTTCGTGTCGCCCTCGGAAATAGACGTTGTGGAC
GGCTGTGGCAACACTGCGTTACAGATCGTCTGCGGCGGCTCCGTTAAGAAGAACACTTTG
GAAATAGTAAAACTGTTGCTCCAAAAAAAGGCTGATCCGTTGAAGCAAGATGGGCACAAC
ATATCGGCGTGGAAGATGGCGCGCGAACACTCCGAAATACGGGACGCGATGAAGGACTAT
GTCCCCGCCGCCGCGTACGAGGAGGACACCAAGTCGGAAATGGACGATGAGTTCGAATCC
GCTGATGAGGAGGACTACCGGATGGGTTCCGGATCGGGCGCTGTGAGTCTGCCGGAGCTG
GGCGCATACGTGGAGGCGCTGAGCGCGGCGCTGGAGGCGCGCGGCGCGTGGCGCGCGCTC
GCGCACCGCCTCGGCCTCGCCGCCGCGCTCGACTGGTGCGCGCGACAGCACGCGCCCGCG
CGCACGCTGCTGCTGCACCTCAAGGAATGCAGAAACGACATATCCTCGAAAACGTTGGCT
GTAATTCTAGAAGATATGGGAGAATTAGAGGCTGCTTCAATTATAAGGAGACACATAGAG
TGA
>M. sexta-Cdc42 (MsCdc42); Msex2.04668-RA
(SEQ ID NO: 35)
ATGCAGGCGATCAAGTGTGTCGTCGTCGGAGACGGTGCCGTCGGTAAAACATGTCTGCTC
ATCAGCTACACGACAAATGCCTTCCCCGGAGAATACATACCTACAGTATTCGACAATTAT
TCAGCGAATGTGATGGTGGACGGGAAGCCGATCAACCTGGGCCTGTGGGATACGGCGGGG
CAGGAGGACTACGACCGGCTGCGGCCGCTGTCCTACCCACAGACCGACGTGTTCCTTATA
TGCTTCTCGCTCGTCAACCCGGCTTCGTTCGAGAACGTTCGGGCTAAGTGGTACCCAGAA
GTGCGGCATCACTGCCCGTCGACGCCCATCATCCTCGTCGGTACCAAGCTGGACTTGCGC
GAAGACAAAGACACCATAGAGAAACTTAAGGACAAGAAGCTCGCGCCTATCACTTACACA
CAGGGTCTGGGCATGTCGAAGGAGATCAACGCGGTGAAGTACCTCGAGTGCTCTGCGCTG
ACGCAGAAGGGTCTGAAGACGGTGTTCGACGAGGCCATCCGCGCCGTGCTGTGTCCCGTG
CCGCCCCCCAAACAGAGCCGGAAGTGCACGCTGCTGTAA
>M. sexta-DSor1 (MsDSor1); Msex2.00725-RB
(SEQ ID NO: 36)
ATGAGTAAGATGTCAAAGAATAAATTAAACTTGACCCTGCCACCAGGGTCAATAGACACA
GCACCAGCCATCACACCATCCAATATGACACCACAGCTGAAGTCCGCAACAGCTACGGAG
CGTCAGGGCTTGGCTGGTAAATCGAAAACCAGCATCGAAGCCCTGACAGAGAGGTTGGAG
CAAATCGAGATGGACGACACACAGAGACGGAGAATAGAAGTGTTTCTGTGTCAGAAGGAG
AAGATCGGGGAGCTTAGTGATGATGATTTTGAAAAGCTTGGAGAGTTAGGCCAAGGCAAC
GGTGGCGTTGTAATGAAAGTCCGTCACAAGTCAACCGGTCTGATAATGGCGCGAAAGTTA
ATCCATCTGGAAGTCAAGCCGGCAATAAAGAAGCAAATCATCAGGGAGTTGAAAGTCTTA
CACGAATGTAACTTTGCGCATATCGTCGGCTTCTACGGGGCCTTTTATAGCGACGGCGAG
ATCTCGATTTGTATGGAGTACATGGACGGTGGGTCCTTAGACCTTATACTGAAGAAGGCC
GGCAAGATTCCTGAATCTATTCTAGGAACAATAACATCCGCCGTGCTGAAAGGTCTGAGC
TACCTCCGGGACAAGCACGCCATCATGCATCGCGACGTGAAACCATCAAACATTCTGGTG
AACAGCAACGGCGAGATCAAGATATGCGATTTCGGCGTGTCCGGTCAGCTGATCGATTCC
ATGGCCAACTCTTTTGTCGGCACTAGGAGTTATATGTCTCCCGAACGTCTCCAAGGCACG
CACTATTCAGTCCAATCTGACATATGGTCGCTCGGGCTGTCGTTGGTAGAGATGGCAATC
GGAATGTATCCCATACCGCCGCCGGACGCGAAGACCCTGGCTGCCATCTTCGGTGGACAG
AATGAAGATCATTCTCCTGGTCAGGCGCCGAACTCGCCCCGTCCGATGGCCATATTCGAG
TTGCTGGACTACATCGTGAACGAGCCGCCGCCGAAGCTGCCCGCCGGAATATTCTCCGAC
GAGTTCAAGGACTTCGTCGACCGCTGCTTGAAGAAGAATCCAGACGAACGAGCCGACTTG
AAGACTTTGATGAATCACGAATGGATACGCAAAGCGGACGCAGAAAAGGTGGACATAGCG
GGGTGGGTGTGCAAGACAATGGACCTCATGCCTTCCACTCCAAACTCTAACGTGTCTCCT
TTTTCTTCATAA
>M. sexta-FOS (MsFOS); Msex2.09858-RA
(SEQ ID NO: 37)
ATGCAGAACATCGATCCTCTGGAGATCGCCAACTTCCTCGCCACGGAGCTGTGGTGCCAG
CAGTTGGCGAACCTCGAGGGCCTCCAGTCGGGGGTCCCGACTCGCACAACGGCCACCATC
ACGCCGACGCAACTGCGCAACTTCGAGCAGACTTACATCGAGCTGACCAACTGCCGCAGC
GAGCCCACCACGCACGCCGGCTTCGTGCCGCCGTCAGTCACGCACGCCAACAACTACGGC
ATCCTGAATCCGACGGCGTATTGTGATTCGGGCCCGACGACGGCGCTGCACGTGTCGCCG
GGCCCGCTCTCCGCCAGCGGCGACAGCAGCAGCAGCCCCGGCCTGCCCACGCCCAAGCGG
CGCAACATGGGCGGCCGCCGGCCCAACAAGGCGCCGCAAGAACTCACGCCCGAGGAAGAA
GAGCGCAGGAAGATCCGCCGCGAGCGAAACAAAATGGCCGCCGCCCGTTGTCGCAAGCGC
AGACTCGACCACACCAACGAGCTGCAAGAGGAAACCGACAAATTAGAGGAAAAGAAGCAT
GCGCTCCAGGAGGAAATCCGCAAACTGAACGCTGACCGGGAGCAGCTGCAAGTGATCCTT
CAGAACCATATGGTATCATGCCGGCTCAACAAGAGATCCATCAGCCCGCCCGATGTGAAG
CCCTTCCAGGACCCGTACGCCTACCCTGAGATACCCGAGGATGGCGTCCGTGTCAAGGTG
GAAGTGGTCGACCCCTCAGTAGACACGGTCTTAGTGTTGGACAATATTTACTCAACGCCG
CCGACTGACAAAAAAATTATGCTGTCGTCGGCCAACCCAGCCGTGGTGACGAGTGGGTCG
CCCGCCGCCCTGGAGACCCCGCCGGCGATAGTGCGTCCCAACAGACCCAACTCCCTCCAG
GTGCCGCTCAGCCTCACACCAGCACAGTTACACAACAACAAGGCGCTGGGAAACAACAAA
ATAGCCGGCATAGAGATAAGCACGCCGAGCAACGGCATCCCGTTCAACTTCGAGAGCCTG
ATGGAGGGCGGCACGGGGCTGACGCCGGTGCACGGCCACGCGCTGCCGCTGCCGCACCCG
TGCGCGCAGCAGCAGCGCGCCGCGCCCGACGCCTCGCCCGCCGAGCCGGCGCCGAGCTCG
CTGGTGAGCCTCTGA
>M. sexta-Jra (MsJra); Msex2.12422-RB
(SEQ ID NO: 38)
ATGGTTCGGCACTCCGGCCACGGCATGGAGACCACTTTCTATGACGAGCAGTATCCCATC
AGCGGCCCCGTGGAGAACCTGAAGCGGCCCCTCACGTTAGACGTGGGGCGCGGCGTGAAG
CGCGCCAGGCTCGGCGGGGCACCCGTACTCTCATCTCCAGACCTACAGATGCTGAAGCTC
GGCTCGCCAGAGCTCGAGAAACTGATCATCCAGAACGGCTTGGTGACGACGGCCACCCCC
ACTCCAGGTGCGCCGGTGCTGTTCCCCGCGGTCGCGCCTACCGAAGAGCAAGAGATGTAC
GCGCGGCCATTCGTCGAGGCGCTAGACAAGCTGCACCACGGCGAGGTGACCCCGATCGGG
CGGCGAGTGTACGCCGACCTGGACCGGCCGCTCGAGCGGTACCCCACGCCCGTGGTGAAG
GACGAGCCGCAGACGGTGCCCAGCGCCGCCAGCTCGCCTCCACTTTCCCCTATTGACATG
GACACGCAGGAGCGGATCAAACTGGAGCGCAAGCGACAGAGGAATCGAGTGGCCGCTTCC
AAATGCAGGCGGCGCAAGCTCGAACGCATCTCCAAGCTGGAGGACAAGGTGAAGATCCTG
AAGGGCGAGAACGCGGAGCTGGCGCAAATGGTGGTGAAGCTAAAAGAGCACGTACACCGA
CTGAAGGAGCAAGTGCTGGAGCACGCCAACGGCGGCTGCCACATCGAGTCGCACTTCTGA
>M. sexta-Caudal (MsCAD1); Msex2.04570-RA
(SEQ ID NO: 39)
ATGGTGAATTACGTTAATCCCCTCGCCATGTACCAAGGCAAGGGCGGGCAATACGGCGGC
GGGTGGTACGGCTGGCAGCATCAGAACTTTGAAGAACAACAATGGTGTGCTTGGAACGGT
GCGCCGGCGGGTGGCGAGTGGGCGCCAGATCCACATCATTTTCCCAAAAGAGAACCTGGA
GAGAGAGAAATAGCAGACATGCCATCACCGGCACGAGGAGACTTGGCAAGTCCAGCAGAA
GGTTCGCCGAGCTCGGGGTCGAGGCCGTCGCAGCCGCCAGCACCGCCGCGTTCCCCATAC
GAATGGATGAAAAAACCCAACTACCAGACACAACCTAACCCAGGTAAAACGCGCACAAAA
GACAAATACAGGGTGGTGTACAGTGATCATCAGAGGCTGGAGTTAGAGAAGGAGTTCCAC
TACAGCAGGTACATCACTATTCGTCGCAAGGCAGAACTCGCCGTCAGCCTTGGCCTTTCC
GAACGACAGGTCAAAATTTGGTTTCAAAATAGACGAGCCAAGGAAAGGAAACAGGTGAAG
AAACGCGAAGAAGTGGTGATGAAAGAGAAAGGCGACCATGCATCTCTACAGCACGCGCAG
CTGCACCATGCCACCATGCTGCATCACCAGCAGATGATGAACGGCATGATGCACCACCAC
CACTACCACCAAGGCGTGTTGCAAGGCGTGCCGGAGCCGCTGGTGCCGGGCGTGCCGCCC
GTGCCGCTGCTGTGA
>M. sexta-Atg8 (MsAtg8); Msex2.12227-RA
(SEQ ID NO: 40)
ATGAAATTCCAATACAAAGAAGAACATTCTTTCGAAAAGAGGAAGACTGAAGGCGAAAAG
ATTCGCAGGAAATACCCGGATCGCGTTCCAGTAATTGTAGAGAAAGCCCCGAAGGCTAGA
TTGGGAGACCTCGATAAGAAGAAGTATTTGGTGCCGTCTGATTTGACTGTCGGACAATTT
TATTTCCTAATTAGGAAACGCATCCATCTTCGGCCTGAGGACGCATTGTTCTTTTTCGTG
AACAATGTTATTCCACCAACATCCGCCACCATGGGCTCTCTGTACCAGGAACATCATGAC
GAAGATTTCTTCCTCTACATTGCATTTTCTGATGAAAATGTTTATGGATTTTAA
>M. sexta-Atg13 (MsAtg13); Msex2.06273-RA
(SEQ ID NO: 41)
ATGGCACCCGAAGTGGCTTTTTCTAACATAAATGACAAGAATGAATTTACTAAATTCACA
AAATTCTTAGCTTATAAAGGCGTTCAAGTTATTGTAGAATCGAGGAAGGGTGTAAAAATA
GATCCTAATAGTAAACCAAGATCATCAGACACTGATTGGTTCAATCTTCAAATACCAGAC
TCTCCAGAGGTTAACCAGGCAACTAAGAATGCATTGCCTTCAGACAAGGTGTTAGAGATT
ATCAAAGCCCAACTGCACGTGGAAATATCAGTACAAACCGAGGATGGCGATGAAATGGTT
TTGGAGTTGTGGACCCTTGAACTTGACGAAACTCAGTTTGACACTTCTGTTAAAGCCACG
AACACAGTTTACTTCAGAATGGGAATATTACTTAAGTCGCTTATAACTATAACAAGAATT
ACTCCAGCGTACCACTTGTCGAGGAAGCAAAGAACAGAGTCGTTCACAATATTCTACAGA
GTATACAATGGCGAACCGAAAGTAAAAGCGCTTGGAGAATCAGTGAAGAAAATTCAAGCT
GGAATGCTCAAAACTCCACTTGGAGGGATAATATTCTCCGTGGCCTACCGCACAAACTTC
TCCATTTCGCCAAACAGATCGGAGAAGGACAAAACATTGCTTTTGAAGAGTGATCATTTC
GAACTGAGTCCAAAACATGTGATATTTGAATCCAAGAAAAAGAAAGACGAGAAAAAAGAA
TACAAGCCTCTGAAACCTGTTGATTTGAATAAGCCACTTCGATTAGCCGCATTTGTAGAC
GAGGATGTTGTAAAAAAGGCCTTTGATGACTTCATGGAGAAGATGCCGATTCCCAAATAC
CGAGTGATACGTAGGGAGAGAAGTCCAGAGAGCGACAAGCCTATAGTATCCAAAAGTACT
CAGGATTTGGACGCTTCTGTGACGTCAAAGAGTCCGACGTCAATGGAAATGCCACCTAAA
AAGTTCACAGGCTTTCGCAATGAGAACGAACCTCCATTAAAACTTCTCCATTTCCCATTC
GCTGATAACCATCCGATAAGAGAACTGGCCGAGTTTTACAAAGAGTTCTTCAACGCCCCC
CATTTGAAGTTAGCTGATGACGTTAGCCTGAAATCGGGCAGTGCTGAATCGGTAAAAGAG
GTGATAGAGATTACAACTGAAGATTTGTCGAAAGATCTCGAATTGTATGAGAACTCGGTG
TTGGAGTTTGATGAGCTCTTGGCAGATATGTGCCGATCGGCTGAGTGGAGTGGGAACTAG
>M. sexta-IAP1 (MsIAP1); Msex2.05607-RA
(SEQ ID NO: 42)
ATGGCATCAGCTGGCGCCGTACCGAATATTCTAGTGTCGTCGTCGCTTTTCAAGACCACT
CCTCGGGATCCTAAAATTCGTCGAAAAACAAGCCCTTTGAAGTTACCACCTTGTGACACA
CATATAGGCTCGCCTCCACCATCATCCTCTCCGACCTCCTCGTCAACTGATAAAACCGAT
AATCATGATACTTTTGGTTTCCTTCCTGACATGCGtgatatgcgCCGCGAAGAAGAACGA
CTGAAAACTTTCGATAAGTGGCCTAGCACGTACGTGACACCTGAAGAGTTAGCTCGTAAT
GGTTTCTACTACCTTGGGCGCGGTGATGAAGTTCGCTGTGCATTCTGTAAAGTAGAAATT
ATGCGATGGGCGGAAGGAGACGACCCTGCGAAAGATCACAAACGATGGGCGCCCCAATGT
CCGTTTTTACGTAATCTTTCGAACGGTACTAATGGCAGCGGAGAAGGTAGTTCGGAAGGT
CGCGACGAGTGTGGTGCGCGAGCAGCGACGCGGGAGCCTGTGCGTATGCCCGGACCTGTG
CATCCCCGCTATGCAACAGAGTTATCGCGTCTCGCCAGTTTCAAGGATTGGCCGCGTTGC
ATGCGCCAAAAACCAGAGGAACTCGCGGAAGCAGGATTTTTCTACACCGGCCAAGGGGAT
AAAACGAAATGTTTCTATTGCGATGGAGGTCTCAAAGATTGGGAAAACGACGATGTTCCA
TGGGAACAGCATGCCCGTTGGTTCGATCGTTGTGCGTATGTGCAACTTGTTAAAGGCCGT
GATTATGTCCAGAGGGTATTATCGGAAGCGTGTGTTATACGTGCCACCGACGAAAAGCCT
GCGCCTGCACCTCAACCGTCCCAACCAAATGTCTCAGTTGTATCAGAAGAAAAACCTGTC
GAAGAGGCTAAGATATGCAAAATATGTTATTCCGAAGAACGAAACATATGCTTTGTGCCG
TGCGGTCATGTGGTCGCTTGTGCAAAGTGTGCCTTATCTACTGATAAATGCCCAATGTGC
CGGAGGGCTTTCACAACTGCACTGCGACTCTATTTTTCGTGA
>M. sexta-Chitin synthase 2 (MSCHS2); AY821560.1
(SEQ ID NO: 43)
ATGGCCGCAACTACACCAGGTTTTAAGAAGTTAGCAGACGATTCTGAGGATTCAGATACA
GAATACACCCCGCTGTATGATGACGGTGATGAAATAGATCAAAGAACTGCACAAGAAACA
AAAGGATGGAATCTATTTCGAGAGATTCCGGTGAAAAAGGAGAGTGGATCTATGGCCACA
AAAAATTGGATAGAAACAAGCGTAAAAATCATAAAAGTGCTTGCCTACATATTGGTTTTT
TGTGCTGTACTGGGTTCCGCAGTCATAGCTAAAGGAACTCTTCTATTTATTACGTCACAA
CTGAAGAAAGACAGACAAATTACTCACTGCAATAGACGACTTGCTTTAGACCAACAGTTC
ATAACGGTACACAGTTTAGAAGAAAGAATAACATGGCTATGGGCAGCACTTATTGTATTC
GGTGTGCCGGAGTTAGGGGTGTTTTTGAGATCCGTCAGGATATGCTTCTTCAAAACTGCC
AAGAAACCAACCAAAACACAGTTTATTATTGCTTTCATAACAGAGACACTACAAGCAATA
GGAATAGCAGCACTTGTATTAATAATTCTACCAGAATTAGACGCTGTGAAAGGAGCCATG
TTGATGAACGCCACGTGCGCTATCCCTGCATTGCTAAACATTTTCACGAGAGACCGAATG
GATTCTAAGTTTTCTATAAAATTGATATTGGATGTATTGGCGATATCGGCACAAGCCACG
GCGTTTGTTGTTTGGCCTCTTATGGAAAGAACGCCAGTTCTATGGACCATACCAGTTGCA
TGTGTGTTAGTGTCTCTAGGCTTCTGGGAGAATTTTGTTGACACCTACAATAAAAGTTAT
GTTTTTACGGTGCTGCAGGAACTACGCGACAACCTCAAGAGGACTCGGTACTACACTCAG
CGGGTGCTATCTGTTTGGAAGATTATAGTGTTTATGGCATGCATTTTAATATCGCTGCAT
ATGCAAAATGACAATCCGTTTACCTTTTTCACTCACGCCAGCAAAGCCTTTGGAGAGAGA
CAGTATGTCGTTAACGAGGTTCTAATAGTAGTCCGAGATGACGAAACCATAGGCTATGAC
GTCACCGGAGGTATATTCGAATTGGACGCGATATGGACCTCAGCATTGTGGGTCGCATTA
ATTCAAGTGGGAGCAGCCTACTTCTGTTTCGGAAGTGGCAAGTTTGCTTGCAAAATTCTT
ATACAAAATTTTAGTTTCACTTTAGCATTGACTCTCGTCGGGCCCGTGGCAATCAACCTC
CTTATTGCTTTCTGCGGAATGAGAAATGCAGACCCTTGCGCTTTCCATAGAACTATACCT
GACAATTTGTTTTACGAGATACCACCTGTGTACTTCTTGCGGGAGTACGTGGGCCACGAG
ATGGCGTGGGTGTGGTTATTGTGGCTCATATCTCAAGCGTGGATCGTGTTTCACACGTGG
CAGCCGCGATGCGAGCGCCTCTCCGCAACTGACAAACTGTTTGCCAAACCGTGGTACATC
GGACCGCTAATCGACCAATCGTTGCTGCTAAACAGGACTAAGGATTTGGATAATGATTGC
CAGGTTGAGGATTTGAAGGGTCTTGGCGACGATTCGTCGGTTGGAAGCGATCTTGCCATC
GTAAAAGATATCAAACCGTTCGATTCGATAACCAGAATACAAGTGTGTGCGACAATGTGG
CACGAGACCAATGAGGAGATGATCGAGTTCTTGAAGTCGATATTCCGCCTCGACGAGGAC
CAGAGCGCGCGCCGCGTGGCGCAGAAGTACCTCGGCATCGTCGACCCCGACTACTACGAA
CTCGAGTGTCATATTTTCATGGATGACGCTTTTGAAATATCCGATCACAGTGCCGAAGAC
TCGCAGGTGAATCGCTTCGTGAAGTGCCTAGTGGATGCGGTGGACGAAGCGGCTTCCGAA
GTGCATCTGACTAACGTGAGGTTAAGGCCCCCCAAGAAATACCCCACCCCGTACGGCGGA
AAACTAATTTGGACCATGCCAGGGAAAAATAAGTTGATTTGCCATTTGAAAGATAAATCC
AAGATTCGGCACAGAAAGAGATGGTCGCAGGTTATGTACATGTACTATTTCCTGGGGCAT
CGCTTGATGGACCTGCCAATATCCGTGGATCGTAAGGAAGTGATTGCCGAGAACACGTAC
CTATTAGCTTTGGATGGAGACATCGACTTCAAGCCGAGCGCTGTGACGTTGCTGGTCGAT
CTTATGAAGAAGGATAAGAACTTGGGCGCCGCTTGCGGTCGCATTCATCCTGTCGGCTCT
GGTTTCATGGCCTGGTACCAGATGTTCGAGTATGCTATTGGTCATTGGCTGCAAAAGGCG
ACTGAACACATGATCGGCTGCGTACTCTGTAGTCCGGGATGCTTCTCCCTCTTCAGAGGA
AAGGCGCTTATGGACGACAACGTCATGAAGAAATACACACTCACTTCTAACGAGGCTCGA
CATTACGTGCAATACGATCAAGGCGAGGACCGTTGGCTGTGTACGTTGCTGCTGCAGCGC
GGGTACCGCGTGGAGTACTCGGCGGCGTCGGACGCCTACACGCACTGCCCCGAGCGGTTC
GACGAGTTCTTCAACCAGCGCCGCCGCTGGGTGCCCTCCACCATGGCCAACATATTCGAT
CTGCTCGCGGACTCCAAACGCACCGTGCAAGTCAACGACAACATTTCCACTCTGTATATC
GTCTATCAGTGCATGCTTATGATGGGTACGATTTTGGGTCCGGGAACAATCTTCCTGATG
ATGATTGGCGCAATAAACGCTATAACAGGCATGAGCAATATGCACGCACTTCTCTTTAAC
CTGGTGCCCGTGCTTACGTTTTTGGTTGTCTGTATGACATGCAAGTCCGAGACTCAGTTG
ATGCTCGCGAACCTCATTACCTGCTTTTATGCGATGGTAATGATGTTTGTGATCGTCAGT
ATCGTCCTACAAATATCACAAGATGGTTGGCTAGCGCCATCTAGTATGTTCACTGCGGCA
ACATTTGGAATATTCTTCGTAACAGCGGCTTTGCATCCACAAGAAATAATATGTTTGTTG
TACATTTCCATATATTACATCACAATTCCGAGCATGTATATGTTGTTGATTATCTACTCC
CTATGCAATTTGAACAACGTCTCGTGGGGAACTCGAGAGGTGGCTCAGAAGAAGACTGCA
AAGGAAATGGAAATGGATAAGAAAGCAGCGGAAGAAGCAAAGAAGAAGATGGATAATCAA
AGCATAATGAAATGGTTCGGCAAGTCGGACGAGACGAGCGGCTCGCTGGAGTTCAGCGTG
GCGGGACTGTTCCGCTGCATGTGCTGCACCAACCCTAAGGACCACAAGGACGACTTGCAT
CTCTTGCAGATCGCCAACTCCATCGAGAAGATCGAAAAGAGATTGTCGGCACTTGGCGCG
GAGGAGTCCGAGCCGGCGCAGGCGCAGACGCGGCGCCGCTCGTCGCTGGGGCTGCGGCGC
GACTCGCTCGCCACCATGCCCGAGTACGCCGACAGCGAGCTGTCCGGAGACATTCCTCGC
GAAGAAAGAGACGATCTTATAAACCCCTATTGGGTGGAGGATCCCAATCTGCAGAAAGGT
GAAGTAGACTTCTTGACGACGGCTGAAATCGAATTCTGGAAGGACTTGATTGATGTTTAT
TTGAGGCCTATCGATGAAAACAAGGAAGAGCAGGAACGTATCAAAACCGATCTAAAGAAC
TTGCGTGACACGATGGTGTTCGCGTTCGCCATGTTGAACTCGTTATTCGTGTTGGTGATA
TTCCTGCTGCAGTTCAACCAGGACCAGTTGCACATAAAGTGGCCGTTCGGGCAGGATGTC
GCGCTTTCTTATGACAAGGAAAGGAATGTTGTATTGGTGGAGCAGGAATTCCTTATGTTG
GAGCCTATAGGTTCCCTGTTCCTCGTGTTCTTCGGGTTTGTAATGTTGATACAGTTCGTG
GCGATGTTGTCCCATCGTTCGTATACTATTACGCATTTGCTCTCCACAACAGAGCTTCAC
TGGTATTTCAGCAGACGTCCGGACCAGATGTCAGATGAAAACCTCTTGGAAAGGAAGGTG
GTAGAAATAGCGAGAGAGTTGCAGAAGTTAAACACGGACGACCTAGACCGCCGCGCGGTC
GAAACTAACGACGTGTCGCGGCGAAAGACTCTACACAACCTAGAGAAGGCGCGAGACACC
AAGCACAGCGTGATGAACCTTGACGCTAACTTCAAGAGGCGACTGACTATACTACAGAGC
GGTGATCCTAACGTGATATCTCGGCTGTCATCGTTGGGCGGCGATGAGGTTACTCGTCGC
GCCACGATACGCGCATTAAAGACGAGGAGGGACTCACTGCTCGCTGAAAAACGACGCTCC
CAGCTGCAAGCGGCGGGCGACGCTACAGGCTACATGTATAACCTGTCAGGCACTGCGGTG
AACGACATGAGCGGCCGAGCTTCGACGGCCAGCGCCTACATTAATAAAGGATACGAACCC
GCTTTCGATAGCGACGACGACGAACCACCGCGTCCGCGCAGGAGCACTGTACGCTTCAGA
GAAAACTACACGTAA
>M. sexta-Beta-fructofuranosidase 1 (MsSuc1); GQ293363.1
(SEQ ID NO: 44)
ATGTACATTAAAACAGCAACATTTTTGCTGTGCGTTTTCCTTGGTAGTGTATCGTCATGT
TGCGTTAATGGGCGGTACTACCCGAGGTACCATTTGTCGCCACCGCATGGCTGGATGAAC
GACCCCAACGGATTCTGCTACTTCAAAGGTGAATACCATATGTTTTACCAGTACAATCCC
ATGTCAAGTTTGGAGGCTGGCATAGCTCATTGGGGTCATGCGAAAAGTAAAGATTTGTGC
CATTGGAAACACTTAGACCTCGCCATCTATCCTGATCAGTGGTACGATCAAACGGGAGTA
TTTTCTGGAAGTGCGCTAGTAGAGAATGACGTCATGTACCTTTATTATACTGGAAATGTA
AATCTTACTGATGAAATGCCATTTGAGGGACAATTCCAAGCTCTTGGTATCAGTACTGAC
GGTGTCCACGTAGAAAAGTATAAAGACAATCCAATAATGTACACGCCAAACCATCAACCT
CACATCCGAGACCCAAAAGTTTGGGAACACGACGGCTCTTATTATATGGTCTTAGGAAAC
GCATATGATGATTATACAAAGGGCCAAATAGTTATGTACGAATCATCAGACAAGATCAAC
TGGCAAGAAGTAACTATACTATATAAATCAAATGGATCTTTCGGTTACATGTGGGAGTGT
CCAGATTTATTCGAAATAGACGGCAAGTTTGTACTTCTGTTCTCTCCTCAAGGCGTGAAG
TCTGTGGGCGATATGTACCAGAATCTGTATCAAGCAGGATACATCGTCGGAGAATTCGAT
TACGATACTCATTCATTCACAATACTAACCGAATTCAGAGAATTGGATCACGGTCATGAT
TTTTACGCTACACAAACAATGAAAGATCCTAGTGGAAGAAGAATAGTCGTTGCTTGGGCA
AGTACTTGGGAGTATGCTTATCCTGAACGAGCAGATGGTTGGGCTGGCATGCTCACACTA
CCTAGAACTTTAACTTTGACAAAAGATTTAAGACTAATCCAAACTCCAATTAGAGAGATC
GATCAAGTTTTTAGAAGAAGACTATATTCAGGAAAAGCCTCAGCAGGCAAAACTGTCGCT
TTACCAGACAAAGCAGGGAAAGTAGAACTGAAATGGGATACACCAAGAAATATAAAGGTA
GTTATAGAATCTCAAAATGAGTGCCAAAACGTAGTAATCAGTTATGATCACGAGGATGGT
ACTATTACTTTGGACAGAGGAGGCGATGACGCAATCCGCCGCACTCACTGGGATCCTCGA
GGTCACCTCAAATGGACCATTTTTATTGACGCAAGCTCCATAGAACTCTCTTGTGGTGAT
GGAGAAGTATGGTTTACAAGCAGATTTTTCCCTGAAGGAGTCGTATCTGTTCGCCTTGGA
GAAGATACTTGTGTTGATAAGTTTACCGTGCATTCTATTCGCCGTACTACTCCAGACCCC
GAGGCTCATTGTCGTTGTGAATCAGAAGAATAA
>M. sexta-Hemolin (MsHEM); M64346.1-UTRs
5′UTR
(SEQ ID NO: 45)
TGTTACATTAATTATAAAAAAAAATACAAAAAAGAAATATATAAGTAAGTACTAATAATA
TGGTCTAGTTTATTATAAGTCTTAGAGACTAACGATAAATAATAAATTTCTCATGAACCA
CCTATTTATTTATTTGAAAAGCATTTTAAATATATTATAGATTTAAACGTAACGAAGTCT
TTAAACTCGCGGGTAAAAACGCATACTTACTTGACTCTACATCGGCGACACACGAAAAAC
TTATTCGTACTGTTTTTTCATTGAATATGTTATGAAAAAGAAAACTATATGTAATCGCAG
TCACATATATAAAACTTTATAAAAAAGTTAACAGAGATAAATAACTTGTTTGGTGTTCGT
CATAGCAATAGAGTAGCCGCGGCGCCTAAGGCTACGAAATAAGCTCATTGAACTGTAATG
GAGTGTGCCTAGTTGTTTCTCTTCTTACCAAGACTCAAGAGGGATTTGGAGCTTCATCTA
CGATTATTATACCTACCTACGTATAGACTATAATGGATAGTAAAAATAGAAAGTAATTTC
TTTTATATCAACTTTTGTATTCATATGTAAGTACTTACTTATAATTATTTATTTTTTTAG
GTGAAA
3′UTR
(SEQ ID NO: 46)
GTTAACAATAAACAATACTGTTAACCGTACGTAGTGTTAAATAATACAAATATATGTATT
TTACAATAAGATTTCCTGTTTTTAAATTATCCTCACGTAACGTATTGGGTATCTAAGCGG
TTGAGCAGTGAGATAAGTCTACCCCAAAGTAGTCCGTTCATCAAACGACATAACCATCTC
GTCATTTAGTTAAAACAAAAAAAGAATAATGATAAAAGATATAAAATTCTGTATAAGAAC
CAATACCTAGCACCTTTCTCACATTGCCAAACATACATTAAAAAACAGTATTATGCTTTT
TTCGCTCTTTTATTCTATATTTTATATTAAAAAATACCTATAGTAAATACTGTTTTCATC
GTCGCCGGTATTCATACACGTAGTATACGTACCTAACTCTGAAACTACTGGTTTGAATTG
AAAAA
>M. sexta-Serine Proteinase homolog 3 (MsSPH-3); AF413067.1
5′UTR
(SEQ ID NO: 47)
AGCGCTTTGGGAACGTTCCGTGGCACGATA
3′UTR
(SEQ ID NO: 48)
GATGGTGGAATGTTTAAAAATACAAGAGTGTGAGGCAGTGATTAATGGTCTGTAATATCC
GATTCTTGCTGGCTTCTGTTTCGTAATTAACGTAAAATCTAATTATCATTAGAATATTTT
GGAAAACCTTAGAGTTACCTTCGGAAAACTTCACCGTTTGCCGCTGGTGTGAAGATCTGT
TCCCCCCATAAACTGATGGGAAATTGTATGTAGGTACTCGAGTTATAATTTCTTTTTATA
CCTGCGTTTAAGTTCCATTACAACTTTCTAATTTTATGAATAAATATCTAATAAACGATT
AACAAAATTAAAAAAAAAAAAAAAAAA
>M. sexta-Peptidoglycan recognition protein 2 (MsPGRP2); GQ293365.1
5′UTR
(SEQ ID NO: 49)
ACGGCATAAATTGTTAGGTCGTCTGAGAGGCGAGTGTTGTATTTTTAATTGCCAAAGAGG
CTGCGAAGTTCGCAATAATCATTAGAAAA
3′UTR
(SEQ ID NO: 50)
AATGACCAAAGATAATACGACTGTTTTATAATTTTTGTTAATAAATGTTGTTGCATTATG
GAAAAAAA
>M. sexta-Beta-1, 3-glucan-recognition protein 2 (MsßGRP2); AY135522.1
3′UTR
(SEQ ID NO: 51)
ATTAAAACTAACGAAAAGACTCGTTTCCATAGTGGTATATTTTCAACTCGTCAATTTCAG
GTACAAGCATGTTTGGTGGAAGGATATTATCGGCCCGAATAGGCACTGTACACCAAGTAC
AACCATGACTCATCAAAGGTGTCGCGTTCTGGAATCACCTGTATTTCCAACAGGCCGGCA
TAATTGTGTCGTCTAGCGAGGGATAACTCAATAGTCTATTTGAACGCCATTCTACTTACC
ATTAGGTGTAGTGAAGATATTTGGGGCTAGACAAAATGGAAGGAAATTTATGAGTTGCAT
CGAATGATTATCACTAAGTATTCGAATGATTCATTTCGTTGTTGGTGCAATAGGATGTCA
AGGGTCAAATGGACATTAAAAGAGAGTTTAAGGTGTTTTTTTACAATTAACGTTGATCTA
TCACTTTACTTGCCTATTAAGTAAAGTATTATTTTGAAAAATACAAAATACTTGAGTTTA
TAGTATCTCTTGCGTTTATGGTACTGTCAAAAATATCAGTATTATTCATTTACAAAATTA
AAATTTAATAATCTAAATTATTCTTCATGTAAATGATCATTTATACCTCTGCCTTGATTA
TG
>M. sexta-Relish family protein 2A (MsREL2A); HM363513.1
5′UTR
(SEQ ID NO: 52)
AGAGTACGTTCGATGGCAGTCTGTCGAACATTGGTAGTTTCCCGTTTGAGTGTTGTTTAC
TCCCTTTGAGGGATTAGTTTATTCTCCACGAATATAAACATCGGAAAATCAAAAACTAAG
TTGATAAAAAGTTGTGTGCTCCGGTATAATTTTTTGGTATCAGTGACGGACAAAGGTGAT
ATAAAA
3′UTR
(SEQ ID NO: 53)
TATATTCATGAGAAGGGGACAACTAAGGATTGAATATCAGCAGAACTTGAGTTATGTTAA
TGAGGTATTTATATTAGCTTAATACTTAAAGGGGAAAATTCGATTAGCTTTTCAAATATA
CTTAAATTTTAGTTTTTGTCAAATATCGGTGTTTCCCATTTTGATATTTTTTTATCCATA
TTTTAATAATAAATATCTTGTCTTAGCAATTTTAATGGGTAATTATAAAGAAACTCACGC
ACCATAGTTGCACTAAACTACAAAATTACACAAAA
>M. sexta-Dorsal (MsDor); HM363515.1
5′UTR
(SEQ ID NO: 54)
ATACAACGATATCACAGGCGTCCGGGGACGGACCAGTTTAAACAAACATTACAGTGAATA
GCGATGTGATTATTTTCTTGCTTGTATAGAGTTAAATTTTTAAATTAGATTTAAATATTA
AAATATTTGCGAATAAAA
3′UTR
(SEQ ID NO: 55)
ACTTAACAATTACATCTATAAATCTCTCCTCTATCAACCTAGTGGGCGTCCTCTACAACT
ACCACGTCTGGTATACAAATAGTGACAACCTGAAATTGTGAGATTTAAATTGTGTAGTTA
TGATAAATAAACAATAAATCCAAAAAAAAAAAAAAAAAAAAAAAAAAA
>M. sexta-Toll receptor (MsTOLL); EF442782.1
5′UTR
(SEQ ID NO: 56)
GAAAAGTTATTCACAATATGACCCGAAGTGTCAGCGCCGCACATGCGCGGGAGCACTCGT
ACTTATACCAGACATTGTGACTAAATACCTAATTTTATGTTTTGCTCATCGCCCATTGCG
CGACATTAAGCAGTATCCAGTAGTCAGTGATCAGTGTTACTACACTATTTTGCTTCTCAG
CGATTAGTCAACACGCGCATATCGTTTACTTCAACAAACAATTATTACGCAAATCGTGAT
ATTTGAATGTAGAGACACGAATCAAAGATTATTCGTTTAACGTTATTTTCGCGGGATGTT
TCTCTGTTGGGGGGTACTGTTGCGTGGTGTGCAGTCAAAGGTTTATTTTATCGGGTATTG
TGCTCTTTAAGTGTTGACGCGGCGGATTATCGTCGGTTAGACTAATAAATCGTGTCGATT
TATGTGTGACCCACTACGGTGTCGTGCGACGTG
3′UTR
(SEQ ID NO: 57)
ATCCAGAACTTGCGTGTACCCTTCGATATACAGGGCTATTTTGACATCGTGTTACTAAAT
GAAACCACATTCTCGTCTTCACCTTTGCTGACATTGTGCCAAAAATCATCCATTAATAAC
GAATATTTCCACCAAAAAAAAAAAAAAAAA
>M. sexta-Scolexin A (MsSCA1); AF087004.1
3′UTR
(SEQ ID NO: 58)
TAGACCATACCGTTGTCATTTTGGGCTGTAGTGTATAGATAATAAATATAGACGCGTGTA
CTGGTGTGACGTACGGAAGTGGAGAGTTGGGAGCGACAGCTCACCGCTCACTCCACTCCC
GGCCGCCGCGCGAGTAGCAGTGTCAGTGATTGCAAAAAAAAAAAAAAAAAAAAAAAAAAA
AAAAAAAAAA
>M. sexta-Hemolymph proteinase 18 (MsHP18); AY672794.1
5′UTR
(SEQ ID NO: 59)
GCCAGACGTTTCAGTTTGTTTCGAACT
3′UTR
(SEQ ID NO: 60)
TTATGAATAAATACAATTTAATAAGCATTATTTATTAAAAAAAAAAAAAAAAAAAAAAAA
>M. sexta-Chymotrypsinogen-like protein 1 (MsCTL1); AM419170.1
5′UTR
(SEQ ID NO: 61)
TACTTCGGGCTACACAATGTACGTGAAAGTA
3′UTR
(SEQ ID NO: 62)
TCTCCGAACCGAACCTGATCTTAACTGTAAATAAAATAAGCGATACCCAAAAAAAAAAAA
AAAAAAAAAAAAAAAAA
>M. sexta-Dredd (MsDRD); Msex2.04297-RC; Currently annotated as Manduca sexta
Caspase-6; HM234679.1 in NCBI
5′UTR
(SEQ ID NO: 63)
TTGGTAACCCTGTGGGAAGTCCCAAAGTCGAAGCTCGAGAACAGTTTTAAGCAGAGGTT
TTAAGTCTAATTTATTGGTCTCTGGTTTTAAGTTAGTGTCATGTGTCAACAATAATAAA
GCTTTATTTTTGGCTCTATTTATAAGTAAGGTATCATCCTTTCAGCGTTAAAACAAAGG
GCGTAGCTCATGTTCATGTAGTTCTTTTAAATAAGTGTGAACTAAGTGTATATGTACAT
TTATTACTGGTGACA
3′UTR
(SEQ ID NO: 64)
CATAATATTTTTATTTAGATATATTGTAAGTACTAAGAATGAAGTGGTGATAGCTTAGTT
GGGAGTGGAATGGACAGCCAAGACAAATGTCAGCAAGTTCAAATCCAAAGGCATACACCT
CTGACTTTTAAAAAAAAATATGTGTGTATTCTTTGTAAATTATCACTTGCTTTACGGTGA
AGGAAAACATCATGAGGAAACTCTCAATAGTAAAGGTATCCCATTGCCCAGCAGTGGGAC
AGTATATTATACAGGGTTGATATTATTATTATTATATACTATGCTTTGCTCATGCGCTCA
TTCACTCCAGCATTAATGACCTTTCCTGGTATTGAAGTTCTGCTAAGTATTTTATGGGAT
AAAAATACCCTGAATGTTAATCCACACACATTCAGTATGTCAAATTTCATCTAAATCCAT
TTAGGTGTGTTCTATTTGATACTAAGTTCCAACAAAAATCTTCATAAACGTCAGGACCCC
TCAGGAGCAACATTATTAATAAGATTAATAATAAATTTTCAATGCATAATAATTTTAATA
TAATTTATATTACTTAATGTTGAAGTTGATATACTTAA
>M. sexta-Relish F (MsRelF); HM363513.1
5′UTR
(SEQ ID NO: 65)
AGAGTACGTTCGATGGCAGTCTGTCGAACATTGGTAGTTTCCCGTTTGAGTGTTGTTTAC
TCCCTTTGAGGGATTAGTTTATTCTCCACGAATATAAACATCGGAAAATCAAAAACTAAG
TTGATAAAAAGTTGTGTGCTCCGGTATAATTTTTTGGTATCAGTGACGGACAAAGGTGAT
ATAAAA
3′UTR
(SEQ ID NO: 66)
TATATTCATGAGAAGGGGACAACTAAGGATTGAATATCAGCAGAACTTGAGTTATGTTAA
TGAGGTATTTATATTAGCTTAATACTTAAAGGGGAAAATTCGATTAGCTTTTCAAATATA
CTTAAATTTTAGTTTTTGTCAAATATCGGTGTTTCCCATTTTGATATTTTTTTATCCATA
TTTTAATAATAAATATCTTGTCTTAGCAATTTTAATGGGTAATTATAAAGAAACTCACGC
ACCATAGTTGCACTAAACTACAAAATTACACAAAAAAAAAAAAAAAAAAAAAAAAAA
>M. sexta-Chitin synthase 2 (MSCHS2); AY821560.1
5′UTR
(SEQ ID NO: 67)
ATTACGCTTTGAGCAGCCACTTTGTAAACACTAGCCTATTAACTCCGTCACTGCTTCGGC
GATAACACTTCTGTATATCTGTTATTGTTTGTTAATTTTTGGCGCAAGGATATATTTAAT
ATGTGCTAAGTTTGTGGCTAATTTATATTATGTTTCTACATTAAAGGTATGTATTAGTGA
TTTTTTGTAACTTATTTAATTACAAACATTATAACATATTTTATTAGAAAAGGGTTTTGT
ATCCTATGTAGCGCCTGTTTCACGATGCCTCTTGAAGTATACTGAAATGGGTACCCGTCG
ATAGTGTTTGCTTTAATAGGCAACGCATAGTGAAAACTTTGTACTTTAACCTTTTCAAAA
GCATGAATAATTAAAAAAAAATATTAGAATAATGTGAAAAAGGTGTACAGTGTGATAAAT
ATGGAGTATACAATATTCTTGTTCCAGTTATAAGATAAGGCACA
3′UTR
(SEQ ID NO: 68)
TATTTGGCCAAAATTGTGAACTGTAAACATGCAATAAAATGATGAACG
>M. sexta-Beta-fructofuranosidase 1 (MsSuc1); GQ293363.1
5′UTR
(SEQ ID NO: 69)
TGAGAACAAAAACATTAGCTCCGCGTTTAAAA
3′UTR
(SEQ ID NO: 70)
TTATCGTGCTAAAGTAATAGTTATTGTTTCACATTATGTTCAAATAAAAAAGTAATTATT
TATGTTCAAATAAAAAAGTAATTATTTAAGTGTCTTGTAAATCTGCGAATAAATATACTT
AATGTATAAAAAAAAAAAAA
>M. sexta-Sickie (MsSck); Msex2.03324RA
(SEQ ID NO: 71)
ATGACGACGTTGACTGCAAATAGGCAAGTGTCGGTGTTGCAGACGTCGATCCCGCTGCCG
GCGTCGGCGGCGGCGGTGCGGCGCGCTCCGCCCGACAAACGACCGCTGCCCGCCACACCA
GTACATGCAGGTGGCAAGAGTGGTCTTTCTGCGAGTTCCAGCCGCTCCACCAGTCCATTA
GGACAGGGTGCAGTCAGTTTTATACCACGTGCGCCTAATTCATCATCTGGATCCCGGCCG
AACTCGTCCCTCCTTGCGCCCAGTAGCAAAATACCCTCCACCGCAACACAAGGGCAACAG
CATTCACAAACACCCACAGCCCAAAATGGAACACCCACAAAGCAGTCTATGCTTGACAAG
TTAAAGCTATTCAATAAAGACAAAATTAGCAATGAAAAACAAAACAGCAAAAGCACTGCA
GTATCAAAACGCACCAGTTCTTCCAGTGGTTTCTCATCGGCAAAGAGTGAGAGATCAGAC
TCGAGTTTAAGCCTTAATGAGTCTTCTAACATACCCAACACACATATCAAATCGTCTAAT
TTAATAGGACCTAAAACAATACGGCAACAAAGCGATACTTTATCAAAAGACAAATCTGGT
AAAAATTTAAAACCTAAGTTAGTTAATTCTAAATCTCCTAGAGATTCTACAACTAACTTG
AATAGGGCTAGTAGCAAAACAACTAATAATGATAAATCGAGGAATAGTCCAAAACTTCCT
GCTAGGGATAAGGAATCGAAACTAGCCACACCTAAAACCATGAGTAACACAAAACTAAAC
CAAGTTGAAGATCAACCGAGGGGTTCCAAAACTAAAGTGGAATCGAAAATGGTAAAGCTT
TCCGGTAGCCAAGTTAAGCTGAGTGAACAAAGATCCGATACTCCACAAGACTGTAAAGAA
AATGTAACTCCTAATGCGAAAAATCATCAGAGTCACTCTCAAACACCATGTGGAGGACAG
GGATTTGGAACTCCAAACCATACAGGAATACCTAAACCGACTGCCGCTGTCAAGGGAACT
TTTAAAATATCAAAAGACGATAGACATGTTATACAGAAAAGTACAAATAGTCAATTAAGT
CCTATACAAAGCAATTCTAGTTTAAATTCTACAAATAACATAAGTGCACTTCCATTCTGT
AGAGAACAATCTAATTTAAGTAAGGATATAACTCAAAAGCAGACACTAGCCGTATCCCCG
ATGCCGGTTATGAGCACAGGGAATCAAAATCATAGTTCGCAAATGTCTGAAAGTTCACAC
TCCAATTCTACGCACAGTACTACAGGCCAACATTCGAACTCTAGCGATAGCAGCGTTATA
TACCGTCCTTCTAGCGAATCAGGTTCTGAAATGTCGAAGACTGCTAGTCACAGTGTGGCA
TCAAATAAACGGCTAGACATGAATACTACTTATATAAATGACGTTATAAACGAAGCGGAG
ATATCAGAAAAGGAGGCAGCACAAAGACGTCACGTCGATAATTCAATCCCCAAGCAATCG
TTTGATCCCAATAAAACGTTAACCGAAATAAGTAGGAGGCTCGAAAGTGATAGTAGATCT
AGCACACCGTCGCATTGTCGTGATAATTCTTTAGGCGAAGACGAGAATCCAATGATGAAT
GTTTTACCAATGAGACCGTTACTTCGCGGATACAACAGTCATTTAACCTTGCCCATGAGA
ACTTCAGGTTTGGCACAGAAAAATATAAGCGTTTATCCACACCACGCAAATACTGTAAAA
GCAAACTTTGGACGGGAAAACATAGGTCTTCGCGATCGTATAAACTATGGGCCTGGTTTT
TCGAATCCTGATTATTGTGATCTGGAAATTGCTTCGGGATACATGTCTGATGGTGATTGT
TTGAGAAGAATTAATATCGGTGAGATGGAATGCGGGAGGAACAACGACATGATGGACGGC
TATATGTCCGAGGGTGGTGCTTCACTCTACGGTCGTCGGATGAACTACCAGCAACCACAA
TTTCAACAAATGGACGAAAGACGAAGTGGTGGTCGCAATAGAGGCATGGAGGGTGGTAGT
GGTGTAGTCTACAGAGTAGTGGGTCGCACACGCAGCAAAGCCGACTCCGGACAGCAGACC
GAGCGCCAGCCTCAGCCTTCCCGGCAGGACACCACGTGGAAGAAGTACACTGACTCGCCC
GGGAATCAGCCAGCACCCGCGCCTCCTAGTCCATCTCACTCGCGCAAGGGTGAGAGACGA
ACTGGCCATCATTCACCGCAGCACCACAAACGAGAAAAACTCACTGCAGCTCAGCAGCTT
GGGATCGCACCTCATCCTCAGTATGCCGCTCCAGCTCAAGCAGCTAACCCTGCTGGTCAG
CATTCCTCTAGAAACCCAGGCCCACAGCTCCAGTCTCCGAGTGGAGGGTCGCGTCCCTCC
AGCGTCTCTAGCGGTGGTAATGGCAGTGCCTGCTCGTCGAAGGCTAAAGTTCCTCAAAAT
TTTGGATATGTGAAAAGACAAAACGGTGTACAGCAGCCGGCACCTCAGGCCAACGGTCCA
CCGCCGCAACATGGAGGCCACACTGGAAGAACCGCCCAGGTGTCTGCAGTGCCAAGAACT
AAAGTCAAAGTTTCGGGAGGAACCCAGACATGCACACAGGATTTACAGATTCACAAAAAT
GGTATCGGACCTAAATCGTATTCTCTGGGCGGCACCGCGGCTGCCCAACTGTCAGCGTCA
GTGCGAGAGAGACTGCTCGGGTCACAATCACTACCAAAACCTGGAACACACGAATTCGCA
GCGTTATTCCATCACCACAGAATTGCACCCAGAGGCGGCATGAAGATCAGCGATGGCAGC
CTTTCTGACACACAGACATACTCTGAAGTGAAATCAGACTATGGGATACCCTACGCGCCC
TGGCTCAGGCATAGCAACACATACTCGGCGAGCGGGCGGTTGTCAGAGGGAGAGTCGATG
GAGTCGCTCACGTCGCTGCACTCGGCGCAGGCGCAGGCGCACAACCACACCTCCTCCCCT
AACTCCCACCGCAGCTCACTCACACACAATAAGCTCATCATGCACCGAGATGCACAGGGC
TCCAGGCTTAACAGGAGCAACAGCATTAGATCGACTAAGTCAGAAAAATTATATCCGTCG
ATGCTTCAAAGGTCTTCAGAGAGCGATTATGAACCGTATTATTGTTTACCTGTGCAGTAT
GGACCAACAGGTCAAGGTCTAAACTATGGTGTGTCGGAGCCGCCGTCGCCGTCGCCGCGC
TCGGCGCTGAGCCCGACGCACGCGCCCGGCAACGTCATGCACACGCCGCGCCACTCACAC
CACTACCCGAAAAAGAACGACGATGTGCACGGTTCGACGGCGTCGCTAGTGTCGACCGCC
TCCTCGCTGGCTGCCGGCGCAGGCGCAGAAGAGAGGCACGCCCATGAGGTGCGAAAGCTG
AGAAGAGAACTGGCCGATGCGAAAGAAAAAGTTCACACTTTAACAACGCAGTTAACAACC
AACGCGCACGTGGTGTCCGCATTCGAGCAGAGTCTGTCGAATATGACGCAGCGCCTGCAG
CAGCTCACCGCAACCGCCGAGAGAAAGGATTCGGAACTGACGGAGCTTCGTCAAACGATT
GAGTTGCTGCGGAAACAGTCGATTCAAGCTGGTCTGACTACTGCTCACATGCAGTCCATG
GGAATCCGTGCCGATGGCGTCAACGTCACTGGACAGCAGGACCCAACAACCCAAACGCAG
CAGTCATCACCACAACGTAACGCACAAACGGGCAACGGTGCGATCACGCGGCATCTATCG
ACCGACAGCGTGTCGAGCATCAACAGCCTGAGCAGCGGCTCCTCAGCGCCTCACGACAAA
AAACACAAGAAAAAAGGATGGCTACGCTCATCGTTCACGAAGGCATTCTCACGGAACGCG
AAGATATCTAAAACAGCGAAGCACTCGTCCCTCGGGCAGCTGTCGTCACAGGACAGCTCG
TCGGGCTCGCATCACTACGACGACCCGCACACGATACGAGAGGGCAGTAATGAGAACAGT
CTAGAACATTCCCACGAGGCTCTTCCTGATACTAAGGAGAAGCCGGCGCCTGTCAAGCCT
GAAGAACAAACCAAAGATAACAAAGAGGAATCTGTCTTGGTGGATGAGCTGAAGCGGCAA
CTGCGCGAGAAGGATCTCGTGTTGACCGACATCAGACTGGAGGCGCTCAGCTCCGCGCAT
CAGTTGGAGAGTCTCAAAGACACCGTTATAAAAATGAGAAACGAGATGTTGAACCTGAAG
CAAAACAACGAGCGTTTGCAGCGGCTGGTGACGTCACGCTCGCTCGCCGGCAGCCAGAGC
TCGCTCGGCACCGGCGGCTCCGCCGTCGAGGACCCGAGGCGGTTCAGCCTCGCGGACCAG
GCCACCATGCACCAGGCGGCGATCGACATTCACGCCCAGCCTCTCGACCTCGACTTCAAC
TGCATGTCAGCCACACCAACCATCGACTTCTCGAAGAAAGGTTCACCCAAGTCCGGCATG
GTGGAGCCCATATATGGGAATAAGGCTGCCTGCGAACTGAACGAGAACAATGAGACGCTG
CTAGGAGCGTTGAATGGGGCCAGCGATTTGTTCTCCAACGGACTCAATGCTGGAGAGCGG
CTGAGTGGAGATTACGATATAAACAGCGTGTTACCGCCGCCTAAAACACGCGAGCTTGCT
ATTGGTGAAAGCTACTCTGATATTGGTGTAGCAGACAGCCAGGGAGACACGACTGATGGA
AAGAAAATAGCGATAGCTGTATATTTAGGTCAACCCGAAACATTCCAAAGATATTTCGAA
GAGGTCCAAGACACATTGACGGAGTCCGAGTGCAGATTCTACGCGAAGCAATCGGCAAGC
GCGTACAATCATTTCGAGAAACAAGCCAGTTTCGAATCGCCGAGAATGTCCACGAATCAC
AGTCCAGAAGTGGAGACACAAGACTACCCGCAAATAAATAAGTCGAACACGAATAGCCTT
AAAAGCAACAAATCTACGCACAGTAGCTCGTATAAGAATGTTTATAATAGTGATTCGACA
ATAAACTGCAACGAGTACACTATAGCGTACACTTATATATCTGGCAAGACGACTTGGCAG
AATTTAGATTATATAGTTAGGAAGTCCTTTAAGGACTACTTGTCTAGGATAGATTTGGGC
ACGAATCTCGGTCTGAACACTGATTCTATAACGTCGTACCATTTGGGTGAAGCGACGCGT
GGGCCGGAGATCGGCTTCCCGGAGCTGTTGCCATGCGGGTACATCATAGGGACCGTGAAC
ACGCTGTACATCTGCTTGCAAGGAGTCGGCAGTCTGGCGTTCGATAGCCTTATACCAAAA
AATATTGTATATAGATACGTTTCTCTGCTATCGGAACACAGGCGAGTGATACTCTGTGGT
CCCAGCGGCACCGGCAAGTCATACTTAGCGGCGAAACTCGCTGAATTTTACGTCCAGAAA
ACACAAAGGCGCGGCAATCCCGCCGAAGCTGTAGCTACATTCAACGTGGACAGAAAGTCG
TGCAACGAGCTGCGCGCGTACCTGGCGAACATCGCGGAGCAGTGCGGCGCGGCGGCCGCG
GGCGAGGAGGCGCCGCTGCCGTCCGTCGTGGTGCTCGACAACCTGCAGCACGCCTCCGCG
CTCGGCGACGCCTTCGCGGGGCTGCTGCCGCCCGACAACAGGAACATGCCCGTTATTATC
GGTACCATGTCCCAAGCGACGTGCAACACCACGAATCTCCAACTACATCACAACTTCAGA
TGGCTCCTCACCGCTAACCACATGGAACCCGTCAAAGGATTCTTAGCTAGGTATCTCCGA
AGAAAGTTATTCTCGCTGGAGCTGCGGCTGGGTCGGCGCGAGCCGGCGCTGGCGGCGGTG
CTGGAGTGGCTGCCGGGCGTGTGGGCCGCGCTCAACGCCTTCCTCGAGGCGCACTCCTCC
AGCGACGTCACCGTCGGGCCGCGGCTCTTCCTCGCCTGCCCCATGGACTTGGAGGCCAGC
CAGGCATGGTTCGCAGACGTGTGGAACTACAGCATAGTGCCGTACGCTTCGGAGGCGGTG
CGCGAGGGCATCGCGCTGTACGGCAGGCGACGACACGCCGCCGTCGACCCGCTGCAGCAC
ATCAAGTCTACATACCCCTGGAGAGAACCCAATCACTCGCATACTTTGAGACCCATAACA
GTTGATGATGTTGGCATCGAAGAGTCAAGCCAAGACTCCGCCGTGAACAACAATCAAGAT
CCTCTGTTGAACATGCTGATGCGGCTACAAGAAGCGGCGAACTACAGCGGAAACCAAAGC
CAGGACTCTGACAACGCCAGCATGGACTCGAACCTCACACACGACAGCTCTGTAGGGAAC
GAGCTTTAA
>M. sexta-Akirin (MsAki); Msex2.12479-RA
(SEQ ID NO: 72)
ATGGCGTGTGCTACACTTAAAAGAAATTTGGATTGGGAATCCATGGCGCAATTGCCTGCT
AAAAGGCGAAGATGTTCGCCATTTGCTGCAAGTTCTAGCACAAGTCCTGGATTTAAAGTG
TCTGAAACCAAGCCATCTACATTCGGAGAGGCCGTTAGTGCACCTGTGAAAATGACCCCA
GAGCGCATGGCTCAAGAGATCTGCGACGAGATCAAGCGGCTGCAGCGGCGCCGGCAACTG
CGGCTGGCTGGCAGCTCCGCCGCTTCGTGCTCATCGTCGAGCGGCAGCGAGGGCGACTGC
TCGCCGCCACATCGCTCCTCGCACACTTCGCACAAGATGCACAACCGTGCGCTCTTCACT
TTCAAACAGGTGCGCATGATCTGCGAGCGGATGCTGCGCGAGCAGGAGGTGGCTCTGCGC
GCGGAGTACGAGTCGGCGCTCAGCACCAAGCTCGCCGAGCAGTACGAGGCGTTCGTGCGG
TTCAACCTTGATCAGGTGCAGCGCAGACCCCCGCCCAGCACGTGCATGCCCCTCGGCATG
GACGCCGAGCATCACATGCACCAGGACCTCGTACCTAGCTATCTGTCCTAA
>M. sexta-Cactus (MsCac); Msex2.02793-RA
(SEQ ID NO: 73)
ATGAGTGCCAAAAAAGGATATGAAACGAAGATTGTCGAGGAAGAAAACATGGATTCCGGA
ATTGTGTCTGGTGAATTGGAATCTTATGAGATTTCGGGTGAAGTGGATTCGGGCGTGATT
GATTGTGATAAGAAATACGAAGGGGTTCCAAGTGAGGTGTTGGAATTGACGGACAAGTTC
AAAAGTGTAAATGTGAGAGAGAAGAGCTGTCCTGATGTTCCACCACTGGCGGACCTGTTC
CACCCTGACAACGACGGAGATACACAACTACACATTGCATCGGTACACGGCTGCGAGAAA
TCAGTGAGCACGATCATCAGGGTGTGCCCTGACAAGGAGTGGCTGGACCTGCCCAACGAC
TACGGCCACACGCCCCTCCACCTCGCGGTGATGAGCGGCAATGCCGTGGTGACAAGGATG
CTGGTGATAGCCGGCGCTTCGCTCGCTATTCGCGACTTCATGGGAGAGACGCCCTTACAC
AAGGCGACCGCAGCGCGAAACCAGGAGTGTCTCAAAGCCCTGCTTGCCCCTGTACCGGAA
CAGCCCAATAGGAAATTGTCTTCAATACTCGACCAGAGGAACTATAACGGTCAATGTTGT
GTCCACCTGGCGGCGTCAATTGGAAGCGTAGAGACGCTACAGACCCTGGTCTACTACGGA
GCCGATATCAATGCCAGGGAGAACCTGGCGGGCTGGACGGCGCTGCACATCGCGGCGCGG
CGCGGCGACGTGCGCGTGGTGCAGTTCCTGCGGTCGCGCTGCGCCGGCGCGGCGACGCGG
CCGCGGGACTACGCCGGCCGCACGCCGCGCCGCCTCGCGCGCCGCACCAAGGCCGCCGCC
GCCTTCGACGACAAGGACGACAGCGACTCCGACTCCGACTCGGACGATGATGATATGTAC
GACAGTGATAGCGAGACGTTGTTCGAAAAACTCCGCGAGAGCCTGAGCACGTCGATCAAC
GTCGCCTGA
>M. sexta-Gloverin (MsGLV); GI110649240
(SEQ ID NO: 74)
CCACGACAACCACTGATGAAGTTATTTTTTATAGCAATTCTTTTCGCTGCCATCGTCGCT
TGCGCGTGCGCTCAAGTGTCGATGCCCCCGCAATACGCTCAGATATATCCAGAATATTAC
AAGTACTCCAAACAAGTCCGCCATCCCAGAGACGTGACCTGGGACAAGCAAGTCGGCAAC
AATGGGAAGGTCTTCGGAACTCTGGGACAGAATGACCAGGGTCTTTTCGGTAAAGGAGGC
TATCAACACCAATTCTTCGATGATCACCGCGGCAAACTGACAGGACAGGGTTACGGGTCC
AGGGTCCTCGGACCTTACGGAGACAGCACCAACTTCGGCGGCCGGCTTGACTGGGCCAAC
AAGAATGCTAACGCTGCTCTTGATGTGACCAAGAGCATTGGCGGTAGGACTGGGCTGACT
GCCAGTGGATCAGGCGTGTGGCAACTTGGGAAGAACACGGATTTATCTGCGGGAGGCACT
CTGTCTCAGACGCTTGGACATGGGAAGCCTGATGTCGGCTTCCAAGGTCTCTTCCAGCAT
AGATGGTGA
>M. sexta- Beta-1 tubulin (MsßTub); AF030547
(SEQ ID NO: 75)
ATGAGGGAAATCGTGCACATCCAGGCTGGCCAATGCGGCAACCAGATCGGAGCTAAGTTC
TGGGAGATCATCTCTGACGAGCATGGCATCGACCCCACCGGCGCTTACCATGGCGACTCG
GACCTGCAGCTGGAGCGCATCAACGTGTACTACAATGAGGCCTCCGGCGGCAAGTACGTG
CCGCGCGCCATCCTCGTGGACCTCGAGCCCGGCACCATGGACTCTGTCCGCTCCGGACCT
TTCGGACAGATCTTCCGCCCGGACAACTTCGTCTTCGGACAGTCCGGCGCCGGTAACAAC
TGGGCCAAGGGACACTACACAGAGGGCGCCGAGCTTGTCGACTCGGTCTTAGACGTCGTA
CGTAAGGAAGCAGAATCATGCGACTGCCTCCAGGGATTCCAACTCACACACTCGCTCGGC
GGCGGTACCGGTTCCGGAATGGGCACCCTCCTTATCTCCAAAATCAGGGAAGAATACCCC
GACAGAATTATGAACACATATTCAGTTGTACCATCACCCAAAGTGTCTGATACAGTAGTA
GAACCTTACAATGCAACACTGTCAGTCCACCAACTCGTAGAAAACACCGACGAAACCTAC
TGTATCGACAATGAGGCTCTCTATGACATCTGCTTCCGCACGCTCAAACTTTCCACACCC
ACATATGGCGACCTTAACCACCTGGTGTCGCTCACAATGTCCGGCGTGACCACCTGCCTC
AGGTTCCCCGGTCAGCTGAATGCGGATCTCCGCAAGCTGGCGGTGAACATGGTGCCCTTC
CCGCGTCTGCACTTCTTCATGCCGGGCTTCGCTCCGCTCACGTCGCGCGGCAGCCAGCAG
TACCGCGCCCTCACCGTGCCCGAACTCACCCAGCAGATGTTCGACGCTAAGAACATGATG
GCGGCGTGCGACCCGCGTCACGGCCGCTACCTCACCGTCGCCGCCATCTTCCGTGGTCGC
ATGTCCATGAAGGAGGTCGACGAGCAGATGCTCAACATCCAGAACAAGAACTCGTCGTAC
TTCGTTGAATGGATCCCCAACAACGTGAAGACCGCCGTGTGCGACATCCCGCCCCGTGGT
CTCAAGATGTCGGCCACTTTCATCGGCAACTCCACCGCTATCCAGGAGCTGTTCAAGCGC
ATCTCTGAACAGTTCACCGCTATGTTCAGGCGCAAGGCTTTCTTGCATTGGTACACCGGC
GAGGGCATGGACGAGATGGAGTTCACCGAGGCCGAGAGCAACATGAACGACCTGGTGTCC
GAGTACCAACAGTACCAGGAGGCCACCGCCGACGAGGACGCCGAGTTCGACGAGGAGCAA
GAGCAGGAGATCGAGGACAACTAG
>P. xylostella-Peptidoglycan recognition protein 2 (PxPGRP2); ACB32179.1
(SEQ ID NO: 76)
ATGACGTTGTCTTTTGGCGTGTTTCTGCTGATATCTTCAGTGTTTTGTTGTTGTGCTCAT
GCAGGGTGTGGCGTGGTGACCAGACAGCAGTGGGATGGGCTGGACCCGATACAGTTGGAG
TACCTGCCCCGGCCCCTGGGGCTGGTGGTGGTCCAGCACACCGCCACCCCCGCGTGTGAC
ACTGACGCCGCGTGTGTGGAGCTGGTGCAGAACATACAGACCAATCATATGGATGTGCTG
AAGTTTTGGGATATTGGACCGAACTTCCTGATTGGTGGGAACGGCAAGGTGTACGAGGGC
CCTGGTTGGCTGCACGTCGGCGCCCACACTTACGGCTACAACAGGAAGTCTATCGGGATC
TCTTTCATTAGGAATTTTAATGCTAAGACCCCAACAAAAGCAGCGTTGAATGCGGCTGAA
GCATTGCTGAAGTGTGGAGTGAGAGAAGGACACCTGTCTCACTCATACGCAGTGGTCGGC
CATAGACAACTGATCGCAACAGAGAGCCCAGGCAGGAAACTGTACCAAATCATCAGGCGC
TGGCCAAACTACCTCGAGGATATTGATAAGATTAAAAACAACAAGTAG
>P. xylostella-Immune Deficiency Protein (PxIMD); Px003008
(SEQ ID NO: 77)
ATGTCTATCCTAAAATCAAAGTTATTCGAAACTATTGCAAAAAGTTTCAAGTCTGATGCA
GTCCCGAAGCCACCTAGAGAACCGGTAGAGACTACAGAGACATCACAAAATAATACCGAA
AATCAACCTTACAATGTCGAAGAAGAGGAAATACCCGAACCAGAAAAGCCTAAGAAAGAA
AAAAAGAATCCCAAGCCTACCAAAAAAACTTTCTTTAATCGTGACAAAACTAACAAACAC
GACGATACCCGCAAACATACAAAATCCGGAAAGGACCAGACATCAATTAATACTCAAGGT
AACTTGAAAATTATACTTCCTGTAACTTAAACGGCCCGTTGACCCCAGTTTACCTTTCGC
CTTTCTTGATATATTTTTGTAATCCAGCCTTACTTTGGTAATACATACTTGCCCCACTTG
TATTTAGTTAATGGTGGCACTAGCTAGATAGTAATGTTAAATGATGATAAGCAGTAGTGA
TTCATCATTCAAATGTATCATTGTCCTTTAATGTTAAGCGCAAATAGATTTTCATTGTTC
TCCCATGTGCTTCATGTTTTATGTATTTATAGGTAGGTACTTAATGTTTTATAAATATTT
TTTTGTTAATTGGGAATCCCCAGTCCCCATTGTCTGGACCAGTTTATATATAATTGAACT
AACAAGAGTGTGCTTTAAATACTATTCTCTGCAATTATGATAATTAAACAACATGAATTT
CTCTTCACTTCCCTTCTCTTATTTAAATAATATTGTAGGAAACTGTAATAACTAATACAA
GATTATAAATTTCATTCTAGCAACTGGTGATGTAATCCATGTGGTAAATTCCAAAGATGT
GCAGGTCGGCCATCAGTATGTGTACAACATGGGAACTCCCGGAGCTAACTCACAGAAGAA
TAACCCATTTGATGATGAAGAAACAGTAGAAAAGACAAATCTAATAACTCTGGTCATGGA
AGCAAAAATTATGGTAATAACACATTTTTAACTAGGCATAAGGTCATAATTTAGCCAGAA
TCATCAGCTTGTCCTGTGGCTCTTGTTGAGCTGGTGGAAAGAATACATAGGTAATGAATA
TTTTGATCAATACTCATTGCAAAAATCACAATAATGCCATTGAAAATCTATAACATGTTC
CTTAAGTATCACTTATCATCAATCCAATTAAGTCATCACACAGATCAATCGGTTAGTTCT
GTTTATTTACTTCTTTCAGCTGGAACATGAATACATGGACTATGTCTCGAAGAACCTCGG
CAGGAACTGGCACAGCTTCTTCAGAACGCTCGGCTTCACGCGGGGGCGCATCGAGACTGT
GGAACTGGATGAGGGCAGAAATGGTGTTGCAGAGGTATGAAAAAAACATACATGTTAATT
TGTGTTGTTTGGCTGATGTGGCAACTAAGTTCGTAACTGCTATGATCAACTGTTTGTGTC
ATAGGTATTTTTTTTGCCCTTCCACACATCTGAGGTAACAAGGACACCTCCTGCACTCAA
AACACAACTGCTATGTCCTACTGATGAGTCCTAGGAAACTCAGAAACCATCTGTTTCCCC
CATTCTCAATTCCATAAAGCTAAAAAGTGGTAGTTACACAATTCACAATTCATAAACATG
CTTTGTCATAGTTAGAAAAGCAGCTCAGCTATGAGGCATCCACTCCACAGTCCACTCACG
AAAGCCCTCTTTAAAAACATAAAATCATCATCATCAGCCCTCAATTGCCCACTGTTGCAT
ATAGGCCTTCTTTTGATTATGCCAAGTTTTTCGTTTCTCTAGTCCACAGACTTGACATAG
TTGTCACAAATTTATTCTACGTACTTGTATTTCCAGGTGCGCTACAAGTTGCTCCTGGAG
TGGGCCCGCACCGACGAGGACCCCACGCTGGGGCGGCTCGCCACGCGGCTGTGGGACGAG
GGAGAGCGGCAGACCGTCAAGGAACTCGCCATCTTGTATAATAATAATTTCAAGCAACAA
TGTTGA
>P. xylostella-Relish (PxRel); Px002858
(SEQ ID NO: 78)
ATGGTAGTGGTGAAAAGCCAATTAAAAATGCAAAGTGACCAGGACACGGACTCGTCCACT
GCCGGTGCTTCGCCGCGCAGCTTCTACATCGAGTCGCCGCACAGCTCGCCGGGACAACAA
GTGCCTTATTTAACTAATTACATGACAGTACTGTCCTGTGCAGATAATAACTTAATGGAT
ACAGGAAGCAATGGACCATTCCTGAGCATCACGGAGCAGCCATGTGACCACTTCAGGTTC
CGCTACAAGAGCGAGATGGTCGGCACCCACGGCTGCATCGTCGGCAAGACCAGCGCCAGC
AACCGCACCAAGACATACCCTTCTGTTGTTCTGCTCAACTACAAAGGCCGCGCCACCATC
AAGTGCAGCCTCGCGCAGCACAACAACCGCAAGCAGCACCCGCACCAGCTGGTCGAGGAT
GACCAGGAGCGCGACCTGAGCGCCGAGGTCAACCCCGAGAAGGGCTATGAAGTTGGATTT
CGTGGCATGGGCATAATACATACAGCGAAGAAAGATGTTCCGGCACTCCTATACAAGAAA
TTGAGTGAGAGACTGCCACATTTCAATGCCCGTGAGCTGAAGGCCCAGTGTGAGAACGAG
GCGCGCAGTATAAACCTCAACATCGTGCGCCTCAAGTTCAGTGCGCACAATGTCGACACG
GACGAGGAGATATGCGCTCCGGTGTTCTCGGAACCTATCCACAACATGAAAAGCGCCGCG
ACGAACGACCTGAAGATCTGCCGCATGAGCCGCACGTCGGGGCGCCCGCGCGGCGGCGAC
GACGTCTACCTACTCACCGAGAAGGTTAACAAAAAGAACATCGACATTCGCTTCGTGCAA
CTGGAGCGCGGCGAGGTGTGCTGGACCGGCAAGGCCAGGTTCCTCATGAGCGACGTGCAC
CACCAGTACGCTATTGTGATCAGAACACCAGCATACAAGAACCCCGAGATTACGTCTGAC
GTAAAAGTGTACGTAGAACTATTCCGCCCGTCCGATGGCCGCTCCAGCGAACGCATAGAG
TTCACGTACAAGGCAGAAGAAGTCTACAAGCAAAGCAAGAAACGGAAGGCCAACTCTTAC
TCCTCTATCGGAAGTTCATCTAGCGGTAATTCTATCAAAAGCGTCAGTGATCTTCCAGCA
ACTGTTATTATGGCCAATGAAATGAATGCGGCTAACAACAACTTTAGTAAAATCTCTTCA
ATGCTGAGCCCAAACAATATACCAGAAATACCGACACAAACGACTGTGGGTCTATCAGAC
GCTCTCTACGACATAACAGTGACAGAAGACCACCAAATGCACATCAGTCCCATGCTATGC
CAACCAGTGGAAGAGTATCCCCTAAAGCTTAACTCGCAGGATATCATACAAGTGAATTTG
AACTCTAAGGACATCGACCAACTGCTCAAAGTCAACAGTGTGCCTGATACCGATAAAGAC
TTTGCTGACTTCAATTTTAGTGACTACTACAAGGCACTCGATAGCAACTTTTTAGCTGAT
GGTGGTGGTGATAGCTTCAGTCAGTGTATCTTCAACTCTATGCAACTGAGGCCTGACTCT
GGGAGAGGCACG
>P. xylostella-Toll receptor (PxToll2); Px006338
(SEQ ID NO: 79)
ATGCCTAAAGTAATAATCGCCAGTTTTGCTTATATAGTAGGTGTGTTTGTCCTGTGTGCT
GGGCTAGAAACAAGTCCAACCTGTTCCAGCATCGAAGGACCTACTCCAGGTGAATTCATA
ATACAAAGAGGTATCGTACCGGACAATGATTCAGCCACCACAGTAAGCTTCCGAGGCTGC
CGAATATCTGACATTCAGCCGAGAGCATTCCATGGGCTACCTTCCCTGCAATACATAGAC
TTATCAAGAAATAGCATTAAAAACCTGAAACTTGGCATTCTTAATGACGTCACTAGACTC
ACTCATCTGAACTTGTCTTATAACTTCATCAGCGATTTAGAAGAGAGTTTGTTCAACCAA
TCGTCAAGGTTGGAGGTGTTGGATCTCCGGTGGAATAAGATTGAAGTTATGAAAGTGGGC
GTTTTCAGTCCATTGAAGAGATTGAAGTATTTGGATCTGTCCGACAACGAAATAGTTGGG
GCCAGCCTGAGCCCCGCTATGTTTGATTCCTGTAAAGCTCTATCCACTATCAATTTTTCA
AGAAATGATATGTCTGGTGCTCCCACTGATTTGCTTCGAGCTGTGGAGGTACTGGACACA
CTGAAACTGGATGGGTGCTTTTTGAAACAAGTTCCGGAATTTGCTACGAGGAGCAATACC
GGCACAATGAAGAAACTTATTTTATCATCGAACCAAGTGAGCACTGTGAAACTTACTACG
TTCATCAGTTTAACAAACCTGGAGGAACTAGATTTGAGTTCAAATGTAATTTCAGAATTG
CATGAAGACGTATTCAAGCCATTAAAAAACTTGAAAATTATTATTTTACGCTCGAACCGA
CTGGAAAAAATTCCTGATAAGTTGTTTTATAATATGTTACGATTAAGGAAGGTAGATTTA
TCTTTTAATTCTTTGATGATAATACCTGTGAATGCCTTTCGTTTTACGACGATAGAAATG
TTGAATATATCGCATAATAAGTTCACATATTTAGTCGACAACTTTTGTTTGGAACTTAGA
AACTCGGGAGTGAAACTGAAAAAGTTTTACTTCAACAGTAATCCCTGGCAGTGTCCTTGC
TTGAGGGATTTATTAAAGGAAATGAAGACGTATAGAATATCGTACAATAATGCTAAATAC
GATGGTAAAAATGCAGTTTGTATTTCAGGAGACATTATTAATACTTGCTTGAGACAACCT
GATGTCAATGAACACTTCAATGATTTGTACTATTCTGACTAA
>P. xylostella-Cactus (PxCac); Px016665
(SEQ ID NO: 80)
ATGAGTTTCAAGAAGGATTTCGACACCTCAAAGAAGATCCAGGAGGATGAAAACACAGAC
TCTGGGTTCCTATCTGGGCCGATAAGTGAGCAGCTGACCTCGGAAGATTGTGATTTAGCG
GAGGAAAGTGAGCGTGCTCGCAGCAGGCTTAGTGAGGAAGATCCTGAGCCTGAGCTGCAG
TTGGACAGTGGGCTGGACCTCTCGGAGTGTCTGTCGAGTGTTAAGCTTAGTGATAGTGCA
GTGTACACACCCACCTCGCAGACCACCCCCACAGTCACTATAGGTGATGAGAAAACTCAT
GACATCCCACCCCTCGCCATCCTGTTCCAGCAGGATGACGATGGAGACACACAACTACAC
ATTGCAGCGGTACATGGGTGCGAAAAATCAGTAGGAACATTAGTAAGAGTTTGCCCTGAC
AAAGATTGGCTAAATGTACCAAATGACTTTGGACAGACCGCCTTACACTTAGCAGCCATG
AGTGGGCATGCAGTAGTCACACGCATGCTGGTGATGGCCGGTGCATCTCTTGGCATTCGA
GACCTTGTTGGCAACACACCTTTACATGTGGCAGCCGCAGCGGGCTACGTCGGCTGTCTC
CAAGCTTTACTGGCTCCTGCTCCAGAACAACAGCAGAGAAGGCTAGCATCCACGTTGAAC
CAGAAAAATTACAATGGTCAAACGTGCGTCCATGTGGCTGCGATGGCCGGCCACGTCGAC
GCGCTGCAGACATTGGTCTATTACGGAGCTAACATCAATGCTGCGGAGGGTCTATGCGGG
TGGACACCTCTACACGTAGCGGCGGCGCGAGGCGACGTCGACACGGCTCGCTACTTGCTC
GAGAAGTGCGCTGGCGTCGATCCCTCTGCCCTGGACTACGCCGGTCGTACGGCCAGGAAA
CTGGCGTTGAAGAATAAAGCGGCCGCCCTGTTTGACGGCAGTGAGGGCAGCGAGGAGGAG
GATAGTGACAGTGAGGATGAGATGCTTCTGGAAAGCGACCAGAGTCTGTTCGACCGGATC
CGTGACGGTATGAACGCCATCAACGTCGCCTGA
>P. xylostella-Dorsal (PxDor); Px000110
(SEQ ID NO: 81)
ATGAACGCGCCCGCCGACTCCGCCGTGGTGACGTTCACCAACCTGGGCATCCAGTGCGTG
AAGCGGAGAGACATCGAGGACGCCCTGGCTGTGAGAGAGGAGATGCGAGTTGACCCCTTC
AAGACCGGATTCAGCCACAAGAACTCCCCGCAAAGCATCGACCTGAACGCCGTCCGACTC
TGCTTCCAAGTGTTCCTGCCGGACGAGCGATCCGGCAAGATCCGCCACGCGCTGCCGCCG
GTCGTGTCCGATGTCATCTATGACAAGAAGGCCATGAGTGACCTGGTTATCACGAGGCTG
AGTCATTGTTCTGCGCCCGCGCAGGGCGGCAAGCAAGTTATATTGCTGTGTGAGAAGGTG
GCCCGCGAAGACATAACCGTAACCTTCTTCGAGAAGTCCGGCGAGCGCGTGACGTGGCAG
GCGGACGCGGCGGACGTGTTCGTGCACAAGCAGGTGGCCATCTGCTTCACCACGCCGCCT
TACCGCGACCCGCATGTGCAGGACCATGTGCAGGCGTACATCCAGCTGCGTCGTCCGACG
GACAACGCGACGAGCGAGCCGCTCCCCTTCGAGCTGCTCCCGTCCAGCGCAGATCCGAAT
TATCTGAAGCGAAAGCGACAGAAACCGATACAGAACTTCAGTCGGTACTTACAGCCGATC
GATAGCGACATGAAGCAGCAGCTGCCGGACTATTTCCAGGACAACATGGCGCTGTCCAGC
ATCCCCTCCGTGAAGCTGGAGCCCCGAGATAAGACTCCTCCTCACAACATGAGCAGCCCG
CCGCTGCTGTTCCCCCCCGCGCACGCCGCGCCCGCACACCATGACCCCTACGCGTGGAAC
ATGCAACTAGACAACATGCAGTCGGGTCTGACGGCGCCCGGGCCGAGCCGCCTGCCGCAG
TACAGCCAGGACATGGCCTGGACCAACCAGATGGGCCACGTGTCCCCTATGCACCAGGCC
ATGTCCCCTAACATGGGTCACGTCTCCCCCATGCATCAAGCCATGTCCCCAAATATGGGC
CATGTCTCTCCCATGCACCAAGCTATGTCTCCAAATATGGTCCAGTCACCTATGGGTCAT
GTGTCCCCTAACATGGGCCATGTATCCCCTAATATGGGTCATGTGTCCCCTAATTTGGGT
CATGTGTCACCTAACCTGTGCCAGCAACCAATGGCTCCTATGGCGCAGCAGCTGATGGAC
CCGTCCCCCAGCGACCCACCCTCCATCACGGGGCTGCTGATGGATCGCCCGGACCAGCCC
TACTCCGGGGAGCTGTCTGGACTCTCCGCCCTGCTGGCTGAGGCAGCCCCCGCAGAGATG
CTCAGCGATAGCCTCAACAGACTGTCTACGGGGGACTTGTTGAGACAAGTTGATATGTGA
>P. xylostella-Hemolin (PxHem); ACN69054.1
(SEQ ID NO: 82)
ATGACTTTAATTTTCAAGAGTGTTTTATTTTTGGGCTTAATATTGACTACTTTTATTGTT
TCCGCTCAGCCTGTGAAACAAGATGGCGGCTCAGCACACGAAGAAATATTGTTCCGTGAG
CACGGCCAGCCGGTGGTGTTGACCTGCGCGCGCGCAGACGACCCCAACCAAGGTGGCATT
AGAACGTGGTTGAGAAACGGGACGCCATTAGAAGACGGCAAAATGTCTCCCGAAATAAAA
TTCCTCGACGACAAATCACTCTGGTGTTGCAGCCCTCACCAGCCGTGGAAGGGGTCTACC
AATGCTTCACCGAAACCCATAAGGGCATTGCAACCTCCCCGAAATTCAGCGTGAAACAGA
CTTATCTCAAAGCTCCAGAGACTACGCCTTCAGTCAATATCAAACCAGCAAAAGGCCTTC
CCTTTAGCTTGGACTGTGACGTCCCTGAAGGATATCCGAAGCCTGAAGTGCAATGGTTCC
TACAACACGGGAAAGATCACACCCTGATTGAGGCAATTATCAATAAACGGATCACACAGG
CTCCGAACGGAGCTCTTTACTTCTCAAATGCTACAACCGAAGATGTGAATGTGGGAGACT
TTAGATACGTCTGTATGGCGAGGAATGATGCGGTAGACTTACCAGTGGTGGTGTCGGAAG
CTGTCATCACAGGTCTGAGCAGCGAGGGTGGTAAGGGTAGATTGGTGGAGCAGTACGTCA
GTAAAGAAGTTAGGGCGGTTGCGGGGGAGACCACAGCGCTATTTTGCATTTTCGGTGGCA
CCCCACTAGCCCACCCAGACTGGACGAAAGACGGCAAGAATGTGAACGGGGCGCCCGGCG
ACCGAGTGACCCGACACAACAGGAGCTCAGGAAGACGACTCATCATCAAGAACACCACTC
TAGAAGATGCTGGAACTTACCAGTGCGCCGTTGACAACGGCGTTGGGACTGAAATGCGTT
CTGTCAAGGTTACTGTTGAAGCGAAACCTTCAATAACGATTGTGAATGAAGTAGCAGCGA
AGCTTGGAGAAGAAGTCAAGATTTGCGAAGCGACCGGAGTCCCAACGCCAAAACTAACGA
TAACTCACAACGCTAAACCGTTGGTTGCGTCAAATAACGTTGTTATAACCAACGATGGAG
TTGTTATAAAGAATATTCAGGCTATTGATCGAGGGTATTATGGGTGTGATGCTGTCAACG
AGCTAGGAAGCGAATTTCGTGAAACTTATCTAAGTATTGCCTGA
>P. xylostella-Sph-3 (PxSph3); XP_004922188.1
(SEQ ID NO: 83)
ATGCAGCTTAATTTCTGTATCAATGTGATCGCGACCATATTGTTGATTGTGACTGGCGGG
GATTCCCAAAAACGAGTGGGTGACATTTGCATTGATCAATACACGAACACGTACGGAAGG
TGTGTTTTCTCGGATCGATGTCCATCAGCTTTACGTAATTATCAACAGAACGGCATTCGG
CCATCAATATGCACTTACAACTTCGACAATGCACTGGTGTGTTGTACTGAACGCGGAAAT
ATTCTTCAAACGGCGAGGCCCCCGCCACCGCCTGATCAGGAAGACAGATTTCAGTCCTCT
GCTGGAAACAACAACAACAACAATAAACCTAACATTAGAGTTAGCGAAAGAAAATGCCGC
GAGTACAGCAAATCAGTAACGTTCACGGTGAGCTTCAGCTCGCTGCTGCCGGAGCCCGAG
TTGCAGTCCATCTCGCGGCCGCGCTGCAGCCGGAGCGGCGTGGGGCTCGTGCTCGGCGGC
CGGGACGCCGCGCCGGAGGAGTTCCCGCACATGGCAGCGATCGGCTTCGCATCAGCGGAA
GGCTACGACTTCAAGTGCGGGGGGTCCCTCATCAGCGCGCGCTGGCCGCTGACCGCGCCC
TGCGCGCGCGCCCGCGCCTCCAGCCGGCCCGTGGTGGCGCGCTTAGGAGATAGGAATATC
AACCCGAAAGCGCAGGACGACGCCACGCCTGTCGACGTGCCAATCCGGAACATCATCGTG
GACGTGGACCTCAGCAACAGCATCCGCCCCGCGTGCCTGTGGCCCGGCGGACCCTTCCAC
GAGGATAAGGCTATAGCTACGGGCTGGGGGGTGGTGAACCAACGCACGCAAGAGAAAGCG
GACCTCCTCCAGAAGGTCTCGCTCACTCTGCTCGAGAACTCATACTGCGACCGTCTGCTG
AGGAACAACCGCAACCGACACTGGCAGGGCTTCCGCGACTCGCAGTTGTGCGCCGGCGAG
GTGCGCGGCGGCATGGACACGTGTCAGGGCGACTCCGGCGCACCGCTCCAGATCGTGTCC
AAGGAGAACCAGTGCATCTACCACCTCATCGGCCTGACCTCCTTCGGCTACAAGTGCGCG
GAGCAGAACAAGCCGTCGGTCTACACCAGGGTGTCGACTTACGTGGACTGGATAGAGTCT
GTGGTGTGGCCGGAGGAGTATGCGGCTTGGGCGGCGGGGAGGAGTAAATAA
>P. xylostella-Transferrin (PxTrs); BAF36848.1
(SEQ ID NO: 84)
ATGATAGTGAAAATAGCCATTTTGGTGATAGCAATAACGTTCAACGATGTGTCTGCGAAA
ACTTCGTACAAGATCTGCGTACCGTCTCAGTTCATGAAGGCATGTGAACAAATGCTTGAA
GTGGAAACGAAGAGCAAAGCGATACTGGAATGTTTGCCGGCCAGAGATCGAGTGGAATGC
CTGACCCTGGTGCAGCAACGGCAGGCGGACCTCGTCCCAGTGGACCCTGAAGACATGTAC
GTGGCGAGTAAGCTGCCCAACCAGGACTTTGTGCTTTTCCAGGAGTTCCGGACCGATGAA
GAGCCGGATGCGGAGTTCCGTTACGAGGCCGTCATAGTTGTTCACAAGGACCTTCCAGTT
ACCAACTTGGACCAGCTTAAGGGCTTGAAGTCATGCCATACTGGAATCAATAGAAATGTG
GGGTACAAGATACCACTAACGATGCTGATGAAGCGCTCCGTGTTCCCTGCGATGACAGAC
CGCAGCATCTCTCCTAAAGAGAACGAGCTGAAGGCTCTCTCGACGTTCTTCAGCAAGTCC
TGCATCGTCGGCCAGTGGTCGCCTGACCCGAAGACCAACACTTTCTGGAAGTCCCAATCC
AGCAAGCTATGCTCCATGTGCGAGGACCCTGCCAAGTGCGACTACCCCGACAACTACAGC
GGCTACGAGGGCGCGCTGCGCTGCCTGGCGCACAACGGCGGCGACGTGGCCTTCACTAAG
GTCATCTATGTGCGGAAGTTCTTTGGGCTCCCAGTAGGCACAAGCCCGGCGACTCCTTCT
TCTGAGAACCCGGACAACTTCGCGTACCTTTGCGCGGACGGGTCCAAGGTCCCTATCAGA
GGAAAGGCATGTTCTTGGGCCGCAAGACCGTGGCAGGGGTTGTTGGGACATCAGGACGTT
CTGGCCAAATTGTCGCCTTTGAGGGAGAAGATTAAGCAGCTGTCTAGAGCTGGAGCAGAA
TCGAAGCCGGAGTGGTTCACCAACGTTCTAGGCCTCTCTGAAAAGATCCACTTGGTCGCC
GACAACATTCCCATTCGTCCCGTCGACTATCTGCAGAAGGCCAACTACACTGAGGTCATC
GAGAGAGGGCACGGCCCGCCTGAACCTGTTGTGAGACTCTGCGTGACGAGCTCGGTGGCG
CTGGCGAAATGCCGCGCCATGTCCGTGTTCGCCTTCAGTAGAGACATCCGCCCCCGGCTG
GACTGTGTGCAAGAGGCTTCGGAAAGCGATTGCTTGAAAAGTGTCCAAGACAATGGCTCA
GACCTGGCGTCAGTAGACGACATGCGGGTAGCGTCAGCATCCAACAAGTACAACCTACAT
CCAGTATTCCACGAGGTATATGGAGTCAGCAAGACCCCTAACTATGCGGTAGCTGTCGTC
AAGAAGAATACTCAGTATGGAAAGATTGAGGATTTGAGGGGAAACGGTCCTGTCACAATC
CTTTATGGAAGCTTCAGTGGCTTTGATGCCCCTCTGTACTACCTTATTAATAAGAAAATC
ATAGGCACTGAACAGTGCCTGAAAAAGCTTGGAGAATTTTTCGCAGCCGGATCTTGCTTA
CCTGGAGTAGGCAAATTAGAGAACAACCCTACAGGAGATAATGTCGATAATCTGAAGAAA
CAATGTTCTGGAGACAACAGCCCAATAAAATGCTTACAAGAAGACAAAGGAGACATAGCA
TTTGTGTCAAGTGCTGACCTGAAAAACCTGGATGCCTCTCAATATGAGCTGCTCTGTCTA
AACAGAGAGAACGGTGGGCGAGACTCAATAACCAACTACGCTACATGCAACATTGCCATG
GCCCCATCCCGAACCTGGCTCTCAGCTAAAGACTTCCTGTCCGATGTGTCCATAGCACAC
ACTCCGCTGAGCTTAGCACAACTACTGGATACCAGAAAGGATCTGTTTAACATTTACGGA
GAGTTTTTGAAGAATAATAATGTTATTTTTAATAATGCTGCCACTGGACTGGCCACAACA
GAAAAGATGGACTTTGAAAAGTTCAAGGCAATCCATGATGTTATCTCATCTTGTGGTGTC
GCATAA
>P. xylostella-Transferrin (PxTrs); BAF36848.1
(SEQ ID NO: 85)
ATGCTGCTTAGGACGATACATTTGTTGTTAATTGTTTGTTGTGCGTGGTGCTATGAAGTG
CCTCCGGCTAAATTGGAAGCTATTTATCCAGCTGGCTTGCGAGTGTCAATACCCGACGAT
GGCTTCTCGCTATTCGCCTTCCACGGCAAGTTGAACGAGGAGATGGAGGGCCTGGAGGCG
GGCCACTGGTCCAGAGACATCACCAGACCCAAGAACAACCGCTGGGTCTTCAGCGATAAA
CAGGCCAGGCTCAAGATAGGGGACAAAGTGTTCTTCTGGACGTATGTTATCAAGAACGGA
CTCGGGTACCGACAGGATGACGGGGTGTGGACTGTTGAAGGATTCGTCGACGTCGAAGGC
AACCCGGTTGACCCCGCGAATGGACAACCCATCTCAGCGCCGACCAGACCTCCAACCCAA
CCAGGCCGGGTGCCAAATGTCCCCATGCCGTGTGACATCTCAGTCACCACGGCATCAGTG
CCAGGGTACATCTGCAAGGGACAGCTGCTCTTTGAAGACAACTTCAATGGGGCTCTGGAG
AAAGGAAAGATATGGACGCCGGAGATTATGATGCCTGATGAACCGGATTACCCGTTCAAC
ATCTACCTGAACGACAGGAACCTGCGCGTGAGGGACGGCCGGCTGTCTATCAAGCCCGTC
ACGCTCGAGTCCAAGTACGGGGAGGAGTTCCTGGCCAAACTAGACTTGTCTGCCAGGTGT
ACTGGTAACGTGGGTACTACCCAATGCAGCAGAGAGTCCATTGGGGCCCAGATCATACCT
CCGATAATCACAGCCAAGGTTACCACCAAGAACAAGTTCAGCTTCAAGTATGGAAGGATT
GAAGTGAGCGCCAGAATGCCGCGCGGTGATTGGTTGATTCCAGATATTCTGCTGGAGCCG
AAAGAAAACCTTTACGGAGTACGCAATTACGCGTCAGGTCTACTCAGCATAGCCTCAGTC
AGAGGAAACACTGCTTACTCGAAGACCCTCAAAGGAGGCCCCATACTGTGTGACAAGGAA
CCGCAGAGAAGTGCCAAGTTGAGCGAAAAAGTTGGATATGACCATTGGAATAAAGCCTTC
CATAACTACACCATGATTTGGGCACCAAGTGGCATCACCATGCTGGTGGACGGCGAGCAG
TACGGGGACATCCGTCCCGGCGACGGCTTCAGCCAGGACCCGGCGGTGAGCAGCGTGGTG
GCCGCGCCGCAGTGGCTGAAGGGCACCAGCATGGCGCCCTTTGATGTTATGTTCTACATA
TCCCTTGGTCTCCGCGTGGGCGGAGTGAACGACTTCCCCGACACTCCTGAGAAGCCGTGG
AAGAACAAGGCCACTAAAGCCATGCTGAATTTCTGGAACGCCCGGGAACAGTGGCAGAGC
AGCTGGTTTGAGGACACCACTGCACTCCTCATAGACTATGTCAGGGTTTATGCGCTGTGA
>P. xylostella-Gloverin (PxGlv); ACM69342.1
(SEQ ID NO: 86)
ATGTACCGATTTGCAGTTATTTTATCTGTAGTCGCCGCGTGTGCCGTGGCTCAAGTTTCT
CTACCTCCTGGATATAATGATAAATACCCAGGCTTCTACAAATACTCCAAGCTAGCCCGG
CATCCGCGACAAGTGACGTGGGACAAGAATGTCGGCCGTGGGAAGGTGTTCGGCACCCTC
GGCGGCACTGACGATAGTCTCTATGGTAAGGCGGGCTACCGTCAGGACATCTTCAACGAC
CACCGCGGCCACCTGCAGGGTGAGGCTTCTGGCACCAGGGTACTCAGTCCCTACGGAGAC
AGCAGTCACCTGGGCGGTAGACTCGACTATAGCAACAAGCACGCCAACGCCAACCTGGAT
GTCAGCAAGCGGATCGGAGGCGTCACTAGTTGGCAAGCAGAAGGCAAGGCTAGATGGCCG
ATTGGCAAGAACAGTGAGCTATCAGCCGGCGGAATGATCAGACAAGACCACTTCGGCCAC
GGGAGACCAGACTACGGAGTCGTCGGTGGGTTTAAATCTAGGTTTTAA
>P. xylostella-Chitin synthase 1 (PxCHS1); KX420688.1
(SEQ ID NO: 87)
ATGGCGACGTCGGGGGGAGTGCGGGGGCGGCGGGAGGAGGGCAGCGACAACTCGGACGAC
GAGCTGACCCCGCTCCAGCAGGAGATCTACGGCGGCAGCCAACGCACAGTACAAGAAACA
AAAGGATGGGATGTGTTCCGAGAGATCCCGCCGAAGCAGGACAGCGGGTCGATGGAGAGC
CAGCGCTGCCTGGAGATCACCGTGCGCATCATGAAGATCCTGGCCTACCTGGTGACCTTC
GTCGTGGTGCTGGGTTCAGGGGTGCTGGCCAAGGGGTCTGTGCTCTTCATGACCTCGCAG
CTGAAGAAAGATAGAAGACTGGCGTATTGTAATAAGAATTTAGGTAGAGATAAGCAGTTT
ATAGTGACGTTGCCGGACGAGGAGCGGGTGGCGTGGATGTGGGCGCTGTTCATCGCATTT
ATGGTCCCCGAGATCGGGACCCTTATCAGATCTGTCCGGATATGCTTCTTCAAGTCCTCC
AGAACTCCAAGCAGCGCTCAATTTATTGTGATTTTTGTATCGGAATCTCTCCACACCATC
GGATTGGCGCTTTTGATGTTCAAAGTGTTGCCAGAAATCGACGTGGTCAAAGGAGCTATG
ATAACGAATTGCCTCTGCATCATTCCAGCCATTCTGGGGCTATTGTCTAGAAACTCAAGG
GACTCGAAAAGGTTCATGAAAGTTATAGTAGACATGGCTGCGATTGGGGCTCAAGTCACA
GGATTCATATTATGGCCACTGCTGGAGAATAAGCCGGTCTTATGGCTGATACCGATCTCG
TCAATCTGCATATCACTAGGCTGGTGGGAGAACTATGTCACTCGGCAGAGTCCAATCGGT
ATAATCAAGAGCCTCGGCCGCCTCAAGGAGGAGCTGAACCACACGCGCTACTACACGTAC
CGCTTCATCTCCGTGTGGAAGATCCTGCTGTTCCTCATGTGCATCCTCACCAGCATCTGG
CTGGACGGCGACGAGCCCGGCATGTTCTTCCAGCTCTTCAGCGAGGGGTTCGGACCGCAT
AACATTGTTGTCGAAGAGATCCAACTCCAGACGGGAGGCACAATGATCCCGGACTTAGCC
AACGCCACACTAACCGGAGACTCAGTGGAGGTGGCAGCGGCCTACAACTCTGCCGTCTAC
GTCATCCTCATACAAGTGTTTGCCGCTTACTTCTGCTACATATTCGGGAAGTTCGCCAGC
AAGATCCTGATCCAAGGGTTCAGTTACGCCTTCCCGATCAACTTGGTCATACCGCTGGTC
GTGAACTTCTTGATTGCTGCTTGCGGTATCCGGAATGGTGATACGTGCTGGTTCCATGGG
ACTATTCCGGATTATCTGTTCTTTGAGAGCCCACCAGTGTACTCACTAAGCGACTTCATA
TCCCGCCAAATGGCATGGGTTTGGCTGCTATGGCTTCTGTCTCAGACGTGGATCACCATC
CACATCTGGACGCCCAAGGCCGAGCGTCTGGCGTCCACGGAGAAACTGTTCGTACTGCCC
ATGTATAACGGACTGCTCATCGACCAAAGCATGGCGCTAAATCGTAAGAGAGATGACCAG
AAGGATGTTAAGACTGAGGATCTGGCCGAAATCGAAAAGGAGAAGGGCGACGAGTACTAC
GAAACTATTTCTGTGCACACCGACAACACTGGGTCCTCTCCCAGAGCCGTAAAATCTTCC
GATCAGATCACAAGAATCTACGCATGCGCGACGATGTGGCACGAGACGAAGGACGAGATG
ATGGAGTTCCTCAAGTCCATCCTGCGGCCGGACGAGGACCAGTGCGCGCGCCGCGTCGCG
CAGAAGTACCTCAGAGTCGTGGACCCCGACTACTACGAGTTTGAAACCCACATATTCTTG
GACGACGCTTTCGAAATATCGGACCACAGTGACGACGATTCCCAGGTGAATCGATTCGTG
AAACTGTTGGTGGACACGATTGACGAGGCTGCGTCAGAGGTGCACCAGACTAATATTCGT
ATGAGGCCGCCGAAGAAATTACCTGCCCCGTACGGGGGACGGCTGACCTGGGTGCTGCCT
GGGAAGACCAAGATGATCTGCCACTTGAAGGACAAGGCCAAGATTCGACACAGGAAGCGA
TGGTCTCAGGTGATGTACATGTACTACCTGCTCGGCCACCGTCTCATGGAGCTGCCCATC
TCCGTGGACCGCAAGGAGGTGATGGCTGAGAACACGTACCTCCTGACACTGGACGGAGAC
ATCGACTTCCAACCGCACGCTGTCAGGCTGCTGATTGATTTGATGAAGAAGAACAAGAAC
CTGGGCGCTGCTTGCGGACGCATCCATCCTGTTGGCTCTGGGCCAATGGTGTGGTACCAG
ATGTTCGAGTACGCGATCGGTCATTGGCTGCAGAAGGCGACGGAACACATGATTGGCTGC
GTGCTGTGTAGCCCCGGATGCTTCTCGCTCTTCAGAGGGAAGGCTCTCATGGACGACAAC
GTCATGAAGAAATACACGCTGCGATCCGACGAGGCTAGGCATTACGTGCAGTACGATCAA
GGGGAGGATCGTTGGTTATGCACATTGCTGTTACAACGAGGATACCGAGTAGAGTACTCA
GCCGCCTCCGACGCCTACACGCACTGCCCTGAAGGTTTCAGCGAGTTCTATAACCAGCGT
CGTCGCTGGGTACCCTCCACTATCGCCAACATCATGGACTTGCTTGCCGACTACAAACAT
ACCATCAAAATCAACGACAATATCTCCACACCGTACATCGCTTACCAGATGATGTTGATT
GGCGGTACGATCTTGGGCCCCGGAACTATATTCCTTATGTTGGTGGGAGCCTTCGTGGCT
GCGTTTAGAATCGACAACTGGACTTCATTCGAATACAACCTCTACCCGATATTGATCTTC
ATGTTCGTTTGTTTCACGATGAAATCTGAGATACAGTTACTGGTGGCACAAATACTCTCT
ACGGCATATGCAATGATAATGATGGCGGTAATCGTGGGTACAGCTTTACAATTGGGCGAG
GACGGAATAGGTTCGCCATCGGCTATCTTCTTGATATCACTGTCAAGTTCATTCTTCATA
GCCGCTTGTTTGCATCCTCAAGAGTTTTGGTGTATCGTCCCCGGTATCATTTACCTTCTG
TCTATTCCTTCTATGTATCTCCTGTTGATTTTGTATTCGACTATAAATCTTAACGTCGTA
TCTTGGGGTACCCGAGAGGTGCAGGTTAAGAAAACTAAGAAGGAAATCGAGCAAGAAAAG
AAAGAAGCGGAAGACGCAAAGAAGAGTGCGAAACAGAAGTCTTTACTCGGGTTCTTGCAA
GGAGCAAACCAGAATGAGGATGAAGGGTCAATAGAGTTCTCATTCGCGGGTCTATTCAAG
TGCATGTTGTGCACACACCCTAAAGGCAACGAGGAAAAGGTGCAACTGTTGCATATCGCA
TCTACACTTGACAAGCTCGAGAAGAAACTGGAAACTGTTGAAAAGACCCTCGACCCTCAC
GGCCTCCACAGAGGTAGGAAGCTGTCGATAGGCCACCGCGGCAGTACCAACGGAGACCAC
GGGCTGGACGCCCTGGCTGAAGACAATGAGGACCACAACCTCGACTCTGACACCGACACT
CTATCCACGGCACCTAGAGAACAAAGAGACGAATTAATAAATCCATACTGGATTGAGGAC
CCAGAATTAAAGAAGGGAGAGGTAGACTTCTTGAGTCAGTCCGAGATTCACTTCTGGAAG
GATCTGATTGATAAGTATCTGTACCCGATCGATGCCAATAAGGAGGAGCAGGCCCGTATC
TCGCACGACCTGAAAGAGCTGCGAAACTCATCCGTCTTTTCCTTCTTTATGATCAATGCC
CTCTTTGTTCTCATCGTATTCTTGCTGCAACTGAACAAGGACAACCTCCACATAAAGTGG
CCCTTCGGAGTCAAAACTAACATTACGTATGATGAGGTGACGCAAGAGGTGCTGATCTCC
AAGGATACCTGCAACTAGAGCCTATTGGTCTGGTGTTCGTGTTCTTTTTCGCATTGATTT
TAGTCATCCAGTTCACTGCCATGTTGTTCCATCGATTCGGAACTTTGTCGCATATATTAT
CGTCTACGGAACTGAACTGGTTCTGCAATAAGAAGGCGGAAGACTTATCTCAAGACGCAC
TGCTAGATAAGAATGCGATAGCAATAGTGAAGGATCTCCAGAAACTAAACGGGCTCGATG
ACGGGTATGACAATGACTCGGGGTCGGGCCCGCACAATGTGGGAAGGAGAAAGACGATAC
ACAACCTGGAGAAAGCGAGACAGAAGAAGAGGAACATAGGAACGCTCGACGTCGCTTTCA
AGAAGCGATTCTTCAACATGAACGCTAATGAAGGACCAGGAACACCAGTTCTGAACCGCA
AGATGACGTTGCGAAGAGAGACGTTGAAGGCGTTGGAAACGAGGAGGAATTCTGTGATGG
CCGAACGAAGGAAGTCGCAAATGCAAACACTTGGAGCTAACAACGAATATGGAGTCACTG
GAATCTTAAACAACAACCCAGCGGTGATGCCGCGCCACCGGCCGTCGACAGCCAACATTT
CGGTCAAGGACGTCTTCGCGGAACCCAACGGGGGACAAGTGAACCGAGGGTACGAGACCA
CGCACGGCGACGAGGGAGACGGCAACTCCATCAGACTGCAGCCGAGAACCAACCAGGTC
TCCTTCCAGGGGAGATACCAATAA
>P. xylostella-Beta tubulin (PxBTUB); KX420688.1
(SEQ ID NO: 88)
GCGGCAGCCAGCAGTACCGCGCGCTGACCGTGCCCGAGCTCACACAGCAGATGTTCGACG
CCAAGAACATGATGGCGGCTTGCGACCCGCGCCACGGCCGCTACCTCACCGTGGCCGCCA
TCTTCCGCGGACGCATGTCCATGAAGGAGGTCGACGAGCAAATGTTGAACATCCAGAACA
AGAACAGCAGCTACTTCGTCGAATGGATCCCGAACAACGTCAAAACGGCCGTGTGCGACA
TACCGCCTCGTGGACTGAAGATGTCTGCCACCTTTATCGGGAACACGACAGCAATCCAAG
AGCTCTTCAAGAGGATTTCTGAGCAGTTCACTGCTATGTTCAGGAGGGAAGCGTTCCTCC
ACTGGTATACTGGTGAAGGCATGGACGAGATGGAGTTCACAGAGGCGGAGAGCAACATGA
ACGACCTGGTCTCCGAGTACCAGCAGTACCAGGACGCCACGGCTGAAGACGAGGGAGAAT
TCGACGAGGATATTGAAGACGAGTGA
>S. frugiperda-Peptidoglycan recognition protein 1 (SfPGRP1); rep_c7951
(SEQ ID NO: 89)
GCATTAACATCTCAGGATTTGATTCGAAAGTAAGGTCTGAATTTAGTCCTTACATTTACT
TAAAATTAGGTCCTTTTTGGTTTGTACTGAAATGAAAGTTTTTCTGTTCTTGGTCTTAAT
TGTGAAGATAATGGCTGAGGCAAAAGGAGATTGTGATGTGATCCCGATTACGCAGTGGGG
AGATTCACCTCTTAAAAGGGAGGATYCTCTTCCAAATCCAGTGAATATTGTTGTCGTCCA
AYACRCTGTGGTACCGGAGTGTAACAATGATGAAGAGTGTGAGAAAGCAGCCAMTGGAAT
CAGGAGCTACCACATTAACAAACGTGGATTCACTGATATAGGACAATCGTTCCTGATTGG
TGGAAACGGGAGAGTTTATGAAGGAGCCGGCTGGCATCACGTTGGGGCCCATACTTTGGG
ATACAATGCAAGATCTGTGGGGATCTCCTTCATTGGCGATTTTAGAACAAAATTACCAAC
ACCCGAAGCACTGAAAGCCTTCAACAGTCTCCTGGAATGTGGAGTCACGAACAATTATCT
GTCAAAGGACTATCACCTGGTGGCCCATAGTCAGCTCTCTATGACTGACAGTCCYGGAGA
CATGYTGAGGAAGCAGGTGGAATCGTGGCCTCMTTGGCTGGATAATGCCAAAGACATACT
TAAGTAGAARAAGACTAAACGCCGTACTTTGAGCCATTTAATGGTTACTTAACCCAGTCC
TTAGCAATTTGATACAAGGCCAATGTCTCTAAGGGCGGCAGTAAAGGTCAAAACACATTT
AATGAGTGTGTTTAAGATTTTGCTAGTGAAAATTGTTTTGAAGTACGTATTTGATGTAAG
TGATGATATCAGTACCCTTAGTATGAGTTTGCTTTACGTTCCACGAGATGGAAACGAGAG
CGCGTTCGGCGCTCTGATTGGTTCGTTCATTCATGCCGGCCAATCATAGCGCCGAATGGG
CTCTCATTTCGTTTTCGTTCAACGTAAAGTAAATTCGTACTAAGGGTACTGACTTTAAAA
TAAGTTACCAAAAAGAGTATTACCTATTTACATTATTTTATTTATTTTAGGTGTATTGTA
ATTCAAGTATTAAATTAATTAGTGTAGATTAATKSCATGCATTTTATATTTGATTTCATT
GAATAA
S. frugiperda-Attacin (SfAtta); rep_c9395
(SEQ ID NO: 90)
ATCATTCCAGATCCTCTCTCATACATCCAACACTTGAAGCAAACCAATCCACATACATTA
TAGCAACATGTTCGCTCTCAAGTTGGTACTAGCTGCAGTGCTGGTGGTCGCAAGCGCCAG
ACATCTACCACAGGACCACTCAACGTACGACCAAGTACAACTCCTCGGGTTCGACGAAGA
TGGACGACCAGTGTTTGAGCACGAAGACTTACTCCCAGAACTAGAGGAGTCCTACCAGCC
AGAGCACCTGGCGAGGACTCGCAGACAGGCGCAGGGCAGCGTCACCCTCAACTCCGACGG
CGGCATAGGCCTGGGCGCTAAGATCCCGCTCGCACACAACGACAAGAATGTGGTGAGCGC
CATCGGCTCCATGGACTTCAACAACAAGTTGCAGCCTGCTTCCAAGGGCTTCGGTCTGGC
TCTGGACAACGTCAACGGGCACGGACTGACGGTGATGAAGGAAAGTATCCCCGGGTTCGG
GGACAGGCTGTCGGGCGCTGGCAAGCTGAACGTGTTCCACAACGACAACCACAACGTGGC
CGTGACCGGCTCTCTCGCCAGGAACATGCCCAGCATCCCGAACGTGCCCAACTTCAACAC
GTACGGCGGGGGCGTCGACTACATGTACAAGAACAAGGTGGGAGCGTCTCTGGGCATGGC
CAGTACTCCGTTCTTGGACCGCAAGGACTACTCCGCGATGGGCAACCTGAACCTGTTCCG
CAGCCCGACCACTACCGTGGACTTCAGCGGCGGCTTTAAGAAGTTCGAATCTCCCTTCAT
GAGCAGCGGCTGGAAGCCTAACTTCGGCCTTACTTTCGGCAGATCTTTCTAGATATATTT
TGTAATCTAAATTTAACTTTAACTTTGTTGTATAATATTTTGTCGAATTAAGATCAGTAT
TGTTCATACTAATATTATATTATCAGTGTTTCTTATAAATTAA
S. frugiperda-CtypeLectin15 (SfCTL15); Joint2_rep_c488
(SEQ ID NO: 91)
AGTTTTCGTGCTTAGCACACAGAGCGCCGAACGCGCTCTCGTTTCAATTACTTTAAACGT
AAAGCAAACTCGCACTAAGGTTACTGTGACCTTTGTAAACATGTACAATGCAATACTATG
TTGTTATTTTCGATACAATCATATAAAAGACAACGTTTAAAAAAAAAGCATAGCACAGAC
CACAGTACAAGTATCGAGACCAAACTTTTGTTAAAACTTATTTCGTTCTGTGCAGCAACT
CTAATGCGAATAATGTGCAAATTATAATAAATATGTTTTGTATATAAACATGTGTTATCT
CAAGTTCAAGACATTCCGGCTCCTACAGTCAACAGATACAGTGCACAAAAATGTTAAAAA
TATATTTAATTTGTTTGTCTTACTTGTTCATGTTAAACTTAGACAGGGCACAATGCACCC
CCCAGTATTGGTTCAACATGGATGCGAATGGTTGGCTGAAAGTGCACACGATACCCGCCA
CGTGGGAGGAAGCATTCCTTCGGTGTCACTATGAAGGTGCGGTACTTGCGTCCCCCTTGA
CGCAACAACTGAGTAAGGCTCTTMAAAACAAGTTTGCAGKCWTCGGTAACCCATCAATCC
ACTTGGGGACACATGATTTGTACTCCASTGGTTACTACTTTTCTGTAGAAGGAGTACCAA
TGGACAGCTTGGTGCTGAAATGGAGTAATATCAGAGGTACGGGCGAYTGCCTGGCGATGT
CACGCGACGGGGAAGCATTTTTCACGAAATGCGAGCAACCTCGACCCTACATCTGCTACA
AGAAGCTGGACAATCTGACGATGAACATCTGTGGAACATTYGATGACGCKTATCAATTCT
ACGATAAGACTGGCAGCTGCTATAAGAGACACAACGYCTATCAGACATGGCCTGACGCGT
TCAAGATATGCGCTGCCGAGGGAGGSTACCTGGTGATCCTGAACGATGACACAGAGGCCG
CCATCATCAAGGATATGTTCCCCGTACGGCCAGGAAAGCCCAACGAATGGGAGAACTTCC
ACGTGGGKCTGCGGGCATGGGGACCGGAACGTACTTGGATCACTATTCATGGAGAAAAAA
TTGATGATGTATTCCATAATTGGAACCCGGGACAGCCGGACAACTACAAGGGAGTCCAGG
ACACTGGCGCTTTCCTTAGARMAGGCACTTTAGACGATCATGCCGCTGGTGACAAATGTA
TGTTTGTCTGTGAGAAGGATCCTAAAGTAAAACGCTTCGAAGAGYTACCCGAAGGATTGG
CMGAAGTTTTAGGACAATAGGCCGCTAGTCAAATATTGTTCATATACCTWAGTTTTAATT
ATGTAAAAATAATARTCGATTGCAGAATTTAATAAAATTTAAACTAAAAAAAAAAAAAAA
AAAAAGGTMAAAACATGTC
S. frugiperda-Galectin4 (SfGlc4); rep_c2653
(SEQ ID NO: 92)
GCCTTGTAGTTCGACAGTCAAATCCAACGGGTGTCAGAAACAATTTCCGTTTTTCCGGCT
ATTGATCGTCATATAAATAACTCCGAAGGAAAAATGACTACAATCGACAATCCGACAGTA
CCGTTCACGAGACCCATTCCTGGGCATCYGCTCCCCGGCCGCAAAATGGTCGTTAAGGGT
GCAATCTCTCCCAGATCAGATAGGTTCTCAATAAACTTGAAATGTGGTAGCGAGGACATC
GCTTTCCACTTCAACCCTCGCTTCAGTGAGCAGAAAATAGTTCGCAACTCTTATATTTCT
GGCAAGTGGGGTCATGAGGAGATCAGTGGAGGCATGCCGTTGGTAAGGGGAGAGCATTTT
GAAGCGCAATTTGAATGCAATGAAGATAATTTTTCGGTGGAGTTGAACGGGAAACATTTC
TGCAATTACTCTTATCGCATCCCAATCCATAAGATCACCCACGTCAACGTGGACGGTGAC
GTCACGATAAGTCAGATCACCTTCGTGGACGCCTGAGCCATCGCCCGATCTTACAATGGT
ACTTTTTTTTTACTTATGAACTAATGCTGGAAACCAAACTTCTTTATTCAAATATTTTTC
TTTTGTCTTTATGACTATCAGTGTTTTTATTGCAATAAAAAG
S. frugiperda-Lysozyme (SfLys); rep_c18992
(SEQ ID NO: 93)
ATCAGTGTGGTGTCTTCAACCCAAGTGCATTGTATTGCTTGGTAAATAATAACTGASAAC
AAAAATCTTGGTTATTGCTTGAACAGGTTTCGCCTATCAATTAGCCGAATATTGTTACCC
TTGACACGCAATAATTGGTTACTCGATTAAAGCTTGAAGAGGTAAAGTGCCGAAATGACG
CGCGCCATTCTGTTTGTTGTTGTTTTATGCTTTATTGCAAAATGCTATGGAAAAACATTC
ACKGAATGTGAATTAGTTCAGGAGCTAAGAAGGCAAGGATTTCCTGAACATGAGCTTAAA
GATTGGGTRTGYCTGATCGAAGCGGAGAGTTCCAAACGAACCAACGCCATTGGTAACGGC
AATTCAGATGGCTCTCTCGACTATGGCTTATTCCAAATCAAATAACCGCTACTGGTGCAG
CGAYGGTGACCATCCAGGCAAGGGATGCAACGATACCGCTAGTAAAGATCTGTTGCTGGA
TGACATCACAATAGCGTCTCAGTGCGCTAAGACCATTTTCGGTGTCCACGGATTTAACGC
CTGGGTCGCATGGGTGAACAAATGCAAAGGAAGGACCTTACCYAACCTTCACTGTTAGTT
ATTTATTGAGAAAATGTAACTAAGGTATATGGTTACTTTGTACCTAAATATAGGTATTAG
GCATATATTGTCCACTCTCATCAAATTTTTACTTTTATATCAGT
S. frugiperda-Hemolymph proteinase 10 (SfHP10); c12881
(SEQ ID NO: 94)
ATTCGCTGTACGCACCTCGGATTGTGCGGTCAACTWTACAAGGCTCGTACCTTGCAGTTG
AACCGACAATCTATTCCTAAAGCCTTTTTAAGGTCAGGAAAAATAGTTCCTACATCTAAA
TGCAGTAGAATTTGCGAAACGAATTTAAATAAAAATGGCGTCGATTGTGTTTGTGATTTT
GTGTGTTACCGTCGCTGCGGTGAAAAGCGCGATTTTAAACCCGTGGAGTAAAGTTGAGGC
CAACAAATGTGGTGTAGAAGCCAGTACTAACTTGGTCCATCACAATCCATGGTTGGTCTA
CATCGAGTATTGGCGTGGAAACTCAGATACTGAGATCCGATGCGCCGGTACTTTAATCGA
CAGCAAACATGTCGTCACAGCTGCCCACTGCGTTAGGACTCTGAAGTTTAGTCATTTGAT
CGCCCGTCTTGGCGAATACGACGTAAATTCTAAGGAGGACTGCGTTCAGGGCGTGTGTGC
CGATCCCATCGTCAGAATCAAGGTGGCTGAGATCATCGTGCATCCTAACTACAGCAACCG
GGAACATGACATTGCAATCTTAAGGCTGGAGGAGGAAGCTCCTTATACCGATTTCACTCC
GGCCCATCTGTCTGCCTTCTGGTGATCTCGCGGAAGACACCCAGTTCTTAGCAGCCGGCT
GGGGTGARATCCCCACGAAAGGCTTCTTCAGCCACGTGAAGAAAATCGTCCCCTACGTAC
TGGAATCGAGAGAGATGCCAAAAGGTGTACCAGTACAATTATATCCCGGAGAACGTGATC
TGTGCCGGT
S. frugiperda-Trypsin like serine protease (SfTSP); rep_c48453
(SEQ ID NO: 95)
CAAGTAGCAACAAAATGCGTGTCCTCGCTTGCTTGGCCCTTCTCTTAGCTGTGGTAGCAG
CCGTCCCCTCCAATCCCCAGAGGATTGTGGGTGGTTCGGTCACCACCATTGACCGGTACC
CCACCATTGCATCCCTGCTGTACTCGTGGAACTTGAGTTCCTACTGGCAGGCGTGCGGTG
GTTCCATCTTGAACAACCGTGCCATCCTTACTGCTGCCCACTGCACAGTTGGTGACGCCG
CCAACAGATGGAGAATCCGTCTTGGCTCCACCTGGGCCAACAGCGGTGGTGTCGTTCACA
ACGTCAACACTAACATCGTCCACCCCTCATACAACTCTGCAACTTTGAACAACGACATCG
CTATCCTCCGCTCCGCCACCACCTTCTCCTTCAACAACAATGTTCAGGCTGCCTCCATTG
CTGGTGCCAACTACTTGCCCGGTGACAACACCGCCGCCTGGGCCGCTGGATGGGGAACTA
CCTCCGCTGGTGGCTCTAGCTCTGAGCAGCTCCATCACGTTGAGCTGCGCATCATCAACC
AGGCTACTTGCAAAAACAATTACGCTACCCGCGGTATCACCATCACCTACAACATGTTGT
GCTCTGGCTGGCCCACCGGTGGTCGCGACCAGTGCCAGGGTGACTCTGGTGGTCCTCTCT
ACCACAACGGCATCGTTGTTGGTGTCTGCTCTTTCGGTATTGGCTGTGCTCAGGCTGCCT
TCCCCGGTGTCAACGCTCGTGTATCCCGCTACACTGCCTGGATCTCTTCCAACGCATAAG
ATGTTACTTGGTGCTAATAATATTTTTTTGTAATAAAATGTTACTTTTATCCTCC
S. frugiperda-C Type Lectin 6 (SfCTL6); Joint2_rep_c448
(SEQ ID NO: 96)
AACAGTTTTCTATTGGCAGTCAAAGACTTCAGTCGAAAAATAATCCTCATCAGAGTCGTG
AAGCAAGGTGCCCAAAATATAATGTAACCTAGATACCTATTAATAAATTATTTGTCAACC
AAAACGTTACGTTCAAAGTCCTTAAAATCAAAATATCTTATGATTAGTTTTGATTTAAAA
ATAGAGGTTCGAAATCGCCAACCCAAATAGGTTTAGTTTACGATTCAGGAAAAATCCTAA
CGTAGGGAAACATTATTTTACAAGACTTTTGGCTTAAAAACTTTGAGAACCAATGTCAAA
TTTGATAATAACTAATGAGGTATAAAAGCTTGATCCTATTAGGACTTATTTTCATAACAC
CATCGAGTTTGTATTTAATRRAGACGTGKGTTAACTAACAACATGAAGACCGGTGTAAAA
TATTCTGTTMTTTGGATATTCTCTCTATTYTGCTATATAGAGGCAACATTTCGTTGTGAC
TACACGTACAGCAAGGAAGCGAAGGGCTGGTTCAAACATGTGGTGATACCAGCTACTTGG
GCTGACGCACGWCTGCACTGCACGTTGGAAGGTGCAACGCTGGCTTCTCCACTCAACCAG
GCTATMAGTAATGAGATGCAGTCCMTCCTGGCRAACCTCTCGGCGCTGCAATCAGAAGTC
TTCACTGGAATTCACRCGACTKTTTCACGRMRCAACTTATATCATACYATYGAAGGTATA
CCTCTTAGTAAAATTCCATTAGATTGGGCAACAAATGAGCCAAATGGTGGGAGAGATGAA
AACTGTATCACGTTTAACTCCGATGGCCAAGCGGCAGACAGATCCTGTAARGAGACTCGA
CCTTACATCTGCTACCGACACACWACTAAAGTGACTGTGKCCAATGAATGTGGGACTGTA
GATCCTGAATACAATTTGGATAAAAGAACGGGCKCYTGCTATAAGTTCCACACRGTACCT
CGCACGTTCGAGCGTGCCAACTTCGCGTGTTCTGCTGAAGGTGGMCACCTTGCCATAATC
AACRGTGATRTCGAAGCTGCAGTACTGAAGGAACTCTTCGAAAGGAATCCACCTGCAAAG
ATGTTCGGTACTTTCTGGAAAGACGTTGCTTTCATTGGTTTCCATGACTGGGGTGAATAT
GGTGACTGGAGGACAATTCACGGTCAAACGCTAGTAGAGGCAGGATACAGCAAGTTTTCA
TCTGGAGAGCCAAATAATTCCTCGACGGGAGAATTCTGTGGTTCCATCTATCGGAATGGT
ATGCTCAACGACCTTTGGTGTGGGCATCCTAAACCATTCATCTGTGAGAAGGACCCGAAA
TATCCAGCCGTATGTTGTGTTACAGAATCAGAACCAGAATTGGACCCTACTCACTTCTTA
GAGTAATTCAAATTGGATCTTTTTTTATAATTCTACATCATTAGAAATATGATGTTTAGT
TGAATCTGCTTTTGAAGATTGATT
S. frugiperda-Serine protease homolog 13 (SfSph13); rep_c1904
(SEQ ID NO: 97)
GAAKAGTGTCAATTTATTTAATTGTCAAAATCGCGTTGAATGTAAGATTGCTATAGAAAT
TATTTGTAAAAACTTTCTCAAAAATTTAATTCTAAAATGTAAAGGAACCTAAAATCAGAT
ACCGATAACACATTTTGTAAATGGTTTAATATTGAAACGAAACTTCTAAACATTTTTGGT
GAAATACACATAATAATATAACTTCTTCGAAAACTAGGTAGATAGACTTCAACTCAGTTT
TTATTAGCGAGCTAGAAGATACAGAGATTATCTAAGATGGTGTGTGATGGTCCGGATCCT
CCAGAGAACAATCCTCACGAAATTGTTCCACCTCCTAGGAATGAATTGATAATGAATGGT
AATCAAGGGGATGACAGTGATGAAGAACATGAATATTTTGGCTATGAACCTTTGGCGCAA
GGTCCAGAACTAGCAGTCTCAGATCACGATAGTGATGATGACCCAGAGAGTGCTGAAACA
CCTCCAGCTGATGTTCCAAATATAGAGCCAATGGAAAATGTACTAAGCCGGGAAGTTTGG
AGTGCCCCGAGRCATACAGATGCTATACAAATGGATAATGAACGGGCTCAACAGGTGATG
AGAGCTATGGCAAATTTTGCTCTACCCCAGGCCTCAWTTCCAGAATGGGCTCAGAGCATC
TCTGAAGAACAATGGAAGCAAACTCTGAATGAACAAATAGAAAGATTGAAAAATAAAAGA
TAAACTAATTATATGTAGTATAATAATAATTAAGATTTAAGTGATAAAAATAACAATTAT
AACTTATATGTTTAAAAAATGTATATAAACTGTAAACTTTAAGCTTATTCACATTACTTT
AAATATAGAAAAAATATGTGTACCTATTTATTGTTTGGCTACATAATCCAATATAGATTA
AATTGCAAATGTTTAATGTAATTATTTAGTTGGATCAAAAATAACAGCACTAAAGGTCAC
TAGTTTAGTTCTTTCATTTCGAATGAAAAAGTAAAATGGAGAATCTGCATAGAAATTTTC
AGCTGACCGTGTTAATGTTCCACTTGTTGCGGCTACTGCTTCTGTTCCAAACTCATTAAT
TGTTAACTTTACATCATGAATTATGGAATCCACATGTATTCCAGGGTTGGGTAGACTGTC
CAAAATGTTTGTCCCATTTCCTTCGTTTTCTGAGTATCTGTCTGATTCTCCTTCAGATTT
ATTCACAAACTGATTGGTGTTATTTACCATAAGTGCGAAGTTTGCTTGGCCTGGTATAAA
CATACTTTGTATCCCAATAGATTGCAAGGTGCTCTCTAGCTTTGTGGTGCTTTTAATTTT
CATCTTAGGGAAACGAATAACACAATTTTTCACATGCATTTCATTGATGGTATCATCAAT
AACTTTGTTGTTAATATTTTCCATGAGTTCCAATAATGTTAATCGYTTATGAGATAATGG
TTTTAAAGCGTACATAAAAGTTTCACTGTCGTTATATGGCAATGCTATCATGTGGAAATC
ATGTTCCTCAGAAACCTTGTAACGAAACTCCCCAAAATTCAACATCATGTCAGCCATAAC
TTCATCTGTGGCCCTTTTGAATTCCATCTTTCTTGTGAATTGCGGTGCAAAAGGCTGTTT
CCATGTGCCACTAAAGTACAATGCAGTTAAAAGTACTACTTTAGTATGGGGAGGCAGAGA
ATCTTTCAAAAAATCGTCTATATTTCCTTCTGTGTGCTTGCTCACCCACTCATTGATGCT
GTTTTTAGATCCTTCGGTTTCTTGAAAATCAGTACGTAACACATCGCCTCCGTAAACACC
ATGCAGATAACTTCTGTACAACTCTCGCAATTCTACTTGGTTGTCTACAAATATCGCGTC
AGCGTACAAAGTTTTTGAAAACTCGTTCATATTTAAACTCTGTAGTAGTTCGCCGAACTG
TTCGTGGTTCTTGCGGTTTCGTAGAATATCTTGGGAAAATCCGAGTGTCTCTGCGATTTC
ATCATGTGTCATCCCAACACTTCCGAGTAATGTCATGGCAAGCAGACCAGCGACACCTAT
TGGTGATATCACGATGTTTTCGTTTTTCTTTTCGTTCATCATTTTGACTAGTAAATTATA
TCCAAAATTATTTATAGCTTTTGGTATCGAGTTATCTTTCGTATTTGTTGTTTCTAAGTT
ATCATTTTCAGCAGTTGTGGTTGGTATTATCGTGCTTGAAATCTGACTGTCCTCTTCATT
TTGTCCAGAATATTGAAAATATAATGTTGTTATAATTAAAAATAACAATGACTTGCTATC
CATGTTGTGGTGCTTAAATAAGGTATTAAACGTAAACTTCACACTAACAGGAACAGGTTC
CTATTATTTCCCACTTAAATCGTGAACTTACGGCATAGACACTATACACGTCTACTCGTT
TGACCAACTCTGAGAGCAACTCAAAAACAACCTTGTGTGTGTATGAATAACGAACTAAAA
T
S. frugiperda-Cecropin (SfCec); rep_c42380
(SEQ ID NO: 98)
TTCGTGTCGTATCACTAGAGTTCGAAATACAAAATAATAATACATTTATTATTTTGCCAT
AATTAATAATAAAGTTATTTTATTTCATAATAATAATGAATTTCACAAAGATATTTTGTT
TGTTTTTGTCTTGCTTTGTTTTGATGGCGACCGTGTCAGGAGCTCCTGAACCGAGGTGGA
AATTCTTCAAGAAAGTGGAGAAGTTGGGCCAAAACATCCGCKATGGTATCATAAAGGCAG
GACCCGCAGTGGCCGTGGTGGGATCAGCRGCAGCCATWGGAAAGTGAKCCCTACGACCTG
AGACATGAAGACTAATATCCAYTAAAATAASAATATTGAGGCKTATAATATTAATTTATT
RTRTTTGTAAATTAAATTATTTGTAAGATAA
S. frugiperda-Relish (SfRel); c13122
(SEQ ID NO: 99)
ATAATATGAAGAGTGCTGCGACTAACGATTTAAGAATCTGTCGTATAAGTCGGTCGTCCG
GAATTGCTTCTGGCGGCGAAGATGTCTTCATTCTGGTGGAAAAAGTTAATAAGAAAAACA
TAATGATTCGATTCTTCGAGTTGGATGAAAACGGAGAAAAGAGGTTGGACCAATGTTGGG
CGATTTGTGCAAAGCGATGTTCATCACCAATACGCAATCGTCTTTCGTACTCCTCCATAC
AAAAACCCTGAGACGCCAGTCGATGTGGAAGTGTATATCGAACTGGTTCGTCCATCGGAC
GGCCGTACCAGTGAACCGAAGCAATTCAAGTACAGAGCCAACCAAGCGTACAAACAGATC
AAGAAGAGGAAGACTGGATCTTCCTACTGTTCTATTGGCAGCTCATCTAGTGGATCGTTG
AAAAGTGGTTGCGACATTCCCATATCTGTTGTCAATCATCAACCAGAAGAAGTTCCCATG
GATCGCGAGCCACCGGTGCCGTCGTCGATGTATGTTTTACCCCAGGTGCATGATTCGACG
ACACCAAACCAATGCGATCTGGCCAGTGCTCTGTACTCTGCTCCTGGTTCGGAGACCAGC
CAGTCTCCTATATCGAGTCCGATGTGGAGTGAGCCTCACAGCGTGATGCTGCCGATGTCG
CCCATTGCCAACCTTCAGCTCAACTCCGCAGACTTCGAACAGATCACAGTACCCACT
S. frugiperda-Toll (SfToll);joint2_ c3284
(SEQ ID NO: 100)
AAAACCGAATAACCGGTATACCGGATAACTTCTTCAAGGATTTGAAGAATATAGAGCATT
TAGATTTGAGCTTCAATAAGATAACTAAATTGACCAGTGGAGTGTTGTCACCAATGAAAC
AATTGAAGTATTTGAACCTGAATCGAAAYCATTTGGAAGTTTTRCCTGAATTCCTCTTCG
CTGGCCTCAGRAAACTAGARAAWGTRWCAATMAACGAAAATCTACTCACTTCCATAGATT
CTTTAGCATTCCAAGGRGCWACGGCWTTGCATACAATATCTTTATACGGCAACAGATTAA
CATTGAAGTCCAATGAACACATCCAAGACTATATGGACTTAGATCTCTACTCACCCTTYA
ACACTTTGAGCGAATTAAAAAATTTGAACCTTAGTAAGAAYAAYATAAGCTCTATATTCG
ATGATTGGMGGATWGTGYTRCTCAATYTGGAGTTGCTGGATCTATCTTATAAYCATATTG
GAGAGTTATTGCCGGACACCTGYCAATTCCTAAGCAACAARRTYACAGTGGACCTGCAGC
ACAATGACATAAGTACTGTTATTTTATATCCTACGTCTCATATGGACTTCACAGAACCCA
GCATGCCATCAAACAATGTGTTTCTCTTAGACAACAACCCATTTAACTGTGATTGCCACA
TATATAACCTAGCTATGCGTCTACAAGGCAAAAAGCTGCCCTACGAACCTACTTTCAACG
TAGGCAAGGCCGAATGCAAATCCCCACGCCACTTAAGTGGGGATTTGTTGTCACATGTAT
CCCCACTCGACTTATACTGCGAAGAAATGTCGCCTGATGTTCGTTTTAATTGTAGTTCGG
TTAAAATGAGGCCTGCGTACAATGATTTGGCGTACGACTGTGATGATGTACCACCTTACT
TCTCTGATGACATTAAAAACTACTCTTTGAAATTACGACACCCACCAAAAGACTTACGCA
ACCTAACACTAAGTCTACTAAACCTAACCGGCATAGGATTACAAGAAATCCCMTTTACRC
CGTCAGAATCGGTAAAAGTAATCGATTTATCGAACAACAATCTCACAGAGATACCKATMM
GATTCTTAGAATCGAATACGACRTTGTWTTTATCGAATAATCCATTTGTTTGCGATTGTT
CTTCRAAAGATGATATTTTAGCTTTAMGRGCRAGTTAYAATGTGAATGATTTGGATATAG
TYAGCTGTTCAAACGGCGTTTTGGTAACAGATTTAGAAATATCATCGCTATGCTATACAA
GAACTTTAATAGCAGAAATAGGTGGTATAYTAATATTCTTAYTAATAATCTTAGTRTTTA
TAATAACATTYATATTTCCTAGACAAATGGTACTRTATTGTGGTCATAGGCTMTTTCCKT
GCTGGTATATCGACGATCCAGAAACTACGGCAAAAGAATATGACATATTCATATCTTATG
CTCATAAAGACCAAAAATATAGTGAATAAGCTACTCCCGTAAACTAGAAAATGATTTCAA
GTTAAAAGTCTGCGTTCATTACCGAGATTGGGAAGTCGGTGATTTCATTCCGGATCAGAT
CAATCGATCAGTATCGAATTCGAGAAAAACGATTATTCTTTTATCGAATAGCTTTCTCGA
TTCGACTTTCGCGAATTTGGAATTTCGTACCGCGCATAATTTAGCTTTGAAAGAGGGAAG
AGAGAGGGTCATTTTAATACTTTTAGAGGACGTTAGTAAACATGAGAAGTTATCTGAAGA
ATTAAAGTATCATATGAATATGAATACGTATCTTACATGGGATGATATTAGATTTGATGA
AAAGTTAAAACGAAGGACGATACCACAGAAATATAATAGAAAGAAATTCGTAGCGCCGGC
CATTTTAAAACCTATATTCAGACAGGCTACTGAGAATAATTTGAAAAAGGCACTCGATGT
ACACTTTGAATAGTGCAGGCCAATTGGTGAATTTAGCTCAAAATAAGAAGAATATTGATA
TGGTATAGCTTTTCCCAAATAAGGCTCTTTTAAACATGTTAAGCCCTCTCTCACCCGACC
TCTCTGACTACCGCACTCAGAGACATTTTAATGTATWTTTTTTTTTTAATTGGGTACAAG
CCCGCCACAACATCCCAGACCGCT
S. frugiperda-Beta 1, 3 glucanase recognition protein (SfBGRP2); EF641300
(SEQ ID NO: 101)
ATGTGGTCGGTGTTAGCCGGAGTATTGGCGATCGCGTCGCTAGGCGCGGCTTGCACCCCC
AGTTTGACCACCGTCAGTGGTACCCACGCACCGGTCACCGTCTGCTCTGGTGCTCTGATC
TTCGCTGATGGCTTCGACACTTTTGACCTCGAGAAATGGCAGCACGAGAATACTTTAGCT
GGTGGCGGTAACTGGGAGTTCCAATACTACGGCAACAATCGCACCAACTCTTTCGTGCGC
AGTGGAAGTTTGTTCATCCGTCCCTCTCTAACATCAGACGAGTTCGGAGAAGCTTTCCTC
TCATCTGGACACTGGAACGTCGAGGGTGGTGCTCCTGCTGATAGATGCACAAATCCACAA
TGGTGGGGTTGCGAGAGAACAGGCACGCCGACCAACATTTTGAACCCAATCAAGAGTGCT
CGTGTCCGTACCGTCAATTCCTTCAGCTTCCGTTACGGACGCCTCGAAGTCCGCGCTAAA
ATGCCCGCCGGAGATTGGATTTGGCCAGCTATCTGGTTGATGCCTGCGTACAACACTTAC
GGTACTTGGCCCGCATCAGGAGAGATTGACTTAGTTGAGTCCCGAGGCAACCGTAACATG
TTCCACAATGGTGTCCATATCGGTACACAGGAAGCAGGCTCGACCTTGCACTACGGACCT
TACCCAGCGATGAACGGTTGGGAGCGCGCCCATTGGGTCAGAAGGAACCCTGCTGGCTAC
AACAGCAACTTCCACCGTTACCAACTTGAATGGACACCAACTTACTTGCGATTCAGTATC
GACGACATGGAGCTTGGACGTGTAACCCCTGGCAATGGCGGCTTCTGGGAATACGGTGGT
TTCAACAGCAACCCTAACATTGAGAACCCATGGAGATTCGGAAGCAGAATGGCGCCTTTC
GATGAGAAGTTCTACCTTATCATGAATGTGGCTGTCGGTGGTACCAACGGATTCTTCCCT
GATGGCGTCAGCAACCCATCACCCAAACCCTGGTGGAACGGATCACCAACCGCCCCAAGA
GACTTCTGGAACGCGAGATCAGCTTGGTTGAACACCTGGAACCTGAATGTCAACGATGGA
CAGGACGCATCCATGCAAGTCGACTACGTCCGCATCTGGGCTTTGTAA
S. frugiperda-c20042 (Sfc20042); Un-annotated
(SEQ ID NO: 102)
ATCAGTCGTCCCTCGTACATCCAACACTTCAAACCAAATATCTCCATATACATAGTAACA
ACATGTTCGCCCTAAAGTTGGTACTAGCTGCAGTGCTGGTGGTCGCAAGCGCCAGACATC
TACCACAGGACCACTCAACGTACCGAACATGTACAGCTGCTCGGGTTCGACGAAGATGGA
CGGCCAGTGTTTGAGCACGAAGACCTGCTCGCAGAACCAGAGGAGTTCTATCAGCCAGAG
CACCTGGCGAGGACTCGCAGACAGGCACAGGGCAGCGTCACCCTCAACTCCGACGGCGGC
ATGGGCCTGGGCGCTAAGATCCCGCTCGCACACAACGACAAGAATGTGGTGAGCGCTATC
GGCTCCATGGACTTCAACAACAAGCTGCAGCCTGCTTCCAAGGGCTTCGGTCTGGCTCTG
GACAACGTCAACGGGCACGGACTGACGGTGATGAAGGAAAGTATCCCCGGGTTCGGGGAC
AGGCTGTCGGGCGCTGGCAAGCTGAACGTGTTCCACAACGACAACCACAACGTGGCCCTG
ACCGGCTCTCTTGCCAGGAACATGCCCAGCATCCCGAACGTGCCCAACTTCAACACGTAC
GGCGGGGGCGTCGACTACATGTACAAGAACAAGGTGGGAGCGACTCTGGGCATGGCCAGT
ACTCCGTTCTTGGACCGCAAGGACTACTCCGCGATGGGCAACCTGAACCTGTTCCGCAGC
CCGACCACTACCGTGGACTTCAGCGGCGGCTTCAAGAAGTTCGAATCTCCCTTCATGAGC
AGCGGCTGGAAGCCTAACTTCTCCTTTAATCTTGGCAGGTCATTCTAGAAATATTTTTAA
ACTCTTATTTAAAAATTAAATGTAAAAAATCCWGTTTGTTCATGATAATAAGAATAAATR
ACAGTATTGTTCGTACTATTTACTATGTAATCTATAAATTGTATTAATAAATGAAAATTA
A
S. frugiperda-rc16438 (Sfrc16438); Un-annotated
(SEQ ID NO: 103)
ATCAGTCGTCCCTCGTACATCCAACACTTCAAACCAAATATCTCCATATACATAGTAACA
ACATGTTCGCCCTAAAGTTGGTACTAGCTGCAGTGCTGGTGGTCGCAAGCGCCAGACATC
TACCACAGGACCACTCAACGTACCGAACATGTACAGCTGCTCGGGTTCGACGAAGATGGA
CGGCCAGTGTTTGAGCACGAAGACCTGCTCGCAGAACCAGAGGAGTTCTATCAGCCAGAG
CACCTGGCGAGGACTCGCAGACAGGCACAGGGCAGCGTCACCCTCAACTCCGACGGCGGC
ATGGGCCTGGGCGCTAAGATCCCGCTCGCACACAACGACAAGAATGTGGTGAGCGCTATC
GGCTCCATGGACTTCAACAACAAGCTGCAGCCTGCTTCCAAGGGCTTCGGTCTGGCTCTG
GACAACGTCAACGGGCACGGACTGACGGTGATGAAGGAAAGTATCCCCGGGTTCGGGGAC
AGGCTGTCGGGCGCTGGCAAGCTGAACGTGTTCCACAACGACAACCACAACGTGGCCCTG
ACCGGCTCTCTTGCCAGGAACATGCCCAGCATCCCGAACGTGCCCAACTTCAACACGTAC
GGCGGGGGCGTCGACTACATGTACAAGAACAAGGTGGGAGCGACTCTGGGCATGGCCAGT
ACTCCGTTCTTGGACCGCAAGGACTACTCCGCGATGGGCAACCTGAACCTGTTCCGCAGC
CCGACCACTACCGTGGACTTCAGCGGCGGCTTCAAGAAGTTCGAATCTCCCTTCATGAGC
AGCGGCTGGAAGCCTAACTTCTCCTTTAATCTTGGCAGGTCATTCTAGAAATATTTTTAA
ACTCTTATTTAAAAATTAAATGTAAAAAATCCWGTTTGTTCATGATAATAAGAATAAATR
ACAGTATTGTTCGTACTATTTACTATGTAATCTATAAATTGTATTAATAAATGAAAATTA
ACTATMTAAMWAAAAAAAAAAAAAAAAAAAAAAAAAACATGTC
S. frugiperda-j2rc2367 (Sfrc2367); Un-annotated
(SEQ ID NO: 104)
ATCAGTCGTCCCTCGTACATCCAACACTTCAAACCAAATATCTCCATATACATAGTAACA
ACATGTTCGCCCTAAAGTTGGTACTAGCTGCAGTGCTGGTGGTCGCAAGCGCCAGACATC
TACCACAGGACCACTCAACGTACCGAACATGTACAGCTGCTCGGGTTCGACGAAGATGGA
CGGCCAGTGTTTGAGCACGAAGACCTGCTCGCAGAACCAGAGGAGTTCTATCAGCCAGAG
CACCTGGCGAGGACTCGCAGACAGGCACAGGGCAGCGTCACCCTCAACTCCGACGGCGGC
ATGGGCCTGGGCGCTAAGATCCCGCTCGCACACAACGACAAGAATGTGGTGAGCGCTATC
GGCTCCATGGACTTCAACAACAAGCTGCAGCCTGCTTCCAAGGGCTTCGGTCTGGCTCTG
GACAACGTCAACGGGCACGGACTGACGGTGATGAAGGAAAGTATCCCCGGGTTCGGGGAC
AGGCTGTCGGGCGCTGGCAAGCTGAACGTGTTCCACAACGACAACCACAACGTGGCCCTG
ACCGGCTCTCTTGCCAGGAACATGCCCAGCATCCCGAACGTGCCCAACTTCAACACGTAC
GGCGGGGGCGTCGACTACATGTACAAGAACAAGGTGGGAGCGACTCTGGGCATGGCCAGT
ACTCCGTTCTTGGACCGCAAGGACTACTCCGCGATGGGCAACCTGAACCTGTTCCGCAGC
CCGACCACTACCGTGGACTTCAGCGGCGGCTTCAAGAAGTTCGAATCTCCCTTCATGAGC
AGCGGCTGGAAGCCTAACTTCTCCTTTAATCTTGGCAGGTCATTCTAGAAA
S. frugiperda-Chitin synthase B (SfChsB); AY52599)
(SEQ ID NO: 105)
ATGGCGAGACCAAGACCTTATGGTTTTAGGGCTTTAGATGAGGAGAGTGATGACAATTCG
GAGTTGACTCCGTTGCACGATGATAATGATGACCTAGGACAAAGAACAGCTCAAGAGGCA
AAAGGATGGAATCTGTTTCGAGAGATTCCGGTGAAGAAGGAGAGTGGGTCTATGGCCTCA
ACTGCCGGGATAGACTTCAGTGTAAAGATCCTTAAAGTCCTGGCGTATATTTTTATATTT
GGCATAGTGCTCGGATCTGCGGTTGTGTCTAAGGGTACGCTGCTTTTTATCACATCACAA
CTGAAAAAGGGCAAAGCAATCGTTCACTGTAATAGACAGTTAGAACTGGACAAGCAGTTT
ATAACAATCCATTCGTTGCAAGAGCGTGTGACGTGGCTATGGGCAGCCTTCATAGCATTC
AGTATTCCAGAAGTTGGCGTTTTCTTGAGATCAGTCAGAATATGCTTCTTCAAAACAGCA
CCGAAGCCTTCTGTTTTACAGTTTTTGACGGCCTTCGTAGTAGACACCCTTCATACAATA
GGCATTGGATTACTGGTGCTTTTCATCCTGCCAGAATTAGACGTGGTTAAAGGAACAATG
CTAATGAATGCTATGTGCTTCATGCCTGGAATACTAAACGCTGTGACCAGAGACCGCACG
GACTCTCGATACATGTTGAAAATGGCACTAGATGTACTAGCTATCTCCGCTCAAGCCACC
GCGTTCGTCGTCTGGCCTCTGCTAAAAGGCGTTAGTATGCTCTGGACGATTCCTGTCGCA
TGCGTATTCATCTCACTCGGATGGTGGGAAAATTTCGTCGGCGATATCGGAAAACAATGG
CCAGTCCTGGAACCTGTACAAGAACTTCGTGACAATTTAAAGAAGACTCGTTACTACACA
CAGAGGGTGTTGTCTTTGTGGAAGATATTCATATTCATGTGTTGCATCCTGATATCTTTG
GCGGCACAAGATGACAGCCCGCTTTCTTTCTTCACGGAGTTTGCTACTGGATTTGGTGAG
CGCTTCTACAAAGTTCATGAGGTTCGAGCGATACAGGACGAATTTGAAGGTTTTCTGGGC
TACAAAATTATGGACTTATACTTCGATCAAATGCCAGCGGCATGGGCCACCCCACTGTGG
GTGGTGCTGATCCAGGTCCTGGCTTCTTTAGTCTGTTTTATGGCAAGTTTGTCTGCCTGC
AAGATTCTGATACAAAACTTCAGCTTTACATTTGCGTTGAGTCTTGTTGGACCTGTCACC
ATCAACTTGTTGATTTGGCTTTGCGGCGAGAGGAACGCAGATCCCTGCGCATATAGTAAT
ACGATACCAGATTATCTGTTCTTCGACATACCACCGGTGTATTTCCTGAAGGAGTTTGTG
GTGAAAGAGATGTCGTGGATTTGGTTGCTGTGGCTGGTGTCGCAGGCGTGGGTGACGGCC
CACAACTGGCGCTCCCGGGCCGAGCGTCTCGCCGCCAGCGACAAGCTCTTCAACAGGCCT
TGGTACTGCAGCCCCGTCCTCGACGTCTCCATGCTGTTGAACAGAACCAAGAATGAAGAA
GCGGAAATAACGATAGAGGATCTAAAAGAAACAGAGAGTGAGGGTGGGTCTATGATGAGC
GGATTTGAAGCAAAGAAAGACATAAAGCCTTCTGACAACATTACGAGGATATATGTCTGC
GCGACTATGTGGCACGAAACGAAAGAAGAAATGATGGACTTCTTGAAGTCTATCCTGCGT
TTCGATGAGGATCAGAGCGCGCGTCGCGTCGCACAGAAGTACTTGGGCATTGTAGATCCT
GATTACTATGAACTCGAAGTACACATCTTCATGGACGATGCTTTCGAAGTGTCGGACCAC
AGCGCGGACGACTCGAAAGTGAATCCCTTCGTGACGTGTCTCGTGGAGACTGTCGACGAG
GCTGCTTCAGAGGTCCATCTCACCAACGTGAGGTTGAGGCCACCGAAGAAATTCCCCACA
CCGTACGGCGGCCGACTGGTCTGGACTCTCCCAGGAAAGAACAAAATGATATGCCACCTC
AAAGACAAGTCCAAAATACGACACAGGAAAAGATGGTCTCAAGTGATGTACATGTACTAC
CTATTGGGCCACCGCCTGATGGACGTGCCGATCTCAGTGGACCGCAAGGAAGTCATCGCA
GGGAACACCTACTTACTGGCTTTGGACGGCGACATTGACTTCAAACCGACAGCAGTCACG
TTACTAATCGATTTGATGAAGAAGGATAAGAATTTAGGAGCAGCGTGCGGGCGCATCCAT
CCTGTGGGCTCAGGCTTCATGGCATGGTATCAAATGTTCGAGTACGCTATTGGTCATTGG
CTGCAAAAGGCGACTGAACACATGATTGGCTGTGTACTCTGTAGCCCTGGATGCTTCTCC
CTCTTCAGAGGAAAGGCTTTGATGGACGACAACGTTATGAAGAAATATACCTTAACTTCC
CACGAGGCACGACACTATGTGCAATACGATCAAGGCGAGGACCGTTGGTGCACGCTACTG
CTGCAGCGCGGGTACCGCGTGGAGTACAGCGCGGTGTCGGACGCGTACACGCACTGCCCC
GAGCACTTCGACGAGTTCTTCAACCAGCGCCGCCGCTGGGTGCCCTCCACGCTCGCCAAC
ATCTTCGACCTGCTCGGCAGCGCCAAGCTCACCGTCAAGTCCAACGACAACATCTCCACC
CTCTATATAGTCTATCAGTTCATGTTGATAGTGGGTACGGTGTTGGGTCCCGGCACGATC
TTCCTGATGATGGGGGGAGCCATGAACGCCATCATTCAGATCAGCAACGCGTACGCGATG
ATGTTGAACCTCGTACCACTCGTCATCTTCCTTATAGTCTGTATGACTTGTCAGTCAAAG
ACGCAGCTCTTCCTCGCTAACCTCATAACATGCGCATACGCAATGGTGATGATGATCGTG
ATAGTGGGGATAGTTCTGCAGATAGTGGAGGATGGATGGCTGGCTCCGTCCAGTATGTTC
ACAGCTTTAATATTCGGTACATTCTTCGTCACCGCGGCACTACACCCGCAAGAGATCAAA
TGTTTGTTGTTCATAGCAGTGTACTATGTAACCATCCCTAGTATGTACATGTTGTTGATC
ATATACTCCATCTGTAATCTCAACAACGTATCCTGGGGTACCAGGGAGACACCGCAGAAG
AAAACTGCTAAGGAAATGGAAATGGAACAGAAGGAAGCAGAAGAAGCGAAGAAAAAAATG
GAGAGTCAGGGTTTGAAGAAGTTGTTTGCCAAGGGAGAAGAGAAGAGTGGTTCGTTAGAG
TTCAGTGTGGCGGGCCTGTTGCGATGTATGTGCTGCACCAATCCAGAGGATCATAAGGAC
GATCTCAACATGATGCAGATCTCACACGCGTTGGAGAAGATAAATAAGAGATTGGATCAA
CTCGATGTCCCTCCTGAGCCGACCCACCAGCCCTCGCATCCGCACACACACGTGGAGACG
GTCGGTGTTCGTGATTACGAAGACAGCGAGATTTCCACTGAAATTCCTAAGGAAGAACGA
GACGACCTGATTAACCCCTACTGGATCGAGGACGTGGAACTCCAGAAGGGCGAGGTAGAC
TTCCTCACCACCGCTGAGACCAACTTCTGGAAGGATGTCATCGATGAATACTTACTGCCT
ATTGATGAGGACAAGCGTGAAATTGAACGTATAAGAAAAGATTTGAAGAACTTGCGAGAT
AAGATGGTGTTTGCGTTCGTGATGTTGAACTCTCTGTTCGTGCTCGTCATCTTCCTGCTG
CAGCTCAGCCAGGACCAGCTGCACTTCAAGTGGCCATTCGGACAGAAGTCCAGCATGGAG
TACGATAATGATATGAATATGTTCATCATAACCCAAGACTACTTAACGCTGGAACCTATC
GGTTTCGTGTTCCTCCTGTTCTTCGGCTCCATCATCATGATCCAGTTCACCGCCATGTTG
TTCCATCGCCTGGACACGCTGGCCCATCTGCTGTCCACCACCAAGCTGGATTGGTATTTC
AGTAAGAZGCCGGACGACCTATCAGACGATGCGCTAATAGACTCTTGGGCGTTGACAATA
GCGAAGGATCTTCAACGTCTGAACACCGACGACTTGGATAAACGAAATAACAACGAACAC
GTGTCCAGGAGGAAGACCATATATAACTTGGAGAAAGGGAAGGAAACCAAACCGGCTGTT
ATCAACCTCGATGCCAACGCCAAGAGGAGATTGACTATCCTGCAGAATGAAGACTCAGAA
TTGATCTCCCGCCTGCCATCTCTGGGACCTAATTTGGCAACTCGTCGTGCCACGGTGCGT
GCAATAAACACTCGACGCGCATCTGTCATGGCGGAGCGACGCAGGTCTCAGTTCCAAGCG
CGACCTTCCGGGGGATCATACATGTATAATAACCCTCAAAACACGATTCAGCTGGACGAT
ATGGTCGGGGGGCCGTCGACGTCGGGAGTGTACGTGAACCGAGGGTACGAGCCCGCCCTG
GACAGCGACATCGAGGACACGCCCGTGCCCACCAGACGATCCGTTGTACACTTCACCGAC
CATTTCGCGTGATAACCACCAAAATCTGACTAACATCTCCATATTACATTTTCTACTCTG
TTACGAAACGATAAAGTTTAAAGTGTATTAAATAAATTGGACAGATTTGAGTAGGTTTCA
CGTTTGTGTTTAAATAATTTATGAAAATAACATACCTATTGCTTTATGACCGCTTTAATA
TTAAAAGAAACTCAATATATGCATTAA
T. castaneum-Peptidoglycan recognition protein LC (TcPGRPLC); DT786101.1
(SEQ ID NO: 106)
ATATAAAAAAAAAAAAATATTAGGCAATTTATTTACACAAATAGCACAATTTATCAATTC
ACAAGTTGTGTATTTTATTATAAATACTAAATCAACAATAGCGACTAACAATTAACACAA
TTTTATTTCACTTCCTGTCACTATCGCGAGTAATAAATTCCCGCATCAAAATGCGGCCAA
TTTTTGATCTCTTTGTAAACATTTGGCCCCGGACTTTCCGTTCTAAACGTCTGATTGTGA
GCTACCAGTTTATAATCCCTGGCCAATTTTCCACTCTTTACCCCCTCATCTAGCAATTTC
TTCGCCACGCTGATCATCTCCGTCGTCAAATGATCATGAAAAAAATTCCCAATAAAACTA
ATCCCGATCGAATCATCCATGTGAAAATTCCTGATATCCCAGCCTCGTCCAACATACGCA
TTCCCATCTCCACCAATTACGAAATTGTACCCAATATCGGGACTTTTCAAATTGCCCACA
TGGTAGTCCTGCATGGACTGCACCCTTTGCGAACATGCAGGAAAGTCGCTACAAGTCGGG
GTAACAGTGTGTGAAACGATGACAAAATGAGTGGGATGTGGCAGTGGTTTAGAGAAGTTT
AAGGTGGCACGGCCTCCCCAAATTTTTTTCTCGATGATGGCACCGGGGCCCAAAGGAAGT
CTTGGAGTCGCAGTGTTAGTGTCGGGAGTTTCGGGAGACTTTTCAGTTTTCCGTGGGAGT
ATTACAACGCAAGTIGTGGTGACGAGGATTACGATTACGACTAGAGAAATGCCCAAATAT
TTCACAGGTTTGGAGTATAAAAAGGAGTTTTTTTGGTTTTTCAGCGGGGGGGAGAGTCGG
AAACAGGGGCTTTTCAGACTTGACGAAGTTTACCAATGGAATCAGTTGAATTAACAAATC
CTTTTCCCTAAAGTCCTCTAAAAACAGATGATGGGGCCCCAT
T. castaneum-Peptidoglycan recognition protein 2 (TcPGRP2); XM_965754.3
(SEQ ID NO: 107)
ATGAGTGGCAGTGACCCTTTAACAAATACCCAACAATCCGATCAAGATTATTACCATCCA
CTCTGTTATTCAATTCAAGTGGACGACGAAAATGAACAATCAGCTCTCCTGCCCGCATTT
CATCAAAGGAAAAGTTTGCGAGTTCAGGATAAAATCTTTATTGTATTTTTATTTTCAATT
CTAATTACCGGACTAGCCATTGGCCTCTATCTCCTTGCAACTGAGGGACACGAATGGAAA
GCTGCAGGAGTCTATAATATTACAGTTCGGGAACAGTGGCAAGCTCACGTCCCTTCATCA
ACAATGCCAAAGTTGGAACTTCCCGTAAGAAGAGTTTTATTTCTTCCTGCAAATACCACT
AGCTGCGGCAGCAAATCCCACTGTGCCAAAGTCCTCCAGGAACTACAATTACAGCATATG
CTGCAGTGGAAAGAACCTGACATCTCCTACAATTTCATAATGACTGCAGATGGCAGAATT
TTCGAGGGGAGAGGATGGGACTTTGAAACTTCTGTTCAAAATTGTACGGTTAATGATACT
GTGACAGTTGCTTTTTTGGACGAATTAGATGCGAAAGCACCGACGTTTAGACAAGCTGAA
GCGGCAAAAATGTTCCTGGAAGTGGCAGTAACAGAGGGAAAATTAGAACGGTGTTTTAAT
ACCGCGGTCTGGGGAGGAAATAAATTCTTCATTGATTTGGCTCGAAATGTTCAAGACGTC
TTATCGGAATGCGAGGGAATTACTTAA
T. castaneum-Beta 1-3 glucan binding protein (TcßGRP2); XM_966587.4
(SEQ ID NO: 108)
ATGTGCGTTTGCAAGACTGTGCTATTAATCGTTGGGCTGGGGGGTTGTTTTGCGGGTCCG
GTCCGTAATTATGGGCCATTGAGACATTACAACGTTCCGCGACCCTCAATCCAAGCATTC
AGGCCCCGTGGCTTCAAAGTGAGCATCCCTCATACTGAAGGCATCCAATTATTCGCCTTT
CATGGAAATATTAATAAACCCCTGCACGGTCTCGAGGCCGGACAATTTTCCCAAGACGTC
CTCCAAAGGGAGGGAGACGAGTGGGTGTTCCAAGACTCCAGTGCCAAATTAAACGTGGGA
GATAAAATCTATTATTGGCTCTTTATCATTAAAGAAGACCTGGGCTACAGATACGATCAC
GGCGAGTACGAAGTGAAAGTTTTAGCCACCCGTGACTTCGATTCTCCTCAAACAACCTCT
GTGACGCCGAATCTTGCCCCCAATCTCGGCATTTGCGAAAAGGTGATGGTAAATCTCACG
CGGAAGCTGCTCGATTTGCAGCAAGAAATTGAGTCCCTTAGGGAGACGAACGATATTTTG
GAGGATATGGTTCAGAAGCACACTGATACGGCGACTACTCTCACTTTGGACGGCTTGATG
ATAAAGGATGACGACGAACTTGTTTCGGTGATTCAAGCAATTATTAAAGATAAACTTGGA
CTAAAAAGCAGGATTCAAAATGTGACGAGGCAGGAGAATGGAATGGTCAAGTTTCAAGTG
GCGAGTTTGAGAGAAAAGTTGGAGGTTGTCAAAGCGGCCAAAAGAAAACTCAAGTCGTCG
AGCTTTACGATCACGTATTAA
T. castaneum-midgut protein (TcMDGP); XM_971351
(SEQ ID NO: 109)
ATGTATCCGTTGAAGAGGATGCCCAGTGAAGAAATCAGTATCAGTGATCTGCCTAGCGAA
ATGAAAGAAGTTTTACTAGAAATTAGCCCGAACTTTGATGAAAATCTGAAACGGGCTTTC
AGGAACGAAGGAGTGAGGCTGCAGAGAGTGCAGAACAATGGACGATTTATTCATCAGCTG
GACGACGTTCTCTTATCCATAGACGATACCAAAATCGAGTTACGCAACCTGAAATTCCCC
TGGATCCCCGACTTCCGCATCGTGGACTTGTCCAGCGACCTGCCCATGTCATGCCTCGAC
CTAAACCTAAACCTGGGCAATTTGCGAATTGAGGGCGAGTACGAAGCCAACAACACCACA
CTCAGGCGATGGCTCCCGGTATCTCACATTGGTCGAATCGTGATCGGTTTTAACAACGTC
CGAGCGAACGGAAAAGTCGGACTCGTGCTCGAGCAGGATTCTTTCGTTCCGCAGAATTAT
GATATTAGATATGAGCCGACGGATGTTGTTATTAGGGTTAGCTATCACGTGGATGGCGAG
AATGAGGTGCAAAATGAGATTAGCAATTCAGATATTGAGGCCACGCTAGGCAAGACCGTG
TGGGTGCAGTTGACTGAGATATTGTCCAACCTGTTGCATAGGCAATTGGGCGAGGTTGTA
GTGGAGTTTTCCGTGACGGAACTCCTCGTCGATAGGGACGAGGAATACAGGGAATACGCC
AAGGGACAAGCAGCGCGCGCCAATAAACTCCTGGACTCGCTTTTGTGCTCAGCCAAGGAC
TATTTAGTCGCGAAGGACTTGAGGACGGTCAAAACGCCACCCTTCGACGTCGTCTTCAAA
GGGAAAGTCTCGGGGGTGCAGCAGGGGACCTTCAGCACGGGGGAAGGGTTCCTCCAAGAC
CTCGCCACTTTGACGCGGAGACACAGCTTTAGTTTGTACGAAGACAAACACAAACTCACG
ATATATGGGGGGATCAGGCTGAGGGAGTTTAAACTCGGGTATGGGGGCTACCAGGGCCAG
TACGAGGAAACGGCCGTCTCAGGCAGCATCAAAGGCTCGCTCTACAAGAACGAGATTTTC
GTCAAGATCACTGTGAAAAAAGAAGGCGAGCGGTGCTCGACTCAGTTAGATTCCGTCCAA
GTTGTTGTTGTAAAGTAA
T. castaneum-Chitin synthase 2 (TcCHS2); EFA 10719.1
(SEQ ID NO: 110)
ATGGCGGCGCGTCATCGCTTTGCCACAGGGAGCCCTGAGGAAACAGAGCCCCTGTATTCG
TCGACGCAAATGCCCGAAAAAGTCCGGGAAAAATGGAACGTCTTCGACGACCCCCCAAGA
GAGCCCACTTCGGTTCCGAAGTCAAAAGAACCTACATCGAGTGGGGGGTGAAGTTTTTGA
AAGTTGTGACAATCATAACTGTGTTTTTTGTGGTCCTTGGTGCTGCAGTGGTTTCGAAAG
GGACAACCTTGTTCATGACGTCACAAATAAAAAAGAATGTGACAAGGGCTTATTGCAACA
AAAAGATAGACCGCAACCTCCAATTCGTCGTCTCCCTCCCCGAAGTGGAGCGCGTGCAAT
GGATCTGGCTCCTCATTTTCGCTTACTTGATCCCCGAAGTGGGTACCTGGATCCGCGCCG
TCCGCAAATGCCTCTACAAGCTCTGGAAAATGCCCTCCCTCTCCGAATTCCTCTCCCTCT
TCGGCACGGAAACGTGCCCCGCCATCGGAAGCGCAATTTTGATATTCGTCGTCCTCCCCG
AGCTGGACGTCGTCAAAGGGGCGATGCTCACAAACGCGGTTTGTTTCGTGCCCGGAGTTG
TGGCAATGTTCTCGCGCAAACCGTGCTCCATAAACGAGAACCTGAAAATGGGGCTGGACA
TCGCCAGCATAACTGCACAGGCGTCAAGCTTCGTGGTGTGGCCCTTGGTTGAAAATAACC
CGACCTTGTACCTAATCCCCGTTTCCGTGATTTTGATTTCGGTGGGTTGGTGGGAGAATT
TCGTGTCGGAAACGTCCTACTTACCGTTTATCCGGGCTCTGGGCAAGAGCAAAAAGGAGT
TTAAGACAAGGACGTACTTCATTTACGCGTTCGTGGCCCCGGTTAAATGCCTGGCGTTTT
TTTGCACCGCTTTGGTCATTTTTTACTGCCAGGAGGGCAGTGTTGACTTTTTATTTGATA
ATTTTTCAGCCGCGTTTCAGGATCATAACATTGAAATTACCGAAGTCGCGCCCGTCTTGC
CGGGGAATTACGCAAATGCAGTTCGGTCGGGAGCCGAAACCCCATCCACACAAGCAGTTA
CATGACGGGGATTTGGGTTTGGTTGATTAACATTTCGGCGACTTATATTTGCTACGCGTT
TGGGAAATTCTCCTGCAAAGTCATGATCCAGAGCGTTAGCTTCGCTTTTCCGATCAATTT
GTCGGTCCCTGTCCTCTTATCCGGACTGATCGCAATGTGTGGCATGTACTACAGGGATGA
GTGTTCTTTCGCTGAGTCAATTCCTCCATATTTGTTTTTCGTTCCTCCACCTCTCATGTT
CCTACAAGATTTTCTCTCGCACCAACACGCCTGGATTTGGCTGGTTTGGTTGCTGTCACA
AGCCTGGATTTCGGTGCACATTTGGTCCCCAAACTGTGACAAACTTTCAAGCACCGAACA
GTTGTTTATTCGGCCCATGTATGACGCGTTTTTGATCGATCAAAGCCTGGCGTTAAACCG
GAGACGTGACGAAAATCCCAGAAATTACAGAAGCGACGAAGGGCCTCAAATTACAGAGCT
CGAGCCGGAAACGATCGAGAGTCAGGACGCCATAACCCGGATTTACGCCTGCGGGACAAT
GTGGCACGAAACTCCCGAAGAAATGATGGAATTTTTGAAATCGGTGTTCCGCTTGGACCA
AGACCAGTGTTCCCACAGGATTGTGAGGGAGCATTTGGGACTCAAGCATGACAATTACTA
CGAATTGGAGACTCATATATTTTTCGATGATGCGTTTATTCGGACCAGTGAAGACGATAA
TGATCCCCACGTCAACGAATACGTTGAGTCACTTGCGTCCATTATCGACGAGGCTGCGAC
TAAGGTTCACGGTACCACGGTGAGGGTGCGTCCGCCCAAAGTGTACCCCACGCCTTACGG
CGGACGCCTGGTCTGGACGCTCCCAGGGAAAACAAAAATGATCGCCCACTTGAAGGACAA
GAAGAAGATTAGGGCGAAAAAGCGCTGGTCTCAGTGCATGTACATGTACTTTTTGCTCGG
ATTCAGATTGCAAGCCAACGACGAACTCTCCGCCCACAGCAAGGAAATCCGCGGCGAAAA
CACCTACATCCTCGCCCTGGACGGCGACATCGATTTCCAACCCGAAGCCCTGCACCTCTT
GGTGGACTACATGAAGAAGAACAAAACGTTGGGGGCGGCCTGCGGCCGCATCCACCCCAT
CGGCAGCGGCGGCATGGTCTGGTACCAAATGTTCGAATACGCCGTCGGTCACTGGATGCA
AAAAGCCACCGAGCACGTCATAGGCTGCGTCCTCTGCAGCCCCGGCTGTTTCTCCCTGTT
CCGGGGAAAAGCCCTCATGGACAAAAGCGTCATGAAGAAGTACACCACTCGATCGACCCA
AGCCAAGCACTACGTGCAGTACGATCAAGGGGAGGACCGGTGGTTGTGCACTTTGTTACT
CCAGAGGGGCTACCGTGTGGAATACTCCGCAGCCTCGGACGCTTTCACGCACTGTCCGGA
AGGCTTCAACGAGTTTTACAACCAGCGGAGGCGCTGGATGCCGTCCACTATGGCCAACAT
TTTGGACCTTTTGATGGATTACGAGCACACGGTCAAAATCAACGAAAATATTTCCATGCT
GTACATCGGGTACCAAATTATTTTAATGATCGGTACGGTCATTGGCCCCGGTACTATTTT
CCTCATGTTGGTCGGCGCCTTCGTGGCTGCCTTTGGGCTCGACCAATGGAGCAGTTTCTA
CTGGAATTTACTACCAATCGCAGTTTTTATCCTAGTATGTGCCACTTGTAGCTCCGATAT
CCAATTATTTTTCGCCGGCCTTATCAGCGCCATTTACGGCCTGATAATGATGGCTGTTTT
CGTCGGTGTGATGCTCCAAATCAGCCAAGACGGCCCACTTGCGCCTTCCTCCCTTTTCTT
CTTCTGCATGGCTGCTGAATTTATAATCGCAGCACTGCTGCATCCGCAAGAATTCAACTG
TTTGAAATACGGGGTCATTTACTACGTCACGGTCCCCAGCATGTACCTCCTCCTAGTCAT
CTACTCGGTCTTCAATATGAACAACGTGTCCTGGGGGACGCGCGAAGTGACAGTCGTGCC
CAAGCCTGACCCCAACGCCGTCCAGAAAATCGAAGAGAAAAAACCGGAGAAGAAAGACAA
AGTTTTGACGTTTCTGGGCGCGAATGCCCAGGACGACGAAGGCGGGCTTGAATTTTCGGT
CAACAAACTTTTCAAATGCATGATTTGTACGTACAAGGCCGATAACAAGGAAAACGAGCA
GTTGAGGAAAATACAAGAGTCGTTGAGAGACTTGAATAGGAAAATCGAGTCGCTGGAAAA
AATGCAATATCCGGATTTGAGGTCTCCTGCCGTTAGCAACGTTACAACGTTCATGGAGGG
CTCAAAGGCGACGGTTAAGAACAACGTGGAGGATAACTACATGGAGGCTCCGCAAGACAA
TGTTTCGCAACCGTCGGATGAGGTCATGGAGAATAGTTGGTTCTACGATGGGCCTTTGAT
TAGGGGGGAAGTGCATTACTTGAATAGGAATGAGGAAACGTTTTGGAATGAACTGATAGA
GCAGTATTTACACCCGATTGAGGATGACAAGAAGAAGGTTTCGGCTGAATTGAAAGATTT
GAGGGACAAAATGGTGTTTACTTTCCTGATGTTGAATTCGCTCTACGTTATTGTGATTTT
TTTGCTCACTTTGAAGAAGGATTTGCTCCATCTGGACTGGCCGTTTGACCCCAAAGTGAA
CTTCACGTATTTCGAGGACAAAAATGAGATTGGCGTTTACATAACATACCTCCAGCTGGA
GCCCATCGGTTTCGTGTTCCTCATATTTTTCGCCCTGCTTATGGTGATCCAGTTCTTCGC
AATGATGATCCACCGCTTCGGCACCTTCTCCCAAATCATCACAAAAACACAATTAGACTT
CGATCTGTGCAGCAAACCAATCGACGAAATGACTGTGGACGAACTCAGGTCCCGCGACCC
TATAAAAATAGTCGCAGATTTACAAAAACTAAAAGGTATAAACAACGAATACGAGGACCA
AACGGAAGTTCCGGTCGAAATGCGAAAAACAGTAAGTAATTTGGCGCAAACGAGCGGTGG
TGGTGAGAACAAGCCCATATTTTATTTGGATGAAGCGTTCCAAAGGCGAGTCACTCAAAT
AGGGAGCACTTCAAGCAACAACCCGAGCATTAGCGCCTTTAGGAAGAAAAGCCTTGCTTA
CGTCCAACAAAGAATGAGCATAGCCCCAAATCGGGTGTCACAAGCCCGGCCTAGTGTGCA
GTTACGGTACCCCAATGGAAAAGCCAACGAAAATTTCGTTTTTGACGAAAACGGGTCAGA
CGTGGAGGCATAA
>M. sexta-Peptidoglycan recognition protein 2 (MsPGRP2); GQ293365.1
(SEQ ID NO: 111)
TAATCATTAGAAAAATGGCGAGCTTCGCTTTAATAGTTATCCTTAGCGTAATTGGCTTTA
TATCGGCCTATCCTAGTCCTGAAGGTTACAGTTCTGCCTTCAACTTTCCATTCGTAACCA
AGGAGCAGTGGGGCGGCAGGGAGGCACGCACGTCGACGCCACTCAACCACCCAGTGCAGT
TCGTGGTGATCCACCACAGTTACATTCCCGGCGTGTGCCTCAGCCGGGACGAGTGCGCGC
GCAGCATGCGCTCCATGCAGAACTTCCACATGAACAGTAACGGGTGGAGTGATATTGGAT
ACAACTTCGCTGTCGGCGGTGAAGGGTCGGTGTACGAGGGCCGCGGCTGGGACGCGGTCG
GCGCACACGCAGCTGGCTATAACAGTAACAGTATCGGCATCGTGCTCATCGGCGATTTTG
TTTCAAACCTCCCGCCGGCGGTGCAAATGCAAACCACACAAGAATTGATCGCAGCGGGCG
TGCGACTCGGTTACATCAGGCCCAACTACATGCTCATCGGGCATCGTCAGGTCTCCGCCA
CTGAGTGCCCAGGAACCAGACTCTTCAACGAAATCACCAACTGGAACAACTTCGTGAGG
>M. sexta-Beta-1, 3-glucan-recognition protein 2 (MsßGRP2); AY135522.1
(SEQ ID NO: 112)
GAGCGTCTGTTTGTTCGCAACCATTGCGGGCTGCTTGGGCCAGCGAGGGGGTCCATACAA
GGTGCCTGATGCGAAACTCGAAGCTATCTACCCCAAAGGCTTGAGAGTCTCTGTGCCAGA
TGATGGCTACTCCCTATTTGCCTTCCACGGCAAGCTCAATGAGGAGATGGAAGGTTTAGA
GGCTGGCCATTGGTCCAGAGACATCACCAAAGCGAAGCAGGGCAGATGGATATTCAGAGA
TAGGAATGCTGAGCTGAAGCTTGGAGACAAAATTTACTTCTGGACTTACGTTATTAAGGA
TGGATTGGGATACAGGCAGGACAATGGAGAATGGACTGTTACAGAATTCGTCAATGAGAA
CGGTACAGTGGTGGACACTAGTACAGCGCCGCCACCAGTAGCACCCGCCGTTTCAGAGGA
AGATCAATCGCCAGGTCCTCAGTGGAGACCTTGCGAAAGATCCCTGACTGAGTCCTTGGC
CCGCGAACGCGTTTGCAAAGGCAGCCTTGTCTTTAGCGAGGACTTTGATGGTTCCAGTTT
GGCCGACTTGGGCAATTGGACCGCTGAAGTCAGATTCCCTGGCGAACCGGACTACCCGTA
CAACTTGTACACTACGGACGGCACTGTGGGATTCGAAAGTGGGTCTCTGGTGGTGAGACC
CGTCATGACCGAGTCCAAATACCACGAGGGCATCATATACGACCGCCTCGACCTTGAGAG
ATGTACAGGACAGCTGGGTACGCTGGAATGCAGGCGAGAGAGCAGCGGCGGTCAGATTGT
ACCACCTGTGATGACAGCTAAACTGGCCACTCGACGCAGCTTCGCGTTCAAGTTCGGCAG
GATCGATATAAAGGCGAAGATGCCGCGCGGGGACTGGTTGATACCAGAACTCAACCTCGA
ACCTTTAGATAACATATACGGCAACCAGCGATACGCTTCGGGTCTCATGCGGGTCGCGTT
CGTGAGAGGAAACGATGTATACGCCAAGAAGCTCTACGGAGGTCCGATAATGTCCGACGC
GGACCCGTTCAGGTCCATGCTGTTGAAGGACAAGCAAGGGTTGGCCAACTGGAATAATGA
TTACCACGTCTACTCGCTGCTGTGGAAGCCTAACGGTTTAGAGCTGATGGTGGACGGTGA
AGTGTACGGCACCATCGACGCTGGCGATGGCTTCTACCAGATTGCGAAGAACAACCTCGT
GAGCCACGCCTCGCAGTGGCTCAAGGGCACCGTCATGGCGCCGTTTGATGAAAAGTTCTT
CATCACTCTGGGTCTTCGCGTGGCGGGTATCCACGACTTCACGGACGGTCCGGGCAAACC
TTGGGAGAACAAGGG
>M. sexta-Relish family protein 2A (MsREL2A); HM363513.1
(SEQ ID NO: 113)
ATGTCCTCTTGTCCAAGCGACTATGATCCCAGTGAATCGTCCAAATCTCCACAAAGTATT
TGGGAGTCAGGAGGATACAGTTCTCCGTCGCAACAAGTTCCTCAATTGACTTCTAACTTA
ACAGAATTGTCTGTTGATCACAGCTATAGATACAATGGAAATGGACCATATCTACAGATC
ACAGAGCAACCACAGAAATACTTTCGGTTCCGTTATGTTAGCGAGATGGTGGGAACACAT
GGATGTTTGCTTGGCAAATCTTATACAACAAACAAAGTTAAAACTCATCCGACAGTTGAA
CTCGTGAATTACACCGGTCGAGCCCTGATAAAGTGCCAACTATCGCAAAACAAGAGCGAA
GACGAACACCCGCACAAACTGCTCGATGAACAAGACAGAGACATGAGCCACCACGTTCCC
GAGCACGGCAGTTATAGAGTGGTATTTGCTGGTATGGGTATAATTCATGCTGCCAAAAAG
GAAGTTGCGGGGTGGCTCTATAGAAAATATATACAGCAGAACAAGAATGAAAAGTTTAAT
AAGAAAGAGCTCGAAGCGCATTGTGAGAGGATGTCCAAAGAGATCGATTTAAATATAGTT
AGACTGAAGTTTAGCGCTCACGATATTGACACTGGCATTGAAATTTGCCGGCCAGTGTTC
TCTGAACCCATTTATAATTTGAAGTGTGCGTCTACGAATGATTTGAAAATATGCCGCATA
AGCCGTTGTTACGGTAGACCGAGAGGCGGCGAAGATATCTTCATATTTGTCGAAAAGGTC
AACAAGAAAAACATCCAAGTTCGGTTCTTTAGACTGGAAAACGGGGAGCGCACCTGGTCA
GCGATGGCGAACTTTCTGCTAAGCGATGTTCACCACCAATACGCTATCGCTTTTAGAACG
CCACCGTACGTCAATCACCAAATTTCTGAAGACGTGCAAGTTTTTATAGAACTCGTACGC
CCTTCAGACGGTAGGACGAGCGCTCCCATGGAGTTCACATACAAGGCTGAGCAAATCTAT
AAACAGAACAAGAAACGTAAAACTACTTCGTCGTACTCGTCGCTCGACAGCTCCTCAGGT
TCGGCCGGTTCAATTAAAAGCATCAGCGAACTGCCCGCGCCCGTTGTTTTTGCTGAAAAC
GTAAGTTTTTTCTATGACACATTACTCATTCTTCAACCCATGACGAATCTATAA
>M. sexta-Spatzle (MsSPZ1A); GQ249944.1
(SEQ ID NO: 114)
ACGAACAACCTGACAGACGGATAGCGGGACGGTCAGCACAATACGAACATTTAAGAACAA
ACGAGAGGTCTCTCCCGGTCTACAGCGAGACCCAGAGGATACAAGCAGAAGAGAGAAGAA
GACACAGTTCGAGACTAGAAGAACCGAGACAACGTGCTGAGAATGGTTCATATAAGATAT
TGAATAACCCTCCGAAACCCTGTATTACTAATAGGAGAAGTCAAATTGATTCGTCGAATG
ATAGGGTAGTGTTCCCCGGTCCGACTTCAGAAAGGTCGTACGTACCCGAAGTGCCAGAGG
AATGCAAGAAAATCGGCATATGCGACAGTATACCGAATTACCCAGAAGAACACGTAGCTA
ATATTATATCTCGACTTGGAGACAAAGGAAAAGTATTACAAATAGACGAACTGGACGTAT
CAGACACTCCAGATATCGCCCAGAGGTTGGGTCCGCAGGAGGACAACATGGAACTATGTA
GCTTTAGAGAAAAGATTTTTTACCCCAAGGCAGCGCCAGACAAAGATGGAAATTGGTTCT
TCGTTGTGAATTCAAAAGAAAACCCAGTACAGGGTTATAAAGTTGAAATTTGCGACCGTC
AGCAATTACCATGCGCGGAGTTCGCGAGCTTCCAACAGGGATATGAAGCGAGGTGCATCC
AGAAATACGTTCGCCGGACCATGTTGGCGTTGGATCCCAAGGGTCAGATGACCGACATGC
CCCTTAAAGTGCCCAGCTGTTGCTCATGCGTGGCCAAATTGAC
>M. sexta-Toll receptor (MsTOLL); EF442782.1
(SEQ ID NO: 115)
ATGCAGGCTCGGCGGTGGTGCGCGGCACTGCTATTAATGCAGATGCTGAGCTGGCTCGGA
GTCAGTGGACACTTACCGCGTCCCGAGTGCGCGCCAGCCGCAGATTGCCAACTTATACGA
GACAACATAATCGATGGATATGCACAATTCTACTTCAACGTATCAGGACATGAAGTGAAA
TTTGAACATTACATCGGAAACGACTTCGATGTCGAATTGTCATGCAATTACATCGCCATG
GACAACGCAATGCTGCCGCGGTTCTCAACGACCTTTTCAGTCAACGTAATAGTGGTTAAA
GAATGTGCTTTGCCAAGAAGTGGGTCAATCGATGCCGCTGTCGCTGCACTTAATATCAAC
GTTTTGACGGAGCTGACTCTGGACAAATTCCTAGAGCCGGCGGTGATCACGCGCGCACAT
CTTACCAGTTTACAACGACTAGAGAGGCTGGAGCTACACGGTAACTCAAACACAAGCCTC
GCCCCCGGCGCACTGGCCGCGCTCTCCGCCGCGTCCGCACTGAAATGTCTTGTATTGCAT
GCAGTACGCGTGCCCGCCGCTGACCTGGCGCGCTTGCCGTCGTCACTGCAAGAACTAGCG
TTGTTGGATGTGGGCGCTGCGAGTATGCATTTAGATTCATCGGTTAATTTGACGTCACTC
TTCGTAATCGATACACATTATCCTGTCGTCGTGAATGTGAGCAACGCCGTTGCGCTCAGA
GACTTGCACATAAATACCCCAAGTACTGTGTTGACCGAAGACGTGCTCCCGTCGTCACTC
AACTCACTTGAACTAGAGGGGTGGAACGAAACGCATCCGGTGCCTAAGACACGTTGTGTA
CTACTTAAGGAACTTAATGTAATCGGCACCGACAATGATGCCTATCCGGTGACTCT
>M. sexta-Valine Rich Midgut Protein (MsVMP1); NCBI accession number not assigned
as yet
(SEQ ID NO: 116)
ACGGACCTTCCGTTGGACCNGCCATCATCGGCGCTGGAGACATCGCTGTCGGCCCTGCTA
TCGTCGACTTCCCTTTCCCCGACGGCGGTGCCGTGTCTGCCCCCGTTGAGCCTTCCCCCA
TCGCCATCGGACCCGCTATCGTCGGTGAATCCCCTATCTCCGTCGGACCTGCCATCGTTG
AGGCCGGAGACATCGCTGTTGGACCCGCTATCATCGACTTCCCCCTTCCCGACGGTGGCG
CCGTGTCCGCCCCCGTTGAGGTTTCTCCCGTCGACTCCGTCGTCGTCGGCCCTGCCGCCG
GCTCTCAGAGCTCTCCCCTCGTCCAGATCATCATCAACGTTAAGGCCCCCGCTGGTGCCG
GCCCCGTTGTCGATGCCGTCGCTGACAAGCCCATGGACATCATTGATGTTATGCCCGTCG
TCGACCCTGCTGATTTCGTGGACCTCACCCCCGTTGTAGAGCCTGTAGAAGTCGTCGACA
TTGTCGATGTCATGCCCGTGGTTGACCCCATCAACATCATCGATGTTATGCCTGTTGTTA
AGCCCGTAAACCCCCTTGCCCGTTCTT
>M. sexta-Chitin synthase 2 (MSCHS2); AY821560.1
(SEQ ID NO: 117)
ATGGCCGCAACTACACCAGGTTTTAAGAAGTTAGCAGACGATTCTGAGGATTCAGATACA
GAATACACCCCGCTGTATGATGACGGTGATGAAATAGATCAAAGAACTGCACAAGAAACA
AAAGGATGGAATCTATTTCGAGAGATTCCGGTGAAAAAGGAGAGTGGATCTATGGCCACA
AAAAATTGGATAGAAACAAGCGTAAAAATCATAAAAGTGCTTGCCTACATATTGGTTTTT
TGTGCTGTACTGGGTTCCGCAGTCATAGCTAAAGGAACTCTTCTATTTATTACGTCACAA
CTGAAGAAAGACAGACAAATTACTCACTGCAATAGACGACTTGCTTTAGACCAACAGTTC
ATAACGGTACACAGTTTAGAAGAAAGAATAACATGGCTATGGGCAGCACTTATTGTATTC
GGTGTGCCGGAGTTAGGGGTGTTTTTGAGATCCGTCAGGATATGCTTCTTCAAAACTGCC
AAGAAACCAACCAAAACACAGTTTATTATTGCTTTCATAACAGAGACACTACAAGCAATA
GGAATAGCAGCACTTGTATTAATAATTCTACCAGAATTAGACGCTGTGAAAGGAGCCATG
TTGATGAACGCCACGTGCGCTATCCCTGCATTGCTAAACATTTTCACGAGAGACCGAATG
GATTCTAAGTTTTCTATAAAATTGATATTGGATGTATTGGCGATATCGGCACAAGCCACG
GCGTTTGTTGTTTGGCCTCTTATGGAAAGAACGCCAGTTCTATGGACCATACCAGTTGCA
TGTGTGTTAGTGTCTCTAGGCTTCTGGGAGAATTTTGTTGACACCTACAATAAAAGTTAT
GTTTTTACGGTGCTGCAGGAACTACGCGACAACCTCAAGAGGACTCGGTACTACACTCAG
CGGGTGCTATCTGTTTGGAAGATTATAGTGTTTATGGCATGCATTTTAATATCGCTGCAT
ATGCAAAATGACAATCCGTTTACCTTTTTCACTCACGCCAGCAAAGCCTTTGGAGAGAGA
CAGTATGTCGTTAACGAGGTTCTAATAGTAGTCCGAGATGACGAAACCATAGGCTATGAC
GTCACCGGAGGTATATTCGAATTGGACGCGATATGGACCTCAGCATTGTGGGTCGCATTA
ATTCAAGTGGGAGCAGCCTACTTCTGTTTCGGAAGTGGCAAGTTTGCTTGCAAAATTCTT
ATACAAAATTTTAGTTTCACTTTAGCATTGACTCTCGTCGGGCCCGTGGCAATCAACCTC
CTTATTGCTTTCTGCGGAATGAGAAATGCAGACCCTTGCGCTTTCCATAGAACTATACCT
GACAATTTGTTTTACGAGATACCACCTGTG
>M. sexta-Beta-fructofuranosidase 1 (MsSuc1); GQ293363.1
(SEQ ID NO: 118)
TTGCTGTGCGTTTTCCTTGGTAGTGTATCGTCATGTTGCGTTAATGGGCGGTACTACCCG
AGGTACCATTTGTCGCCACCGCATGGCTGGATGAACGACCCCAACGGATTCTGCTACTTC
AAAGGTGAATACCATATGTTTTACCAGTACAATCCCATGTCAAGTTTGGAGGCTGGCATA
GCTCATTGGGGTCATGCGAAAAGTAAAGATTTGTGCCATTGGAAACACTTAGACCCGCCA
TCTATCCTGATCAGTGGTACGATCAAACGGGAGTATTTTCTGGAAGTGCGCTAGTAGAGA
ATGACGTCATGTACCTTTATTATACTGGAAATGTAAATCTTACTGATGAAATGCCATTTG
AGGGACAATTCCAAGCTCTTGGTATCAGTACTGACGGTGTCCACGTAGAAAAGTATAAAG
ACAATCCAATAATGTACACGCCAAACCATCAACCTCACATCCGAGACCCAAAAGTTTGGG
AACACGACGGCTCTTATTATATGGTCTTAGGAAACGCATATGATGATTATACAAAGGGCC
AAATAGTTATGTACGAATCATCAGACAAGATCAACTGGCAAGAAGTAACTATACTATATA
AATCAAATGGATCTTTCGGTTACATGTGGGAGTGTCCAGATTTATTCGAAATAGACGGCA
AGTTTGTACTTCTGTTCTCTCCTCAAGGCGTGAAGTCTGTGGGCGATATGTACCAGAATC
TGTATCAAGCAGGATACATCGTCGGAGAATTCGATTACGATACTCATTCATTCACAATAC
TAACCGAATTCAGAGAATTGGATCACGGTCATGATTTTTACGCTACACAAACAATGAAAG
ATCCTAGTGGAAGAAGAATAGTCGTTGCTTGGGCAAGT
>M. sexta-Beta-1 tubulin (MsßTub); AF030547
(SEQ ID NO: 119)
ATGAGGGAAATCGTGCACATCCAGGCTGGCCAATGCGGCAACCAGATCGGAGCTAAGTTC
TGGGAGATCATCTCTGACGAGCATGGCATCGACCCCACCGGCGCTTACCATGGCGACTCG
GACCTGCAGCTGGAGCGCATCAACGTGTACTACAATGAGGCCTCCGGCGGCAAGTACGTG
CCGCGCGCCATCCTCGTGGACCTCGAGCCCGGCACCATGGACTCTGTCCGCTCCGGACCT
TTCGGACAGATCTTCCGCCCGGACAACTTCGTCTTCGGACAGTCCGGCGCCGGTAACAAC
TGGGCCAAGGGACACTACACAGAGGGCGCCGAGCTTGTCGACTCGGTCTTAGACGTCGTA
CGTAAGGAAGCAGAATCATGCGACTGCCTCCAGGGATTCCAACTCACACACTCGCTCGGC
GGCGGTACCGGTTCCGGAATGGGCACCCTCCTTATCTCCAAAATCAGGGAAGAATACCCC
GACAGAATTATGAACACATATTCAGTTGTACCATCACCCAAAGTGTCTGATACAGTAGTA
GAACCTTACAATGCAACACTGTCAGTCCACCAACTCGTAGAAAACACCGACGAAACCTAC
TGTATCGACAATGAGGCTCTCTATGACATCTGCTTCCGCACGCTCAAACTTTCCACACCC
ACATATGGCGACCTTAACCACCTGGTGTCGCTCACAATGTCCGGCGTGACCACCTGCCTC
AGGTTCCCCGGTCAGCTGAATGCGGATCTCCGCAAGCTGGCGGTGAACATGGTGCCCTTC
CCGCGTCTGCACTTCTTCATGCCGGGCTTCGCTCCGCTCACGTCGCGCGGCAGCCAGCAG
TACCGCGCCCTCACCGTGCCCGAACTCACCCAGCAGATGTTCGACGCTAAGAACATGATG
GCGGCGTGCGACCCGCGTCACGGCCGCTACCTCACCGTCGCCGCCATCTTCCGTGGTCGC
ATGTCCATGAAGGAGGTCGACGAGCAGATGCTCAACATCCAGAACAAGAACTCGTCGTAC
TTCGTTGAATGGATCCCCAACAACGTGAAGACCGCCGTGTGCGACATCCCGCCCCGTGGT
CTCAAGATGTCGGCCACTTTCATCGGCAACTCCACCGCTATCCAGGAGCTGTTCAAGCGC
ATCTCTGAACAGTTCACCGCTATGTTCAGGCGCAAGGCTTTCTTGCATTGGTACACCGGC
GAGGGCATGGACGAGATGGAGTTCACCGAGGCCGAGAGCAACATGAACGACCTGGTGTCC
GAGTACCAACAGTACCAGGAGGCCACCGCCGACGAGGACGCCGAGTTCGACGAGGAGCAA
GAGCAGGAGATCG
>P. xylostella-Peptidoglycan recognition protein 2 (PxPGRP2); ACB32179.1
(SEQ ID NO: 120)
CCCGATACAGTTGGAGTACCTGCCCCGGCCCCTGGGGCTGGTGGTGGTCCAGCACACCGC
CACCCCCGCGTGTGACACTGACGCCGCGTGTGTGGAGCTGGTGCAGAACATACAGACCAA
TCATATGGATGTGCTGAAGTTTTGGGATATTGGACCGAACTTCCTGATTGGTGGGAACGG
CAAGGTGTACGAGGGCCCTGGTTGGCTGCACGTCGGCGCCCACACTTACGGCTACAACAG
GAAGTCTATCGGGATCTCTTTCATTAGGAATTTTAATGCTAAGACCCCAACAAAAGCAGC
GTTGAATGCGGCTGAAGCATTGCTGAAGTGTGGAGTGAGAGAAGGACACCTGTCTCACTC
ATACGCAGTGGTCGGCCATAGACAACTGATCGCAACAGAGAGCCCAGGCAGGAAACTGTA
CCAAATCATCAGGCGCTGG
>P. xylostella-Immune Deficiency Protein (PxIMD); Px003008
(SEQ ID NO: 121)
TCCCGAAGCCACCTAGAGAACCGGTAGAGACTACAGAGACATCACAAAATAATACCGAAA
ATCAACCTTACAATGTCGAAGAAGAGGAAATACCCGAACCAGAAAAGCCTAAGAAAGAAA
AAAAGAATCCCAAGCCTACCAAAAAAACTTTCTTTAATCGTGACAAAACTAACAAACACG
ACGATACCCGCAAACATACAAAATCCGGAAAGGACCAGACATCAATTAATACTCAAGGTA
ACTTGAAAATTATACTTCCTGTAACTTAAACGGCCCGTTGACCCCAGTTTACCTTTCGCC
TTTCTTGATATATTTTTGTAATCCAGCCTTACTTTGGTAATACATACTTGCCCCACTTGT
ATTTAGTTAATGGTGGCACTAGCTAGATAGTAATGTTAAATGATGATAAGCAGTAGTGAT
TCATCATTCAAATGTATCATTGTCCTTTAATGTTAAGCGCAAATAGATTTTCATTGTTCT
CCCATGTGCTTCATGTTTTATGTATTTATAGGTAGGTACTTAATGTTTTATAAATATTTT
TTTGTTAATTGGGAATCCCCAGTCCCCATTGTCTGGACCAGTTTATATATAATTGAACTA
ACAAGAGTGTGCTTTAAATACTATTCTCTGCAATTATGATAATTAAACAACATGAATTTC
TCTTCACTTCCCTTCTCTTATTTAAATAATATTGTAGGAAACTGTAATAACTAATACAAG
ATTATAAATTTCATTCTAGCAACTGGTGATGTAATCCATGTGGTAAATTCCAAAGATGTG
CAGGTCGGCCATCAGTATGTGTACAACATGGGAACTCCCGGAGCTAACTCACAGAAGAAT
AACCCATTTGATGATGAAGAAACAGTAGAAAAGACAAATCTAATAACTCTGGTCATGGAA
GCAAAAATTATGGTAATAACACATTTTTAACTAGGCATAAGGTCATAATTTAGCCAGAAT
CATCAGCTTGT
>P. xylostella-Cactus (PxCac); Px016665
(SEQ ID NO: 122)
CAATGCTGCGGAGGGTCTATGCGGGTGGACACCTCTACACGTAGCGGCGGCGCGAGGCGA
CGTCGACACGGCTCGCTACTTGCTCGAGAAGTGCGCTGGCGTCGATCCCTCTGCCCTGGA
CTACGCCGGTCGTACGGCCAGGAAACTGGCGTTGAAGAATAAAGCGGCCGCCCTGTTTGA
CGGCAGTGAGGGCAGCGAGGAGGAGGATAGTGACAGTGAGGATGAGATGCTTCTGGAAAG
CGACCAGAGTCTGTTCGACCGGATCCGTGACGGTATGAACGCCATCAACGTCGCCTGA
>P. xylostella-Dorsal (PxDor); Px000110
(SEQ ID NO: 123)
TAACCTGTGCCAGCAACCAATGGCTCCTATGGCGCAGCAGCTGATGGACCCGTCCCCCAG
CGACCCACCCTCCATCACGGGGCTGCTGATGGATCGCCCGGACCAGCCCTACTCCGGGGA
GCTGTCTGGACTCTCCGCCCTGCTGGCTGAGGCAGCCCCCGCAGAGATGCTCAGCGATAG
CCTCAACAGACTGTCTACGGGGGACTTGTTGAGACAAGTTGATATGTGA
>P. xylostella-Beta 1-3 glucan recognition protein 2 (PxßGRP2); Q8MU95.1
(SEQ ID NO: 124)
GAAGGATTGAAGTGAGCGCCAGAATGCCGCGCGGTGATTGGTTGATTCCAGATATTCTGC
TGGAGCCGAAAGAAAACCTTTACGGAGTACGCAATTACGCGTCAGGTCTACTCAGCATAG
CCTCAGTCAGAGGAAACACTGCTTACTCGAAGACCCTCAAAGGAGGCCCCATACTGTGTG
ACAAGGAACCGCAGAGAAGTGCCAAGTTGAGCGAAAAAGTTGGATATGACCATTGGAATA
AAGCCTTCCATAACTACACCATGATTTGGGCACCAAGTGGCATCACCATGCTGGTGGACG
GCGAGCAGTACGGGGACATCCGTCCCGGCGACGGCTTCAGCCAGGACCCGGCGGTGAGCA
GCGTGGTGGCCGCGCCGCAGTGGCTGAAGGGCACCAGCATGGCGCCCTTTGATGTTATGT
TCTACATATCCCTTGGTCTCCGCGTGGGCGGAGTGAACGACTTCCCCGACACTCCTGAGA
AGCCGTGGAAGAACAAGGCCACTAAAGCCATGCTGAATTTCTGGAACGCCCGGGAACAGT
GGCAGAGCAGCTGGTTTGAGGACACCACTGCACTCCTCATAGACTATGTCAGGGTTTATG
CGCTGTGA
>P. xylostella-Chitin synthase 1 (PxCHS1); KX420688.1
(SEQ ID NO: 125)
CGTATCTCGCACGACCTGAAAGAGCTGCGAAACTCATCCGTCTTTTCCTTCTTTATGATC
AATGCCCTCTTTGTTCTCATCGTATTCTTGCTGCAACTGAACAAGGACAACCTCCACATA
AAGTGGCCCTTCGGAGTCAAAACTAACATTACGTATGATGAGGTGACGCAAGAGGTGCTG
ATCTCCAAGGATACCTGCAACTAGAGCCTATTGGTCTGGTGTTCGTGTTCTTTTTCGCAT
TGATTTTAGTCATCCAGTTCACTGCCATGTTGTTCCATCGATTCGGAACTTTGTCGCATA
TATTATCGTCTACGGAACTGAACTGGTTCTGCAATAAGAAGGCGGAAGACTTATCTCAAG
ACGCACTGCTAGATAAGAATGCGATAGCAATAGTGAAGGATCTCCAGAAACTAAACGGGC
TCGATGACGGGTATGACAATGACTCGGGGTCGGGCCCGCACAATGTGGGAAGGAGAAAGA
CGATACACAACCTGGAGAAAGCGAGACAGAAGAAGAGGAACATAGGAACGCTCGACGTCG
CTTTCAAGAAGCGATTCTTCAACATGAACGCTAATGAAGGACCAGGAACACCAGTTCTGA
ACCGCAAGATGACGTTGCGAAGAGAGACGTTGAAGGCGTTGGAAACGAGGAGGAATTCTG
TGATGGCCGAACGAAGGAAGTCGCAAATGCAAACACTTGGAGCTAACAACGAATATGGAG
TCACTGGAATCTTAAACAACAACCCAGCGGTGATGCCGCGCCACCGGCCGTCGACAGCCA
ACATTTCGGTCAAGGACGTCTTCGCGGAACCCAACGGGGGACAAGTGAACCGAGGGTACG
AGACCACGCACGGCGACGAGGGAGACGGCAACTCCATCAGACTGCAGCCGAGAACCAACC
AGGTCTCCTTCCAGGGGAGATACCAATAA
>P. xylostella-Beta tubulin (PxBTUB); KX420688.1
(SEQ ID NO: 126)
GAAGGAGGTCGACGAGCAAATGTTGAACATCCAGAACAAGAACAGCAGCTACTTCGTCGA
ATGGATCCCGAACAACGTCAAAACGGCCGTGTGCGACATACCGCCTCGTGGACTGAAGAT
GTCTGCCACCTTTATCGGGAACACGACAGCAATCCAAGAGCTCTTCAAGAGGATTTCTGA
GCAGTTCACTGCTATGTTCAGGAGGGAAGCGTTCCTCCACTGGTATACTGGTGAAGGCAT
GGACGAGATGGAGTTCACAGAGGCGGAGAGCAACATGAACGACCTGGTCTCCGAGTACCA
GCAGTACCAGGACGCCACGGCTGAAGACGAGGGAGAATTCGACGAGGATATTGAAGACGA
GTGA
>S. frugiperda-Peptidoglycan recognition protein 1 (SfPGRP1); rep_c7951
(SEQ ID NO: 127)
CCTGATTGGTGGAAACGGGAGAGTTTATGAAGGAGCCGGCTGGCATCACGTTGGGGCCCA
TACTTTGGGATACAATGCAAGATCTGTGGGGATCTCCTTCATTGGCGATTTTAGAACAAA
ATTACCAACACCCGAAGCACTGAAAGCCTTCAACAGTCTCCTGGAATGTGGAGTCACGAA
CAATTATCTGTCAAAGGACTATCACCTGGTGGCCCATAGTCAGCTCTCTATGACTGACAG
TCCYGGAGACATGYTGAGGAAGCAGGTGGAATCGTGGCCTCMTTGGCTGGATAATGCCAA
AGACATACTTAAGTAGAARAAGACTAAACGCCGTACTTTGAGCCATTTAATGGTTACTTA
ACCCAGTCCTTAGCAATTTGATACAAGGCCAATGTCTCTAAGGGCGGCAGTAAAGGTCAA
AACACATTTAATGAGTGTGTTTAAGATTTTGCTAGTGAAAATTGTTTTGAAGTACGTATT
TGATGTAAGTGATGATATCAGTACCCTTAGTATGAGTTTGCTTTACGTTCCACGAGATGG
AAACGAGAGCGCGTTCGGCGCTCTGATTGGTTCGTTCATTCATGCCGGCC
>S. frugiperda-Attacin (SfAtta); rep_c9395
(SEQ ID NO: 128)
GCCAGACATCTACCACAGGACCACTCAACGTACGACCAAGTACAACTCCTCGGGTTCGAC
GAAGATGGACGACCAGTGTTTGAGCACGAAGACTTACTCCCAGAACTAGAGGAGTCCTAC
CAGCCAGAGCACCTGGCGAGGACTCGCAGACAGGCGCAGGGCAGCGTCACCCTCAACTCC
GACGGCGGCATAGGCCTGGGCGCTAAGATCCCGCTCGCACACAACGACAAGAATGTGGTG
AGCGCCATCGGCTCCATGGACTTCAACAACAAGTTGCAGCCTGCTTCCAAGGGCTTCGGT
CTGGCTCTGGACAACGTCAACGGGCACGGACTGACGGTGATGAAGGAAAGTATCCCCGGG
TTCGGGGACAGGCTGTCGGGCGCTGGCAAGCTGAACGTGTTCCACAACGACAACCACAAC
GTGGCCGTGACCGGCTCTCTCGCCAGGAACATGCCCAGCATCCCGAACGTGCCCAACTTC
AACACGTACGGCGGGGGCGTCGACTACATGTACAAGAACAAGGTGGGAGCGTCTCTGGGC
ATGGCCAGTACTCCGTTCTTGGACCGCAAGGACTACTCCGCGATGGGCAACCTGAACCTG
TTCCGCAGCCCGACCACTACCGTGGACTTCAGCGGCGGCTTTAAGAAGTTCGAATCTCCC
TTCATGAGCAGCGGCTGGAAGCCTAACTTCGGCCTTACTTTCGGCAGATCTTTCTAGATA
TATTTTGTAATCTAAATTTAACTTTAACTTTGTTGTATAATATTTTGTCGAATTAAGATC
AGTATTGTTCATACTAATATTATATTATCAGTGTTTCTTATAAATTAA
>S. frugiperda-Hemolymph proteinase 10 (SfHP10); c12881
(SEQ ID NO: 129)
CAAGGCTCGTACCTTGCAGTTGAACCGACAATCTATTCCTAAAGCCTTTTTAAGGTCAGG
AAAAATAGTTCCTACATCTAAATGCAGTAGAATTTGCGAAACGAATTTAAATAAAAATGG
CGTCGATTGTGTTTGTGATTTTGTGTGTTACCGTCGCTGCGGTGAAAAGCGCGATTTTAA
ACCCGTGGAGTAAAGTTGAGGCCAACAAATGTGGTGTAGAAGCCAGTACTAACTTGGTCC
ATCACAATCCATGGTTGGTCTACATCGAGTATTGGCGTGGAAACTCAGATACTGAGATCC
GATGCGCCGGTACTTTAATCGACAGCAAACATGTCGTCACAGCTGCCCACTGCGTTAGGA
CTCTGAAGTTTAGTCATTTGATCGCCCGTCTTGGCGAATACGACGTAAATTCTAAGGAGG
ACTGCGTTCAGGGCGTGTGTGCCGATCCCATCGTCAGAATCAAGGTGGCTGAGATCATCG
TGCATCCTAACTACAGCAACCGGGAACA
>S. frugiperda-Trypsin like serine protease (SfTSP); rep_c48453
(SEQ ID NO: 130)
AGCAACAAAATGCGTGTCCTCGCTTGCTTGGCCCTTCTCTTAGCTGTGGTAGCAGCCGTC
CCCTCCAATCCCCAGAGGATTGTGGGTGGTTCGGTCACCACCATTGACCGGTACCCCACC
ATTGCATCCCTGCTGTACTCGTGGAACTTGAGTTCCTACTGGCAGGCGTGCGGTGGTTCC
ATCTTGAACAACCGTGCCATCCTTACTGCTGCCCACTGCACAGTTGGTGACGCCGCCAAC
AGATGGAGAATCCGTCTTGGCTCCACCTGGGCCAACAGCGGTGGTGTCGTTCACAACGTC
AACACTAACATCGTCCACCCCTCATACAACTCTGCAACTTTGAACAACGACATCGCTATC
CTCCGCTCCGCCACCACCTTCTCCTTCAACAACAATGTTCAGGCTGCCTCCATTGCTGGT
GCCAACTACTTGCCCGGTGACAACACCGCCGCCTGGGCCGCTGGATGGGGAACTACCTCC
GCTGGTGGCTCTAGCTCTGAGCAGCTCCATCACGTTGAGCTGCGCATCATCAACCAGGCT
ACTTGCAAAAACAATTACGCTACCCGCGGTATCACCATCACCTACAACATGTTGTGCTCT
GGCTGGCCCACCGGTGGTCGCGACCAGTGCCAGGGTGACTCTGGTGGTCCTCTCTACCAC
AACGGCATCGTTGTTGGTGTCTGCTCTTTCGGTATTGGCTGTGCTCAG
>S. frugiperda-C Type Lectin 6 (SfCTL6); Joint2_ rep_c448
(SEQ ID NO: 131)
AACAGTTTTCTATTGGCAGTCAAAGACTTCAGTCGAAAAATAATCCTCATCAGAGTCGTG
AAGCAAGGTGCCCAAAATATAATGTAACCTAGATACCTATTAATAAATTATTTGTCAACC
AAAACGTTACGTTCAAAGTCCTTAAAATCAAAATATCTTATGATTAGTTTTGATTTAAAA
ATAGAGGTTCGAAATCGCCAACCCAAATAGGTTTAGTTTACGATTCAGGAAAAATCCTAA
CGTAGGGAAACATTATTTTACAAGACTTTTGGCTTAAAAACTTTGAGAACCAATGTCAAA
TTTGATAATAACTAATGAGGTATAAAAGCTTGATCCTATTAGGACTTATTTTCATAACAC
CATCGAGTTTGTATTTAATRRAGACGTGKGTTAACTAACAACATGAAGACCGGTGTAAAA
TATTCTGTTMTTTGGATATTCTCTCTATTYTGCTATATAGAGGCAACATTTCGTTGTGAC
TACACGTACAGCAAGGAAGCGAAGGGCTGGTTCAAACATGTGGTGATACCAGCTACTTGG
GCTGACGCACGWCTGCACTGCACGTTGGAAGGTGCAACGCTGGCTTCTCCACTCAACCAG
GCTATMAGTAATGAGATGCAGTCCMTCCTGGCRAACCTCTCGGCGCTGCAATCAGAAGTC
TTCACTGGAATTCACRCGACTKTTTCACGRMRCAACTTATATCATACYATYGAAGGTATA
CCTCTTAGTAAAATTCCATTAGATTGGGCAACAAATGAGCCAAATGGTGGGAGAGATGAA
AACTGTATCACGTTTAACTCCGATGGCCAAGCGGCAGACAGATCCTGTAARGAGACTCGA
CCTTACATCTGCTACCGACACACWACTAAAGTGACTGTGKCCAATGAATGTGGGACTGTA
GATCCTGAATACAATTTGGATAAAAGAACGGGCKCYTGCTATAAGTTCCACACRGTACCT
CGCACGTTCGAGCGTGCCAACTTCGCGTGTTCTGCTGAAGGTG
>S. frugiperda-Cecropin (SfCec); rep_c42380
(SEQ ID NO: 132)
TTCGTGTCGTATCACTAGAGTTCGAAATACAAAATAATAATACATTTATTATTTTGCCAT
AATTAATAATAAAGTTATTTTATTTCATAATAATAATGAATTTCACAAAGATATTTTGTT
TGTTTTTGTCTTGCTTTGTTTTGATGGCGACCGTGTCAGGAGCTCCTGAACCGAGGTGGA
AATTCTTCAAGAAAGTGGAGAAGTTGGGCCAAAACATCCGCKATGGTATCATAAAGGCAG
GACCCGCAGTGGCCGTGGTGGGATCAGCRGCAGCCATWGGAAAGTGAKCCCTACGACCTG
AGACATGAAGACTAATATCCAYTAAAATAASAATATTGAGGCKTATAATATTAATTTATT
RTRTTTGTAAATTAAATTATTTGTAAGATAA
>S. frugiperda-Beta 1, 3 glucanase recognition protein (SfßGRP2); EF641300
(SEQ ID NO: 133)
GCAACAATCGCACCAACTCTTTCGTGCGCAGTGGAAGTTTGTTCATCCGTCCCTCTCTAA
CATCAGACGAGTTCGGAGAAGCTTTCCTCTCATCTGGACACTGGAACGTCGAGGGTGGTG
CTCCTGCTGATAGATGCACAAATCCACAATGGTGGGGTTGCGAGAGAACAGGCACGCCGA
CCAACATTTTGAACCCAATCAAGAGTGCTCGTGTCCGTACCGTCAATTCCTTCAGCTTCC
GTTACGGACGCCTCGAAGTCCGCGCTAAAATGCCCGCCGGAGATTGGATTTGGCCAGCTA
TCTGGTTGATGCCTGCGTACAACACTTACGGTACTTGGCCCGCATCAGGAGAGATTGACT
TAGTTGAGTCCCGAGGCAACCGTAACATGTTCCACAATGGTGTCCATATCGGTACACAGG
AAGCAGGCTCGACCTTGCACTACGGACCTTACCCAGCGATGAACGGTTGGGAGCGCGCCC
ATTGGGTCAGAAGGAACCCT
>S. frugiperda-joint2_rep_c16438 (Sfrc16438); Un-annotated
(SEQ ID NO: 134)
TCGTTCTCCTCGTCGCTTTCTTGGGGACCTCATGGTTTACGGGAGATGTTTCTGCGAGTC
CGCGGCCGCAAGAGCCGCGTGTGGATCAAAATCCGAATCAGGTGTCACCTTATGGAGGGT
CCGGGTACCACGCACCTCCGCAGTACCAACCGCAGTACCAACCACAGCCGTACTACCCAC
AGCCGCAGTACTACCCACAGCCGTACTACCCACCTCCTCAGTATTACCCACCGCAGCCAC
AAACACCTGAGAATGCTCCACTCATAAACACATGGAATGGTTTCCACGACTGGGCTCAGA
ATATCGTTCAAAGTGCTTTGGGGCAGAAATTCCCGAAAGGTAGACAGTAACTTTTTAATT
GTCAATTGAAGATAAGGCCCATTTCACCAACTGCTGTTTAATTTTAAGGAGCTCCTAAAC
TAACATAGGTGACACTTAGCGATATTCTGGATTTTTTGTGAACGTATAAATAATATCCAA
ATGTAAAAGATAAGAGGCCAAGAA
>S. frugiperda-Chitin synthase B (SfChsB); AY52599)
(SEQ ID NO: 135)
GAATTTAGGAGCAGCGTGCGGGCGCATCCATCCTGTGGGCTCAGGCTTCATGGCATGGTA
TCAAATGTTCGAGTACGCTATTGGTCATTGGCTGCAAAAGGCGACTGAACACATGATTGG
CTGTGTACTCTGTAGCCCTGGATGCTTCTCCCTCTTCAGAGGAAAGGCTTTGATGGACGA
CAACGTTATGAAGAAATATACCTTAACTTCCCACGAGGCACGACACTATGTGCAATACGA
TCAAGGCGAGGACCGTTGGTGCACGCTACTGCTGCAGCGCGGGTACCGCGTGGAGTACAG
CGCGGTGTCGGACGCGTACACGCACTGCCCCGAGCACTTCGACGAGTTCTTCAACCAGCG
CCGCCGCTGGGTGCCCTCCACGCTCGCCAACATCTTCGACCTGCTCGGCAGCGCCAAGCT
CACCGTCAAGTCCAACGACAACATCTCCACCCTCTATATAGTCTATCAGTTCATGTTGAT
AGTGGGTACGGTGTTGGGTCCCGGCACGATCTTCCTGATGATGGGGGGAGCCATGAACGC
CATCATTCAGATCAGCAACGCGTACGCGATGATGTTGAACCTCGTACCACTCGTCATCTT
CCTTATAGTCTGTATGACTTGTCAGTCAAAGACGCAGCTCTTCCTCGCTAACCTCATAAC
ATGCGCATACGCAATGGTGATGATGATCGTGATAGTGGGGATAGTTCTGCAGATAGTGGA
GGATGGATGGCTGGCTCCGTCCAGTATGTTCACAGCTTTAATATTCGGTACATTCTTCGT
CACCGCGGCACTACACCCGCAAGAGATCAAATGTTTGTTGTTCATAGCAGTGTACTATGT
AACCATCCCTAGTATGTACATGTTGTTGATCATATACTCCATCTGTAATCTCAACAACGT
ATCCTGGGGTACCAGGGAGACACCGCAGAAGAAAACTGCTAAGGAAATG
>T. castaneum-Peptidoglycan recognition protein 2 (TcPGRP2); XM_965754.3
(SEQ ID NO: 136)
ATGAGTGGCAGTGACCCTTTAACAAATACCCAACAATCCGATCAAGATTATTACCATCCA
CTCTGTTATTCAATTCAAGTGGACGACGAAAATGAACAATCAGCTCTCCTGCCCGCATTT
CATCAAAGGAAAAGTTTGCGAGTTCAGGATAAAATCTTTATTGTATTTTTATTTTCAATT
CTAATTACCGGACTAGCCATTGGCCTCTATCTCCTTGCAACTGAGGGACACGAATGGAAA
GCTGCAGGAGTCTATAATATTACAGTTCGGGAACAGTGGCAAGCTCACGTCCCTTCATCA
ACAATGCCAAAGTTGGAACTTCCCGTAAGAAGAGTTTTATTTCTTCCTGCAAATACCACT
AGCTGCGGCAGCAAATCCCACTGTGCCAAAGTCCTCCAGGAACTACAATTACAGCATATG
CTGCAGTGGAAAGAACCTGACATCTCCTACAATTTCATAATGACTGCAGATGGCAGAATT
TTCGAGGGGAGAGGATGGGACTTTGAAACTTCTGTTCAAAATTGTACGGTTAATGATACT
GTGACAGTTGCTTTTTTGGACGAATTAGATGCGAAAGCACCGACGTTTAGACAAGCTGAA
GCGGCAAAAATGTTCCTGGAAGT
>T. castaneum-Beta 1-3 glucan binding protein (TcßGRP2); XM_966587.4
(SEQ ID NO: 137)
ACATTACAACGTTCCGCGACCCTCAATCCAAGCATTCAGGCCCCGTGGCTTCAAAGTGAG
CATCCCTCATACTGAAGGCATCCAATTATTCGCCTTTCATGGAAATATTAATAAACCCCT
GCACGGTCTCGAGGCCGGACAATTTTCCCAAGACGTCCTCCAAAGGGAGGGAGACGAGTG
GGTGTTCCAAGACTCCAGTGCCAAATTAAACGTGGGAGATAAAATCTATTATTGGCTCTT
TATCATTAAAGAAGACCTGGGCTACAGATACGATCACGGCGAGTACGAAGTGAAAGTTTT
AGCCACCCGTGACTTCGATTCTCCTCAAACAACCTCTGTGACGCCGAATCTTGCCCCCAA
TCTCGGCATTTGCGAAAAGGTGATGGTAAATCTCACGCGGAAGCTGCTCGATTTGCAGCA
AGAAATTGAGTCCCTTAGGGAGACGAACGATATTTTGGAGGATATGGTTCAGAAGCACAC
TGATACGGCGACTACTCTCACTTTGGACGGCTTGATGATAAAGGATGACGACGAACTTGT
TTCGGTGATTCAAGCAATTATTAAAGATAAACTTGGACTAAAAAGCAGGATTCAAAATGT
GACGAGGCAGGAGAATGGAATGGTCAAGTTTCAAGTGGCGAGTTTGAGAGAAAAGTTGGA
GGTTGTCAAAGCGGCCAAAAGAAAACTCAAGTCGTCGAGCTTTACGATCACGTATTAA
>T. castaneum-midgut protein (TcMDGP); XM_971351
(SEQ ID NO: 138)
CCGTTGAAGAGGATGCCCAGTGAAGAAATCAGTATCAGTGATCTGCCTAGCGAAATGAAA
GAAGTTTTACTAGAAATTAGCCCGAACTTTGATGAAAATCTGAAACGGGCTTTCAGGAAC
GAAGGAGTGAGGCTGCAGAGAGTGCAGAACAATGGACGATTTATTCATCAGCTGGACGAC
GTTCTCTTATCCATAGACGATACCAAAATCGAGTTACGCAACCTGAAATTCCCCTGGATC
CCCGACTTCCGCATCGTGGACTTGTCCAGCGACCTGCCCATGTCATGCCTCGACCTAAAC
CTAAACCTGGGCAATTTGCGAATTGAGGGCGAGTACGAAGCCAACAACACCACACTCAGG
CGATGGCTCCCGGTATCTCACATTGGTCGAATCGTGATCGGTTTTAACAACGTCCGAGCG
AACGGAAAAGTCGGACTCGTGCTCGAGCAGGATTCTTTCGTTCCGCAGAATTATGATATT
AGATATGAGCCGACGGATGTTGTTATTAGGGTTAGCTATCACGTGGATGGCGAGAATGAG
GTGCAAAATGAGATTAGCAATTCAGATATTGAGGCCACGCTAGGCAAGACCGTGTGGGTG
CAGTTGACTGAGATATTGTCCAACCTGTTGCATAGGCAATTGGGCGAGGTTGTAGTGGAG
T
>T. castaneum-Chitin synthase 2 (TcCHS2); EFA 10719.1
(SEQ ID NO: 139)
ATGGCGGCGCGTCATCGCTTTGCCACAGGGAGCCCTGAGGAAACAGAGCCCCTGTATTCG
TCGACGCAAATGCCCGAAAAAGTCCGGGAAAAATGGAACGTCTTCGACGACCCCCCAAGA
GAGCCCACTTCGGTTCCGAAGTCAAAAGAACCTACATCGAGTGGGGGGTGAAGTTTTTGA
AAGTTGTGACAATCATAACTGTGTTTTTTGTGGTCCTTGGTGCTGCAGTGGTTTCGAAAG
GGACAACCTTGTTCATGACGTCACAAATAAAAAAGAATGTGACAAGGGCTTATTGCAACA
AAAAGATAGACCGCAACCTCCAATTCGTCGTCTCCCTCCCCGAAGTGGAGCGCGTGCAAT
GGATCTGGCTCCTCATTTTCGCTTACTTGATCCCCGAAGTGGGTACCTGGATCCGCGCCG
TCCGCAAATGCCTCTACAAGCTCTGGAAAATGCCCTCCCTCTCCGAATTCCTCTCCCTCT
TCGGCACGGAAACGTGCCCCGCCATCGGAAGCGCAATTTTGATATTCGTCGTCCTCCCCG
AGCTGGACGTCGTCAAAGGGGCGATGCTCACAAACGCGGTTTGTTTCGTGCCCGGAGTTG
TGGCAATGTTCTCGCGCAAACCGTGCTCCATAAACGAGAACCTGAAAATGGGGCTGGACA
TCGCCAGCATAACTGCACAGGCGTCAAGCTTCGTGGTGTGGCCCTTGGTTGAAAATAACC
CGACCTTGTACCTAATCCCCGTTTCCGTGATTTTGATTTCGGTGGGT

The breadth and scope of the present disclosure should not be limited by any of the above-described exemplary embodiments, but should be defined only in accordance with the following claims and their equivalents.

### REFERENCES

1. http World Wide Web internet site “fao.org/3/a-av013e.pdf”.
2. Polaszek A. (1998). African Cereal Stem Borers: Economic Importance, Taxonomy Natural Enemies and Control. Wallingford, UK: CABI. 530 pp.
3. http World Wide Web internet site “cnbc.com/2015/05/08/insects-feast-on-plants-endangering-crops-and-costing-billions.html”.
4. Khan Z R. et al. (2014). Achieving food security for one million sub-Saharan African poor through push-pull innovation by 2020. Philosophical Transactions of the Royal Society B: Biological Sciences, 369 (1639), 20120284.
5. Tabashnik B E. et al. (2013). Insect resistance to Bt crops: lessons from the first billion acres. Nat. Biotech. 31, 510-521.
6. Campagne et al. (2013). Dominant inheritance of field-evolved resistance to Bt corn in Busseola fusca. PLoS ONE 8(7):e69675.
7. http World Wide Web internet site “dtnpf.com/agriculture/web/ag/news/article/2016/08/10/rootworm-resistance-pyramided-bt”.
8. Terenius O. et al. (2011). RNA interference in Lepidoptera: An overview of successful and unsuccessful studies and implications for experimental design. Journal of Insect Physiology 57, 231-245.
9. Gayatri Priya N. et al. (2012). Host Plant Induced Variation in Gut Bacteria of Helicoverpa armigera. PLoS ONE. 7(1):e30768.
10. Peñuelas J and Terradas J (2014). The foliar microbiome. Trends Plant Sci. Volume 19, Issue 5, 278-280.
11. Casanova-Torres and Goodrich-Blair (2013). Immune Signaling and Antimicrobial Peptide Expression in Lepidoptera. Insects. 4, 320-338.
12. Tang X. et al. (2012). Complexity and Variability of Gut Commensal Microbiota in Polyphagous Lepidopteran Larvae. PLoS ONE. 7(7):e36978.
13. Ryu J, et al. (2008). Innate immune homeostasis by the homeobox gene caudal and commensal-gut mutualism in Drosophila. Science. 319, 777-82.
14. Shrestha S. et al. (2009). An inhibitor of NF-kB encoded in Cotesia plutella bracovirus inhibits expression of antimicrobial peptides and enhances pathogenicity of Bacillus thuringiensis. Journal of Asia-Pacific Entomology. 12, 277-283.
15. Buchon N. et al. (2013). Gut homeostasis in a microbial world: insights from Drosophila melanogaster. Nat. Rev. Microbiol. 11, 615-626.
16. Karlin S and Altschul S F (1990). Method for assessing the statistical significance of molecular sequence features by using general scoring schemes. Proc. Natl. Acad. Sci. USA. 87, 2264-2268.
17. Watson J M. et al. (2005) RNA silencing platforms in plants. FEBS Letters. 579, 5982-8987.
18. Zhong X. et al. (2012). A Toll-Spatzle Pathway in the Tobacco Hornworm, Manduca sexta. 42(7), 514-524.
19. Zhang X. et al. (2015). Phylogenetic analysis and expression profiling of the pattern recognition receptors: Insights into molecular recognition of invading pathogens in Manduca sexta. Insect Biochem. Mol. Biol. 62, 38-50.
20. Cao X. et al. (2015). The immune signaling pathways of Manduca sexta. Insect Biochem. Mol. Biol. 62, 64-74.
21. Wang et al. (2006). Interaction of β-1,3-Glucan with Its Recognition Protein Activates Hemolymph Proteinase 14, an Initiation Enzyme of the Prophenoloxidase Activation System in Manduca sexta. J. Biol. Chem. 281(14), 9271-9278.
22. Ward N and Moreno-Hagelsieb G (2014). Quickly Finding Orthologs as Reciprocal Best Hits with BLAT, LAST, and UBLAST: How Much Do We Miss? PLoS ONE 9(7), e101850.
23. Timmons L. et al. (2001). Ingestion of bacterially expressed ds RNAs can produce specific and potent genetic interference in Caenorhabditis elegans. Gene 263, 103-112.
24. Kanost M R. et al. (2016). Multifaceted Biological Insights From a Draft Genome Sequence of the Tobacco Hornworm Moth, Manduca Sexta. Insect Biochem. Mol. Biol. 76, 118-147.
25. Odman-Naresh J. et al. (2013). A lepidopteran-specific gene family encoding valine-rich midgut proteins. PLoS ONE 8:e82015.
26. Engel P and Moran N A (2013). The gut microbiota of insects—diversity in structure and function. FEMS microbiology reviews. 37(5), 699-735.
27. Siepel A. et al. (2005), Evolutionarily conserved elements in vertebrate, insect, worm, and yeast genomes Genome Res. 15,1034-1050.
28. Pauchet Y. et al. (2010). Pyrosequencing the Manduca sexta larval midgut transcriptome: messages for digestion, detoxification and defence. Insect Mol Biol. 19, 61-75.
29. Kim S H and Lee W J (2014). Role of DUOX in gut inflammation: lessons from Drosophila model of gut-microbiota interactions. Front. Cell. Infect. Microbiol. 3, 116.
30. Lee W J and Hase K (2014). Gut microbiota-generated metabolites in animal health and disease. Nat. Chem. Biol. 10, 416-424.
31. Brummett et al. (2017). The immune properties of Manduca sexta transferrin. Insect Biochem Mol Biol. 81, 1-9.
32. Xia Xu et al. (2012). Manduca sexta Gloverin Binds Microbial Components and is Active against Bacteria and Fungi. Dev Comp Immunol. 38(2), 275-284.
33. Gunaratna R T and Jiang 1-1 (2013). A comprehensive analysis of the Manduca sexta immunotranscriptome. Dev. Comp. Immunol. 39, 388-398,
34. Broderick N A. et al. (2009). Contributions of gut bacteria to Bacillus thuringiensis-induced mortality vary across a range of Lepidoptera. BMC Biol. 7:11.
35. Gregory R. Richards (2008). Xenorhabdus nematophila lrhA is necessary for motility, lipase activity, toxin expression, and virulence in Manduca sexta insects. Journal of Bacteriology. 190, 4870-4879.
36. Timmons and Fire (1998). A. Specific interference by ingested dsRNA. Nature. 395, 854.
37. Kamath R S. et al. (2000). Effectiveness of specific RNA-mediated interference through ingested double-stranded RNA in Caenorhabditis elegans. Genome Biol. 2, 1-10.
38. Cao et al. (2018). A systematic study of RNAi effects and dsRNA stability in Tribolium castaneum and Acyrthosiphon pisum, following injection and ingestion of analogous dsRNAs. Int. J. Mol. Sci. 19, 1079.
39. Rajkunar A P³ et al. (2015). Experimental validation of methods for differential gene expression analysis and sample pooling in RNA-seq. BMWC Genomics. 16(1), 548.
40. Zhang et al. (2015). Full crop protection from an insect pest by expression of long double-stranded RNAs in plastids. Science. 347, 991.
41. Kim S R, Yao R, Han Q, Christensen B M and Li J (2005). Identification and molecular characterization of a prophenoloxidase involved in Aedes aegypti chorion melanization. Insect molecular biology. 2005; 14(2):185-194.
42. Packey C D and Sartor R B (2009). Commensal Bacteria, Traditional and Opportunistic Pathogens, Dysbiosis and Bacterial Killing in Inflammatory Bowel Diseases. Current opinion in infectious diseases. 2009; 22(3):292-301.
43. Arakane et al. (2005). The Tribolium chitin synthase genes TcCHS1 and TcCHS2 are specialized for synthesis of epidermal cuticle and midgut peritrophic matrix. Insect Molecular Biology 14(5), 453-463.
44. Morris et al. (2009). Tribolium castaneum larval gut transcriptome and proteome: A resource for the study of the coleopteran gut J. Proteome Res. 8(8):3889-98.

What is claimed is:

1. A method of silencing an insect immune response gene, an insect gene encoding structural components of an insect midgut, or both, the method comprising providing for ingestion:
a. an isolated double stranded RNA (dsRNA) molecule, or a dsRNA molecule in a host cell, in a transgenic or transplastomic plant or cell, organelle, or part thereof, in a microbial conduit, or in an insecticidal composition, wherein the dsRNA molecule comprises a nucleic acid sequence complementary to about 21 to 2000 contiguous nucleotides of a target gene sequence comprising a nucleic acid sequence of SEQ ID NO: 76, wherein the dsRNA molecule silences the target gene when ingested by an insect;
b. an siRNA molecule derived from the processing of the dsRNA molecule;
c. a polynucleotide, a construct, or a dsRNA encoding segment encoding the dsRNA molecule; or
d. a combination of (a)-(d).

2. The method of claim 1, wherein the microbial conduit comprises plant growth promoting organisms, normal commensal and/or symbiotic microorganisms associated with a target insect pest or parasite, and/or natural enemies of the target pest or pest target host or host cultivation range etc. from an insect or parasite, and/or natural enemies of the target pest engineered or identified from natural populations containing microbial conduit to produce and/or deliver dsRNA and/or drive transmission of such microbial conduits into natural populations of insect pests as a control option.

3. The method of claim 1, wherein the dsRNA molecule is bound to a synthetic carrier.

4. The method of claim 3, wherein the synthetic carrier comprises chitosan, liposomes, carbon quantum dots, biodegradable particles of plant, or soil.

5. The method of claim 1, wherein ingestion of the dsRNA, the siRNA molecule, the polynucleotide, construct, or dsRNA encoding segment encoding the dsRNA molecule, or any combination thereof silences the target gene to thereby induce (a) a melanotic response; (b) results in perturbation of gut microbial homeostasis; (c) results in defective clearance of opportunistic microbes; (d) results in defective containment of gut microbes, or any combination of (a) to (d).

6. The method of claim 1, wherein the host cell is a bacterial cell, a yeast cell, or a fungal cell.

7. The method of claim 1, wherein the target gene sequence includes at least one of a protein coding region, a 5′ untranslated region (UTR), a 3′ UTR, or any combination thereof.

8. The method of claim 1, wherein the dsRNA molecule comprises a single RNA strand comprising an inversely repeated sequence with a spacer in between and where the single RNA strand can anneal to itself to form a hairpin loop structure; or wherein the dsRNA molecule comprises two separate complementary RNA stands annealed together.

9. The method of claim 1, wherein the target gene is selected from the group consisting of Manduca sexta-Peptidoglycan recognition protein 2 (MsPGRP2), Plutella xylostella PGRP2, and Tribolium castaneum PGRP2.

10. The method of claim 1, wherein the dsRNA comprises a nucleic acid sequence of SEQ ID NO: 76, or a fragment of at least about 21 nucleotides thereof; optionally.

11. The method of claim 1, wherein the dsRNA molecule causes impeded growth, developmental progression, and/or mortality and the like of DBM, optionally wherein the DBM is a Bt resistant strain.

12. The method of claim 1, wherein the construct encoding the dsRNA comprises a gene silencing sequence operably linked to one or more promoters for expression of a dsRNA molecule that silences the target gene when ingested by an insect, optionally wherein the construct further comprises an additional transcription regulatory region or an additional transcriptional regulatory element.

13. The method of claim 12, wherein the silencing of the target gene results in reduced appetite and/or developmental defects resulting in incomplete development and/or mortality and/or decreased reproductive success of the insect, optionally wherein the reduced appetite and/or developmental defects and/or mortality and/or reduced reproductive fitness of the insect is observed after sustained feeding for at least 24 hours.

14. The method of claim 1, wherein the construct is an expression vector, and the expression vector can target single or multiple insect RNAi target genes or chimeric RNAi target genes.

15. The method of claim 1, wherein the insect is of the order Lepidoptera, Coleoptera, Hemiptera, Blattodea, or Diptera.

16. The method of claim 1, wherein the insect is Manduca sexta (M. sexta) (tobacco hornworm), Spodoptera frugiperda (fall armyworm), Ostrinia nubilalis (European corn borer), Plutella xylostella (Diamondback moth), Leptinotarsa decemlineata Say (Colorado potato beetle), Diabrotica spp. (Corn rootworm complex), Tribolium castaneum (Red flour beetle), Popillia japonica (Japanese beetle), Agrilus planipennis (Emerald ash borer), Diaphorina citri (Asian citrus psyllid), Cimex lectularius (Bed bug), a cockroach or termite, or insect pests such as mosquitoes and flies.

17. The method of claim 1, wherein the plant is selected from the group consisting of Zea mays L (corn), Sorghum bicolor (sorghum), Setaria italica (fox tail millet), Pennisetum glaucum (Pearl millet), Solanum tuberosum (potato), Oryza sativa (rice), Lycopersicon esculentum (tomato), Solanum melongena (eggplant), cultivars of the Brassica oleracea family, Citrus sinensis (Orange), trees of the Oleaceae family, and crops of Rosaceae.

18. A method of protecting a plant from an insect pest of the plant, the method comprising topically applying to the plant
a. an isolated double stranded RNA (dsRNA) molecule, or a dsRNA in a host cell, in a transgenic or transplastomic plant or cell, organelle, or part thereof, in a microbial conduit, or in an insecticidal composition, and providing the plant in the diet of the insect pest, wherein the dsRNA molecule comprises a nucleic acid sequence complementary to about 21 to 2000 contiguous nucleotides of a target gene sequence comprising a nucleic acid sequence of SEQ ID NO: 76, and wherein the double stranded RNA molecule silences the target gene when ingested by an insect;
b. an siRNA molecule derived from processing of the dsRNA molecule;
c. a polynucleotide, a construct, or a dsRNA encoding segment encoding the dsRNA molecule; or
d. a combination of (a)-(c).

19. A method of producing a transgenic or transplastomic plant, the method comprising:
a. transforming the plant with a polynucleotide encoding a dsRNA, a construct or a dsRNA encoding segment encoding the dsRNA, or both to generate a transformed plant cell;
b. regenerating a plant from the transformed plant cell and/or organelle to generate a transformed plant; and
c. growing the transformed plant under conditions suitable for expression of said dsRNA;
wherein the transformed plant of (c) is resistant to a plant pest insect compared to an untransformed plant and wherein the dsRNA molecule comprises a nucleic acid sequence complementary to about 21 to 2000 contiguous nucleotides of a target gene sequence comprising a nucleic acid sequence of SEQ ID NO: 76.

20. A method of improving crop yield, the method comprising growing a population of transgenic or transplastomic plants comprising a polynucleotide encoding a dsRNA, a construct or a dsRNA encoding segment encoding the dsRNA molecule, wherein the dsRNA comprises a nucleic acid sequence complementary to about 21 to 2000 contiguous nucleotides of a target gene sequence comprising a nucleic acid sequence of SEQ ID NO: 76 and wherein the population of transformed plants produces higher yields in the presence of pest insect infestation than a control population of untransformed plants.


SEQUENCE LISTING (139 sequences)

SEQ ID NO: 1 - DNA, 1242 bp, Manduca sexta
  source 1..1242: mol_type = genomic DNA
atggtttcaaaaagtatcgtcgctttggctgcgtgcgtcgcaatgtgcgtagcccagccagtggagaagatgcctgtgctgaaggaccaacccgctgaagtcctcttccgggagtctcaggccaccgttctcgaatgtgttaccgagaatggcgataaagatgtcaaatattcttggcaaaaagacggcaaagaattcaaatggcaggaacacaatatcgcccagcgcaaagacgaaggcagcctggtcttcctcaagcccgaggctaaagatgaaggccaatacagatgtttcgctgagtcggccgccggagtcgccacctcccacatcatctcctttagaaggacctacatggtcgtacctactacttttaagactgtagaaaagaaaccggtagaagggtcatggctcaaacttgagtgcagcatccccgaaggttatcctaaacctactattgtatggagaaagcagcttggtgaagacgaaagtatagcagattctatactggcacgtcgtattacacaatctccagagggagacctgtacttcacgagcgtcgagaaagaagacgtaagcgaaagctataaatacgtttgcgctgctaagtcaccggctattgatggggatgtccctcttgttggatacactattaaaagcttagaaaagaatacaaatcagaaaaacggtgagctggtcccgatgtacgtcagtaatgatatgatagctaaggccggagacgttactatgatctactgcatgtatggtggagtcccaatggcttaccccaactggttcaaagacggtaaggacgtgaacggcaaaccgagcgaccgcatcacccgccacaacagaacctccggcaaaagactgttcatcaaggagacgctgctcgaagatcagggcacttttacttgcgacgtgaacaacgaagtcggcaagccacagaaacattccgtcaaacttaccgtagtcagtggacccagatttacgaagaaaccagaaaagcaagtcatcgctaagcagggccaggactttgtaatcccctgtgaagtatccgccttaccggccgcccctgtctcctggacgttcaacgccaagcccatcagcggcagccgcgtggtagccagcccgagcggactgaccatcaagggcatccagaagtctgacaagggttattatggctgccaggcccacaacgagcacggagatgcctacgctgagacgctcgtgattgttgcttaa

SEQ ID NO: 2 - DNA, 918 bp, Manduca sexta
  source 1..918: mol_type = genomic DNA
atgttgttgcttctgtattgtcttgtggcggcctccgcgccgttctttattgcagcggaccaaggcagccctgacctgcctttagctaccgaaccaccaacagaatgcggaacaatagcacctgatgatagcttagtattagatgggtccgttggtaaaagtgacaaattaccttggtatgctattatctacacaaccaccacccggccatacaagcagatcggtggaggaaccctcatcactccttcagtagtaatctcagccgctcactgtttctggcgcaatggtgaggttccatctaaggataattacgccgtggcgctcggcaagacccatagtgcttggaatagccatgccgatgtaaacgctcacaagtctgatgtaaaagaaatacacataccaccgcagtttaagggaaggaacactaattatcggaatgatatagcaatcgtggtcatgtcagaccctgtgacctacaaagtggacatccgccctatctgtttgaacttcgatgtacaatttgaaagactgcaattaaaagacggcattatggggaagatcggcacatggaatgtaagtcgtgagacactgaaactatcgaaaacattaaaagtggtggagaatccatacattgacgcagcgacttgtattagtgagtctccggcaagcttcagaaattccatcactgcggacaaaatatgcatcggatacgttaacggcaccgggctatgtagaggtgatggcggcgctggggtggctttccctagccaggaacaaggagtgcaacgttactacctcagaggtgttatatctacagcccataccagcgatgatggcaacttatgtgcagatggatttgtaactgccactgctataggccatcacgaacattttatcaaacagtttataagcgtttag

SEQ ID NO: 3 - DNA, 591 bp, Manduca sexta
  source 1..591: mol_type = genomic DNA
atggcgagcttcgctttaatagttatccttagcgtaattggctttatatcggcctatcctagtcctgaaggttacagttctgccttcaactttccattcgtaaccaaggagcagtggggcggcagggaggcacgcacgtcgacgccactcaaccacccagtgcagttcgtggtgatccaccacagttacattcccggcgtgtgcctcagccgggacgagtgcgcgcgcagcatgcgctccatgcagaacttccacatgaacagtaacgggtggagtgatattggatacaacttcgctgtcggcggtgaagggtcggtgtacgagggccgcggctgggacgcggtcggcgcacacgcagctggctataacagtaacagtatcggcatcgtgctcatcggcgattttgtttcaaacctcccgccggcggtgcaaatgcaaaccacacaagaattgatcgcagcgggcgtgcgactcggttacatcaggcccaactacatgctcatcgggcatcgtcaggtctccgccactgagtgcccaggaaccagactcttcaacgaaatcaccaactggaacaacttcgtgaggatatga

SEQ ID NO: 4 - DNA, 1449 bp, Manduca sexta
  source 1..1449: mol_type = genomic DNA
atgtggatcaagagcgtctgtttgttcgcaaccattgcgggctgcttgggccagcgagggggtccatacaaggtgcctgatgcgaaactcgaagctatctaccccaaaggcttgagagtctctgtgccagatgatggctactccctatttgccttccacggcaagctcaatgaggagatggaaggtttagaggctggccattggtccagagacatcaccaaagcgaagcagggcagatggatattcagagataggaatgctgagctgaagcttggagacaaaatttacttctggacttacgttattaaggatggattgggatacaggcaggacaatggagaatggactgttacagaattcgtcaatgagaacggtacagtggtggacactagtacagcgccgccaccagtagcacccgccgtttcagaggaagatcaatcgccaggtcctcagtggagaccttgcgaaagatccctgactgagtccttggcccgcgaacgcgtttgcaaaggcagccttgtctttagcgaggactttgatggttccagtttggccgacttgggcaattggaccgctgaagtcagattccctggcgaaccggactacccgtacaacttgtacactacggacggcactgtgggattcgaaagtgggtctctggtggtgagacccgtcatgaccgagtccaaataccacgagggcatcatatacgaccgcctcgaccttgagagatgtacaggacagctgggtacgctggaatgcaggcgagagagcagcggcggtcagattgtaccacctgtgatgacagctaaactggccactcgacgcagcttcgcgttcaagttcggcaggatcgatataaaggcgaagatgccgcgcggggactggttgataccagaactcaacctcgaacctttagataacatatacggcaaccagcgatacgcttcgggtctcatgcgggtcgcgttcgtgagaggaaacgatgtatacgccaagaagctctacggaggtccgataatgtccgacgcggacccgttcaggtccatgctgttgaaggacaagcaagggttggccaactggaataatgattaccacgtctactcgctgctgtggaagcctaacggtttagagctgatggtggacggtgaagtgtacggcaccatcgacgctggcgatggcttctaccagattgcgaagaacaacctcgtgagccacgcctcgcagtggctcaagggcaccgtcatggcgccgtttgatgaaaagttcttcatcactctgggtcttcgcgtggcgggtatccacgacttcacggacggtccgggcaaaccttgggagaacaagggcaccaaggccatgatcaacttctggaacaatcggttccgctggttccccacgtggcacgacaccagtcttaaagtcgactacgtcagagtctatgctctttag

SEQ ID NO: 5 - DNA, 1194 bp, Manduca sexta
  source 1..1194: mol_type = genomic DNA
atgtcctcttgtccaagcgactatgatcccagtgaatcgtccaaatctccacaaagtatttgggagtcaggaggatacagttctccgtcgcaacaagttcctcaattgacttctaacttaacagaattgtctgttgatcacagctatagatacaatggaaatggaccatatctacagatcacagagcaaccacagaaatactttcggttccgttatgttagcgagatggtgggaacacatggatgtttgcttggcaaatcttatacaacaaacaaagttaaaactcatccgacagttgaactcgtgaattacaccggtcgagccctgataaagtgccaactatcgcaaaacaagagcgaagacgaacacccgcacaaactgctcgatgaacaagacagagacatgagccaccacgttcccgagcacggcagttatagagtggtatttgctggtatgggtataattcatgctgccaaaaaggaagttgcggggtggctctatagaaaatatatacagcagaacaagaatgaaaagtttaataagaaagagctcgaagcgcattgtgagaggatgtccaaagagatcgatttaaatatagttagactgaagtttagcgctcacgatattgacactggcattgaaatttgccggccagtgttctctgaacccatttataatttgaagtgtgcgtctacgaatgatttgaaaatatgccgcataagccgttgttacggtagaccgagaggcggcgaagatatcttcatatttgtcgaaaaggtcaacaagaaaaacatccaagttcggttctttagactggaaaacggggagcgcacctggtcagcgatggcgaactttctgctaagcgatgttcaccaccaatacgctatcgcttttagaacgccaccgtacgtcaatcaccaaatttctgaagacgtgcaagtttttatagaactcgtacgcccttcagacggtaggacgagcgctcccatggagttcacatacaaggctgagcaaatctataaacagaacaagaaacgtaaaactacttcgtcgtactcgtcgctcgacagctcctcaggttcggccggttcaattaaaagcatcagcgaactgcccgcgcccgttgtttttgctgaaaacgtaagttttttctatgacacattactcattcttcaacccatgacgaatctataa

SEQ ID NO: 6 - DNA, 1317 bp, Manduca sexta
  source 1..1317: mol_type = genomic DNA
atgcttgtgacgttatgcggcgggaactatagtggattgtcgttaacaaaaactaatcattatatgtcaccaaaatcatatgtgccaggaaatggttatgacgccgccgtaatcctaggtaccacggagcagaatgacagcgaaccctcaaacttgaatattagtgatgtttttgaagccatcacgctcgctgatccgtcgttcggcgcgggcgtgccgtcggtagaggagacgatggcgcacacgcagccccagccgctgcaaatgccgtacgtggtcgtcgtgcagcagcccgccagcaaagcgctcagatttcgatatgagtgcgagggcagatcagccggttcgattcccggcgcgtcgagcacgcccgagaaccgaaccttccccgccatcaagataatcggctacaccggcaccgtctccatcgtagtgtcgtgtgtcaccaaagatgagccttgcaggccgcacccacacaacctggtcgggcgcgaccactgcgaccgcggcgtgttctccgtccgcatcgagatcaccgacgagaataacgaataccagtttcggaacctgggcatacagtgcgtcaagcggcgcgacatcggcgaggcgctgcggatccgagaggacctgcgcgtcgatccgttcaaaaccggcttcacccaccggaaccacccgcaaggcatcgatctgaatgcagtgcggctcgcgttccaagtgttcctgccgcactccagcggcaagatgcggcgcacgctcgcgcccgtcgtgtccgacgtcatctacgacaagaaggccatgagcgacctgctcatcgtgcgcgcgagccactgcgccggcccggcgcgcggcggcacgcaggtcgtactgctctgtgaaaaggtgactcgcgaggacaccgtggtggtgttctaccaggaggacaacaaccgcgtgctgtgggaggagatggcgatcatcatcgtggtgcacaaacagtatgccatagcgttcgagacgccgccatacaagaacccaaacattactgataatgtcaatgtacgattccagctgagaaggctctccgacaagatgacgagcaactcgctgccgttcgagtacattcccgaataccaagattaccctagttacaggcaggataactcagaaagaaatccccaatcgcagccaattacgcacaaggtaacggtggaggactttgaatcgacaactaaaagatattttacgcgaagcactgacaacagtaattacggttgggatgcggttccggtcacgtacaatggaagaaagaaggtttgctattgccccaagaggagctaa

SEQ ID NO: 7 - DNA, 888 bp, Manduca sexta
  source 1..888: mol_type = genomic DNA
atggcctggatccagcatttactcgtttggctcttcgttatgtcaacatcagcatacaaatgcaaagactgcttcagtttcgcatcacaatatccgtcgtacgatagtcaagtatacgaacaacctgacagacggatagcgggacggtcagcacaatacgaacatttaagaacaaacgagaggtctctcccggtctacagcgagacccagaggatacaagcagaagagagaagaagacacagttcgagactagaagaaccgagacaacgtgctgagaatggttcatataagatattgaataaccctccgaaaccctgtattactaataggagaagtcaaattgattcgtcgaatgatagggtagtgttccccggtccgacttcagaaaggtcgtacgtacccgaagtgccagaggaatgcaagaaaatcggcatatgcgacagtataccgaattacccagaagaacacgtagctaatattatatctcgacttggagacaaaggaaaagtattacaaatagacgaactggacgtatcagacactccagatatcgcccagaggttgggtccgcaggaggacaacatggaactatgtagctttagagaaaagattttttaccccaaggcagcgccagacaaagatggaaattggttcttcgttgtgaattcaaaagaaaacccagtacagggttataaagttgaaatttgcgaccgtcagcaattaccatgcgcggagttcgcgagcttccaacagggatatgaagcgaggtgcatccagaaatacgttcgccggaccatgttggcgttggatcccaagggtcagatgaccgacatgccccttaaagtgcccagctgttgctcatgcgtggccaaattgacaatcatatga

SEQ ID NO: 8 - DNA, 2892 bp, Manduca sexta
  source 1..2892: mol_type = genomic DNA
atgcaggctcggcggtggtgcgcggcactgctattaatgcagatgctgagctggctcggagtcagtggacacttaccgcgtcccgagtgcgcgccagccgcagattgccaacttatacgagacaacataatcgatggatatgcacaattctacttcaacgtatcaggacatgaagtgaaatttgaacattacatcggaaacgacttcgatgtcgaattgtcatgcaattacatcgccatggacaacgcaatgctgccgcggttctcaacgaccttttcagtcaacgtaatagtggttaaagaatgtgctttgccaagaagtgggtcaatcgatgccgctgtcgctgcacttaatatcaacgttttgacggagctgactctggacaaattcctagagccggcggtgatcacgcgcgcacatcttaccagtttacaacgactagagaggctggagctacacggtaactcaaacacaagcctcgcccccggcgcactggccgcgctctccgccgcgtccgcactgaaatgtcttgtattgcatgcagtacgcgtgcccgccgctgacctggcgcgcttgccgtcgtcactgcaagaactagcgttgttggatgtgggcgctgcgagtatgcatttagattcatcggttaatttgacgtcactcttcgtaatcgatacacattatcctgtcgtcgtgaatgtgagcaacgccgttgcgctcagagacttgcacataaataccccaagtactgtgttgaccgaagacgtgctcccgtcgtcactcaactcacttgaactagaggggtggaacgaaacgcatccggtgcctaagacacgttgtgtactacttaaggaacttaatgtaatcggcaccgacaatgatgcctatccggtgactctcccggacgagtggctgtccaactgcggacagctgagggatcttgaaatgatctccgtgccgattagcgccgtacttccggcgcggatgctggctaacgcaattaggcttgaaacgattactatctggaactgtaacctgaccgcgttgccgtcgggcctgttagacgacacgctgaacctcgccacactcgacttgtccaacaatcaacttgcatcgttgcccagaaaattatttgagcacacgaagttactacgcagtctcatactatcgaacaatcagctgacgagcgaggtagtgtggacgctgtcgaccgtcacctcgcttgttgaactaaagctcagcaataataaccgcataggcgacttatgttcccacgactcagtgtcagcaggtccctcaccactgagttcactgacggggctaaactatctccatctgagcaacacaggagtatcacacgtgtgctccgactggcgagaaaagctaacctacctcacgaatctcaacttaagggacaaccccatcactctctacaatttggcggatctacagtttcgtcgaatctgggtggacgctcgtgtgtatttaggacatttcaagcagcagtttacgcgcacagattacgaactcgccagtaataataacactgaggcggtcgtaactttatccggttcgttagaatgcgactgcaattcatactgggcagcacaggtgttccgtatgaaagcttggcaggcatccagctccatgatatattgtgaaaaaaaaccggtcattgaggtggatcccgacacctttacctgccttgagccagcgaagtgtgctgcgctggcggatagttgtacgtgccgtatccgcgacgacattcagtacaaacaagtggtcgttgtgcactgcaccggactcgccgagttcccgcgcctgccactaaccacggacaagtggatcctgcacctgccccacaacaacatatcatacctcgccgcggccgacgtatcgccgaacatcgtggaactcgatctaagaaacaattcaatcaagaatatcgacgtacaggcatcagcaaaactagccttcgtccggctgcaattgggtggtaacccgatcgagtgtgactgcgaggcgttgaagctgctggcgccgctgctcaaacctgactcaaagctgcttgacagaaaggacgtgaaatgtgagaacgacgcgcagattaccttggcgatgctgaaattatgtactaagtcatccaatgggctgatgtacttgctgttcctgttgctgttactcgcatttgtcgtaaccggactgctcgcacgaaccgcaattcgcctgcgcatcaaaatgatcctcatgagactgggttggatgtcgagactactggagcccgcggacgacgatcgcccgtacgacgcgtttgtgtctttcgcacacgaggatgaggagctggtgatggagcagctggcggcacggctcgagagcggctcgcggccgtaccgactgtgtctgcactaccgcgattgggcgccaggcgagtggatcccggcgcaaatagcggcttcggtgcgggcctctcggcgcacggtggcggttgtgtcggcgcactacttacagtcgggctgggcgcttgccgagatccgggaggcgaccgcagcctcgctgcaggaaggcatgccacgtctcatcatcgtgctgctcgacgagaccgaccggttgatgctcgatatagaccctgagttgcacgcctatgtgcgcaacaatacctacgtgcgctggcatgatccatggttttgggagaagctgaagcaggcgctgcctccaccgcgggaacaacggtcgccaatagctccgtcgctaccagcgctggcgctgtcccatgacagcctaactctgcgcacgtactctcccagagaaagtgacccagcgcccgctgctaagccggcgcacactccgcacgagacggacgagccagcgccgggcgcgagtccttgctacaaatga

SEQ ID NO: 9 - DNA, 840 bp, Manduca sexta
  source 1..840: mol_type = genomic DNA
cggcagtcggttgtgttggcagtggcggcggtgctcttcgggtgcgcgtgcgcagcgcccaatcctggcgccaacgacatacaacttaatcaaaaattaagtatcgaagctaagggggcaaagcagccaattgatacgagggcagtgaaggaacggtatccatacgcagttcggagtttcggaggcttctgcggaggaaccattatcagtcccacctggatcctgaccgccggccactgctcgatactctatgcggggagcggcctaccggccggcaccaacattaccgaggtatctagcttgtaccgcttccccaagcggctcgtcatacacccgctcttctccataggacccgtctggctcaacgctacggagttcaacctcaaacaggcggctgcacgatgggacttcttgttgatagaactggaggaaccgctgccgttggacggcaagatcctggcggctgcgaagctcgacgaccagcccgacctccccgcaggcctcgacgtgggctatccgagctacagcaccgacacctacgaggctaagatacaaagcgagatgcacggaaagaagctttcggttcaatctaacgaggtgtgctcgaagctagagcagttcaaggcggaggacatgttgtgcgccaagggacgtccaccgcgatacgacttcgtctgcttcagcgacagtggcagtgggctagtagacaacaatggtcgcctagtcggcgtggtgtcgtgggccgagaacaacgctttcgagtgccgcaacggcaacctggcggtcttctcgcgagtgtccagcgtacgcgagtggatccgacaagtcaccaacatataa

SEQ ID NO: 10 - DNA, 1200 bp, Manduca sexta
  source 1..1200: mol_type = genomic DNA
atggtttatattttaataattttagtgatttgcaattttagttgtattagttgtcagtccgggacagtggaaagcaggattcattttaaagatgaagggccggaatgttatgatgcaaataaaaagggcacctgtgttagtgctcacagatgccttgatgtagttagaaaacttaaagacggagagaaacccacgatatgtggctaccaaggcacggaaccaatggtgtgttgcacagactgtactctggttgataatattagtaatttggtcgtaagttccatatccgggtacctgtggaaggatggtcagaaagcgtgggacaaatgtctggaatacgttgacaagctgtcgtacccatgcgcttcaacctactcccactacctcagctccgtttgggagaaagataaggagtgcagtatggttcagtttgttggcgtgaggcgattcgcctcgtataacggacaaccggcgaaacggaacgagtaccctcacatggctctgctcggctacggcgacgaccaggagacggcgcagtggctctgcggcggctcagtgatcagtgatcaattcatcctcacggctgcacactgcatctttacaaatctattgggtccagtacgtttcgcagcgctaggaatactgcagcgatcggatccagtagagttatggcaagtttacaagatcggcggcatagttccccatccgcagtataagtcacctatcaagtaccacgacattgctctcctgaagactgaaaacaaaataaagtttaatgagaacgtgctgccagcgtgtttgttcatagagggcagagtgggtgggagtgagcaggctaaagcgaccggttggggcgcgcttggacataaacagacggcagctgacgtactgcaagtggttgaccttcaaaagttcagtgacgaagagtgcggaagtacctaccgtccttaccggcatttgcctcaaggctacgacagcgccacgcagatgtgctacggcgacaagggaaaactgaatatggacacctgtgagggcgacagcggcggtcctctacagttccaaaactcctcgctcctctgcatacacatagtagcgggagtgacgtcattcggcgacgcgtgcgggtttgcgggcggcgccgggatgtacacacgagtgtcgtactatattccctggatcgagagcgttgtatggccgtga

SEQ ID NO: 11 - DNA, 2046 bp, Manduca sexta
  source 1..2046: mol_type = genomic DNA
atggctttgaaacttttaactttgatagccctgacttgtgcggctgcgaatgcagctaaatcttcatacaaactatgcgtgccagcagcatacatgaaggactgcgagcagatgcttgaagtacccacgaagtctaaagtggccttggaatgtgtaccggctagagacagggtggaatgcctcagctttgttcagcagcgacaggcggacttcgtccccgtcgaccctgaggacatgtacgtggcctccaagatccccaaccaggacttcgtcgtcttccaggagtacaggactgatgaagagcctgatgcgccattccgttatgaagccgttattgtggttcacaaagacctacccatcaacaacttggatcagctgaagggactgaggtcttgccacaccggagtcaatcgtaacgtcgggtacaagatcccactaacgatgttgatgaaacgtgccgtgttcccgaaaatgaacgaccacagcatttcgccgaaagagaacgaactgaaagcgctatcgacgttcttcgcaaagtcgtgcatcgtcggcaaatggtcgcctgaccccaaaaccaactcggcttggaaatcacaatacagccatttgtgttcaatgtgcgaacacccggagcgttgtgactatcccgacaattacagcgggtacgagggcgcgttgagatgcctcgcccacaacaacggggaggtcgcgttcaccaaagtcatattcacacgtaaattctttgggcttccagtaggtaccactccagcgagtccatcaaacgaaaatcccgaagagttcagatatctctgcgtggacggatctaaagcccccatcactggcaaggcttgttcatgggctgccagaccttggcaaggactgatcggtcacaatgacgtacttgccaaactcgctccgctcagagagaaggttaagcaacttgctgattctggtgcagctgacaaaccggagtggttcaccaaagtccttggtctatcagagaagatccaccatgtcgctgacaatatcccaatcaagcccatcgactacctgaacaaggctaactacacggaggtcattgaaagaggacatggagctcccgagctggtcgtcaggctatgtgtgacgtcaaacgtggcattatctaagtgccgggctatgtccgtgttcgcattcagtagagacatcaggccgatcctagactgtgttcaagaaaacagcgaagatgcctgtcttaagagcgtccaagacaacggttcagatcttgcctcagtagacgatatgagagtagctgcagcggctaagaagtacaacttacatccagttttccacgaagtgtatggagagctaaagacgcccaactacgcagtggctgttgtcaagaagggcactgcctacaacaagatcgacgacttaaggggaaagaaatcttgccacagctcttacagtactttcagcggtctgcacgcgcctctcttctaccttattaacaagagggccattcaatctgaccactgcgtgaagaacttgggagaattcttctcaggcggatcttgcttgcctggtgtcgacaaacccgaaaacaacccaagcggtgatgatgtgtctaaattgaagaagcaatgtggatccgacagcagcgcttggaagtgcttggaagaggacagaggagacgtcgcatttgtttcaagtgccgatctgtcccacttcgacgccaaccaatacgagctgctctgcctgaaccgcgacgctggcggtagagatgttctctccagtttcgccacttgcaacgtcgccatggccccgtccaggacctgggtggctgcgaaggacttcctgtctgacgtatctatcgcccacacaccattgagcctcgcccaaatgctcgctacgagacctgacctcttcaacatttacggagagttcttgaagaacaacaatgttattttcaataatgccgctaaaggcttagcaacaactgagaaacttgacttcgagaagttcaagaccatccacgacgtcatctcttcatgtggtctcgcctaa

SEQ ID NO: 12 - DNA, 2112 bp, Manduca sexta
  source 1..2112: mol_type = genomic DNA
atgaagactgtcataatcctagcggggttggtggccctggccctcggcagcgaagtgcctgtcaagcactccttcaaagttaaggatgttgatgcggctttcgtcgaacgtcaaaagaaggtcttagatcttttccaagatgtcgaccaagtaaatcctaacgatgagtactacaagattggcaaggaatacaacatcgaggctaacatcgacaattactcgaacaagaaggccgtcgaagaattcttgcagttatacaggacaggtttcttgcctaagtactatgaattttcacccttctatgacagactaagggacgaggccattggtgttttccacctcttttactacgctaaagattttgatacgttctacaaatctgccgcatgggcgcgtgtgtacctcaacgaaggacagttcttatacgcctactacattgctgtgattcagcgtaaagatactcagggcttcgttgtaccagcaccgtatgaagtctaccctcaattcttcgcaaacttgaacactatgctcaaagtctaccgtaccaaaatgcaggatggagttgttagtgccgatttagctgcacaacacggcatcgtaaaggagaaaaactactacgtatactatgccaattactccaactcattagtgtacaacaacgaggaacagagactgtcgtacttcactgaggacatcggcttgaattcgtactactactacttccactctcacttgcctttctggtggaattctgagagatacggagcactaaaatcgcgccgtggtgaaatctactattacttctatcagcaattaattgcacgttattactttgaacgtctctcgaacggcctgggtgacattcccgaattctcatggtactcaccagtcaagtctggctactatccactgatgtcttcttattactaccccttcgctcaaaggcccaactactggaacgtgcacagcgaagaaaactacgagaaagtacgattcttggacacgtatgaaatgtcattccttcagttcctccaaaacggacacttcaaagcgtttgaccagaagattgacttccacgatttcaaagctatcaactttgttggaaactactggcaagataatgctgacctgtacggtgaggaagttactaaggactaccaacgttcatatgaaattatagcccgccaagtgcttggtgctgcacctaaaccattcgacaagtacacattcatgcccagcgctttagacttctaccagacgtctctgcgtgacccaatgttctaccaactttacaacagaattctgaagtacatatatgagtacaagcagtacctgcaaccgtactcttcagaaaaactggcattcaagggtgtcaaggtggtcgatgttgtagtagacaaactggttaccttcttcgagtactacgactttgatgcgtccaacagcgttttctggagcaaagaggaggttaaatctagctacccccatgatttcaagatccgtcagccacgccttaaccacaagccattctctgtctctatcgacatcaaatctgaagctgccgttgatgccgttgtcaagatattcatggcacctaaatacgacgataatggattccctctgaaattagaaaacaactggaacaaattcttcgagctggactggttcacatacaaatttgttgctggtgacaacaaaatcgtgaggaactcaaacgacttcttgatcttcaaggacgactctgttcccatgactgagttgtacaaattattagaacaaaataaggttccacacgacatgtccgaggattacggctacctgcctaaaagactgatgctgccaagaggtactgagggtggtttcccattccagttcttcgttttcgtatatccattcaacgctgacagcaaagatcttgcaccgttcgaggccttcatccaggacaacaaacctttgggctatccattcgaccgtcccgttgttgacgcttacttcaagcaacacaacatgttcttcaaggacgtcttcgtataccatgacggcgagtacttcccgtacaagttcaatgttccttcccatgtgatgcactcaaacgttgttcctaaacactga

SEQ ID NO: 13 - DNA, 846 bp, Manduca sexta
  source 1..846: mol_type = genomic DNA
atgtacgtgaaagtagcacttctgttggtagccctcattgctgggagctgggccttcccaaagctcgaagatgagcaggacatgtccatcttcttcacgcagctcgattcgagcgcgcgtatcgtgggtggtacccaggcccccagcggaagtcaccctcacatggtggcgatgaccaccggtaccttcatcaggagcttcagctgtggaggctcagttgtcggtagacgttccgttctgactgcggctcattgcatcgctgctgttttcagtttcggttccctcgccagtaccctccgcttgacggtcggcaccaacttctggaaccagggaggcaccatgtacaccgtcgctcgcaacataacccacccccactacgtctctgcgaccatcaagaacgacatcggtctgttcatcactcacaacaacatcatcgacacgactgtcgtccgcagcatccctcttaactttgactatgtgcccggtggtgttctcactagagtcgccggatggggcaggatcaggaccggcggtgccatctctccctctctgctggagatcattgtgcctactatcagtggaagcgcatgcgtagccagtgcaatccaagctggcatcgatctgaacatgagaccacctcccgtcgagcctcacatcgagctgtgcaccttccacggtcctaacgtaggcacttgtaatggtgactccggcagcgctcttgcccgcctagacaacggccagcagatcggtgtggtatcgtggggcttcccgtgcgcacgcggcggtcccgacatgttcgtcagggtcagcgcctaccaatcctggctgcagcagagcatcgtataa

SEQ ID NO: 14 - DNA, 579 bp, Manduca sexta
  variation 27
  source 1..579: mol_type = genomic DNA
atcattgacggaccttccgttggaccngccatcatcggcgctggagacatcgctgtcggccctgctatcgtcgacttccctttccccgacggcggtgccgtgtctgcccccgttgagccttcccccatcgccatcggacccgctatcgtcggtgaatcccctatctccgtcggacctgccatcgttgaggccggagacatcgctgttggacccgctatcatcgacttcccccttcccgacggtggcgccgtgtccgcccccgttgaggtttctcccgtcgactccgtcgtcgtcggccctgccgccggctctcagagctctcccctcgtccagatcatcatcaacgttaaggcccccgctggtgccggccccgttgtcgatgccgtcgctgacaagcccatggacatcattgatgttatgcccgtcgtcgaccctgctgatttcgtggacctcacccccgttgtagagcctgtagaagtcgtcgacattgtcgatgtcatgcccgtggttgaccccatcaacatcatcgatgttatgcctgttgttaagcccgtaaacccccttgcccgttcttaaggg

SEQ ID NO: 15 - DNA, 1479 bp, Manihot esculenta
  source 1..1479: mol_type = genomic DNA
atggatccttgcaagttccgtccatcaagctcaaacaatacccccttctggaccaccgatgctggtgctccagtatggaacaacaattcctccatgactgttggaaccagaggtccaatccttttggaggactatcatatgatagagaaacttgccaactttaccagagagaggattccagagcgtgtcgtccatgctaggggaatgagtgcaaagggcttctttgaagtcacccacgatgtctctcaccttacttgtgctgatttccttcgagcccctggagttcaaacccctgtcatcgtccgtttctccactgttatccacgagcgtggcagccctgaaacactcagggatcctcgaggttttgcgactaagttctacaccagagagggcaactttgatattgtgggaaacaacttccctgtcttcttcatccgtgatggaataaaattcccagatgtgatacacgcttttaagcccaatcccaagtctcacatccaagaatactggaggatctttgacttcttatcacaccatcctgagagcttgagcaccttcgcctggttcttcgatgatgttggaattccccaagattacagacacatggaaggtttcggtgttcacacctttactttcatcaacaaggctggaaaagtaacctacgtgaaatttcactggaaacccacttgcggggtcaagtgtttgatggatgatgaggcacttaagatcggaggtgccaaccacagccatgctacgcaggatttatacgactccattgccgctggcaactatcctgagtggagactcttcatccagacaatggatccagctgatgaagacaaattcgactttgatccacttgatatgaccaagatctggcctgaggatatttttcctctacagcaaattggccgtttggtcttgaacaggaacatcgataactggtttgctgagaatgaaatgctcgcattcgaccctggtcatattgttcctggcattcactattcaaacgacaagttgtttcagctcagaacctttgcatatgctgacactcagaggcaccgtctcggacccaactataagatgctccctgttaatgctcccaagtgtgcttatcacaacaatcattacgatggtttcatgaatttcatgcacagggatgaggaggtggattacttcccatccaggtatgatccagttcgccatgctgagagaagccccattcctaacgctatctgtagtggaaggcgtgaaaagtgcgtcattgaaaaggagaacaatttcaagcaacctggagagagatatcgatcctgggcacctgatagacaagaaagattcctgtgcagattggttaacgccttatcagagccacgtatcacctttgagattcgcagtatctgggtctcttactggtctaagtgcgacgcgtctctgggtcaaaagctggcttctcgtctcaacgtgaggccaaatatatga

SEQ ID NO: 16 - DNA, 611 bp, Manduca sexta
  source 1..611: mol_type = genomic DNA
ggcaaagaattcaaatggcaggaacacaatatcgcccagcgcaaagacgaaggcagcctggtcttcctcaagcccgaggctaaagatgaaggccaatacagatgtttcgctgagtcggccgccggagtcgccacctcccacatcatctcctttagaaggacctacatggtcgtacctactacttttaagactgtagaaaagaaaccggtagaagggtcatggctcaaacttgagtgcagcatccccgaaggttatcctaaacctactattgtatggagaaagcagcttggtgaagacgaaagtatagcagattctatactggcacgtcgtattacacaatctccagagggagacctgtacttcacgagcgtcgagaaagaagacgtaagcgaaagctataaatacgtttgcgctgctaagtcaccggctattgatggggatgtccctcttgttggatacactattaaaagcttagaaaagaatacaaatcagaaaaacggtgagctggtcccgatgtacgtcagtaatgatatgatagctaaggccggagacgttactatgatctactgcatgtatggtggagtcccaatggcttaccccaactggttcaa

SEQ ID NO: 17 - DNA, 645 bp, Manduca sexta
  source 1..645: mol_type = genomic DNA
atgttgttgcttctgtattgtcttgtggcggcctccgcgccgttctttattgcagcggaccaaggcagccctgacctgcctttagctaccgaaccaccaacagaatgcggaacaatagcacctgatgatagcttagtattagatgggtccgttggtaaaagtgacaaattaccttggtatgctattatctacacaaccaccacccggccatacaagcagatcggtggaggaaccctcatcactccttcagtagtaatctcagccgctcactgtttctggcgcaatggtgaggttccatctaaggataattacgccgtggcgctcggcaagacccatagtgcttggaatagccatgccgatgtaaacgctcacaagtctgatgtaaaagaaatacacataccaccgcagtttaagggaaggaacactaattatcggaatgatatagcaatcgtggtcatgtcagaccctgtgacctacaaagtggacatccgccctatctgtttgaacttcgatgtacaatttgaaagactgcaattaaaagacggcattatggggaagatcggcacatggaatgtaagtcgtgagacactgaaactatcgaaaacattaaaagtggtggagaatccatacattgacgcagcgact

SEQ ID NO: 18 - DNA, 584 bp, Manduca sexta
  source 1..584: mol_type = genomic DNA
atggcgagcttcgctttaatagttatccttagcgtaattggctttatatcggcctatcctagtcctgaaggttacagttctgccttcaactttccattcgtaaccaaggagcagtggggcggcagggaggcacgcacgtcgacgccactcaaccacccagtgcagttcgtggtgatccaccacagttacattcccggcgtgtgcctcagccgggacgagtgcgcgcgcagcatgcgctccatgcagaacttccacatgaacagtaacgggtggagtgatattggatacaacttcgctgtcggcggtgaagggtcggtgtacgagggccgcggctgggacgcggtcggcgcacacgcagctggctataacagtaacagtatcggcatcgtgctcatcggcgattttgtttcaaacctcccgccggcggtgcaaatgcaaaccacacaagaattgatcgcagcgggcgtgcgactcggttacatcaggcccaactacatgctcatcgggcatcgtcaggtctccgccactgagtgcccaggaaccagactcttcaacgaaatcaccaactggaacaacttcgtgag

SEQ ID NO: 19 - DNA, 1334 bp, Manduca sexta
  source 1..1334: mol_type = genomic DNA
gcgtctgtttgttcgcaaccattgcgggctgcttgggccagcgagggggtccatacaaggtgcctgatgcgaaactcgaagctatctaccccaaaggcttgagagtctctgtgccagatgatggctactccctatttgccttccacggcaagctcaatgaggagatggaaggtttagaggctggccattggtccagagacatcaccaaagcgaagcagggcagatggatattcagagataggaatgctgagctgaagcttggagacaaaatttacttctggacttacgttattaaggatggattgggatacaggcaggacaatggagaatggactgttacagaattcgtcaatgagaacggtacagtggtggacactagtacagcgccgccaccagtagcacccgccgtttcagaggaagatcaatcgccaggtcctcagtggagaccttgcgaaagatccctgactgagtccttggcccgcgaacgcgtttgcaaaggcagccttgtctttagcgaggactttgatggttccagtttggccgacttgggcaattggaccgctgaagtcagattccctggcgaaccggactacccgtacaacttgtacactacggacggcactgtgggattcgaaagtgggtctctggtggtgagacccgtcatgaccgagtccaaataccacgagggcatcatatacgaccgcctcgaccttgagagatgtacaggacagctgggtacgctggaatgcaggcgagagagcagcggcggtcagattgtaccacctgtgatgacagctaaactggccactcgacgcagcttcgcgttcaagttcggcaggatcgatataaaggcgaagatgccgcgcggggactggttgataccagaactcaacctcgaacctttagataacatatacggcaaccagcgatacgcttcgggtctcatgcgggtcgcgttcgtgagaggaaacgatgtatacgccaagaagctctacggaggtccgataatgtccgacgcggacccgttcaggtccatgctgttgaaggacaagcaagggttggccaactggaataatgattaccacgtctactcgctgctgtggaagcctaacggtttagagctgatggtggacggtgaagtgtacggcaccatcgacgctggcgatggcttctaccagattgcgaagaacaacctcgtgagccacgcctcgcagtggctcaagggcaccgtcatggcgccgtttgatgaaaagttcttcatcactctgggtcttcgcgtggcgggtatccacgacttcacggacggtccgggcaaaccttgggagaacaagggc

SEQ ID NO: 20 - DNA, 512 bp, Manduca sexta
  source 1..512: mol_type = genomic DNA
agtgtgcgtctacgaatgatttgaaaatatgccgcataagccgttgttacggtagaccgagaggcggcgaagatatcttcatatttgtcgaaaaggtcaacaagaaaaacatccaagttcggttctttagactggaaaacggggagcgcacctggtcagcgatggcgaactttctgctaagcgatgttcaccaccaatacgctatcgcttttagaacgccaccgtacgtcaatcaccaaatttctgaagacgtgcaagtttttatagaactcgtacgcccttcagacggtaggacgagcgctcccatggagttcacatacaaggctgagcaaatctataaacagaacaagaaacgtaaaactacttcgtcgtactcgtcgctcgacagctcctcaggttcggccggttcaattaaaagcatcagcgaactgcccgcgcccgttgtttttgctgaaaacgtaagttttttctatgacacattactcattcttcaacccatgacgaatctataa

SEQ ID NO: 21 - DNA, 789 bp, Manduca sexta
  source 1..789: mol_type = genomic DNA
atgcttgtgacgttatgcggcgggaactatagtggattgtcgttaacaaaaactaatcattatatgtcaccaaaatcatatgtgccaggaaatggttatgacgccgccgtaatcctaggtaccacggagcagaatgacagcgaaccctcaaacttgaatattagtgatgtttttgaagccatcacgctcgctgatccgtcgttcggcgcgggcgtgccgtcggtagaggagacgatggcgcacacgcagccccagccgctgcaaatgccgtacgtggtcgtcgtgcagcagcccgccagcaaagcgctcagatttcgatatgagtgcgagggcagatcagccggttcgattcccggcgcgtcgagcacgcccgagaaccgaaccttccccgccatcaagataatcggctacaccggcaccgtctccatcgtagtgtcgtgtgtcaccaaagatgagccttgcaggccgcacccacacaacctggtcgggcgcgaccactgcgaccgcggcgtgttctccgtccgcatcgagatcaccgacgagaataacgaataccagtttcggaacctgggcatacagtgcgtcaagcggcgcgacatcggcgaggcgctgcggatccgagaggacctgcgcgtcgatccgttcaaaaccggcttcacccaccggaaccacccgcaaggcatcgatctgaatgcagtgcggctcgcgttccaagtgttcctgccgcactccagcggcaagatgcggcgcacgctcgcgcccgtcgtgtccgacgtcatctacgacaag

SEQ ID NO: 22 - DNA, 743 bp, Manduca sexta
  source 1..743: mol_type = genomic DNA
cgaacaacctgacagacggatagcgggacggtcagcacaatacgaacatttaagaacaaacgagaggtctctcccggtctacagcgagacccagaggatacaagcagaagagagaagaagacacagttcgagactagaagaaccgagacaacgtgctgagaatggttcatataagatattgaataaccctccgaaaccctgtattactaataggagaagtcaaattgattcgtcgaatgatagggtagtgttccccggtccgacttcagaaaggtcgtacgtacccgaagtgccagaggaatgcaagaaaatcggcatatgcgacagtataccgaattacccagaagaacacgtagctaatattatatctcgacttggagacaaaggaaaagtattacaaatagacgaactggacgtatcagacactccagatatcgcccagaggttgggtccgcaggaggacaacatggaactatgtagctttagagaaaagattttttaccccaaggcagcgccagacaaagatggaaattggttcttcgttgtgaattcaaaagaaaacccagtacagggttataaagttgaaatttgcgaccgtcagcaattaccatgcgcggagttcgcgagcttccaacagggatatgaagcgaggtgcatccagaaatacgttcgccggaccatgttggcgttggatcccaagggtcagatgaccgacatgccccttaaagtgcccagctgttgct

SEQ ID NO: 23 - DNA, 890 bp, Manduca sexta
  source 1..890: mol_type = genomic DNA
caagtggtcgttgtgcactgcaccggactcgccgagttcccgcgcctgccactaaccacggacaagtggatcctgcacctgccccacaacaacatatcatacctcgccgcggccgacgtatcgccgaacatcgtggaactcgatctaagaaacaattcaatcaagaatatcgacgtacaggcatcagcaaaactagccttcgtccggctgcaattgggtggtaacccgatcgagtgtgactgcgaggcgttgaagctgctggcgccgctgctcaaacctgactcaaagctgcttgacagaaaggacgtgaaatgtgagaacgacgcgcagattaccttggcgatgctgaaattatgtactaagtcatccaatgggctgatgtacttgctgttcctgttgctgttactcgcatttgtcgtaaccggactgctcgcacgaaccgcaattcgcctgcgcatcaaaatgatcctcatgagactgggttggatgtcgagactactggagcccgcggacgacgatcgcccgtacgacgcgtttgtgtctttcgcacacgaggatgaggagctggtgatggagcagctggcggcacggctcgagagcggctcgcggccgtaccgactgtgtctgcactaccgcgattgggcgccaggcgagtggatcccggcgcaaatagcggcttcggtgcgggcctctcggcgcacggtggcggttgtgtcggcgcactacttacagtcgggctgggcgcttgccgagatccgggaggcgaccgcagcctcgctgcaggaaggcatgccacgtctcatcatcgtgctgctcgacgagaccgaccggttgatgctcgatatagaccctgagttgcacgcctatgtgcgcaacaatacctacgtgcg

SEQ ID NO: 24 - DNA, 725 bp, Manduca sexta
  source 1..725: mol_type = genomic DNA
tcgaagctaagggggcaaagcagccaattgatacgagggcagtgaaggaacggtatccatacgcagttcggagtttcggaggcttctgcggaggaaccattatcagtcccacctggatcctgaccgccggccactgctcgatactctatgcggggagcggcctaccggccggcaccaacattaccgaggtatctagcttgtaccgcttccccaagcggctcgtcatacacccgctcttctccataggacccgtctggctcaacgctacggagttcaacctcaaacaggcggctgcacgatgggacttcttgttgatagaactggaggaaccgctgccgttggacggcaagatcctggcggctgcgaagctcgacgaccagcccgacctccccgcaggcctcgacgtgggctatccgagctacagcaccgacacctacgaggctaagatacaaagcgagatgcacggaaagaagctttcggttcaatctaacgaggtgtgctcgaagctagagcagttcaaggcggaggacatgttgtgcgccaagggacgtccaccgcgatacgacttcgtctgcttcagcgacagtggcagtgggctagtagacaacaatggtcgcctagtcggcgtggtgtcgtgggccgagaacaacgctttcgagtgccgcaacggcaacctggcggtcttctcgcgagtgtccagcgtacgcgagtggatccgacaagtc

SEQ ID NO: 25 - DNA, 777 bp, Manduca sexta
  source 1..777: mol_type = genomic DNA
gatggtcagaaagcgtgggacaaatgtctggaatacgttgacaagctgtcgtacccatgcgcttcaacctactcccactacctcagctccgtttgggagaaagataaggagtgcagtatggttcagtttgttggcgtgaggcgattcgcctcgtataacggacaaccggcgaaacggaacgagtaccctcacatggctctgctcggctacggcgacgaccaggagacggcgcagtggctctgcggcggctcagtgatcagtgatcaattcatcctcacggctgcacactgcatctttacaaatctattgggtccagtacgtttcgcagcgctaggaatactgcagcgatcggatccagtagagttatggcaagtttacaagatcggcggcatagttccccatccgcagtataagtcacctatcaagtaccacgacattgctctcctgaagactgaaaacaaaataaagtttaatgagaacgtgctgccagcgtgtttgttcatagagggcagagtgggtgggagtgagcaggctaaagcgaccggttggggcgcgcttggacataaacagacggcagctgacgtactgcaagtggttgaccttcaaaagttcagtgacgaagagtgcggaagtacctaccgtccttaccggcatttgcctcaaggctacgacagcgccacgcagatgtgctacggcgacaagggaaaactgaatatggacacctgtgagggcgacagcggcggtcctctacagttccaaaactcctcgctcctctgc

SEQ ID NO: 26 - DNA, 1298 bp, Manduca sexta
  source 1..1298: mol_type = genomic DNA
atggctttgaaacttttaactttgatagccctgacttgtgcggctgcgaatgcagctaaatcttcatacaaactatgcgtgccagcagcatacatgaaggactgcgagcagatgcttgaagtacccacgaagtctaaagtggccttggaatgtgtaccggctagagacagggtggaatgcctcagctttgttcagcagcgacaggcggacttcgtccccgtcgaccctgaggacatgtacgtggcctccaagatccccaaccaggacttcgtcgtcttccaggagtacaggactgatgaagagcctgatgcgccattccgttatgaagccgttattgtggttcacaaagacctacccatcaacaacttggatcagctgaagggactgaggtcttgccacaccggagtcaatcgtaacgtcgggtacaagatcccactaacgatgttgatgaaacgtgccgtgttcccgaaaatgaacgaccacagcatttcgccgaaagagaacgaactgaaagcgctatcgacgttcttcgcaaagtcgtgcatcgtcggcaaatggtcgcctgaccccaaaaccaactcggcttggaaatcacaatacagccatttgtgttcaatgtgcgaacacccggagcgttgtgactatcccgacaattacagcgggtacgagggcgcgttgagatgcctcgcccacaacaacggggaggtcgcgttcaccaaagtcatattcacacgtaaattctttgggcttccagtaggtaccactccagcgagtccatcaaacgaaaatcccgaagagttcagatatctctgcgtggacggatctaaagcccccatcactggcaaggcttgttcatgggctgccagaccttggcaaggactgatcggtcacaatgacgtacttgccaaactcgctccgctcagagagaaggttaagcaacttgctgattctggtgcagctgacaaaccggagtggttcaccaaagtccttggtctatcagagaagatccaccatgtcgctgacaatatcccaatcaagcccatcgactacctgaacaaggctaactacacggaggtcattgaaagaggacatggagctcccgagctggtcgtcaggctatgtgtgacgtcaaacgtggcattatctaagtgccgggctatgtccgtgttcgcattcagtagagacatcaggccgatcctagactgtgttcaagaaaacagcgaagatgcctgtcttaagagcgtccaagacaacggttcagatcttgcctcagtagacgatatgagagtagctgcagc

SEQ ID NO: 27 - DNA, 1371 bp, Manduca sexta
  source 1..1371: mol_type = genomic DNA
ctgtcataatcctagcggggttggtggccctggccctcggcagcgaagtgcctgtcaagcactccttcaaagttaaggatgttgatgcggctttcgtcgaacgtcaaaagaaggtcttagatcttttccaagatgtcgaccaagtaaatcctaacgatgagtactacaagattggcaaggaatacaacatcgaggctaacatcgacaattactcgaacaagaaggccgtcgaagaattcttgcagttatacaggacaggtttcttgcctaagtactatgaattttcacccttctatgacagactaagggacgaggccattggtgttttccacctcttttactacgctaaagattttgatacgttctacaaatctgccgcatgggcgcgtgtgtacctcaacgaaggacagttcttatacgcctactacattgctgtgattcagcgtaaagatactcagggcttcgttgtaccagcaccgtatgaagtctaccctcaattcttcgcaaacttgaacactatgctcaaagtctaccgtaccaaaatgcaggatggagttgttagtgccgatttagctgcacaacacggcatcgtaaaggagaaaaactactacgtatactatgccaattactccaactcattagtgtacaacaacgaggaacagagactgtcgtacttcactgaggacatcggcttgaattcgtactactactacttccactctcacttgcctttctggtggaattctgagagatacggagcactaaaatcgcgccgtggtgaaatctactattacttctatcagcaattaattgcacgttattactttgaacgtctctcgaacggcctgggtgacattcccgaattctcatggtactcaccagtcaagtctggctactatccactgatgtcttcttattactaccccttcgctcaaaggcccaactactggaacgtgcacagcgaagaaaactacgagaaagtacgattcttggacacgtatgaaatgtcattccttcagttcctccaaaacggacacttcaaagcgtttgaccagaagattgacttccacgatttcaaagctatcaactttgttggaaactactggcaagataatgctgacctgtacggtgaggaagttactaaggactaccaacgttcatatgaaattatagcccgccaagtgcttggtgctgcacctaaaccattcgacaagtacacattcatgcccagcgctttagacttctaccagacgtctctgcgtgacccaatgttctaccaactttacaacagaattctgaagtacatatatgagtacaagcagtacctgcaaccgtactcttcagaaaaactggcattcaagggtgtcaagg

SEQ ID NO: 28 - DNA, 831 bp, Manduca sexta
  source 1..831: mol_type = genomic DNA
atgtacgtgaaagtagcacttctgttggtagccctcattgctgggagctgggccttcccaaagctcgaagatgagcaggacatgtccatcttcttcacgcagctcgattcgagcgcgcgtatcgtgggtggtacccaggcccccagcggaagtcaccctcacatggtggcgatgaccaccggtaccttcatcaggagcttcagctgtggaggctcagttgtcggtagacgttccgttctgactgcggctcattgcatcgctgctgttttcagtttcggttccctcgccagtaccctccgcttgacggtcggcaccaacttctggaaccagggaggcaccatgtacaccgtcgctcgcaacataacccacccccactacgtctctgcgaccatcaagaacgacatcggtctgttcatcactcacaacaacatcatcgacacgactgtcgtccgcagcatccctcttaactttgactatgtgcccggtggtgttctcactagagtcgccggatggggcaggatcaggaccggcggtgccatctctccctctctgctggagatcattgtgcctactatcagtggaagcgcatgcgtagccagtgcaatccaagctggcatcgatctgaacatgagaccacctcccgtcgagcctcacatcgagctgtgcaccttccacggtcctaacgtaggcacttgtaatggtgactccggcagcgctcttgcccgcctagacaacggccagcagatcggtgtggtatcgtggggcttcccgtgcgcacgcggcggtcccgacatgttcgtcagggtcagcgcctaccaatcctggctgcag

SEQ ID NO: 29 - DNA, 571 bp, Manduca sexta
  variation 27
  source 1..571: mol_type = genomic DNA
atcattgacggaccttccgttggaccngccatcatcggcgctggagacatcgctgtcggccctgctatcgtcgacttccctttccccgacggcggtgccgtgtctgcccccgttgagccttcccccatcgccatcggacccgctatcgtcggtgaatcccctatctccgtcggacctgccatcgttgaggccggagacatcgctgttggacccgctatcatcgacttcccccttcccgacggtggcgccgtgtccgcccccgttgaggtttctcccgtcgactccgtcgtcgtcggccctgccgccggctctcagagctctcccctcgtccagatcatcatcaacgttaaggcccccgctggtgccggccccgttgtcgatgccgtcgctgacaagcccatggacatcattgatgttatgcccgtcgtcgaccctgctgatttcgtggacctcacccccgttgtagagcctgtagaagtcgtcgacattgtcgatgtcatgcccgtggttgaccccatcaacatcatcgatgttatgcctgttgttaagcccgtaaacccccttgcccgtt

SEQ ID NO: 30 - DNA, 710 bp, Manihot esculenta
  source 1..710: mol_type = genomic DNA
atggatccttgcaagttccgtccatcaagctcaaacaatacccccttctggaccaccgatgctggtgctccagtatggaacaacaattcctccatgactgttggaaccagaggtccaatccttttggaggactatcatatgatagagaaacttgccaactttaccagagagaggattccagagcgtgtcgtccatgctaggggaatgagtgcaaagggcttctttgaagtcacccacgatgtctctcaccttacttgtgctgatttccttcgagcccctggagttcaaacccctgtcatcgtccgtttctccactgttatccacgagcgtggcagccctgaaacactcagggatcctcgaggttttgcgactaagttctacaccagagagggcaactttgatattgtgggaaacaacttccctgtcttcttcatccgtgatggaataaaattcccagatgtgatacacgcttttaagcccaatcccaagtctcacatccaagaatactggaggatctttgacttcttatcacaccatcctgagagcttgagcaccttcgcctggttcttcgatgatgttggaattccccaagattacagacacatggaaggtttcggtgttcacacctttactttcatcaacaaggctggaaaagtaacctacgtgaaatttcactggaaacccacttgcggggtcaagtgtttgatgga

SEQ ID NO: 31 - DNA, 772 bp, Manduca sexta
  source 1..772: mol_type = genomic DNA
atgacttctttgaaaagcaaattagcagaattcttgaaggggttaaaatcagatgcaaccccaagccccgaggccatcgacagaacccagggtaaatccacaaactgagcaatgaaaatgacgcaccctcggatagtgaacctgaagaaatcattatagaagatatgacgatacgaagaaaaagaaaaaggcaagttacaagaaaccgttctttagctccaaacctggtgcattccccgaaaagaaaaaagacaagaataccaaagacgattacagcaactttgtgaatactcaagccactggtgatgtcatcaatattgtaggctgtaacaatttccgctggggtaataattattatttgggaaataccaagaaacaggctcctcctaagaaatatttccaagaagaggaagacagtgaaccagaagatgatattcagaaatgcaacttaatcaaactgctatttgaagctgaaaataagccagaacatgagtacctggactacatttcccagaacatgaatgaaaagtggcacagattctttgtgaaactaggttttacaccaggaaggatcaaaacatccatcatagataatgccagttatggtatttcagaggctcgatacgcattgctacttgagtgggtgaataaaaaacgggactctaatcttggacagctatcgaatttattgtggaaacacggcgagaggcgaattgtcaaagaattagccattatgtactctgcaagcaaggccaaatctgatgatgaatag

SEQ ID NO: 32 - DNA, 666 bp, Manduca sexta
  source 1..666: mol_type = genomic DNA
atgactctatcggaatcaaaatttaaacaattaaaagaacaaattattttacatgcaagtgcaactgaaagacatgctcaaattttgaactcattaaaggatttgtttaaagaagatattaattctgttcgaagatttgaacaaatcagcaatatagcacaattattaaaagttttagaaataagagatgtgttgtcagaggatgatgttgctcctttgaaagatgtggcacgccaactaccaaatagctcagaaatgctgcggaaaattgctgaatatgaagaaaatcacaagtgtggagagtttatttctgttcctaagtcacctccaaaacaaaaagaacatagtcattctggatgggatattaacagcatacagaattctgaatattctgtaaaaaaggaaaggatatttgaagttatttctgaagaaattggcacccattggagaaatctagcgagatatttgaaaacaagggaatgtacaataattgaaattgacagtaaaaacataccactttcaagtaaagctatggagattttgaaattgcattgtaaaaaagcaaatccacaaaagtggttttttgacttgtgtaatgcattggacaaagtgcataggactgacatagtgttatcactgcaagaaattatgtctatgaatatttag

SEQ ID NO: 33 - DNA, 1632 bp, Manduca sexta
  source 1..1632: mol_type = genomic DNA
atgttgtcttccgatgccagaagttccctaaattccaagagtgataatgaaacgtttatattaaacttggattcgatttcaaagattgaaaaacaattacaagataatccttatgatatgatctctttagtgttcctgttgtacgacgtgccggatacggcgctgcagagattgatggtgtatcaacgagtgacgagtgacgtcagtggaacaaatattaatttgttgcaagaatggtactgtcacgccagcagtcgccctgactggcaacatgaattattagaagcattaatgatatgccaattgaactccattgtgaagagccttggtttccatatacccactatgagagtattttaccaatcaaatgatcccttcagcagcaagtacataaatcccgtaaagaaagtcctttatcatgcctgtgaaaatataaactcaactaatcttttgaaattaaaaaaatcacttctttcatatgatataaatgtaatggaatatacaacttgtgagcttatatttttagagctcctgtccaataaatttattactattaataatattggccaaaagattaaaacaaaccagaactgcatatgtaatgtagaaaaccttgtgaagatcttagacaacttaaatggattgaaaaaagtggctatgaatttgagatattttcaaagtaaattcaatgatgaagaggtggattcattcgcgtctgtcaatggaagcagctcaccatcacttccagtgcctcctcttgatggtacaaaattaaagcaagatgacaagaattatggagctgaagatttctcagagctattcgacataataaataatatgccagaaccgcttgaaaacaattttaaatctgatacaatgtcaactaaacagaatagatatgagataaaaaatccggaacaactaggtgtttgcatagttataaaccaagagaatttttatccatccaaaaatagtattgaagaccaccaaattgttccattagaagagagaaaaggatctagtgtggataaaaggactttggaaagaaccatgacatcattaaaatttcaagttcacagttgctctgatttagatcatgatgaaatgatagaattcatcaaaaagaaaattaataaacatgttacatcaaatgacagtatttttatgttgtgtatactgtcacatggtgtaagggaccatgtgtatgctgctgactctgtgaaaattaaagtagagtctatacaaaatctgttggattcagatgaaatgagccacttgagaggcataccgaaggtgtttattatacaagcttgccaagttgaggatacacctcatcctacatttgctgctgacaatgtccaaacaaattactacttgggaaaattagacttccttatttactgggccactgcacctgaatatgaagccttcagacatgagcagacggggtcattattcattcaagctctttgtaaactgttgcgtcaaagagctaaacatgatcacttacatgaaatctttacacaagtaaataacaatgttaccaatctgtgcactaagttgcagcgtgcacaagtacctctcttcaaaagtactctgaggaaaaatttatatttacaagtgccagaataa

SEQ ID NO: 34 - DNA, 2823 bp, Manduca sexta
  source 1..2823: mol_type = genomic DNA
atgtcctcttgtccaagcgactatgatcccagtgaatcgtccaaatctccacaaagtatttgggagtcaggaggatacagttctccgtcgcaacaagttcctcaattgacttctaacttaacagaattgtctgttgatcacagctatagatacaatggaaatggaccatatctacagatcacagagcaaccacagaaatactttcggttccgttatgttagcgagatggtgggaacacatggatgtttgcttggcaaatcttatacaacaaacaaagttaaaactcatccgacagttgaactcgtgaattacaccggtcgagccctgataaagtgccaactgtcgcaaaacaagagcgaagacgaacacccgcacaaactgctcgatgaacaagacagagacatgagccaccacgttcccgagcacggcagttatagagtggtatttgccggtatgggtataattcatactgccaaaaaggaagttgcagggtggctctatagaaaatatatacagcagaacaagaatgaaaagtttaataagaaagagctcgaagcgcattgtgagaggatgtccaaagagatcgatttaaacatagttagactgaagtttagcgctcacgatattgacactggcattgaaatttgccggccagtgttctctgaacccatttataatttgaagtgtgcgtctacgaatgatttgaaaatatgccgcataagccgttgttacggtaggccgagaggcggcgaagatatcttcatatttgtcgaaaaggtcaacaagaaaaacatccaagttcggttctttagactggaaaacggggagcgcacctggtcagcgatggcgaactttctgctaagcgatgttcaccaccaatacgctatcgcttttagaacgccaccgtacgtcaatcaccaaatttctgaagacgtgcaagtttttatagaactcgtacgcccttcagacggtaggacgagcgctcccatggagttcacatacaaggctgagcaaatctataaacagaacaagaaacgtaaaactacttcgtcgtactcgtcgctcgacagctcctcaggttcggccggttcaattaaaagcatcagcgaactgcccgcacccgttgtttttgctgaaaacttacctgaaaacaatgagcgtattatagatatacccgtacaacagaatatgctgtaccaggttctgccaagtcaatgtgatttagccgacgcgttcatggaggtggacagcaagggaagtccggccagcgtcgtggacccgatgtgggccggcgccgacgtcgggctgcagatggcgtcgagcctgcccgccatcaaactcggctccaccgaactagagagcttggcgcaccgcagccgggacggagtgcccatggacaaagaattccttgacaattatctcagctctctcaactcgcttggtgaacttaacgacgatgatgagatcaatcatatgcaatatgtgcgctcgttacagatggtccacgccgactcggcacgacgaaaggctgaaccgcaacccaaagacactgtaaccaaaccgactcaatcttccccgaaggacacagactatggcgcccagtcgggacgccccactgaatattctgcttattataaaatggaagacggcgtggaagtcaaaaaactggtgaaagaattgtgtagtatcatacagaacaaagcgggatataaaaaacaagaagtgagaaacaaattggagaggctgttcacctaccgtctgtccaacggagatacattccttcatatgacattgtgcagcaatcaaagtagttttgaatacatcgtcaaaatcatacatagcgtgaaaatgacacatctattggactattgtaataataaacagcaaaccatattacacatggctattgtaaacgacctgcctcgaatggtctctttacttattgcaaaaggttgcaatccaatgaataaagacagcgagggcgacaatgcggtgcactacgctgtgaggagcgaatgttgtttagaagcattattggacgccatcaaaaataacaatgttcggtgcgatttgaacgactgcaataacgagaagcagacggcgctgcacctgtcgacgagcggcgcgagcgcgcggcagctggcggcgcgcggcgccgacccgcgcgtgcgcgacgcgcagggccgcacgccgctgcacctcgccgcctacgacgacaactgcgacgtcgtcagggcgctgctcgagttcgtgtcgccctcggaaatagacgttgtggacggctgtggcaacactgcgttacagatcgtctgcggcggctccgttaagaagaacactttggaaatagtaaaactgttgctccaaaaaaaggctgatccgttgaagcaagatgggcacaacatatcggcgtggaagatggcgcgcgaacactccgaaatacgggacgcgatgaaggactatgtccccgccgccgcgtacgaggaggacaccaagtcggaaatggacgatgagttcgaatccgctgatgaggaggactaccggatgggttccggatcgggcgctgtgagtctgccggagctgggcgcatacgtggaggcgctgagcgcggcgctggaggcgcgcggcgcgtggcgcgcgctcgcgcaccgcctcggcctcgccgccgcgctcgactggtgcgcgcgacagcacgcgcccgcgcgcacgctgctgctgcacctcaaggaatgcagaaacgacatatcctcgaaaacgttggctgtaattctagaagatatgggagaattagaggctgcttcaattataaggagacacatagagtga

SEQ ID NO: 35 - DNA, 579 bp, Manduca sexta
  source 1..579: mol_type = genomic DNA
atgcaggcgatcaagtgtgtcgtcgtcggagacggtgccgtcggtaaaacatgtctgctcatcagctacacgacaaatgccttccccggagaatacatacctacagtattcgacaattattcagcgaatgtgatggtggacgggaagccgatcaacctgggcctgtgggatacggcggggcaggaggactacgaccggctgcggccgctgtcctacccacagaccgacgtgttccttatatgcttctcgctcgtcaacccggcttcgttcgagaacgttcgggctaagtggtacccagaagtgcggcatcactgcccgtcgacgcccatcatcctcgtcggtaccaagctggacttgcgcgaagacaaagacaccatagagaaacttaaggacaagaagctcgcgcctatcacttacacacagggtctgggcatgtcgaaggagatcaacgcggtgaagtacctcgagtgctctgcgctgacgcagaagggtctgaagacggtgttcgacgaggccatccgcgccgtgctgtgtcccgtgccgccccccaaacagagccggaagtgcacgctgctgtaa

SEQ ID NO: 36 - DNA, 1212 bp, Manduca sexta
  source 1..1212: mol_type = genomic DNA
atgagtaagatgtcaaagaataaattaaacttgaccctgccaccagggtcaatagacacagcaccagccatcacaccatccaatatgacaccacagctgaagtccgcaacagctacggagcgtcagggcttggctggtaaatcgaaaaccagcatcgaagccctgacagagaggttggagcaaatcgagatggacgacacacagagacggagaatagaagtgtttctgtgtcagaaggagaagatcggggagcttagtgatgatgattttgaaaagcttggagagttaggccaaggcaacggtggcgttgtaatgaaagtccgtcacaagtcaaccggtctgataatggcgcgaaagttaatccatctggaagtcaagccggcaataaagaagcaaatcatcagggagttgaaagtcttacacgaatgtaactttgcgcatatcgtcggcttctacggggccttttatagcgacggcgagatctcgatttgtatggagtacatggacggtgggtccttagaccttatactgaagaaggccggcaagattcctgaatctattctaggaacaataacatccgccgtgctgaaaggtctgagctacctccgggacaagcacgccatcatgcatcgcgacgtgaaaccatcaaacattctggtgaacagcaacggcgagatcaagatatgcgatttcggcgtgtccggtcagctgatcgattccatggccaactcttttgtcggcactaggagttatatgtctcccgaacgtctccaaggcacgcactattcagtccaatctgacatatggtcgctcgggctgtcgttggtagagatggcaatcggaatgtatcccataccgccgccggacgcgaagaccctggctgccatcttcggtggacagaatgaagatcattctcctggtcaggcgccgaactcgccccgtccgatggccatattcgagttgctggactacatcgtgaacgagccgccgccgaagctgcccgccggaatattctccgacgagttcaaggacttcgtcgaccgctgcttgaagaagaatccagacgaacgagccgacttgaagactttgatgaatcacgaatggatacgcaaagcggacgcagaaaaggtggacatagcggggtgggtgtgcaagacaatggacctcatgccttccactccaaactctaacgtgtctcctttttcttcataa

SEQ ID NO: 37 - DNA, 1155 bp, Manduca sexta
  source 1..1155: mol_type = genomic DNA
atgcagaacatcgatcctctggagatcgccaacttcctcgccacggagctgtggtgccagcagttggcgaacctcgagggcctccagtcgggggtcccgactcgcacaacggccaccatcacgccgacgcaactgcgcaacttcgagcagacttacatcgagctgaccaactgccgcagcgagcccaccacgcacgccggcttcgtgccgccgtcagtcacgcacgccaacaactacggcatcctgaatccgacggcgtattgtgattcgggcccgacgacggcgctgcacgtgtcgccgggcccgctctccgccagcggcgacagcagcagcagccccggcctgcccacgcccaagcggcgcaacatgggcggccgccggcccaacaaggcgccgcaagaactcacgcccgaggaagaagagcgcaggaagatccgccgcgagcgaaacaaaatggccgccgcccgttgtcgcaagcgcagactcgaccacaccaacgagctgcaagaggaaaccgacaaattagaggaaaagaagcatgcgctccaggaggaaatccgcaaactgaacgctgaccgggagcagctgcaagtgatccttcagaaccatatggtatcatgccggctcaacaagagatccatcagcccgcccgatgtgaagcccttccaggacccgtacgcctaccctgagatacccgaggatggcgtccgtgtcaaggtggaagtggtcgacccctcagtagacacggtcttagtgttggacaatatttactcaacgccgccgactgacaaaaaaattatgctgtcgtcggccaacccagccgtggtgacgagtgggtcgcccgccgccctggagaccccgccggcgatagtgcgtcccaacagacccaactccctccaggtgccgctcagcctcacaccagcacagttacacaacaacaaggcgctgggaaacaacaaaatagccggcatagagataagcacgccgagcaacggcatcccgttcaacttcgagagcctgatggagggcggcacggggctgacgccggtgcacggccacgcgctgccgctgccgcacccgtgcgcgcagcagcagcgcgccgcgcccgacgcctcgcccgccgagccggcgccgagctcgctggtgagcctctga

SEQ ID NO: 38 - DNA, 720 bp, Manduca sexta
  source 1..720: mol_type = genomic DNA
atggttcggcactccggccacggcatggagaccactttctatgacgagcagtatcccatcagcggccccgtggagaacctgaagcggcccctcacgttagacgtggggcgcggcgtgaagcgcgccaggctcggcggggcacccgtactctcatctccagacctacagatgctgaagctcggctcgccagagctcgagaaactgatcatccagaacggcttggtgacgacggccacccccactccaggtgcgccggtgctgttccccgcggtcgcgcctaccgaagagcaagagatgtacgcgcggccattcgtcgaggcgctagacaagctgcaccacggcgaggtgaccccgatcgggcggcgagtgtacgccgacctggaccggccgctcgagcggtaccccacgcccgtggtgaaggacgagccgcagacggtgcccagcgccgccagctcgcctccactttcccctattgacatggacacgcaggagcggatcaaactggagcgcaagcgacagaggaatcgagtggccgcttccaaatgcaggcggcgcaagctcgaacgcatctccaagctggaggacaaggtgaagatcctgaagggcgagaacgcggagctggcgcaaatggtggtgaagctaaaagagcacgtacaccgactgaaggagcaagtgctggagcacgccaacggcggctgccacatcgagtcgcacttctga

SEQ ID NO: 39 - DNA, 735 bp, Manduca sexta
  source 1..735: mol_type = genomic DNA
atggtgaattacgttaatcccctcgccatgtaccaaggcaagggcgggcaatacggcggcgggtggtacggctggcagcatcagaactttgaagaacaacaatggtgtgcttggaacggtgcgccggcgggtggcgagtgggcgccagatccacatcattttcccaaaagagaacctggagagagagaaatagcagacatgccatcaccggcacgaggagacttggcaagtccagcagaaggttcgccgagctcggggtcgaggccgtcgcagccgccagcaccgccgcgttccccatacgaatggatgaaaaaacccaactaccagacacaacctaacccaggtaaaacgcgcacaaaagacaaatacagggtggtgtacagtgatcatcagaggctggagttagagaaggagttccactacagcaggtacatcactattcgtcgcaaggcagaactcgccgtcagccttggcctttccgaacgacaggtcaaaatttggtttcaaaatagacgagccaaggaaaggaaacaggtgaagaaacgcgaagaagtggtgatgaaagagaaaggcgaccatgcatctctacagcacgcgcagctgcaccatgccaccatgctgcatcaccagcagatgatgaacggcatgatgcaccaccaccactaccaccaaggcgtgttgcaaggcgtgccggagccgctggtgccgggcgtgccgcccgtgccgctgctgtga

SEQ ID NO: 40 - DNA, 354 bp, Manduca sexta
  source 1..354: mol_type = genomic DNA
atgaaattccaatacaaagaagaacattctttcgaaaagaggaagactgaaggcgaaaagattcgcaggaaatacccggatcgcgttccagtaattgtagagaaagccccgaaggctagattgggagacctcgataagaagaagtatttggtgccgtctgatttgactgtcggacaattttatttcctaattaggaaacgcatccatcttcggcctgaggacgcattgttctttttcgtgaacaatgttattccaccaacatccgccaccatgggctctctgtaccaggaacatcatgacgaagatttcttcctctacattgcattttctgatgaaaatgtttatggattttaa

SEQ ID NO: 41 - DNA, 1260 bp, Manduca sexta
  source 1..1260: mol_type = genomic DNA
atggcacccgaagtggctttttctaacataaatgacaagaatgaatttactaaattcacaaaattcttagcttataaaggcgttcaagttattgtagaatcgaggaagggtgtaaaaatagatcctaatagtaaaccaagatcatcagacactgattggttcaatcttcaaataccagactctccagaggttaaccaggcaactaagaatgcattgccttcagacaaggtgttagagattatcaaagcccaactgcacgtggaaatatcagtacaaaccgaggatggcgatgaaatggttttggagttgtggacccttgaacttgacgaaactcagtttgacacttctgttaaagccacgaacacagtttacttcagaatgggaatattacttaagtcgcttataactataacaagaattactccagcgtaccacttgtcgaggaagcaaagaacagagtcgttcacaatattctacagagtatacaatggcgaaccgaaagtaaaagcgcttggagaatcagtgaagaaaattcaagctggaatgctcaaaactccacttggagggataatattctccgtggcctaccgcacaaacttctccatttcgccaaacagatcggagaaggacaaaacattgcttttgaagagtgatcatttcgaactgagtccaaaacatgtgatatttgaatccaagaaaaagaaagacgagaaaaaagaatacaagcctctgaaacctgttgatttgaataagccacttcgattagccgcatttgtagacgaggatgttgtaaaaaaggcctttgatgacttcatggagaagatgccgattcccaaataccgagtgatacgtagggagagaagtccagagagcgacaagcctatagtatccaaaagtactcaggatttggacgcttctgtgacgtcaaagagtccgacgtcaatggaaatgccacctaaaaagttcacaggctttcgcaatgagaacgaacctccattaaaacttctccatttcccattcgctgataaccatccgataagagaactggccgagttttacaaagagttcttcaacgccccccatttgaagttagctgatgacgttagcctgaaatcgggcagtgctgaatcggtaaaagaggtgatagagattacaactgaagatttgtcgaaagatctcgaattgtatgagaactcggtgttggagtttgatgagctcttggcagatatgtgccgatcggctgagtggagtgggaactag

SEQ ID NO: 42 - DNA, 1062 bp, Manduca sexta
  source 1..1062: mol_type = genomic DNA
atggcatcagctggcgccgtaccgaatattctagtgtcgtcgtcgcttttcaagaccactcctcgggatcctaaaattcgtcgaaaaacaagccctttgaagttaccaccttgtgacacacatataggctcgcctccaccatcatcctctccgacctcctcgtcaactgataaaaccgataatcatgatacttttggtttccttcctgacatgcgtgatatgcgccgcgaagaagaacgactgaaaactttcgataagtggcctagcacgtacgtgacacctgaagagttagctcgtaatggtttctactaccttgggcgcggtgatgaagttcgctgtgcattctgtaaagtagaaattatgcgatgggcggaaggagacgaccctgcgaaagatcacaaacgatgggcgccccaatgtccgtttttacgtaatctttcgaacggtactaatggcagcggagaaggtagttcggaaggtcgcgacgagtgtggtgcgcgagcagcgacgcgggagcctgtgcgtatgcccggacctgtgcatccccgctatgcaacagagttatcgcgtctcgccagtttcaaggattggccgcgttgcatgcgccaaaaaccagaggaactcgcggaagcaggatttttctacaccggccaaggggataaaacgaaatgtttctattgcgatggaggtctcaaagattgggaaaacgacgatgttccatgggaacagcatgcccgttggttcgatcgttgtgcgtatgtgcaacttgttaaaggccgtgattatgtccagagggtattatcggaagcgtgtgttatacgtgccaccgacgaaaagcctgcgcctgcacctcaaccgtcccaaccaaatgtctcagttgtatcagaagaaaaacctgtcgaagaggctaagatatgcaaaatatgttattccgaagaacgaaacatatgctttgtgccgtgcggtcatgtggtcgcttgtgcaaagtgtgccttatctactgataaatgcccaatgtgccggagggctttcacaactgcactgcgactctatttttcgtga

SEQ ID NO: 43 - DNA, 4575 bp, Manduca sexta
  source 1..4575: mol_type = genomic DNA
atggccgcaactacaccaggttttaagaagttagcagacgattctgaggattcagatacagaatacaccccgctgtatgatgacggtgatgaaatagatcaaagaactgcacaagaaacaaaaggatggaatctatttcgagagattccggtgaaaaaggagagtggatctatggccacaaaaaattggatagaaacaagcgtaaaaatcataaaagtgcttgcctacatattggttttttgtgctgtactgggttccgcagtcatagctaaaggaactcttctatttattacgtcacaactgaagaaagacagacaaattactcactgcaatagacgacttgctttagaccaacagttcataacggtacacagtttagaagaaagaataacatggctatgggcagcacttattgtattcggtgtgccggagttaggggtgtttttgagatccgtcaggatatgcttcttcaaaactgccaagaaaccaaccaaaacacagtttattattgctttcataacagagacactacaagcaataggaatagcagcacttgtattaataattctaccagaattagacgctgtgaaaggagccatgttgatgaacgccacgtgcgctatccctgcattgctaaacattttcacgagagaccgaatggattctaagttttctataaaattgatattggatgtattggcgatatcggcacaagccacggcgtttgttgtttggcctcttatggaaagaacgccagttctatggaccataccagttgcatgtgtgttagtgtctctaggcttctgggagaattttgttgacacctacaataaaagttatgtttttacggtgctgcaggaactacgcgacaacctcaagaggactcggtactacactcagcgggtgctatctgtttggaagattatagtgtttatggcatgcattttaatatcgctgcatatgcaaaatgacaatccgtttacctttttcactcacgccagcaaagcctttggagagagacagtatgtcgttaacgaggttctaatagtagtccgagatgacgaaaccataggctatgacgtcaccggaggtatattcgaattggacgcgatatggacctcagcattgtgggtcgcattaattcaagtgggagcagcctacttctgtttcggaagtggcaagtttgcttgcaaaattcttatacaaaattttagtttcactttagcattgactctcgtcgggcccgtggcaatcaacctccttattgctttctgcggaatgagaaatgcagacccttgcgctttccatagaactatacctgacaatttgttttacgagataccacctgtgtacttcttgcgggagtacgtgggccacgagatggcgtgggtgtggttattgtggctcatatctcaagcgtggatcgtgtttcacacgtggcagccgcgatgcgagcgcctctccgcaactgacaaactgtttgccaaaccgtggtacatcggaccgctaatcgaccaatcgttgctgctaaacaggactaaggatttggataatgattgccaggttgaggatttgaagggtcttggcgacgattcgtcggttggaagcgatcttgccatcgtaaaagatatcaaaccgttcgattcgataaccagaatacaagtgtgtgcgacaatgtggcacgagaccaatgaggagatgatcgagttcttgaagtcgatattccgcctcgacgaggaccagagcgcgcgccgcgtggcgcagaagtacctcggcatcgtcgaccccgactactacgaactcgagtgtcatattttcatggatgacgcttttgaaatatccgatcacagtgccgaagactcgcaggtgaatcgcttcgtgaagtgcctagtggatgcggtggacgaagcggcttccgaagtgcatctgactaacgtgaggttaaggccccccaagaaataccccaccccgtacggcggaaaactaatttggaccatgccagggaaaaataagttgatttgccatttgaaagataaatccaagattcggcacagaaagagatggtcgcaggttatgtacatgtactatttcctggggcatcgcttgatggacctgccaatatccgtggatcgtaaggaagtgattgccgagaacacgtacctattagctttggatggagacatcgacttcaagccgagcgctgtgacgttgctggtcgatcttatgaagaaggataagaacttgggcgccgcttgcggtcgcattcatcctgtcggctctggtttcatggcctggtaccagatgttcgagtatgctattggtcattggctgcaaaaggcgactgaacacatgatcggctgcgtactctgtagtccgggatgcttctccctcttcagaggaaaggcgcttatggacgacaacgtcatgaagaaatacacactcacttctaacgaggctcgacattacgtgcaatacgatcaaggcgaggaccgttggctgtgtacgttgctgctgcagcgcgggtaccgcgtggagtactcggcggcgtcggacgcctacacgcactgccccgagcggttcgacgagttcttcaaccagcgccgccgctgggtgccctccaccatggccaacatattcgatctgctcgcggactccaaacgcaccgtgcaagtcaacgacaacatttccactctgtatatcgtctatcagtgcatgcttatgatgggtacgattttgggtccgggaacaatcttcctgatgatgattggcgcaataaacgctataacaggcatgagcaatatgcacgcacttctctttaacctggtgcccgtgcttacgtttttggttgtctgtatgacatgcaagtccgagactcagttgatgctcgcgaacctcattacctgcttttatgcgatggtaatgatgtttgtgatcgtcagtatcgtcctacaaatatcacaagatggttggctagcgccatctagtatgttcactgcggcaacatttggaatattcttcgtaacagcggctttgcatccacaagaaataatatgtttgttgtacatttccatatattacatcacaattccgagcatgtatatgttgttgattatctactccctatgcaatttgaacaacgtctcgtggggaactcgagaggtggctcagaagaagactgcaaaggaaatggaaatggataagaaagcagcggaagaagcaaagaagaagatggataatcaaagcataatgaaatggttcggcaagtcggacgagacgagcggctcgctggagttcagcgtggcgggactgttccgctgcatgtgctgcaccaaccctaaggaccacaaggacgacttgcatctcttgcagatcgccaactccatcgagaagatcgaaaagagattgtcggcacttggcgcggaggagtccgagccggcgcaggcgcagacgcggcgccgctcgtcgctggggctgcggcgcgactcgctcgccaccatgcccgagtacgccgacagcgagctgtccggagacattcctcgcgaagaaagagacgatcttataaacccctattgggtggaggatcccaatctgcagaaaggtgaagtagacttcttgacgacggctgaaatcgaattctggaaggacttgattgatgtttatttgaggcctatcgatgaaaacaaggaagagcaggaacgtatcaaaaccgatctaaagaacttgcgtgacacgatggtgttcgcgttcgccatgttgaactcgttattcgtgttggtgatattcctgctgcagttcaaccaggaccagttgcacataaagtggccgttcgggcaggatgtcgcgctttcttatgacaaggaaaggaatgttgtattggtggagcaggaattccttatgttggagcctataggttccctgttcctcgtgttcttcgggtttgtaatgttgatacagttcgtggcgatgttgtcccatcgttcgtatactattacgcatttgctctccacaacagagcttcactggtatttcagcagacgtccggaccagatgtcagatgaaaacctcttggaaaggaaggtggtagaaatagcgagagagttgcagaagttaaacacggacgacctagaccgccgcgcggtcgaaactaacgacgtgtcgcggcgaaagactctacacaacctagagaaggcgcgagacaccaagcacagcgtgatgaaccttgacgctaacttcaagaggcgactgactatactacagagcggtgatcctaacgtgatatctcggctgtcatcgttgggcggcgatgaggttactcgtcgcgccacgatacgcgcattaaagacgaggagggactcactgctcgctgaaaaacgacgctcccagctgcaagcggcgggcgacgctacaggctacatgtataacctgtcaggcactgcggtgaacgacatgagcggccgagcttcgacggccagcgcctacattaataaaggatacgaacccgctttcgatagcgacgacgacgaaccaccgcgtccgcgcaggagcactgtacgcttcagagaaaactacacgtaa

SEQ ID NO: 44 - DNA, 1473 bp, Manduca sexta
  source 1..1473: mol_type = genomic DNA
atgtacattaaaacagcaacatttttgctgtgcgttttccttggtagtgtatcgtcatgttgcgttaatgggcggtactacccgaggtaccatttgtcgccaccgcatggctggatgaacgaccccaacggattctgctacttcaaaggtgaataccatatgttttaccagtacaatcccatgtcaagtttggaggctggcatagctcattggggtcatgcgaaaagtaaagatttgtgccattggaaacacttagacctcgccatctatcctgatcagtggtacgatcaaacgggagtattttctggaagtgcgctagtagagaatgacgtcatgtacctttattatactggaaatgtaaatcttactgatgaaatgccatttgagggacaattccaagctcttggtatcagtactgacggtgtccacgtagaaaagtataaagacaatccaataatgtacacgccaaaccatcaacctcacatccgagacccaaaagtttgggaacacgacggctcttattatatggtcttaggaaacgcatatgatgattatacaaagggccaaatagttatgtacgaatcatcagacaagatcaactggcaagaagtaactatactatataaatcaaatggatctttcggttacatgtgggagtgtccagatttattcgaaatagacggcaagtttgtacttctgttctctcctcaaggcgtgaagtctgtgggcgatatgtaccagaatctgtatcaagcaggatacatcgtcggagaattcgattacgatactcattcattcacaatactaaccgaattcagagaattggatcacggtcatgatttttacgctacacaaacaatgaaagatcctagtggaagaagaatagtcgttgcttgggcaagtacttgggagtatgcttatcctgaacgagcagatggttgggctggcatgctcacactacctagaactttaactttgacaaaagatttaagactaatccaaactccaattagagagatcgatcaagtttttagaagaagactatattcaggaaaagcctcagcaggcaaaactgtcgctttaccagacaaagcagggaaagtagaactgaaatgggatacaccaagaaatataaaggtagttatagaatctcaaaatgagtgccaaaacgtagtaatcagttatgatcacgaggatggtactattactttggacagaggaggcgatgacgcaatccgccgcactcactgggatcctcgaggtcacctcaaatggaccatttttattgacgcaagctccatagaactctcttgtggtgatggagaagtatggtttacaagcagatttttccctgaaggagtcgtatctgttcgccttggagaagatacttgtgttgataagtttaccgtgcattctattcgccgtactactccagaccccgaggctcattgtcgttgtgaatcagaagaataa

SEQ ID NO: 45 - DNA, 606 bp, Manduca sexta
  source 1..606: mol_type = genomic DNA
tgttacattaattataaaaaaaaatacaaaaaagaaatatataagtaagtactaataatatggtctagtttattataagtcttagagactaacgataaataataaatttctcatgaaccacctatttatttatttgaaaagcattttaaatatattatagatttaaacgtaacgaagtctttaaactcgcgggtaaaaacgcatacttacttgactctacatcggcgacacacgaaaaacttattcgtactgttttttcattgaatatgttatgaaaaagaaaactatatgtaatcgcagtcacatatataaaactttataaaaaagttaacagagataaataacttgtttggtgttcgtcatagcaatagagtagccgcggcgcctaaggctacgaaataagctcattgaactgtaatggagtgtgcctagttgtttctcttcttaccaagactcaagagggatttggagcttcatctacgattattatacctacctacgtatagactataatggatagtaaaaatagaaagtaatttcttttatatcaacttttgtattcatatgtaagtacttacttataattatttatttttttaggtgaaa

SEQ ID NO: 46 - DNA, 425 bp, Manduca sexta
  source 1..425: mol_type = genomic DNA
gttaacaataaacaatactgttaaccgtacgtagtgttaaataatacaaatatatgtattttacaataagatttcctgtttttaaattatcctcacgtaacgtattgggtatctaagcggttgagcagtgagataagtctaccccaaagtagtccgttcatcaaacgacataaccatctcgtcatttagttaaaacaaaaaaagaataatgataaaagatataaaattctgtataagaaccaatacctagcacctttctcacattgccaaacatacattaaaaaacagtattatgcttttttcgctcttttattctatattttatattaaaaaatacctatagtaaatactgttttcatcgtcgccggtattcatacacgtagtatacgtacctaactctgaaactactggtttgaattgaaaaa

SEQ ID NO: 47 - DNA, 30 bp, Manduca sexta
  source 1..30: mol_type = genomic DNA
agcgctttgggaacgttccgtggcacgata

SEQ ID NO: 48 - DNA, 327 bp, Manduca sexta
  source 1..327: mol_type = genomic DNA
gatggtggaatgtttaaaaatacaagagtgtgaggcagtgattaatggtctgtaatatccgattcttgctggcttctgtttcgtaattaacgtaaaatctaattatcattagaatattttggaaaaccttagagttaccttcggaaaacttcaccgtttgccgctggtgtgaagatctgttccccccataaactgatgggaaattgtatgtaggtactcgagttataatttctttttatacctgcgtttaagttccattacaactttctaattttatgaataaatatctaataaacgattaacaaaattaaaaaaaaaaaaaaaaaa

SEQ ID NO: 49 - DNA, 89 bp, Manduca sexta
  source 1..89: mol_type = genomic DNA
acggcataaattgttaggtcgtctgagaggcgagtgttgtatttttaattgccaaagaggctgcgaagttcgcaataatcattagaaaa

SEQ ID NO: 50 - DNA, 68 bp, Manduca sexta
  source 1..68: mol_type = genomic DNA
aatgaccaaagataatacgactgttttataatttttgttaataaatgttgttgcattatggaaaaaaa

SEQ ID NO: 51 - DNA, 602 bp, Manduca sexta
  source 1..602: mol_type = genomic DNA
attaaaactaacgaaaagactcgtttccatagtggtatattttcaactcgtcaatttcaggtacaagcatgtttggtggaaggatattatcggcccgaataggcactgtacaccaagtacaaccatgactcatcaaaggtgtcgcgttctggaatcacctgtatttccaacaggccggcataattgtgtcgtctagcgagggataactcaatagtctatttgaacgccattctacttaccattaggtgtagtgaagatatttggggctagacaaaatggaaggaaatttatgagttgcatcgaatgattatcactaagtattcgaatgattcatttcgttgttggtgcaataggatgtcaagggtcaaatggacattaaaagagagtttaaggtgtttttttacaattaacgttgatctatcactttacttgcctattaagtaaagtattattttgaaaaatacaaaatacttgagtttatagtatctcttgcgtttatggtactgtcaaaaatatcagtattattcatttacaaaattaaaatttaataatctaaattattcttcatgtaaatgatcatttatacctctgccttgattatg

SEQ ID NO: 52 - DNA, 186 bp, Manduca sexta
  source 1..186: mol_type = genomic DNA
agagtacgttcgatggcagtctgtcgaacattggtagtttcccgtttgagtgttgtttactccctttgagggattagtttattctccacgaatataaacatcggaaaatcaaaaactaagttgataaaaagttgtgtgctccggtataattttttggtatcagtgacggacaaaggtgatataaaa

SEQ ID NO: 53 - DNA, 297 bp, Manduca sexta
  source 1..297: mol_type = genomic DNA
tatattcatgagaaggggacaactaaggattgaatatcagcagaacttgagttatgttaatgaggtatttatattagcttaatacttaaaggggaaaattcgattagcttttcaaatatacttaaattttagtttttgtcaaatatcggtgtttcccattttgatatttttttatccatattttaataataaatatcttgtcttagcaattttaatgggtaattataaagaaactcacgcaccatagttgcactaaactacaaaattacacaaaaaaaaaaaaaaaaaaaaaaaaaa

SEQ ID NO: 54 - DNA, 138 bp, Manduca sexta
  source 1..138: mol_type = genomic DNA
atacaacgatatcacaggcgtccggggacggaccagtttaaacaaacattacagtgaatagcgatgtgattattttcttgcttgtatagagttaaatttttaaattagatttaaatattaaaatatttgcgaataaaa

SEQ ID NO: 55 - DNA, 168 bp, Manduca sexta
  source 1..168: mol_type = genomic DNA
acttaacaattacatctataaatctctcctctatcaacctagtgggcgtcctctacaactaccacgtctggtatacaaatagtgacaacctgaaattgtgagatttaaattgtgtagttatgataaataaacaataaatccaaaaaaaaaaaaaaaaaaaaaaaaaaa

SEQ ID NO: 56 - DNA, 453 bp, Manduca sexta
  source 1..453: mol_type = genomic DNA
gaaaagttattcacaatatgacccgaagtgtcagcgccgcacatgcgcgggagcactcgtacttataccagacattgtgactaaatacctaattttatgttttgctcatcgcccattgcgcgacattaagcagtatccagtagtcagtgatcagtgttactacactattttgcttctcagcgattagtcaacacgcgcatatcgtttacttcaacaaacaattattacgcaaatcgtgatatttgaatgtagagacacgaatcaaagattattcgtttaacgttattttcgcgggatgtttctctgttggggggtactgttgcgtggtgtgcagtcaaaggtttattttatcgggtattgtgctctttaagtgttgacgcggcggattatcgtcggttagactaataaatcgtgtcgatttatgtgtgacccactacggtgtcgtgcgacgtg

SEQ ID NO: 57 - DNA, 150 bp, Manduca sexta
  source 1..150: mol_type = genomic DNA
atccagaacttgcgtgtacccttcgatatacagggctattttgacatcgtgttactaaatgaaaccacattctcgtcttcacctttgctgacattgtgccaaaaatcatccattaataacgaatatttccaccaaaaaaaaaaaaaaaaa

SEQ ID NO: 58 - DNA, 190 bp, Manduca sexta
  source 1..190: mol_type = genomic DNA
tagaccataccgttgtcattttgggctgtagtgtatagataataaatatagacgcgtgtactggtgtgacgtacggaagtggagagttgggagcgacagctcaccgctcactccactcccggccgccgcgcgagtagcagtgtcagtgattgcaaaaaaaaaaaaaaaaaaaaaaaaaaaaaaaaaaaaa

SEQ ID NO: 59 - DNA, 27 bp, Manduca sexta
  source 1..27: mol_type = genomic DNA
gccagacgtttcagtttgtttcgaact

SEQ ID NO: 60 - DNA, 60 bp, Manduca sexta
  source 1..60: mol_type = genomic DNA
ttatgaataaatacaatttaataagcattatttattaaaaaaaaaaaaaaaaaaaaaaaa

SEQ ID NO: 61 - DNA, 31 bp, Manduca sexta
  source 1..31: mol_type = genomic DNA
tacttcgggctacacaatgtacgtgaaagta

SEQ ID NO: 62 - DNA, 77 bp, Manduca sexta
  source 1..77: mol_type = genomic DNA
tctccgaaccgaacctgatcttaactgtaaataaaataagcgatacccaaaaaaaaaaaaaaaaaaaaaaaaaaaaa

SEQ ID NO: 63 - DNA, 251 bp, Manduca sexta
  source 1..251: mol_type = genomic DNA
ttggtaaccctgtgggaagtcccaaagtcgaagctcgagaacagttttaagcagaggttttaagtctaatttattggtctctggttttaagttagtgtcatgtgtcaacaataataaagctttatttttggctctatttataagtaaggtatcatcctttcagcgttaaaacaaagggcgtagctcatgttcatgtagttcttttaaataagtgtgaactaagtgtatatgtacatttattactggtgaca

SEQ ID NO: 64 - DNA, 578 bp, Manduca sexta
  source 1..578: mol_type = genomic DNA
cataatatttttatttagatatattgtaagtactaagaatgaagtggtgatagcttagttgggagtggaatggacagccaagacaaatgtcagcaagttcaaatccaaaggcatacacctctgacttttaaaaaaaaatatgtgtgtattctttgtaaattatcacttgctttacggtgaaggaaaacatcatgaggaaactctcaatagtaaaggtatcccattgcccagcagtgggacagtatattatacagggttgatattattattattatatactatgctttgctcatgcgctcattcactccagcattaatgacctttcctggtattgaagttctgctaagtattttatgggataaaaataccctgaatgttaatccacacacattcagtatgtcaaatttcatctaaatccatttaggtgtgttctatttgatactaagttccaacaaaaatcttcataaacgtcaggacccctcaggagcaacattattaataagattaataataaattttcaatgcataataattttaatataatttatattacttaatgttgaagttgatatacttaa

SEQ ID NO: 65 - DNA, 186 bp, Manduca sexta; identical to SEQ ID NO: 52
  source 1..186: mol_type = genomic DNA

SEQ ID NO: 66 - DNA, 297 bp, Manduca sexta; identical to SEQ ID NO: 53
  source 1..297: mol_type = genomic DNA

SEQ ID NO: 67 - DNA, 464 bp, Manduca sexta
  source 1..464: mol_type = genomic DNA
attacgctttgagcagccactttgtaaacactagcctattaactccgtcactgcttcggcgataacacttctgtatatctgttattgtttgttaatttttggcgcaaggatatatttaatatgtgctaagtttgtggctaatttatattatgtttctacattaaaggtatgtattagtgattttttgtaacttatttaattacaaacattataacatattttattagaaaagggttttgtatcctatgtagcgcctgtttcacgatgcctcttgaagtatactgaaatgggtacccgtcgatagtgtttgctttaataggcaacgcatagtgaaaactttgtactttaaccttttcaaaagcatgaataattaaaaaaaaatattagaataatgtgaaaaaggtgtacagtgtgataaatatggagtatacaatattcttgttccagttataagataaggcaca

SEQ ID NO: 68 - DNA, 48 bp, Manduca sexta
  source 1..48: mol_type = genomic DNA
tatttggccaaaattgtgaactgtaaacatgcaataaaatgatgaacg

SEQ ID NO: 69 - DNA, 32 bp, Manduca sexta
  source 1..32: mol_type = genomic DNA
tgagaacaaaaacattagctccgcgtttaaaa

SEQ ID NO: 70 - DNA, 140 bp, Manduca sexta
  source 1..140: mol_type = genomic DNA
ttatcgtgctaaagtaatagttattgtttcacattatgttcaaataaaaaagtaattatttatgttcaaataaaaaagtaattatttaagtgtcttgtaaatctgcgaataaatatacttaatgtataaaaaaaaaaaaa

SEQ ID NO: 71 - DNA, 6369 bp, Manduca sexta
  source 1..6369: mol_type = genomic DNA
atgacgacgttgactgcaaataggcaagtgtcggtgttgcagacgtcgatcccgctgccggcgtcggcggcggcggtgcggcgcgctccgcccgacaaacgaccgctgcccgccacaccagtacatgcaggtggcaagagtggtctttctgcgagttccagccgctccaccagtccattaggacagggtgcagtcagttttataccacgtgcgcctaattcatcatctggatcccggccgaactcgtccctccttgcgcccagtagcaaaataccctccaccgcaacacaagggcaacagcattcacaaacacccacagcccaaaatggaacacccacaaagcagtctatgcttgacaagttaaagctattcaataaagacaaaattagcaatgaaaaacaaaacagcaaaagcactgcagtatcaaaacgcaccagttcttccagtggtttctcatcggcaaagagtgagagatcagactcgagtttaagccttaatgagtcttctaacatacccaacacacatatcaaatcgtctaatttaataggacctaaaacaatacggcaacaaagcgatactttatcaaaagacaaatctggtaaaaatttaaaacctaagttagttaattctaaatctcctagagattctacaactaacttgaatagggctagtagcaaaacaactaataatgataaatcgaggaatagtccaaaacttcctgctagggataaggaatcgaaactagccacacctaaaaccatgagtaacacaaaactaaaccaagttgaagatcaaccgaggggttccaaaactaaagtggaatcgaaaatggtaaagctttccggtagccaagttaagctgagtgaacaaagatccgatactccacaagactgtaaagaaaatgtaactcctaatgcgaaaaatcatcagagtcactctcaaacaccatgtggaggacagggatttggaactccaaaccatacaggaatacctaaaccgactgccgctgtcaagggaacttttaaaatatcaaaagacgatagacatgttatacagaaaagtacaaatagtcaattaagtcctatacaaagcaattctagtttaaattctacaaataacataagtgcacttccattctgtagagaacaatctaatttaagtaaggatataactcaaaagcagacactagccgtatccccgatgccggttatgagcacagggaatcaaaatcatagttcgcaaatgtctgaaagttcacactccaattctacgcacagtactacaggccaacattcgaactctagcgatagcagcgttatataccgtccttctagcgaatcaggttctgaaatgtcgaagactgctagtcacagtgtggcatcaaataaacggctagacatgaatactacttatataaatgacgttataaacgaagcggagatatcagaaaaggaggcagcacaaagacgtcacgtcgataattcaatccccaagcaatcgtttgatcccaataaaacgttaaccgaaataagtaggaggctcgaaagtgatagtagatctagcacaccgtcgcattgtcgtgataattctttaggcgaagacgagaatccaatgatgaatgttttaccaatgagaccgttacttcgcggatacaacagtcatttaaccttgcccatgagaacttcaggtttggcacagaaaaatataagcgtttatccacaccacgcaaatactgtaaaagcaaactttggacgggaaaacataggtcttcgcgatcgtataaactatgggcctggtttttcgaatcctgattattgtgatctggaaattgcttcgggatacatgtctgatggtgattgtttgagaagaattaatatcggtgagatggaatgcgggaggaacaacgacatgatggacggctatatgtccgagggtggtgcttcactctacggtcgtcggatgaactaccagcaaccacaatttcaacaaatggacgaaagacgaagtggtggtcgcaatagaggcatggagggtggtagtggtgtagtctacagagtagtgggtcgcacacgcagcaaagccgactccggacagcagaccgagcgccagcctcagccttcccggcaggacaccacgtggaagaagtacactgactcgcccgggaatcagccagcacccgcgcctcctagtccatctcactcgcgcaagggtgagagacgaactggccatcattcaccgcagcaccacaaacgagaaaaactcactgcagctcagcagcttgggatcgcacctcatcctcagtatgccgctccagctcaagcagctaaccctgctggtcagcattcctctagaaacccaggcccacagctccagtctccgagtggagggtcgcgtccctccagcgtctctagcggtggtaatggcagtgcctgctcgtcgaaggctaaagttcctcaaaattttggatatgtgaaaagacaaaacggtgtacagcagccggcacctcaggccaacggtccaccgccgcaacatggaggccacactggaagaaccgcccaggtgtctgcagtgccaagaactaaagtcaaagtttcgggaggaacccagacatgcacacaggatttacagattcacaaaaatggtatcggacctaaatcgtattctctgggcggcaccgcggctgcccaactgtcagcgtcagtgcgagagagactgctcgggtcacaatcactaccaaaacctggaacacacgaattcgcagcgttattccatcaccacagaattgcacccagaggcggcatgaagatcagcgatggcagcctttctgacacacagacatactctgaagtgaaatcagactatgggataccctacgcgccctggctcaggcatagcaacacatactcggcgagcgggcggttgtcagagggagagtcgatggagtcgctcacgtcgctgcactcggcgcaggcgcaggcgcacaaccacacctcctcccctaactcccaccgcagctcactcacacacaataagctcatcatgcaccgagatgcacagggctccaggcttaacaggagcaacagcattagatcgactaagtcagaaaaattatatccgtcgatgcttcaaaggtcttcagagagcgattatgaaccgtattattgtttacctgtgcagtatggaccaacaggtcaaggtctaaactatggtgtgtcggagccgccgtcgccgtcgccgcgctcggcgctgagcccgacgcacgcgcccggcaacgtcatgcacacgccgcgccactcacaccactacccgaaaaagaacgacgatgtgcacggttcgacggcgtcgctagtgtcgaccgcctcctcgctggctgccggcgcaggcgcagaagagaggcacgcccatgaggtgcgaaagctgagaagagaactggccgatgcgaaagaaaaagttcacactttaacaacgcagttaacaaccaacgcgcacgtggtgtccgcattcgagcagagtctgtcgaatatgacgcagcgcctgcagcagctcaccgcaaccgccgagagaaaggattcggaactgacggagcttcgtcaaacgattgagttgctgcggaaacagtcgattcaagctggtctgactactgctcacatgcagtccatgggaatccgtgccgatggcgtcaacgtcactggacagcaggacccaacaacccaaacgcagcagtcatcaccacaacgtaacgcacaaacgggcaacggtgcgatcacgcggcatctatcgaccgacagcgtgtcgagcatcaacagcctgagcagcggctcctcagcgcctcacgacaaaaaacacaagaaaaaaggatggctacgctcatcgttcacgaaggcattctcacggaacgcgaagatatctaaaacagcgaagcactcgtccctcgggcagctgtcgtcacaggacagctcgtcgggctcgcatcactacgacgacccgcacacgatacgagagggcagtaatgagaacagtctagaacattcccacgaggctcttcctgatactaaggagaagccggcgcctgtcaagcctgaagaacaaaccaaagataacaaagaggaatctgtcttggtggatgagctgaagcggcaactgcgcgagaaggatctcgtgttgaccgacatcagactggaggcgctcagctccgcgcatcagttggagagtctcaaagacaccgttataaaaatgagaaacgagatgttgaacctgaagcaaaacaacgagcgtttgcagcggctggtgacgtcacgctcgctcgccggcagccagagctcgctcggcaccggcggctccgccgtcgaggacccgaggcggttcagcctcgcggaccaggccaccatgcaccaggcggcgatcgacattcacgcccagcctctcgacctcgacttcaactgcatgtcagccacaccaaccatcgacttctcgaagaaaggttcacccaagtccggcatggtggagcccatatatgggaataaggctgcctgcgaactgaacgagaacaatgagacgctgctaggagcgttgaatggggccagcgatttgttctccaacggactcaatgctggagagcggctgagtggagattacgatataaacagcgtgttaccgccgcctaaaacacgcgagcttgctattggtgaaagctactctgatattggtgtagcagacagccagggagacacgactgatggaaagaaaatagcgatagctgtatatttaggtcaacccgaaacattccaaagatatttcgaagaggtccaagacacattgacggagtccgagtgcagattctacgcgaagcaatcggcaagcgcgtacaatcatttcgagaaacaagccagtttcgaatcgccgagaatgtccacgaatcacagtccagaagtggagacacaagactacccgcaaataaataagtcgaacacgaatagccttaaaagcaacaaatctacgcacagtagctcgtataagaatgtttataatagtgattcgacaataaactgcaacgagtacactatagcgtacacttatatatctggcaagacgacttggcagaatttagattatatagttaggaagtcctttaaggactacttgtctaggatagatttgggcacgaatctcggtctgaacactgattctataacgtcgtaccatttgggtgaagcgacgcgtgggccggagatcggcttcccggagctgttgccatgcgggtacatcatagggaccgtgaacacgctgtacatctgcttgcaaggagtcggcagtctggcgttcgatagccttataccaaaaaatattgtatatagatacgtttctctgctatcggaacacaggcgagtgatactctgtggtcccagcggcaccggcaagtcatacttagcggcgaaactcgctgaattttacgtccagaaaacacaaaggcgcggcaatcccgccgaagctgtagctacattcaacgtggacagaaagtcgtgcaacgagctgcgcgcgtacctggcgaacatcgcggagcagtgcggcgcggcggccgcgggcgaggaggcgccgctgccgtccgtcgtggtgctcgacaacctgcagcacgcctccgcgctcggcgacgccttcgcggggctgctgccgcccgacaacaggaacatgcccgttattatcggtaccatgtcccaagcgacgtgcaacaccacgaatctccaactacatcacaacttcagatggctcctcaccgctaaccacatggaacccgtcaaaggattcttagctaggtatctccgaagaaagttattctcgctggagctgcggctgggtcggcgcgagccggcgctggcggcggtgctggagtggctgccgggcgtgtgggccgcgctcaacgccttcctcgaggcgcactcctccagcgacgtcaccgtcgggccgcggctcttcctcgcctgccccatggacttggaggccagccaggcatggttcgcagacgtgtggaactacagcatagtgccgtacgcttcggaggcggtgcgcgagggcatcgcgctgtacggcaggcgacgacacgccgccgtcgacccgctgcagcacatcaagtctacatacccctggagagaacccaatcactcgcatactttgagacccataacagttgatgatgttggcatcgaagagtcaagccaagactccgccgtgaacaacaatcaagatcctctgttgaacatgctgatgcggctacaagaagcggcgaactacagcggaaaccaaagccaggactctgacaacgccagcatggactcgaacctcacacacgacagctctgtagggaacgagctttaa

SEQ ID NO: 72 - DNA, 591 bp, Manduca sexta
  source 1..591: mol_type = genomic DNA
atggcgtgtgctacacttaaaagaaatttggattgggaatccatggcgcaattgcctgctaaaaggcgaagatgttcgccatttgctgcaagttctagcacaagtcctggatttaaagtgtctgaaaccaagccatctacattcggagaggccgttagtgcacctgtgaaaatgaccccagagcgcatggctcaagagatctgcgacgagatcaagcggctgcagcggcgccggcaactgcggctggctggcagctccgccgcttcgtgctcatcgtcgagcggcagcgagggcgactgctcgccgccacatcgctcctcgcacacttcgcacaagatgcacaaccgtgcgctcttcactttcaaacaggtgcgcatgatctgcgagcggatgctgcgcgagcaggaggtggctctgcgcgcggagtacgagtcggcgctcagcaccaagctcgccgagcagtacgaggcgttcgtgcggttcaaccttgatcaggtgcagcgcagacccccgcccagcacgtgcatgcccctcggcatggacgccgagcatcacatgcaccaggacctcgtacctagctatctgtcctaa

SEQ ID NO: 73 - DNA, 969 bp, Manduca sexta
  source 1..969: mol_type = genomic DNA
atgagtgccaaaaaaggatatgaaacgaagattgtcgaggaagaaaacatggattccggaattgtgtctggtgaattggaatcttatgagatttcgggtgaagtggattcgggcgtgattgattgtgataagaaatacgaaggggttccaagtgaggtgttggaattgacggacaagttcaaaagtgtaaatgtgagagagaagagctgtcctgatgttccaccactggcggacctgttccaccctgacaacgacggagatacacaactacacattgcatcggtacacggctgcgagaaatcagtgagcacgatcatcagggtgtgccctgacaaggagtggctggacctgcccaacgactacggccacacgcccctccacctcgcggtgatgagcggcaatgccgtggtgacaaggatgctggtgatagccggcgcttcgctcgctattcgcgacttcatgggagagacgcccttacacaaggcgaccgcagcgcgaaaccaggagtgtctcaaagccctgcttgcccctgtaccggaacagcccaataggaaattgtcttcaatactcgaccagaggaactataacggtcaatgttgtgtccacctggcggcgtcaattggaagcgtagagacgctacagaccctggtctactacggagccgatatcaatgccagggagaacctggcgggctggacggcgctgcacatcgcggcgcggcgcggcgacgtgcgcgtggtgcagttcctgcggtcgcgctgcgccggcgcggcgacgcggccgcgggactacgccggccgcacgccgcgccgcctcgcgcgccgcaccaaggccgccgccgccttcgacgacaaggacgacagcgactccgactccgactcggacgatgatgatatgtacgacagtgatagcgagacgttgttcgaaaaactccgcgagagcctgagcacgtcgatcaacgtcgcctga

SEQ ID NO: 74 - DNA, 549 bp, Manduca sexta
  source 1..549: mol_type = genomic DNA
ccacgacaaccactgatgaagttattttttatagcaattcttttcgctgccatcgtcgcttgcgcgtgcgctcaagtgtcgatgcccccgcaatacgctcagatatatccagaatattacaagtactccaaacaagtccgccatcccagagacgtgacctgggacaagcaagtcggcaacaatgggaaggtcttcggaactctgggacagaatgaccagggtcttttcggtaaaggaggctatcaacaccaattcttcgatgatcaccgcggcaaactgacaggacagggttacgggtccagggtcctcggaccttacggagacagcaccaacttcggcggccggcttgactgggccaacaagaatgctaacgctgctcttgatgtgaccaagagcattggcggtaggactgggctgactgccagtggatcaggcgtgtggcaacttgggaagaacacggatttatctgcgggaggcactctgtctcagacgcttggacatgggaagcctgatgtcggcttccaaggtctcttccagcatagatggtga

SEQ ID NO: 75 - DNA, 1344 bp, Manduca sexta
  source 1..1344: mol_type = genomic DNA
atgagggaaatcgtgcacatccaggctggccaatgcggcaaccagatcggagctaagttctgggagatcatctctgacgagcatggcatcgaccccaccggcgcttaccatggcgactcggacctgcagctggagcgcatcaacgtgtactacaatgaggcctccggcggcaagtacgtgccgcgcgccatcctcgtggacctcgagcccggcaccatggactctgtccgctccggacctttcggacagatcttccgcccggacaacttcgtcttcggacagtccggcgccggtaacaactgggccaagggacactacacagagggcgccgagcttgtcgactcggtcttagacgtcgtacgtaaggaagcagaatcatgcgactgcctccagggattccaactcacacactcgctcggcggcggtaccggttccggaatgggcaccctccttatctccaaaatcagggaagaataccccgacagaattatgaacacatattcagttgtaccatcacccaaagtgtctgatacagtagtagaaccttacaatgcaacactgtcagtccaccaactcgtagaaaacaccgacgaaacctactgtatcgacaatgaggctctctatgacatctgcttccgcacgctcaaactttccacacccacatatggcgaccttaaccacctggtgtcgctcacaatgtccggcgtgaccacctgcctcaggttccccggtcagctgaatgcggatctccgcaagctggcggtgaacatggtgcccttcccgcgtctgcacttcttcatgccgggcttcgctccgctcacgtcgcgcggcagccagcagtaccgcgccctcaccgtgcccgaactcacccagcagatgttcgacgctaagaacatgatggcggcgtgcgacccgcgtcacggccgctacctcaccgtcgccgccatcttccgtggtcgcatgtccatgaaggaggtcgacgagcagatgctcaacatccagaacaagaactcgtcgtacttcgttgaatggatccccaacaacgtgaagaccgccgtgtgcgacatcccgccccgtggtctcaagatgtcggccactttcatcggcaactccaccgctatccaggagctgttcaagcgcatctctgaacagttcaccgctatgttcaggcgcaaggctttcttgcattggtacaccggcgagggcatggacgagatggagttcaccgaggccgagagcaacatgaacgacctggtgtccgagtaccaacagtaccaggaggccaccgccgacgaggacgccgagttcgacgaggagcaagagcaggagatcgaggacaactag

SEQ ID NO: 76 - DNA, 588 bp, Plutella xylostella
  source 1..588: mol_type = genomic DNA
atgacgttgtcttttggcgtgtttctgctgatatcttcagtgttttgttgttgtgctcatgcagggtgtggcgtggtgaccagacagcagtgggatgggctggacccgatacagttggagtacctgccccggcccctggggctggtggtggtccagcacaccgccacccccgcgtgtgacactgacgccgcgtgtgtggagctggtgcagaacatacagaccaatcatatggatgtgctgaagttttgggatattggaccgaacttcctgattggtgggaacggcaaggtgtacgagggccctggttggctgcacgtcggcgcccacacttacggctacaacaggaagtctatcgggatctctttcattaggaattttaatgctaagaccccaacaaaagcagcgttgaatgcggctgaagcattgctgaagtgtggagtgagagaaggacacctgtctcactcatacgcagtggtcggccatagacaactgatcgcaacagagagcccaggcaggaaactgtaccaaatcatcaggcgctggccaaactacctcgaggatattgataagattaaaaacaacaagtag

SEQ ID NO: 77 - DNA, 1986 bp, Plutella xylostella
  source 1..1986: mol_type = genomic DNA
atgtctatcctaaaatcaaagttattcgaaactattgcaaaaagtttcaagtctgatgcagtcccgaagccacctagagaaccggtagagactacagagacatcacaaaataataccgaaaatcaaccttacaatgtcgaagaagaggaaatacccgaaccagaaaagcctaagaaagaaaaaaagaatcccaagcctaccaaaaaaactttctttaatcgtgacaaaactaacaaacacgacgatacccgcaaacatacaaaatccggaaaggaccagacatcaattaatactcaaggtaacttgaaaattatacttcctgtaacttaaacggcccgttgaccccagtttacctttcgcctttcttgatatatttttgtaatccagccttactttggtaatacatacttgccccacttgtatttagttaatggtggcactagctagatagtaatgttaaatgatgataagcagtagtgattcatcattcaaatgtatcattgtcctttaatgttaagcgcaaatagattttcattgttctcccatgtgcttcatgttttatgtatttataggtaggtacttaatgttttataaatatttttttgttaattgggaatccccagtccccattgtctggaccagtttatatataattgaactaacaagagtgtgctttaaatactattctctgcaattatgataattaaacaacatgaatttctcttcacttcccttctcttatttaaataatattgtaggaaactgtaataactaatacaagattataaatttcattctagcaactggtgatgtaatccatgtggtaaattccaaagatgtgcaggtcggccatcagtatgtgtacaacatgggaactcccggagctaactcacagaagaataacccatttgatgatgaagaaacagtagaaaagacaaatctaataactctggtcatggaagcaaaaattatggtaataacacatttttaactaggcataaggtcataatttagccagaatcatcagcttgtcctgtggctcttgttgagctggtggaaagaatacataggtaatgaatattttgatcaatactcattgcaaaaatcacaataatgccattgaaaatctataacatgttccttaagtatcacttatcatcaatccaattaagtcatcacacagatcaatcggttagttctgtttatttacttctttcagctggaacatgaatacatggactatgtctcgaagaacctcggcaggaactggcacagcttcttcagaacgctcggcttcacgcgggggcgcatcgagactgtggaactggatgagggcagaaatggtgttgcagaggtatgaaaaaaacatacatgttaatttgtgttgtttggctgatgtggcaactaagttcgtaactgctatgatcaactgtttgtgtcataggtattttttttgcccttccacacatctgaggtaacaaggacacctcctgcactcaaaacacaactgctatgtcctactgatgagtcctaggaaactcagaaaccatctgtttcccccattctcaattccataaagctaaaaagtggtagttacacaattcacaattcataaacatgctttgtcatagttagaaaagcagctcagctatgaggcatccactccacagtccactcacgaaagccctctttaaaaacataaaatcatcatcatcagccctcaattgcccactgttgcatataggccttcttttgattatgccaagtttttcgtttctctagtccacagacttgacatagttgtcacaaatttattctacgtacttgtatttccaggtgcgctacaagttgctcctggagtgggcccgcaccgacgaggaccccacgctggggcggctcgccacgcggctgtgggacgagggagagcggcagaccgtcaaggaactcgccatcttgtataataataatttcaagcaacaatgttga

SEQ ID NO: 78 - DNA, 1572 bp, Plutella xylostella
  source 1..1572: mol_type = genomic DNA
atggtagtggtgaaaagccaattaaaaatgcaaagtgaccaggacacggactcgtccactgccggtgcttcgccgcgcagcttctacatcgagtcgccgcacagctcgccgggacaacaagtgccttatttaactaattacatgacagtactgtcctgtgcagataataacttaatggatacaggaagcaatggaccattcctgagcatcacggagcagccatgtgaccacttcaggttccgctacaagagcgagatggtcggcacccacggctgcatcgtcggcaagaccagcgccagcaaccgcaccaagacatacccttctgttgttctgctcaactacaaaggccgcgccaccatcaagtgcagcctcgcgcagcacaacaaccgcaagcagcacccgcaccagctggtcgaggatgaccaggagcgcgacctgagcgccgaggtcaaccccgagaagggctatgaagttggatttcgtggcatgggcataatacatacagcgaagaaagatgttccggcactcctatacaagaaattgagtgagagactgccacatttcaatgcccgtgagctgaaggcccagtgtgagaacgaggcgcgcagtataaacctcaacatcgtgcgcctcaagttcagtgcgcacaatgtcgacacggacgaggagatatgcgctccggtgttctcggaacctatccacaacatgaaaagcgccgcgacgaacgacctgaagatctgccgcatgagccgcacgtcggggcgcccgcgcggcggcgacgacgtctacctactcaccgagaaggttaacaaaaagaacatcgacattcgcttcgtgcaactggagcgcggcgaggtgtgctggaccggcaaggccaggttcctcatgagcgacgtgcaccaccagtacgctattgtgatcagaacaccagcatacaagaaccccgagattacgtctgacgtaaaagtgtacgtagaactattccgcccgtccgatggccgctccagcgaacgcatagagttcacgtacaaggcagaagaagtctacaagcaaagcaagaaacggaaggccaactcttactcctctatcggaagttcatctagcggtaattctatcaaaagcgtcagtgatcttccagcaactgttattatggccaatgaaatgaatgcggctaacaacaactttagtaaaatctcttcaatgctgagcccaaacaatataccagaaataccgacacaaacgactgtgggtctatcagacgctctctacgacataacagtgacagaagaccaccaaatgcacatcagtcccatgctatgccaaccagtggaagagtatcccctaaagcttaactcgcaggatatcatacaagtgaatttgaactctaaggacatcgaccaactgctcaaagtcaacagtgtgcctgataccgataaagactttgctgacttcaattttagtgactactacaaggcactcgatagcaactttttagctgatggtggtggtgatagcttcagtcagtgtatcttcaactctatgcaactgaggcctgactctgggagaggcacg

SEQ ID NO: 79 - DNA, 1242 bp, Plutella xylostella
  source 1..1242: mol_type = genomic DNA
atgcctaaagtaataatcgccagttttgcttatatagtaggtgtgtttgtcctgtgtgctgggctagaaacaagtccaacctgttccagcatcgaaggacctactccaggtgaattcataatacaaagaggtatcgtaccggacaatgattcagccaccacagtaagcttccgaggctgccgaatatctgacattcagccgagagcattccatgggctaccttccctgcaatacatagacttatcaagaaatagcattaaaaacctgaaacttggcattcttaatgacgtcactagactcactcatctgaacttgtcttataacttcatcagcgatttagaagagagtttgttcaaccaatcgtcaaggttggaggtgttggatctccggtggaataagattgaagttatgaaagtgggcgttttcagtccattgaagagattgaagtatttggatctgtccgacaacgaaatagttggggccagcctgagccccgctatgtttgattcctgtaaagctctatccactatcaatttttcaagaaatgatatgtctggtgctcccactgatttgcttcgagctgtggaggtactggacacactgaaactggatgggtgctttttgaaacaagttccggaatttgctacgaggagcaataccggcacaatgaagaaacttattttatcatcgaaccaagtgagcactgtgaaacttactacgttcatcagtttaacaaacctggaggaactagatttgagttcaaatgtaatttcagaattgcatgaagacgtattcaagccattaaaaaacttgaaaattattattttacgctcgaaccgactggaaaaaattcctgataagttgttttataatatgttacgattaaggaaggtagatttatcttttaattctttgatgataatacctgtgaatgcctttcgttttacgacgatagaaatgttgaatatatcgcataataagttcacatatttagtcgacaacttttgtttggaacttagaaactcgggagtgaaactgaaaaagttttacttcaacagtaatccctggcagtgtccttgcttgagggatttattaaaggaaatgaagacgtatagaatatcgtacaataatgctaaatacgatggtaaaaatgcagtttgtatttcaggagacattattaatacttgcttgagacaacctgatgtcaatgaacacttcaatgatttgtactattctgactaa

SEQ ID NO: 80 - DNA, 1053 bp, Plutella xylostella
  source 1..1053: mol_type = genomic DNA
atgagtttcaagaaggatttcgacacctcaaagaagatccaggaggatgaaaacacagactctgggttcctatctgggccgataagtgagcagctgacctcggaagattgtgatttagcggaggaaagtgagcgtgctcgcagcaggcttagtgaggaagatcctgagcctgagctgcagttggacagtgggctggacctctcggagtgtctgtcgagtgttaagcttagtgatagtgcagtgtacacacccacctcgcagaccacccccacagtcactataggtgatgagaaaactcatgacatcccacccctcgccatcctgttccagcaggatgacgatggagacacacaactacacattgcagcggtacatgggtgcgaaaaatcagtaggaacattagtaagagtttgccctgacaaagattggctaaatgtaccaaatgactttggacagaccgccttacacttagcagccatgagtgggcatgcagtagtcacacgcatgctggtgatggccggtgcatctcttggcattcgagaccttgttggcaacacacctttacatgtggcagccgcagcgggctacgtcggctgtctccaagctttactggctcctgctccagaacaacagcagagaaggctagcatccacgttgaaccagaaaaattacaatggtcaaacgtgcgtccatgtggctgcgatggccggccacgtcgacgcgctgcagacattggtctattacggagctaacatcaatgctgcggagggtctatgcgggtggacacctctacacgtagcggcggcgcgaggcgacgtcgacacggctcgctacttgctcgagaagtgcgctggcgtcgatccctctgccctggactacgccggtcgtacggccaggaaactggcgttgaagaataaagcggccgccctgtttgacggcagtgagggcagcgaggaggaggatagtgacagtgaggatgagatgcttctggaaagcgaccagagtctgttcgaccggatccgtgacggtatgaacgccatcaacgtcgcctga

SEQ ID NO: 81 - DNA, 1380 bp, Plutella xylostella
  source 1..1380: mol_type = genomic DNA
atgaacgcgcccgccgactccgccgtggtgacgttcaccaacctgggcatccagtgcgtgaagcggagagacatcgaggacgccctggctgtgagagaggagatgcgagttgaccccttcaagaccggattcagccacaagaactccccgcaaagcatcgacctgaacgccgtccgactctgcttccaagtgttcctgccggacgagcgatccggcaagatccgccacgcgctgccgccggtcgtgtccgatgtcatctatgacaagaaggccatgagtgacctggttatcacgaggctgagtcattgttctgcgcccgcgcagggcggcaagcaagttatattgctgtgtgagaaggtggcccgcgaagacataaccgtaaccttcttcgagaagtccggcgagcgcgtgacgtggcaggcggacgcggcggacgtgttcgtgcacaagcaggtggccatctgcttcaccacgccgccttaccgcgacccgcatgtgcaggaccatgtgcaggcgtacatccagctgcgtcgtccgacggacaacgcgacgagcgagccgctccccttcgagctgctcccgtccagcgcagatccgaattatctgaagcgaaagcgacagaaaccgatacagaacttcagtcggtacttacagccgatcgatagcgacatgaagcagcagctgccggactatttccaggacaacatggcgctgtccagcatcccctccgtgaagctggagccccgagataagactcctcctcacaacatgagcagcccgccgctgctgttcccccccgcgcacgccgcgcccgcacaccatgacccctacgcgtggaacatgcaactagacaacatgcagtcgggtctgacggcgcccgggccgagccgcctgccgcagtacagccaggacatggcctggaccaaccagatgggccacgtgtcccctatgcaccaggccatgtcccctaacatgggtcacgtctcccccatgcatcaagccatgtccccaaatatgggccatgtctctcccatgcaccaagctatgtctccaaatatggtccagtcacctatgggtcatgtgtcccctaacatgggccatgtatcccctaatatgggtcatgtgtcccctaatttgggtcatgtgtcacctaacctgtgccagcaaccaatggctcctatggcgcagcagctgatggacccgtcccccagcgacccaccctccatcacggggctgctgatggatcgcccggaccagccctactccggggagctgtctggactctccgccctgctggctgaggcagcccccgcagagatgctcagcgatagcctcaacagactgtctacgggggacttgttgagacaagttgatatgtga

SEQ ID NO: 82 - DNA, 1244 bp, Plutella xylostella
  source 1..1244: mol_type = genomic DNA
atgactttaattttcaagagtgttttatttttgggcttaatattgactacttttattgtttccgctcagcctgtgaaacaagatggcggctcagcacacgaagaaatattgttccgtgagcacggccagccggtggtgttgacctgcgcgcgcgcagacgaccccaaccaaggtggcattagaacgtggttgagaaacgggacgccattagaagacggcaaaatgtctcccgaaataaaattcctcgacgacaaatcactctggtgttgcagccctcaccagccgtggaaggggtctaccaatgcttcaccgaaacccataagggcattgcaacctccccgaaattcagcgtgaaacagacttatctcaaagctccagagactacgccttcagtcaatatcaaaccagcaaaaggccttccctttagcttggactgtgacgtccctgaaggatatccgaagcctgaagtgcaatggttcctacaacacgggaaagatcacaccctgattgaggcaattatcaataaacggatcacacaggctccgaacggagctctttacttctcaaatgctacaaccgaagatgtgaatgtgggagactttagatacgtctgtatggcgaggaatgatgcggtagacttaccagtggtggtgtcggaagctgtcatcacaggtctgagcagcgagggtggtaagggtagattggtggagcagtacgtcagtaaagaagttagggcggttgcgggggagaccacagcgctattttgcattttcggtggcaccccactagcccacccagactggacgaaagacggcaagaatgtgaacggggcgcccggcgaccgagtgacccgacacaacaggagctcaggaagacgactcatcatcaagaacaccactctagaagatgctggaacttaccagtgcgccgttgacaacggcgttgggactgaaatgcgttctgtcaaggttactgttgaagcgaaaccttcaataacgattgtgaatgaagtagcagcgaagcttggagaagaagtcaagatttgcgaagcgaccggagtcccaacgccaaaactaacgataactcacaacgctaaaccgttggttgcgtcaaataacgttgttataaccaacgatggagttgttataaagaatattcaggctattgatcgagggtattatgggtgtgatgctgtcaacgagctaggaagcgaatttcgtgaaacttatctaagtattgcctga

SEQ ID NO: 83 - DNA, 1191 bp, Plutella xylostella
  source 1..1191: mol_type = genomic DNA
atgcagcttaatttctgtatcaatgtgatcgcgaccatattgttgattgtgactggcggggattcccaaaaacgagtgggtgacatttgcattgatcaatacacgaacacgtacggaaggtgtgttttctcggatcgatgtccatcagctttacgtaattatcaacagaacggcattcggccatcaatatgcacttacaacttcgacaatgcactggtgtgttgtactgaacgcggaaatattcttcaaacggcgaggcccccgccaccgcctgatcaggaagacagatttcagtcctctgctggaaacaacaacaacaacaataaacctaacattagagttagcgaaagaaaatgccgcgagtacagcaaatcagtaacgttcacggtgagcttcagctcgctgctgccggagcccgagttgcagtccatctcgcggccgcgctgcagccggagcggcgtggggctcgtgctcggcggccgggacgccgcgccggaggagttcccgcacatggcagcgatcggcttcgcatcagcggaaggctacgacttcaagtgcggggggtccctcatcagcgcgcgctggccgctgaccgcgccctgcgcgcgcgcccgcgcctccagccggcccgtggtggcgcgcttaggagataggaatatcaacccgaaagcgcaggacgacgccacgcctgtcgacgtgccaatccggaacatcatcgtggacgtggacctcagcaacagcatccgccccgcgtgcctgtggcccggcggacccttccacgaggataaggctatagctacgggctggggggtggtgaaccaacgcacgcaagagaaagcggacctcctccagaaggtctcgctcactctgctcgagaactcatactgcgaccgtctgctgaggaacaaccgcaaccgacactggcagggcttccgcgactcgcagttgtgcgccggcgaggtgcgcggcggcatggacacgtgtcagggcgactccggcgcaccgctccagatcgtgtccaaggagaaccagtgcatctaccacctcatcggcctgacctccttcggctacaagtgcgcggagcagaacaagccgtcggtctacaccagggtgtcgacttacgtggactggatagagtctgtggtgtggccggaggagtatgcggcttgggcggcggggaggagtaaataa

SEQ ID NO: 84 - DNA, 2046 bp, Plutella xylostella
  source 1..2046: mol_type = genomic DNA
atgatagtgaaaatagccattttggtgatagcaataacgttcaacgatgtgtctgcgaaaacttcgtacaagatctgcgtaccgtctcagttcatgaaggcatgtgaacaaatgcttgaagtggaaacgaagagcaaagcgatactggaatgtttgccggccagagatcgagtggaatgcctgaccctggtgcagcaacggcaggcggacctcgtcccagtggaccctgaagacatgtacgtggcgagtaagctgcccaaccaggactttgtgcttttccaggagttccggaccgatgaagagccggatgcggagttccgttacgaggccgtcatagttgttcacaaggaccttccagttaccaacttggaccagcttaagggcttgaagtcatgccatactggaatcaatagaaatgtggggtacaagataccactaacgatgctgatgaagcgctccgtgttccctgcgatgacagaccgcagcatctctcctaaagagaacgagctgaaggctctctcgacgttcttcagcaagtcctgcatcgtcggccagtggtcgcctgacccgaagaccaacactttctggaagtcccaatccagcaagctatgctccatgtgcgaggaccctgccaagtgcgactaccccgacaactacagcggctacgagggcgcgctgcgctgcctggcgcacaacggcggcgacgtggccttcactaaggtcatctatgtgcggaagttctttgggctcccagtaggcacaagcccggcgactccttcttctgagaacccggacaacttcgcgtacctttgcgcggacgggtccaaggtccctatcagaggaaaggcatgttcttgggccgcaagaccgtggcaggggttgttgggacatcaggacgttctggccaaattgtcgcctttgagggagaagattaagcagctgtctagagctggagcagaatcgaagccggagtggttcaccaacgttctaggcctctctgaaaagatccacttggtcgccgacaacattcccattcgtcccgtcgactatctgcagaaggccaactacactgaggtcatcgagagagggcacggcccgcctgaacctgttgtgagactctgcgtgacgagctcggtggcgctggcgaaatgccgcgccatgtccgtgttcgccttcagtagagacatccgcccccggctggactgtgtgcaagaggcttcggaaagcgattgcttgaaaagtgtccaagacaatggctcagacctggcgtcagtagacgacatgcgggtagcgtcagcatccaacaagtacaacctacatccagtattccacgaggtatatggagtcagcaagacccctaactatgcggtagctgtcgtcaagaagaatactcagtatggaaagattgaggatttgaggggaaacggtcctgtcacaatcctttatggaagcttcagtggctttgatgcccctctgtactaccttattaataagaaaatcataggcactgaacagtgcctgaaaaagcttggagaatttttcgcagccggatcttgcttacctggagtaggcaaattagagaacaaccctacaggagataatgtcgataatctgaagaaacaatgttctggagacaacagcccaataaaatgcttacaagaagacaaaggagacatagcatttgtgtcaagtgctgacctgaaaaacctggatgcctctcaatatgagctgctctgtctaaacagagagaacggtgggcgagactcaataaccaactacgctacatgcaacattgccatggccccatcccgaacctggctctcagctaaagacttcctgtccgatgtgtccatagcacacactccgctgagcttagcacaactactggataccagaaaggatctgtttaacatttacggagagtttttgaagaataataatgttatttttaataatgctgccactggactggccacaacagaaaagatggactttgaaaagttcaaggcaatccatgatgttatctcatcttgtggtgtcgcataa

SEQ ID NO: 85 - DNA, 1440 bp, Plutella xylostella
  source 1..1440: mol_type = genomic DNA
atgctgcttaggacgatacatttgttgttaattgtttgttgtgcgtggtgctatgaagtgcctccggctaaattggaagctatttatccagctggcttgcgagtgtcaatacccgacgatggcttctcgctattcgccttccacggcaagttgaacgaggagatggagggcctggaggcgggccactggtccagagacatcaccagacccaagaacaaccgctgggtcttcagcgataaacaggccaggctcaagataggggacaaagtgttcttctggacgtatgttatcaagaacggactcgggtaccgacaggatgacggggtgtggactgttgaaggattcgtcgacgtcgaaggcaacccggttgaccccgcgaatggacaacccatctcagcgccgaccagacctccaacccaaccaggccgggtgccaaatgtccccatgccgtgtgacatctcagtcaccacggcatcagtgccagggtacatctgcaagggacagctgctctttgaagacaacttcaatggggctctggagaaaggaaagatatggacgccggagattatgatgcctgatgaaccggattacccgttcaacatctacctgaacgacaggaacctgcgcgtgagggacggccggctgtctatcaagcccgtcacgctcgagtccaagtacggggaggagttcctggccaaactagacttgtctgccaggtgtactggtaacgtgggtactacccaatgcagcagagagtccattggggcccagatcatacctccgataatcacagccaaggttaccaccaagaacaagttcagcttcaagtatggaaggattgaagtgagcgccagaatgccgcgcggtgattggttgattccagatattctgctggagccgaaagaaaacctttacggagtacgcaattacgcgtcaggtctactcagcatagcctcagtcagaggaaacactgcttactcgaagaccctcaaaggaggccccatactgtgtgacaaggaaccgcagagaagtgccaagttgagcgaaaaagttggatatgaccattggaataaagccttccataactacaccatgatttgggcaccaagtggcatcaccatgctggtggacggcgagcagtacggggacatccgtcccggcgacggcttcagccaggacccggcggtgagcagcgtggtggccgcgccgcagtggctgaagggcaccagcatggcgccctttgatgttatgttctacatatcccttggtctccgcgtgggcggagtgaacgacttccccgacactcctgagaagccgtggaagaacaaggccactaaagccatgctgaatttctggaacgcccgggaacagtggcagagcagctggtttgaggacaccactgcactcctcatagactatgtcagggtttatgcgctgtga

SEQ ID NO: 86 - DNA, 528 bp, Plutella xylostella
  source 1..528: mol_type = genomic DNA
atgtaccgatttgcagttattttatctgtagtcgccgcgtgtgccgtggctcaagtttctctacctcctggatataatgataaatacccaggcttctacaaatactccaagctagcccggcatccgcgacaagtgacgtgggacaagaatgtcggccgtgggaaggtgttcggcaccctcggcggcactgacgatagtctctatggtaaggcgggctaccgtcaggacatcttcaacgaccaccgcggccacctgcagggtgaggcttctggcaccagggtactcagtccctacggagacagcagtcacctgggcggtagactcgactatagcaacaagcacgccaacgccaacctggatgtcagcaagcggatcggaggcgtcactagttggcaagcagaaggcaaggctagatggccgattggcaagaacagtgagctatcagccggcggaatgatcagacaagaccacttcggccacgggagaccagactacggagtcgtcggtgggtttaaatctaggttttaa

SEQ ID NO: 87 - DNA, 4703 bp, Plutella xylostella
  source 1..4703: mol_type = genomic DNA
atggcgacgtcggggggagtgcgggggcggcgggaggagggcagcgacaactcggacgacgagctgaccccgctccagcaggagatctacggcggcagccaacgcacagtacaagaaacaaaaggatgggatgtgttccgagagatcccgccgaagcaggacagcgggtcgatggagagccagcgctgcctggagatcaccgtgcgcatcatgaagatcctggcctacctggtgaccttcgtcgtggtgctgggttcaggggtgctggccaaggggtctgtgctcttcatgacctcgcagctgaagaaagatagaagactggcgtattgtaataagaatttaggtagagataagcagtttatagtgacgttgccggacgaggagcgggtggcgtggatgtgggcgctgttcatcgcatttatggtccccgagatcgggacccttatcagatctgtccggatatgcttcttcaagtcctccagaactccaagcagcgctcaatttattgtgatttttgtatcggaatctctccacaccatcggattggcgcttttgatgttcaaagtgttgccagaaatcgacgtggtcaaaggagctatgataacgaattgcctctgcatcattccagccattctggggctattgtctagaaactcaagggactcgaaaaggttcatgaaagttatagtagacatggctgcgattggggctcaagtcacaggattcatattatggccactgctggagaataagccggtcttatggctgataccgatctcgtcaatctgcatatcactaggctggtgggagaactatgtcactcggcagagtccaatcggtataatcaagagcctcggccgcctcaaggaggagctgaaccacacgcgctactacacgtaccgcttcatctccgtgtggaagatcctgctgttcctcatgtgcatcctcaccagcatctggctggacggcgacgagcccggcatgttcttccagctcttcagcgaggggttcggaccgcataacattgttgtcgaagagatccaactccagacgggaggcacaatgatcccggacttagccaacgccacactaaccggagactcagtggaggtggcagcggcctacaactctgccgtctacgtcatcctcatacaagtgtttgccgcttacttctgctacatattcgggaagttcgccagcaagatcctgatccaagggttcagttacgccttcccgatcaacttggtcataccgctggtcgtgaacttcttgattgctgcttgcggtatccggaatggtgatacgtgctggttccatgggactattccggattatctgttctttgagagcccaccagtgtactcactaagcgacttcatatcccgccaaatggcatgggtttggctgctatggcttctgtctcagacgtggatcaccatccacatctggacgcccaaggccgagcgtctggcgtccacggagaaactgttcgtactgcccatgtataacggactgctcatcgaccaaagcatggcgctaaatcgtaagagagatgaccagaaggatgttaagactgaggatctggccgaaatcgaaaaggagaagggcgacgagtactacgaaactatttctgtgcacaccgacaacactgggtcctctcccagagccgtaaaatcttccgatcagatcacaagaatctacgcatgcgcgacgatgtggcacgagacgaaggacgagatgatggagttcctcaagtccatcctgcggccggacgaggaccagtgcgcgcgccgcgtcgcgcagaagtacctcagagtcgtggaccccgactactacgagtttgaaacccacatattcttggacgacgctttcgaaatatcggaccacagtgacgacgattcccaggtgaatcgattcgtgaaactgttggtggacacgattgacgaggctgcgtcagaggtgcaccagactaatattcgtatgaggccgccgaagaaattacctgccccgtacgggggacggctgacctgggtgctgcctgggaagaccaagatgatctgccacttgaaggacaaggccaagattcgacacaggaagcgatggtctcaggtgatgtacatgtactacctgctcggccaccgtctcatggagctgcccatctccgtggaccgcaaggaggtgatggctgagaacacgtacctcctgacactggacggagacatcgacttccaaccgcacgctgtcaggctgctgattgatttgatgaagaagaacaagaacctgggcgctgcttgcggacgcatccatcctgttggctctgggccaatggtgtggtaccagatgttcgagtacgcgatcggtcattggctgcagaaggcgacggaacacatgattggctgcgtgctgtgtagccccggatgcttctcgctcttcagagggaaggctctcatggacgacaacgtcatgaagaaatacacgctgcgatccgacgaggctaggcattacgtgcagtacgatcaaggggaggatcgttggttatgcacattgctgttacaacgaggataccgagtagagtactcagccgcctccgacgcctacacgcactgccctgaaggtttcagcgagttctataaccagcgtcgtcgctgggtaccctccactatcgccaacatcatggacttgcttgccgactacaaacataccatcaaaatcaacgacaatatctccacaccgtacatcgcttaccagatgatgttgattggcggtacgatcttgggccccggaactatattccttatgttggtgggagccttcgtggctgcgtttagaatcgacaactggacttcattcgaatacaacctctacccgatattgatcttcatgttcgtttgtttcacgatgaaatctgagatacagttactggtggcacaaatactctctacggcatatgcaatgataatgatggcggtaatcgtgggtacagctttacaattgggcgaggacggaataggttcgccatcggctatcttcttgatatcactgtcaagttcattcttcatagccgcttgtttgcatcctcaagagttttggtgtatcgtccccggtatcatttaccttctgtctattccttctatgtatctcctgttgattttgtattcgactataaatcttaacgtcgtatcttggggtacccgagaggtgcaggttaagaaaactaagaaggaaatcgagcaagaaaagaaagaagcggaagacgcaaagaagagtgcgaaacagaagtctttactcgggttcttgcaaggagcaaaccagaatgaggatgaagggtcaatagagttctcattcgcgggtctattcaagtgcatgttgtgcacacaccctaaaggcaacgaggaaaaggtgcaactgttgcatatcgcatctacacttgacaagctcgagaagaaactggaaactgttgaaaagaccctcgaccctcacggcctccacagaggtaggaagctgtcgataggccaccgcggcagtaccaacggagaccacgggctggacgccctggctgaagacaatgaggaccacaacctcgactctgacaccgacactctatccacggcacctagagaacaaagagacgaattaataaatccatactggattgaggacccagaattaaagaagggagaggtagacttcttgagtcagtccgagattcacttctggaaggatctgattgataagtatctgtacccgatcgatgccaataaggaggagcaggcccgtatctcgcacgacctgaaagagctgcgaaactcatccgtcttttccttctttatgatcaatgccctctttgttctcatcgtattcttgctgcaactgaacaaggacaacctccacataaagtggcccttcggagtcaaaactaacattacgtatgatgaggtgacgcaagaggtgctgatctccaaggatacctgcaactagagcctattggtctggtgttcgtgttctttttcgcattgattttagtcatccagttcactgccatgttgttccatcgattcggaactttgtcgcatatattatcgtctacggaactgaactggttctgcaataagaaggcggaagacttatctcaagacgcactgctagataagaatgcgatagcaatagtgaaggatctccagaaactaaacgggctcgatgacgggtatgacaatgactcggggtcgggcccgcacaatgtgggaaggagaaagacgatacacaacctggagaaagcgagacagaagaagaggaacataggaacgctcgacgtcgctttcaagaagcgattcttcaacatgaacgctaatgaaggaccaggaacaccagttctgaaccgcaagatgacgttgcgaagagagacgttgaaggcgttggaaacgaggaggaattctgtgatggccgaacgaaggaagtcgcaaatgcaaacacttggagctaacaacgaatatggagtcactggaatcttaaacaacaacccagcggtgatgccgcgccaccggccgtcgacagccaacatttcggtcaaggacgtcttcgcggaacccaacgggggacaagtgaaccgagggtacgagaccacgcacggcgacgagggagacggcaactccatcagactgcagccgagaaccaaccaggtctccttccaggggagataccaataa

SEQ ID NO: 88 - DNA, 506 bp, Plutella xylostella
  source 1..506: mol_type = genomic DNA
gcggcagccagcagtaccgcgcgctgaccgtgcccgagctcacacagcagatgttcgacgccaagaacatgatggcggcttgcgacccgcgccacggccgctacctcaccgtggccgccatcttccgcggacgcatgtccatgaaggaggtcgacgagcaaatgttgaacatccagaacaagaacagcagctacttcgtcgaatggatcccgaacaacgtcaaaacggccgtgtgcgacataccgcctcgtggactgaagatgtctgccacctttatcgggaacacgacagcaatccaagagctcttcaagaggatttctgagcagttcactgctatgttcaggagggaagcgttcctccactggtatactggtgaaggcatggacgagatggagttcacagaggcggagagcaacatgaacgacctggtctccgagtaccagcagtaccaggacgccacggctgaagacgagggagaattcgacgaggatattgaagacgagtga

SEQ ID NO: 89 - DNA, 1146 bp, Spodoptera frugiperda
  source 1..1146: mol_type = genomic DNA
gcattaacatctcaggatttgattcgaaagtaaggtctgaatttagtccttacatttacttaaaattaggtcctttttggtttgtactgaaatgaaagtttttctgttcttggtcttaattgtgaagataatggctgaggcaaaaggagattgtgatgtgatcccgattacgcagtggggagattcacctcttaaaagggaggatyctcttccaaatccagtgaatattgttgtcgtccaayacrctgtggtaccggagtgtaacaatgatgaagagtgtgagaaagcagccamtggaatcaggagctaccacattaacaaacgtggattcactgatataggacaatcgttcctgattggtggaaacgggagagtttatgaaggagccggctggcatcacgttggggcccatactttgggatacaatgcaagatctgtggggatctccttcattggcgattttagaacaaaattaccaacacccgaagcactgaaagccttcaacagtctcctggaatgtggagtcacgaacaattatctgtcaaaggactatcacctggtggcccatagtcagctctctatgactgacagtccyggagacatgytgaggaagcaggtggaatcgtggcctcmttggctggataatgccaaagacatacttaagtagaaraagactaaacgccgtactttgagccatttaatggttacttaacccagtccttagcaatttgatacaaggccaatgtctctaagggcggcagtaaaggtcaaaacacatttaatgagtgtgtttaagattttgctagtgaaaattgttttgaagtacgtatttgatgtaagtgatgatatcagtacccttagtatgagtttgctttacgttccacgagatggaaacgagagcgcgttcggcgctctgattggttcgttcattcatgccggccaatcatagcgccgaatgggctctcatttcgttttcgttcaacgtaaagtaaattcgtactaagggtactgactttaaaataagttaccaaaaagagtattacctatttacattattttatttattttaggtgtattgtaattcaagtattaaattaattagtgtagattaatkscatgcattttatatttgatttcattgaataa

SEQ ID NO: 90 - DNA, 943 bp, Spodoptera frugiperda
  source 1..943: mol_type = genomic DNA
atcattccagatcctctctcatacatccaacacttgaagcaaaccaatccacatacattatagcaacatgttcgctctcaagttggtactagctgcagtgctggtggtcgcaagcgccagacatctaccacaggaccactcaacgtacgaccaagtacaactcctcgggttcgacgaagatggacgaccagtgtttgagcacgaagacttactcccagaactagaggagtcctaccagccagagcacctggcgaggactcgcagacaggcgcagggcagcgtcaccctcaactccgacggcggcataggcctgggcgctaagatcccgctcgcacacaacgacaagaatgtggtgagcgccatcggctccatggacttcaacaacaagttgcagcctgcttccaagggcttcggtctggctctggacaacgtcaacgggcacggactgacggtgatgaaggaaagtatccccgggttcggggacaggctgtcgggcgctggcaagctgaacgtgttccacaacgacaaccacaacgtggccgtgaccggctctctcgccaggaacatgcccagcatcccgaacgtgcccaacttcaacacgtacggcgggggcgtcgactacatgtacaagaacaaggtgggagcgtctctgggcatggccagtactccgttcttggaccgcaaggactactccgcgatgggcaacctgaacctgttccgcagcccgaccactaccgtggacttcagcggcggctttaagaagttcgaatctcccttcatgagcagcggctggaagcctaacttcggccttactttcggcagatctttctagatatattttgtaatctaaatttaactttaactttgttgtataatattttgtcgaattaagatcagtattgttcatactaatattatattatcagtgtttcttataaattaa

SEQ ID NO: 91 - DNA, 1399 bp, Spodoptera frugiperda
  source 1..1399: mol_type = genomic DNA
agttttcgtgcttagcacacagagcgccgaacgcgctctcgtttcaattactttaaacgtaaagcaaactcgcactaaggttactgtgacctttgtaaacatgtacaatgcaatactatgttgttattttcgatacaatcatataaaagacaacgtttaaaaaaaaagcatagcacagaccacagtacaagtatcgagaccaaacttttgttaaaacttatttcgttctgtgcagcaactctaatgcgaataatgtgcaaattataataaatatgttttgtatataaacatgtgttatctcaagttcaagacattccggctcctacagtcaacagatacagtgcacaaaaatgttaaaaatatatttaatttgtttgtcttacttgttcatgttaaacttagacagggcacaatgcaccccccagtattggttcaacatggatgcgaatggttggctgaaagtgcacacgatacccgccacgtgggaggaagcattccttcggtgtcactatgaaggtgcggtacttgcgtcccccttgacgcaacaactgagtaaggctcttmaaaacaagtttgcagkcwtcggtaacccatcaatccacttggggacacatgatttgtactccastggttactacttttctgtagaaggagtaccaatggacagcttggtgctgaaatggagtaatatcagaggtacgggcgaytgcctggcgatgtcacgcgacggggaagcatttttcacgaaatgcgagcaacctcgaccctacatctgctacaagaagctggacaatctgacgatgaacatctgtggaacattygatgacgcktatcaattctacgataagactggcagctgctataagagacacaacgyctatcagacatggcctgacgcgttcaagatatgcgctgccgagggaggstacctggtgatcctgaacgatgacacagaggccgccatcatcaaggatatgttccccgtacggccaggaaagcccaacgaatgggagaacttccacgtgggkctgcgggcatggggaccggaacgtacttggatcactattcatggagaaaaaattgatgatgtattccataattggaacccgggacagccggacaactacaagggagtccaggacactggcgctttccttagarmaggcactttagacgatcatgccgctggtgacaaatgtatgtttgtctgtgagaaggatcctaaagtaaaacgcttcgaagagytacccgaaggattggcmgaagttttaggacaataggccgctagtcaaatattgttcatatacctwagttttaattatgtaaaaataatartcgattgcagaatttaataaaatttaaactaaaaaaaaaaaaaaaaaaaaggtmaaaacatgtc

SEQ ID NO: 92 - DNA, 642 bp, Spodoptera frugiperda
  source 1..642: mol_type = genomic DNA
gccttgtagttcgacagtcaaatccaacgggtgtcagaaacaatttccgtttttccggctattgatcgtcatataaataactccgaaggaaaaatgactacaatcgacaatccgacagtaccgttcacgagacccattcctgggcatcygctccccggccgcaaaatggtcgttaagggtgcaatctctcccagatcagataggttctcaataaacttgaaatgtggtagcgaggacatcgctttccacttcaaccctcgcttcagtgagcagaaaatagttcgcaactcttatatttctggcaagtggggtcatgaggagatcagtggaggcatgccgttggtaaggggagagcattttgaagcgcaatttgaatgcaatgaagataatttttcggtggagttgaacgggaaacatttctgcaattactcttatcgcatcccaatccataagatcacccacgtcaacgtggacggtgacgtcacgataagtcagatcaccttcgtggacgcctgagccatcgcccgatcttacaatggtactttttttttacttatgaactaatgctggaaaccaaacttctttattcaaatatttttcttttgtctttatgactatcagtgtttttattgcaataaaaag

SEQ ID NO: 93 - DNA, 704 bp, Spodoptera frugiperda
  source 1..704: mol_type = genomic DNA
atcagtgtggtgtcttcaacccaagtgcattgtattgcttggtaaataataactgasaacaaaaatcttggttattgcttgaacaggtttcgcctatcaattagccgaatattgttacccttgacacgcaataattggttactcgattaaagcttgaagaggtaaagtgccgaaatgacgcgcgccattctgtttgttgttgttttatgctttattgcaaaatgctatggaaaaacattcackgaatgtgaattagttcaggagctaagaaggcaaggatttcctgaacatgagcttaaagattgggtrtgyctgatcgaagcggagagttccaaacgaaccaacgccattggtaacggcaattcagatggctctctcgactatggcttattccaaatcaaataaccgctactggtgcagcgayggtgaccatccaggcaagggatgcaacgataccgctagtaaagatctgttgctggatgacatcacaatagcgtctcagtgcgctaagaccattttcggtgtccacggatttaacgcctgggtcgcatgggtgaacaaatgcaaaggaaggaccttaccyaaccttcactgttagttatttattgagaaaatgtaactaaggtatatggttactttgtacctaaatataggtattaggcatatattgtccactctcatcaaatttttacttttatatcagt

SEQ ID NO: 94 - DNA, 789 bp, Spodoptera frugiperda
  source 1..789: mol_type = genomic DNA
attcgctgtacgcacctcggattgtgcggtcaactwtacaaggctcgtaccttgcagttgaaccgacaatctattcctaaagcctttttaaggtcaggaaaaatagttcctacatctaaatgcagtagaatttgcgaaacgaatttaaataaaaatggcgtcgattgtgtttgtgattttgtgtgttaccgtcgctgcggtgaaaagcgcgattttaaacccgtggagtaaagttgaggccaacaaatgtggtgtagaagccagtactaacttggtccatcacaatccatggttggtctacatcgagtattggcgtggaaactcagatactgagatccgatgcgccggtactttaatcgacagcaaacatgtcgtcacagctgcccactgcgttaggactctgaagtttagtcatttgatcgcccgtcttggcgaatacgacgtaaattctaaggaggactgcgttcagggcgtgtgtgccgatcccatcgtcagaatcaaggtggctgagatcatcgtgcatcctaactacagcaaccgggaacatgacattgcaatcttaaggctggaggaggaagctccttataccgatttcactccggcccatctgtctgccttctggtgatctcgcggaagacacccagttcttagcagccggctggggtgaratccccacgaaaggcttcttcagccacgtgaagaaaatcgtcccctacgtactggaatcgagagagatgccaaaaggtgtaccagtacaattatatcccggagaacgtgatctgtgccggt

SEQ ID NO: 95 - DNA, 835 bp, Spodoptera frugiperda
  source 1..835: mol_type = genomic DNA
caagtagcaacaaaatgcgtgtcctcgcttgcttggcccttctcttagctgtggtagcagccgtcccctccaatccccagaggattgtgggtggttcggtcaccaccattgaccggtaccccaccattgcatccctgctgtactcgtggaacttgagttcctactggcaggcgtgcggtggttccatcttgaacaaccgtgccatccttactgctgcccactgcacagttggtgacgccgccaacagatggagaatccgtcttggctccacctgggccaacagcggtggtgtcgttcacaacgtcaacactaacatcgtccacccctcatacaactctgcaactttgaacaacgacatcgctatcctccgctccgccaccaccttctccttcaacaacaatgttcaggctgcctccattgctggtgccaactacttgcccggtgacaacaccgccgcctgggccgctggatggggaactacctccgctggtggctctagctctgagcagctccatcacgttgagctgcgcatcatcaaccaggctacttgcaaaaacaattacgctacccgcggtatcaccatcacctacaacatgttgtgctctggctggcccaccggtggtcgcgaccagtgccagggtgactctggtggtcctctctaccacaacggcatcgttgttggtgtctgctctttcggtattggctgtgctcaggctgccttccccggtgtcaacgctcgtgtatcccgctacactgcctggatctcttccaacgcataagatgttacttggtgctaataatatttttttgtaataaaatgttacttttatcctcc

SEQ ID NO: 96 - DNA, 1464 bp, Spodoptera frugiperda
  source 1..1464: mol_type = genomic DNA
aacagttttctattggcagtcaaagacttcagtcgaaaaataatcctcatcagagtcgtgaagcaaggtgcccaaaatataatgtaacctagatacctattaataaattatttgtcaaccaaaacgttacgttcaaagtccttaaaatcaaaatatcttatgattagttttgatttaaaaatagaggttcgaaatcgccaacccaaataggtttagtttacgattcaggaaaaatcctaacgtagggaaacattattttacaagacttttggcttaaaaactttgagaaccaatgtcaaatttgataataactaatgaggtataaaagcttgatcctattaggacttattttcataacaccatcgagtttgtatttaatrragacgtgkgttaactaacaacatgaagaccggtgtaaaatattctgttmtttggatattctctctattytgctatatagaggcaacatttcgttgtgactacacgtacagcaaggaagcgaagggctggttcaaacatgtggtgataccagctacttgggctgacgcacgwctgcactgcacgttggaaggtgcaacgctggcttctccactcaaccaggctatmagtaatgagatgcagtccmtcctggcraacctctcggcgctgcaatcagaagtcttcactggaattcacrcgactktttcacgrmrcaacttatatcatacyatygaaggtatacctcttagtaaaattccattagattgggcaacaaatgagccaaatggtgggagagatgaaaactgtatcacgtttaactccgatggccaagcggcagacagatcctgtaargagactcgaccttacatctgctaccgacacacwactaaagtgactgtgkccaatgaatgtgggactgtagatcctgaatacaatttggataaaagaacgggckcytgctataagttccacacrgtacctcgcacgttcgagcgtgccaacttcgcgtgttctgctgaaggtggmcaccttgccataatcaacrgtgatrtcgaagctgcagtactgaaggaactcttcgaaaggaatccacctgcaaagatgttcggtactttctggaaagacgttgctttcattggtttccatgactggggtgaatatggtgactggaggacaattcacggtcaaacgctagtagaggcaggatacagcaagttttcatctggagagccaaataattcctcgacgggagaattctgtggttccatctatcggaatggtatgctcaacgacctttggtgtgggcatcctaaaccattcatctgtgagaaggacccgaaatatccagccgtatgttgtgttacagaatcagaaccagaattggaccctactcacttcttagagtaattcaaattggatctttttttataattctacatcattagaaatatgatgtttagttgaatctgcttttgaagattgatt

SEQ ID NO: 97 - DNA, 2461 bp, Spodoptera frugiperda
  source 1..2461: mol_type = genomic DNA
gaakagtgtcaatttatttaattgtcaaaatcgcgttgaatgtaagattgctatagaaattatttgtaaaaactttctcaaaaatttaattctaaaatgtaaaggaacctaaaatcagataccgataacacattttgtaaatggtttaatattgaaacgaaacttctaaacatttttggtgaaatacacataataatataacttcttcgaaaactaggtagatagacttcaactcagtttttattagcgagctagaagatacagagattatctaagatggtgtgtgatggtccggatcctccagagaacaatcctcacgaaattgttccacctcctaggaatgaattgataatgaatggtaatcaaggggatgacagtgatgaagaacatgaatattttggctatgaacctttggcgcaaggtccagaactagcagtctcagatcacgatagtgatgatgacccagagagtgctgaaacacctccagctgatgttccaaatatagagccaatggaaaatgtactaagccgggaagtttggagtgccccgagrcatacagatgctatacaaatggataatgaacgggctcaacaggtgatgagagctatggcaaattttgctctaccccaggcctcawttccagaatgggctcagagcatctctgaagaacaatggaagcaaactctgaatgaacaaatagaaagattgaaaaataaaagataaactaattatatgtagtataataataattaagatttaagtgataaaaataacaattataacttatatgtttaaaaaatgtatataaactgtaaactttaagcttattcacattactttaaatatagaaaaaatatgtgtacctatttattgtttggctacataatccaatatagattaaattgcaaatgtttaatgtaattatttagttggatcaaaaataacagcactaaaggtcactagtttagttctttcatttcgaatgaaaaagtaaaatggagaatctgcatagaaattttcagctgaccgtgttaatgttccacttgttgcggctactgcttctgttccaaactcattaattgttaactttacatcatgaattatggaatccacatgtattccagggttgggtagactgtccaaaatgtttgtcccatttccttcgttttctgagtatctgtctgattctccttcagatttattcacaaactgattggtgttatttaccataagtgcgaagtttgcttggcctggtataaacatactttgtatcccaatagattgcaaggtgctctctagctttgtggtgcttttaattttcatcttagggaaacgaataacacaatttttcacatgcatttcattgatggtatcatcaataactttgttgttaatattttccatgagttccaataatgttaatcgyttatgagataatggttttaaagcgtacataaaagtttcactgtcgttatatggcaatgctatcatgtggaaatcatgttcctcagaaaccttgtaacgaaactccccaaaattcaacatcatgtcagccataacttcatctgtggcccttttgaattccatctttcttgtgaattgcggtgcaaaaggctgtttccatgtgccactaaagtacaatgcagttaaaagtactactttagtatggggaggcagagaatctttcaaaaaatcgtctatatttccttctgtgtgcttgctcacccactcattgatgctgtttttagatccttcggtttcttgaaaatcagtacgtaacacatcgcctccgtaaacaccatgcagataacttctgtacaactctcgcaattctacttggttgtctacaaatatcgcgtcagcgtacaaagtttttgaaaactcgttcatatttaaactctgtagtagttcgccgaactgttcgtggttcttgcggtttcgtagaatatcttgggaaaatccgagtgtctctgcgatttcatcatgtgtcatcccaacacttccgagtaatgtcatggcaagcagaccagcgacacctattggtgatatcacgatgttttcgtttttcttttcgttcatcattttgactagtaaattatatccaaaattatttatagcttttggtatcgagttatctttcgtatttgttgtttctaagttatcattttcagcagttgtggttggtattatcgtgcttgaaatctgactgtcctcttcattttgtccagaatattgaaaatataatgttgttataattaaaaataacaatgacttgctatccatgttgtggtgcttaaataaggtattaaacgtaaacttcacactaacaggaacaggttcctattatttcccacttaaatcgtgaacttacggcatagacactatacacgtctactcgtttgaccaactctgagagcaactcaaaaacaaccttgtgtgtgtatgaataacgaactaaaat

SEQ ID NO: 98 - DNA, 391 bp, Spodoptera frugiperda
  source 1..391: mol_type = genomic DNA
ttcgtgtcgtatcactagagttcgaaatacaaaataataatacatttattattttgccataattaataataaagttattttatttcataataataatgaatttcacaaagatattttgtttgtttttgtcttgctttgttttgatggcgaccgtgtcaggagctcctgaaccgaggtggaaattcttcaagaaagtggagaagttgggccaaaacatccgckatggtatcataaaggcaggacccgcagtggccgtggtgggatcagcrgcagccatwggaaagtgakccctacgacctgagacatgaagactaatatccaytaaaataasaatattgaggcktataatattaatttattrtrtttgtaaattaaattatttgtaagataa

SEQ ID NO: 99 - DNA, 717 bp, Spodoptera frugiperda
  source 1..717: mol_type = genomic DNA
ataatatgaagagtgctgcgactaacgatttaagaatctgtcgtataagtcggtcgtccggaattgcttctggcggcgaagatgtcttcattctggtggaaaaagttaataagaaaaacataatgattcgattcttcgagttggatgaaaacggagaaaagaggttggaccaatgttgggcgatttgtgcaaagcgatgttcatcaccaatacgcaatcgtctttcgtactcctccatacaaaaaccctgagacgccagtcgatgtggaagtgtatatcgaactggttcgtccatcggacggccgtaccagtgaaccgaagcaattcaagtacagagccaaccaagcgtacaaacagatcaagaagaggaagactggatcttcctactgttctattggcagctcatctagtggatcgttgaaaagtggttgcgacattcccatatctgttgtcaatcatcaaccagaagaagttcccatggatcgcgagccaccggtgccgtcgtcgatgtatgttttaccccaggtgcatgattcgacgacaccaaaccaatgcgatctggccagtgctctgtactctgctcctggttcggagaccagccagtctcctatatcgagtccgatgtggagtgagcctcacagcgtgatgctgccgatgtcgcccattgccaaccttcagctcaactccgcagacttcgaacagatcacagtacccact

SEQ ID NO: 100 - DNA, 2124 bp, Spodoptera frugiperda
  source 1..2124: mol_type = genomic DNA
aaaaccgaataaccggtataccggataacttcttcaaggatttgaagaatatagagcatttagatttgagcttcaataagataactaaattgaccagtggagtgttgtcaccaatgaaacaattgaagtatttgaacctgaatcgaaaycatttggaagttttrcctgaattcctcttcgctggcctcagraaactagaraawgtrwcaatmaacgaaaatctactcacttccatagattctttagcattccaaggrgcwacggcwttgcatacaatatctttatacggcaacagattaacattgaagtccaatgaacacatccaagactatatggacttagatctctactcacccttyaacactttgagcgaattaaaaaatttgaaccttagtaagaayaayataagctctatattcgatgattggmggatwgtgytrctcaatytggagttgctggatctatcttataaycatattggagagttattgccggacacctgycaattcctaagcaacaarrtyacagtggacctgcagcacaatgacataagtactgttattttatatcctacgtctcatatggacttcacagaacccagcatgccatcaaacaatgtgtttctcttagacaacaacccatttaactgtgattgccacatatataacctagctatgcgtctacaaggcaaaaagctgccctacgaacctactttcaacgtaggcaaggccgaatgcaaatccccacgccacttaagtggggatttgttgtcacatgtatccccactcgacttatactgcgaagaaatgtcgcctgatgttcgttttaattgtagttcggttaaaatgaggcctgcgtacaatgatttggcgtacgactgtgatgatgtaccaccttacttctctgatgacattaaaaactactctttgaaattacgacacccaccaaaagacttacgcaacctaacactaagtctactaaacctaaccggcataggattacaagaaatcccmtttacrccgtcagaatcggtaaaagtaatcgatttatcgaacaacaatctcacagagatacckatmmgattcttagaatcgaatacgacrttgtwtttatcgaataatccatttgtttgcgattgttcttcraaagatgatattttagctttamgrgcragttayaatgtgaatgatttggatatagtyagctgttcaaacggcgttttggtaacagatttagaaatatcatcgctatgctatacaagaactttaatagcagaaataggtggtataytaatattcttaytaataatcttagtrtttataataacattyatatttcctagacaaatggtactrtattgtggtcataggctmtttccktgctggtatatcgacgatccagaaactacggcaaaagaatatgacatattcatatcttatgctcataaagaccaaaaatatagtgaataagctactcccgtaaactagaaaatgatttcaagttaaaagtctgcgttcattaccgagattgggaagtcggtgatttcattccggatcagatcaatcgatcagtatcgaattcgagaaaaacgattattcttttatcgaatagctttctcgattcgactttcgcgaatttggaatttcgtaccgcgcataatttagctttgaaagagggaagagagagggtcattttaatacttttagaggacgttagtaaacatgagaagttatctgaagaattaaagtatcatatgaatatgaatacgtatcttacatgggatgatattagatttgatgaaaagttaaaacgaaggacgataccacagaaatataatagaaagaaattcgtagcgccggccattttaaaacctatattcagacaggctactgagaataatttgaaaaaggcactcgatgtacactttgaatagtgcaggccaattggtgaatttagctcaaaataagaagaatattgatatggtatagcttttcccaaataaggctcttttaaacatgttaagccctctctcacccgacctctctgactaccgcactcagagacattttaatgtatwttttttttttaattgggtacaagcccgccacaacatcccagaccgct

SEQ ID NO: 101 - DNA, 1128 bp, Spodoptera frugiperda
  source 1..1128: mol_type = genomic DNA
atgtggtcggtgttagccggagtattggcgatcgcgtcgctaggcgcggcttgcacccccagtttgaccaccgtcagtggtacccacgcaccggtcaccgtctgctctggtgctctgatcttcgctgatggcttcgacacttttgacctcgagaaatggcagcacgagaatactttagctggtggcggtaactgggagttccaatactacggcaacaatcgcaccaactctttcgtgcgcagtggaagtttgttcatccgtccctctctaacatcagacgagttcggagaagctttcctctcatctggacactggaacgtcgagggtggtgctcctgctgatagatgcacaaatccacaatggtggggttgcgagagaacaggcacgccgaccaacattttgaacccaatcaagagtgctcgtgtccgtaccgtcaattccttcagcttccgttacggacgcctcgaagtccgcgctaaaatgcccgccggagattggatttggccagctatctggttgatgcctgcgtacaacacttacggtacttggcccgcatcaggagagattgacttagttgagtcccgaggcaaccgtaacatgttccacaatggtgtccatatcggtacacaggaagcaggctcgaccttgcactacggaccttacccagcgatgaacggttgggagcgcgcccattgggtcagaaggaaccctgctggctacaacagcaacttccaccgttaccaacttgaatggacaccaacttacttgcgattcagtatcgacgacatggagcttggacgtgtaacccctggcaatggcggcttctgggaatacggtggtttcaacagcaaccctaacattgagaacccatggagattcggaagcagaatggcgcctttcgatgagaagttctaccttatcatgaatgtggctgtcggtggtaccaacggattcttccctgatggcgtcagcaacccatcacccaaaccctggtggaacggatcaccaaccgccccaagagacttctggaacgcgagatcagcttggttgaacacctggaacctgaatgtcaacgatggacaggacgcatccatgcaagtcgactacgtccgcatctgggctttgtaa

SEQ ID NO: 102 - DNA, 961 bp, Spodoptera frugiperda
  source 1..961: mol_type = genomic DNA
atcagtcgtccctcgtacatccaacacttcaaaccaaatatctccatatacatagtaacaacatgttcgccctaaagttggtactagctgcagtgctggtggtcgcaagcgccagacatctaccacaggaccactcaacgtaccgaacatgtacagctgctcgggttcgacgaagatggacggccagtgtttgagcacgaagacctgctcgcagaaccagaggagttctatcagccagagcacctggcgaggactcgcagacaggcacagggcagcgtcaccctcaactccgacggcggcatgggcctgggcgctaagatcccgctcgcacacaacgacaagaatgtggtgagcgctatcggctccatggacttcaacaacaagctgcagcctgcttccaagggcttcggtctggctctggacaacgtcaacgggcacggactgacggtgatgaaggaaagtatccccgggttcggggacaggctgtcgggcgctggcaagctgaacgtgttccacaacgacaaccacaacgtggccctgaccggctctcttgccaggaacatgcccagcatcccgaacgtgcccaacttcaacacgtacggcgggggcgtcgactacatgtacaagaacaaggtgggagcgactctgggcatggccagtactccgttcttggaccgcaaggactactccgcgatgggcaacctgaacctgttccgcagcccgaccactaccgtggacttcagcggcggcttcaagaagttcgaatctcccttcatgagcagcggctggaagcctaacttctcctttaatcttggcaggtcattctagaaatatttttaaactcttatttaaaaattaaatgtaaaaaatccwgtttgttcatgataataagaataaatracagtattgttcgtactatttactatgtaatctataaattgtattaataaatgaaaattaa

SEQ ID NO: 103 - DNA, 1003 bp, Spodoptera frugiperda
  source 1..1003: mol_type = genomic DNA
atcagtcgtccctcgtacatccaacacttcaaaccaaatatctccatatacatagtaacaacatgttcgccctaaagttggtactagctgcagtgctggtggtcgcaagcgccagacatctaccacaggaccactcaacgtaccgaacatgtacagctgctcgggttcgacgaagatggacggccagtgtttgagcacgaagacctgctcgcagaaccagaggagttctatcagccagagcacctggcgaggactcgcagacaggcacagggcagcgtcaccctcaactccgacggcggcatgggcctgggcgctaagatcccgctcgcacacaacgacaagaatgtggtgagcgctatcggctccatggacttcaacaacaagctgcagcctgcttccaagggcttcggtctggctctggacaacgtcaacgggcacggactgacggtgatgaaggaaagtatccccgggttcggggacaggctgtcgggcgctggcaagctgaacgtgttccacaacgacaaccacaacgtggccctgaccggctctcttgccaggaacatgcccagcatcccgaacgtgcccaacttcaacacgtacggcgggggcgtcgactacatgtacaagaacaaggtgggagcgactctgggcatggccagtactccgttcttggaccgcaaggactactccgcgatgggcaacctgaacctgttccgcagcccgaccactaccgtggacttcagcggcggcttcaagaagttcgaatctcccttcatgagcagcggctggaagcctaacttctcctttaatcttggcaggtcattctagaaatatttttaaactcttatttaaaaattaaatgtaaaaaatccwgtttgttcatgataataagaataaatracagtattgttcgtactatttactatgtaatctataaattgtattaataaatgaaaattaactatmtaamwaaaaaaaaaaaaaaaaaaaaaaaaaacatgtc

SEQ ID NO: 104 - DNA, 831 bp, Spodoptera frugiperda
  source 1..831: mol_type = genomic DNA
atcagtcgtccctcgtacatccaacacttcaaaccaaatatctccatatacatagtaacaacatgttcgccctaaagttggtactagctgcagtgctggtggtcgcaagcgccagacatctaccacaggaccactcaacgtaccgaacatgtacagctgctcgggttcgacgaagatggacggccagtgtttgagcacgaagacctgctcgcagaaccagaggagttctatcagccagagcacctggcgaggactcgcagacaggcacagggcagcgtcaccctcaactccgacggcggcatgggcctgggcgctaagatcccgctcgcacacaacgacaagaatgtggtgagcgctatcggctccatggacttcaacaacaagctgcagcctgcttccaagggcttcggtctggctctggacaacgtcaacgggcacggactgacggtgatgaaggaaagtatccccgggttcggggacaggctgtcgggcgctggcaagctgaacgtgttccacaacgacaaccacaacgtggccctgaccggctctcttgccaggaacatgcccagcatcccgaacgtgcccaacttcaacacgtacggcgggggcgtcgactacatgtacaagaacaaggtgggagcgactctgggcatggccagtactccgttcttggaccgcaaggactactccgcgatgggcaacctgaacctgttccgcagcccgaccactaccgtggacttcagcggcggcttcaagaagttcgaatctcccttcatgagcagcggctggaagcctaacttctcctttaatcttggcaggtcattctagaaa

SEQ ID NO: 105 - DNA, 4767 bp, Spodoptera frugiperda
  source 1..4767: mol_type = genomic DNA
atggcgagaccaagaccttatggttttagggctttagatgaggagagtgatgacaattcggagttgactccgttgcacgatgataatgatgacctaggacaaagaacagctcaagaggcaaaaggatggaatctgtttcgagagattccggtgaagaaggagagtgggtctatggcctcaactgccgggatagacttcagtgtaaagatccttaaagtcctggcgtatatttttatatttggcatagtgctcggatctgcggttgtgtctaagggtacgctgctttttatcacatcacaactgaaaaagggcaaagcaatcgttcactgtaatagacagttagaactggacaagcagtttataacaatccattcgttgcaagagcgtgtgacgtggctatgggcagccttcatagcattcagtattccagaagttggcgttttcttgagatcagtcagaatatgcttcttcaaaacagcaccgaagccttctgttttacagtttttgacggccttcgtagtagacacccttcatacaataggcattggattactggtgcttttcatcctgccagaattagacgtggttaaaggaacaatgctaatgaatgctatgtgcttcatgcctggaatactaaacgctgtgaccagagaccgcacggactctcgatacatgttgaaaatggcactagatgtactagctatctccgctcaagccaccgcgttcgtcgtctggcctctgctaaaaggcgttagtatgctctggacgattcctgtcgcatgcgtattcatctcactcggatggtgggaaaatttcgtcggcgatatcggaaaacaatggccagtcctggaacctgtacaagaacttcgtgacaatttaaagaagactcgttactacacacagagggtgttgtctttgtggaagatattcatattcatgtgttgcatcctgatatctttggcggcacaagatgacagcccgctttctttcttcacggagtttgctactggatttggtgagcgcttctacaaagttcatgaggttcgagcgatacaggacgaatttgaaggttttctgggctacaaaattatggacttatacttcgatcaaatgccagcggcatgggccaccccactgtgggtggtgctgatccaggtcctggcttctttagtctgttttatggcaagtttgtctgcctgcaagattctgatacaaaacttcagctttacatttgcgttgagtcttgttggacctgtcaccatcaacttgttgatttggctttgcggcgagaggaacgcagatccctgcgcatatagtaatacgataccagattatctgttcttcgacataccaccggtgtatttcctgaaggagtttgtggtgaaagagatgtcgtggatttggttgctgtggctggtgtcgcaggcgtgggtgacggcccacaactggcgctcccgggccgagcgtctcgccgccagcgacaagctcttcaacaggccttggtactgcagccccgtcctcgacgtctccatgctgttgaacagaaccaagaatgaagaagcggaaataacgatagaggatctaaaagaaacagagagtgagggtgggtctatgatgagcggatttgaagcaaagaaagacataaagccttctgacaacattacgaggatatatgtctgcgcgactatgtggcacgaaacgaaagaagaaatgatggacttcttgaagtctatcctgcgtttcgatgaggatcagagcgcgcgtcgcgtcgcacagaagtacttgggcattgtagatcctgattactatgaactcgaagtacacatcttcatggacgatgctttcgaagtgtcggaccacagcgcggacgactcgaaagtgaatcccttcgtgacgtgtctcgtggagactgtcgacgaggctgcttcagaggtccatctcaccaacgtgaggttgaggccaccgaagaaattccccacaccgtacggcggccgactggtctggactctcccaggaaagaacaaaatgatatgccacctcaaagacaagtccaaaatacgacacaggaaaagatggtctcaagtgatgtacatgtactacctattgggccaccgcctgatggacgtgccgatctcagtggaccgcaaggaagtcatcgcagggaacacctacttactggctttggacggcgacattgacttcaaaccgacagcagtcacgttactaatcgatttgatgaagaaggataagaatttaggagcagcgtgcgggcgcatccatcctgtgggctcaggcttcatggcatggtatcaaatgttcgagtacgctattggtcattggctgcaaaaggcgactgaacacatgattggctgtgtactctgtagccctggatgcttctccctcttcagaggaaaggctttgatggacgacaacgttatgaagaaatataccttaacttcccacgaggcacgacactatgtgcaatacgatcaaggcgaggaccgttggtgcacgctactgctgcagcgcgggtaccgcgtggagtacagcgcggtgtcggacgcgtacacgcactgccccgagcacttcgacgagttcttcaaccagcgccgccgctgggtgccctccacgctcgccaacatcttcgacctgctcggcagcgccaagctcaccgtcaagtccaacgacaacatctccaccctctatatagtctatcagttcatgttgatagtgggtacggtgttgggtcccggcacgatcttcctgatgatggggggagccatgaacgccatcattcagatcagcaacgcgtacgcgatgatgttgaacctcgtaccactcgtcatcttccttatagtctgtatgacttgtcagtcaaagacgcagctcttcctcgctaacctcataacatgcgcatacgcaatggtgatgatgatcgtgatagtggggatagttctgcagatagtggaggatggatggctggctccgtccagtatgttcacagctttaatattcggtacattcttcgtcaccgcggcactacacccgcaagagatcaaatgtttgttgttcatagcagtgtactatgtaaccatccctagtatgtacatgttgttgatcatatactccatctgtaatctcaacaacgtatcctggggtaccagggagacaccgcagaagaaaactgctaaggaaatggaaatggaacagaaggaagcagaagaagcgaagaaaaaaatggagagtcagggtttgaagaagttgtttgccaagggagaagagaagagtggttcgttagagttcagtgtggcgggcctgttgcgatgtatgtgctgcaccaatccagaggatcataaggacgatctcaacatgatgcagatctcacacgcgttggagaagataaataagagattggatcaactcgatgtccctcctgagccgacccaccagccctcgcatccgcacacacacgtggagacggtcggtgttcgtgattacgaagacagcgagatttccactgaaattcctaaggaagaacgagacgacctgattaacccctactggatcgaggacgtggaactccagaagggcgaggtagacttcctcaccaccgctgagaccaacttctggaaggatgtcatcgatgaatacttactgcctattgatgaggacaagcgtgaaattgaacgtataagaaaagatttgaagaacttgcgagataagatggtgtttgcgttcgtgatgttgaactctctgttcgtgctcgtcatcttcctgctgcagctcagccaggaccagctgcacttcaagtggccattcggacagaagtccagcatggagtacgataatgatatgaatatgttcatcataacccaagactacttaacgctggaacctatcggtttcgtgttcctcctgttcttcggctccatcatcatgatccagttcaccgccatgttgttccatcgcctggacacgctggcccatctgctgtccaccaccaagctggattggtatttcagtaagaagccggacgacctatcagacgatgcgctaatagactcttgggcgttgacaatagcgaaggatcttcaacgtctgaacaccgacgacttggataaacgaaataacaacgaacacgtgtccaggaggaagaccatatataacttggagaaagggaaggaaaccaaaccggctgttatcaacctcgatgccaacgccaagaggagattgactatcctgcagaatgaagactcagaattgatctcccgcctgccatctctgggacctaatttggcaactcgtcgtgccacggtgcgtgcaataaacactcgacgcgcatctgtcatggcggagcgacgcaggtctcagttccaagcgcgaccttccgggggatcatacatgtataataaccctcaaaacacgattcagctggacgatatggtcggggggccgtcgacgtcgggagtgtacgtgaaccgagggtacgagcccgccctggacagcgacatcgaggacacgcccgtgcccaccagacgatccgttgtacacttcaccgaccatttcgcgtgataaccaccaaaatctgactaacatctccatattacattttctactctgttacgaaacgataaagtttaaagtgtattaaataaattggacagatttgagtaggtttcacgtttgtgtttaaataatttatgaaaataacatacctattgctttatgaccgctttaatattaaaagaaactcaatatatgcattaa

SEQ ID NO: 106 - DNA, 942 bp, Tribolium castaneum
  source 1..942: mol_type = genomic DNA
atataaaaaaaaaaaaatattaggcaatttatttacacaaatagcacaatttatcaattcacaagttgtgtattttattataaatactaaatcaacaatagcgactaacaattaacacaattttatttcacttcctgtcactatcgcgagtaataaattcccgcatcaaaatgcggccaatttttgatctctttgtaaacatttggccccggactttccgttctaaacgtctgattgtgagctaccagtttataatccctggccaattttccactctttaccccctcatctagcaatttcttcgccacgctgatcatctccgtcgtcaaatgatcatgaaaaaaattcccaataaaactaatcccgatcgaatcatccatgtgaaaattcctgatatcccagcctcgtccaacatacgcattcccatctccaccaattacgaaattgtacccaatatcgggacttttcaaattgcccacatggtagtcctgcatggactgcaccctttgcgaacatgcaggaaagtcgctacaagtcggggtaacagtgtgtgaaacgatgacaaaatgagtgggatgtggcagtggtttagagaagtttaaggtggcacggcctccccaaatttttttctcgatgatggcaccggggcccaaaggaagtcttggagtcgcagtgttagtgtcgggagtttcgggagacttttcagttttccgtgggagtattacaacgcaagttgtggtgacgaggattacgattacgactagagaaatgcccaaatatttcacaggtttggagtataaaaaggagtttttttggtttttcagcgggggggagagtcggaaacaggggcttttcagacttgacgaagtttaccaatggaatcagttgaattaacaaatccttttccctaaagtcctctaaaaacagatgatggggccccat

SEQ ID NO: 107 - DNA, 747 bp, Tribolium castaneum
  source 1..747: mol_type = genomic DNA
atgagtggcagtgaccctttaacaaatacccaacaatccgatcaagattattaccatccactctgttattcaattcaagtggacgacgaaaatgaacaatcagctctcctgcccgcatttcatcaaaggaaaagtttgcgagttcaggataaaatctttattgtatttttattttcaattctaattaccggactagccattggcctctatctccttgcaactgagggacacgaatggaaagctgcaggagtctataatattacagttcgggaacagtggcaagctcacgtcccttcatcaacaatgccaaagttggaacttcccgtaagaagagttttatttcttcctgcaaataccactagctgcggcagcaaatcccactgtgccaaagtcctccaggaactacaattacagcatatgctgcagtggaaagaacctgacatctcctacaatttcataatgactgcagatggcagaattttcgaggggagaggatgggactttgaaacttctgttcaaaattgtacggttaatgatactgtgacagttgcttttttggacgaattagatgcgaaagcaccgacgtttagacaagctgaagcggcaaaaatgttcctggaagtggcagtaacagagggaaaattagaacggtgttttaataccgcggtctggggaggaaataaattcttcattgatttggctcgaaatgttcaagacgtcttatcggaatgcgagggaattacttaa

SEQ ID NO: 108 - DNA, 801 bp, Tribolium castaneum
  source 1..801: mol_type = genomic DNA
atgtgcgtttgcaagactgtgctattaatcgttgggctggggggttgttttgcgggtccggtccgtaattatgggccattgagacattacaacgttccgcgaccctcaatccaagcattcaggccccgtggcttcaaagtgagcatccctcatactgaaggcatccaattattcgcctttcatggaaatattaataaacccctgcacggtctcgaggccggacaattttcccaagacgtcctccaaagggagggagacgagtgggtgttccaagactccagtgccaaattaaacgtgggagataaaatctattattggctctttatcattaaagaagacctgggctacagatacgatcacggcgagtacgaagtgaaagttttagccacccgtgacttcgattctcctcaaacaacctctgtgacgccgaatcttgcccccaatctcggcatttgcgaaaaggtgatggtaaatctcacgcggaagctgctcgatttgcagcaagaaattgagtcccttagggagacgaacgatattttggaggatatggttcagaagcacactgatacggcgactactctcactttggacggcttgatgataaaggatgacgacgaacttgtttcggtgattcaagcaattattaaagataaacttggactaaaaagcaggattcaaaatgtgacgaggcaggagaatggaatggtcaagtttcaagtggcgagtttgagagaaaagttggaggttgtcaaagcggccaaaagaaaactcaagtcgtcgagctttacgatcacgtattaa

SEQ ID NO: 109 - DNA, 1158 bp, Tribolium castaneum
  source 1..1158: mol_type = genomic DNA
atgtatccgttgaagaggatgcccagtgaagaaatcagtatcagtgatctgcctagcgaaatgaaagaagttttactagaaattagcccgaactttgatgaaaatctgaaacgggctttcaggaacgaaggagtgaggctgcagagagtgcagaacaatggacgatttattcatcagctggacgacgttctcttatccatagacgataccaaaatcgagttacgcaacctgaaattcccctggatccccgacttccgcatcgtggacttgtccagcgacctgcccatgtcatgcctcgacctaaacctaaacctgggcaatttgcgaattgagggcgagtacgaagccaacaacaccacactcaggcgatggctcccggtatctcacattggtcgaatcgtgatcggttttaacaacgtccgagcgaacggaaaagtcggactcgtgctcgagcaggattctttcgttccgcagaattatgatattagatatgagccgacggatgttgttattagggttagctatcacgtggatggcgagaatgaggtgcaaaatgagattagcaattcagatattgaggccacgctaggcaagaccgtgtgggtgcagttgactgagatattgtccaacctgttgcataggcaattgggcgaggttgtagtggagttttccgtgacggaactcctcgtcgatagggacgaggaatacagggaatacgccaagggacaagcagcgcgcgccaataaactcctggactcgcttttgtgctcagccaaggactatttagtcgcgaaggacttgaggacggtcaaaacgccacccttcgacgtcgtcttcaaagggaaagtctcgggggtgcagcaggggaccttcagcacgggggaagggttcctccaagacctcgccactttgacgcggagacacagctttagtttgtacgaagacaaacacaaactcacgatatatggggggatcaggctgagggagtttaaactcgggtatgggggctaccagggccagtacgaggaaacggccgtctcaggcagcatcaaaggctcgctctacaagaacgagattttcgtcaagatcactgtgaaaaaagaaggcgagcggtgctcgactcagttagattccgtccaagttgttgttgtaaagtaa

SEQ ID NO: 110 - DNA, 4393 bp, Tribolium castaneum
  source 1..4393: mol_type = genomic DNA
atggcggcgcgtcatcgctttgccacagggagccctgaggaaacagagcccctgtattcgtcgacgcaaatgcccgaaaaagtccgggaaaaatggaacgtcttcgacgaccccccaagagagcccacttcggttccgaagtcaaaagaacctacatcgagtggggggtgaagtttttgaaagttgtgacaatcataactgtgttttttgtggtccttggtgctgcagtggtttcgaaagggacaaccttgttcatgacgtcacaaataaaaaagaatgtgacaagggcttattgcaacaaaaagatagaccgcaacctccaattcgtcgtctccctccccgaagtggagcgcgtgcaatggatctggctcctcattttcgcttacttgatccccgaagtgggtacctggatccgcgccgtccgcaaatgcctctacaagctctggaaaatgccctccctctccgaattcctctccctcttcggcacggaaacgtgccccgccatcggaagcgcaattttgatattcgtcgtcctccccgagctggacgtcgtcaaaggggcgatgctcacaaacgcggtttgtttcgtgcccggagttgtggcaatgttctcgcgcaaaccgtgctccataaacgagaacctgaaaatggggctggacatcgccagcataactgcacaggcgtcaagcttcgtggtgtggcccttggttgaaaataacccgaccttgtacctaatccccgtttccgtgattttgatttcggtgggttggtgggagaatttcgtgtcggaaacgtcctacttaccgtttatccgggctctgggcaagagcaaaaaggagtttaagacaaggacgtacttcatttacgcgttcgtggccccggttaaatgcctggcgtttttttgcaccgctttggtcattttttactgccaggagggcagtgttgactttttatttgataatttttcagccgcgtttcaggatcataacattgaaattaccgaagtcgcgcccgtcttgccggggaattacgcaaatgcagttcggtcgggagccgaaaccccatccacacaagcagttacatgacggggatttgggtttggttgattaacatttcggcgacttatatttgctacgcgtttgggaaattctcctgcaaagtcatgatccagagcgttagcttcgcttttccgatcaatttgtcggtccctgtcctcttatccggactgatcgcaatgtgtggcatgtactacagggatgagtgttctttcgctgagtcaattcctccatatttgtttttcgttcctccacctctcatgttcctacaagattttctctcgcaccaacacgcctggatttggctggtttggttgctgtcacaagcctggatttcggtgcacatttggtccccaaactgtgacaaactttcaagcaccgaacagttgtttattcggcccatgtatgacgcgtttttgatcgatcaaagcctggcgttaaaccggagacgtgacgaaaatcccagaaattacagaagcgacgaagggcctcaaattacagagctcgagccggaaacgatcgagagtcaggacgccataacccggatttacgcctgcgggacaatgtggcacgaaactcccgaagaaatgatggaatttttgaaatcggtgttccgcttggaccaagaccagtgttcccacaggattgtgagggagcatttgggactcaagcatgacaattactacgaattggagactcatatatttttcgatgatgcgtttattcggaccagtgaagacgataatgatccccacgtcaacgaatacgttgagtcacttgcgtccattatcgacgaggctgcgactaaggttcacggtaccacggtgagggtgcgtccgcccaaagtgtaccccacgccttacggcggacgcctggtctggacgctcccagggaaaacaaaaatgatcgcccacttgaaggacaagaagaagattagggcgaaaaagcgctggtctcagtgcatgtacatgtactttttgctcggattcagattgcaagccaacgacgaactctccgcccacagcaaggaaatccgcggcgaaaacacctacatcctcgccctggacggcgacatcgatttccaacccgaagccctgcacctcttggtggactacatgaagaagaacaaaacgttgggggcggcctgcggccgcatccaccccatcggcagcggcggcatggtctggtaccaaatgttcgaatacgccgtcggtcactggatgcaaaaagccaccgagcacgtcataggctgcgtcctctgcagccccggctgtttctccctgttccggggaaaagccctcatggacaaaagcgtcatgaagaagtacaccactcgatcgacccaagccaagcactacgtgcagtacgatcaaggggaggaccggtggttgtgcactttgttactccagaggggctaccgtgtggaatactccgcagcctcggacgctttcacgcactgtccggaaggcttcaacgagttttacaaccagcggaggcgctggatgccgtccactatggccaacattttggaccttttgatggattacgagcacacggtcaaaatcaacgaaaatatttccatgctgtacatcgggtaccaaattattttaatgatcggtacggtcattggccccggtactattttcctcatgttggtcggcgccttcgtggctgcctttgggctcgaccaatggagcagtttctactggaatttactaccaatcgcagtttttatcctagtatgtgccacttgtagctccgatatccaattatttttcgccggccttatcagcgccatttacggcctgataatgatggctgttttcgtcggtgtgatgctccaaatcagccaagacggcccacttgcgccttcctcccttttcttcttctgcatggctgctgaatttataatcgcagcactgctgcatccgcaagaattcaactgtttgaaatacggggtcatttactacgtcacggtccccagcatgtacctcctcctagtcatctactcggtcttcaatatgaacaacgtgtcctgggggacgcgcgaagtgacagtcgtgcccaagcctgaccccaacgccgtccagaaaatcgaagagaaaaaaccggagaagaaagacaaagttttgacgtttctgggcgcgaatgcccaggacgacgaaggcgggcttgaattttcggtcaacaaacttttcaaatgcatgatttgtacgtacaaggccgataacaaggaaaacgagcagttgaggaaaatacaagagtcgttgagagacttgaataggaaaatcgagtcgctggaaaaaatgcaatatccggatttgaggtctcctgccgttagcaacgttacaacgttcatggagggctcaaaggcgacggttaagaacaacgtggaggataactacatggaggctccgcaagacaatgtttcgcaaccgtcggatgaggtcatggagaatagttggttctacgatgggcctttgattaggggggaagtgcattacttgaataggaatgaggaaacgttttggaatgaactgatagagcagtatttacacccgattgaggatgacaagaagaaggtttcggctgaattgaaagatttgagggacaaaatggtgtttactttcctgatgttgaattcgctctacgttattgtgatttttttgctcactttgaagaaggatttgctccatctggactggccgtttgaccccaaagtgaacttcacgtatttcgaggacaaaaatgagattggcgtttacataacatacctccagctggagcccatcggtttcgtgttcctcatatttttcgccctgcttatggtgatccagttcttcgcaatgatgatccaccgcttcggcaccttctcccaaatcatcacaaaaacacaattagacttcgatctgtgcagcaaaccaatcgacgaaatgactgtggacgaactcaggtcccgcgaccctataaaaatagtcgcagatttacaaaaactaaaaggtataaacaacgaatacgaggaccaaacggaagttccggtcgaaatgcgaaaaacagtaagtaatttggcgcaaacgagcggtggtggtgagaacaagcccatattttatttggatgaagcgttccaaaggcgagtcactcaaatagggagcacttcaagcaacaacccgagcattagcgcctttaggaagaaaagccttgcttacgtccaacaaagaatgagcatagccccaaatcgggtgtcacaagcccggcctagtgtgcagttacggtaccccaatggaaaagccaacgaaaatttcgtttttgacgaaaacgggtcagacgtggaggcataa

SEQ ID NO: 111 - DNA, 599 bp, Manduca sexta
  source 1..599: mol_type = genomic DNA
taatcattagaaaaatggcgagcttcgctttaatagttatccttagcgtaattggctttatatcggcctatcctagtcctgaaggttacagttctgccttcaactttccattcgtaaccaaggagcagtggggcggcagggaggcacgcacgtcgacgccactcaaccacccagtgcagttcgtggtgatccaccacagttacattcccggcgtgtgcctcagccgggacgagtgcgcgcgcagcatgcgctccatgcagaacttccacatgaacagtaacgggtggagtgatattggatacaacttcgctgtcggcggtgaagggtcggtgtacgagggccgcggctgggacgcggtcggcgcacacgcagctggctataacagtaacagtatcggcatcgtgctcatcggcgattttgtttcaaacctcccgccggcggtgcaaatgcaaaccacacaagaattgatcgcagcgggcgtgcgactcggttacatcaggcccaactacatgctcatcgggcatcgtcaggtctccgccactgagtgcccaggaaccagactcttcaacgaaatcaccaactggaacaacttcgtgagg

SEQ ID NO: 112 - DNA, 1335 bp, Manduca sexta
  source 1..1335: mol_type = genomic DNA
gagcgtctgtttgttcgcaaccattgcgggctgcttgggccagcgagggggtccatacaaggtgcctgatgcgaaactcgaagctatctaccccaaaggcttgagagtctctgtgccagatgatggctactccctatttgccttccacggcaagctcaatgaggagatggaaggtttagaggctggccattggtccagagacatcaccaaagcgaagcagggcagatggatattcagagataggaatgctgagctgaagcttggagacaaaatttacttctggacttacgttattaaggatggattgggatacaggcaggacaatggagaatggactgttacagaattcgtcaatgagaacggtacagtggtggacactagtacagcgccgccaccagtagcacccgccgtttcagaggaagatcaatcgccaggtcctcagtggagaccttgcgaaagatccctgactgagtccttggcccgcgaacgcgtttgcaaaggcagccttgtctttagcgaggactttgatggttccagtttggccgacttgggcaattggaccgctgaagtcagattccctggcgaaccggactacccgtacaacttgtacactacggacggcactgtgggattcgaaagtgggtctctggtggtgagacccgtcatgaccgagtccaaataccacgagggcatcatatacgaccgcctcgaccttgagagatgtacaggacagctgggtacgctggaatgcaggcgagagagcagcggcggtcagattgtaccacctgtgatgacagctaaactggccactcgacgcagcttcgcgttcaagttcggcaggatcgatataaaggcgaagatgccgcgcggggactggttgataccagaactcaacctcgaacctttagataacatatacggcaaccagcgatacgcttcgggtctcatgcgggtcgcgttcgtgagaggaaacgatgtatacgccaagaagctctacggaggtccgataatgtccgacgcggacccgttcaggtccatgctgttgaaggacaagcaagggttggccaactggaataatgattaccacgtctactcgctgctgtggaagcctaacggtttagagctgatggtggacggtgaagtgtacggcaccatcgacgctggcgatggcttctaccagattgcgaagaacaacctcgtgagccacgcctcgcagtggctcaagggcaccgtcatggcgccgtttgatgaaaagttcttcatcactctgggtcttcgcgtggcgggtatccacgacttcacggacggtccgggcaaaccttgggagaacaaggg

SEQ ID NO: 113 - DNA, 1194 bp, Manduca sexta; identical to SEQ ID NO: 5
  source 1..1194: mol_type = genomic DNA

SEQ ID NO: 114 - DNA, 763 bp, Manduca sexta
  source 1..763: mol_type = genomic DNA
acgaacaacctgacagacggatagcgggacggtcagcacaatacgaacatttaagaacaaacgagaggtctctcccggtctacagcgagacccagaggatacaagcagaagagagaagaagacacagttcgagactagaagaaccgagacaacgtgctgagaatggttcatataagatattgaataaccctccgaaaccctgtattactaataggagaagtcaaattgattcgtcgaatgatagggtagtgttccccggtccgacttcagaaaggtcgtacgtacccgaagtgccagaggaatgcaagaaaatcggcatatgcgacagtataccgaattacccagaagaacacgtagctaatattatatctcgacttggagacaaaggaaaagtattacaaatagacgaactggacgtatcagacactccagatatcgcccagaggttgggtccgcaggaggacaacatggaactatgtagctttagagaaaagattttttaccccaaggcagcgccagacaaagatggaaattggttcttcgttgtgaattcaaaagaaaacccagtacagggttataaagttgaaatttgcgaccgtcagcaattaccatgcgcggagttcgcgagcttccaacagggatatgaagcgaggtgcatccagaaatacgttcgccggaccatgttggcgttggatcccaagggtcagatgaccgacatgccccttaaagtgcccagctgttgctcatgcgtggccaaattgac

SEQ ID NO: 115 - DNA, 896 bp, Manduca sexta
  source 1..896: mol_type = genomic DNA
atgcaggctcggcggtggtgcgcggcactgctattaatgcagatgctgagctggctcggagtcagtggacacttaccgcgtcccgagtgcgcgccagccgcagattgccaacttatacgagacaacataatcgatggatatgcacaattctacttcaacgtatcaggacatgaagtgaaatttgaacattacatcggaaacgacttcgatgtcgaattgtcatgcaattacatcgccatggacaacgcaatgctgccgcggttctcaacgaccttttcagtcaacgtaatagtggttaaagaatgtgctttgccaagaagtgggtcaatcgatgccgctgtcgctgcacttaatatcaacgttttgacggagctgactctggacaaattcctagagccggcggtgatcacgcgcgcacatcttaccagtttacaacgactagagaggctggagctacacggtaactcaaacacaagcctcgcccccggcgcactggccgcgctctccgccgcgtccgcactgaaatgtcttgtattgcatgcagtacgcgtgcccgccgctgacctggcgcgcttgccgtcgtcactgcaagaactagcgttgttggatgtgggcgctgcgagtatgcatttagattcatcggttaatttgacgtcactcttcgtaatcgatacacattatcctgtcgtcgtgaatgtgagcaacgccgttgcgctcagagacttgcacataaataccccaagtactgtgttgaccgaagacgtgctcccgtcgtcactcaactcacttgaactagaggggtggaacgaaacgcatccggtgcctaagacacgttgtgtactacttaaggaacttaatgtaatcggcaccgacaatgatgcctatccggtgactct

SEQ ID NO: 116 - DNA, 567 bp, Manduca sexta
  variation 20
  source 1..567: mol_type = genomic DNA
acggaccttccgttggaccngccatcatcggcgctggagacatcgctgtcggccctgctatcgtcgacttccctttccccgacggcggtgccgtgtctgcccccgttgagccttcccccatcgccatcggacccgctatcgtcggtgaatcccctatctccgtcggacctgccatcgttgaggccggagacatcgctgttggacccgctatcatcgacttcccccttcccgacggtggcgccgtgtccgcccccgttgaggtttctcccgtcgactccgtcgtcgtcggccctgccgccggctctcagagctctcccctcgtccagatcatcatcaacgttaaggcccccgctggtgccggccccgttgtcgatgccgtcgctgacaagcccatggacatcattgatgttatgcccgtcgtcgaccctgctgatttcgtggacctcacccccgttgtagagcctgtagaagtcgtcgacattgtcgatgtcatgcccgtggttgaccccatcaacatcatcgatgttatgcctgttgttaagcccgtaaacccccttgcccgttctt

SEQ ID NO: 117 - DNA, 1350 bp, Manduca sexta
  source 1..1350: mol_type = genomic DNA
atggccgcaactacaccaggttttaagaagttagcagacgattctgaggattcagatacagaatacaccccgctgtatgatgacggtgatgaaatagatcaaagaactgcacaagaaacaaaaggatggaatctatttcgagagattccggtgaaaaaggagagtggatctatggccacaaaaaattggatagaaacaagcgtaaaaatcataaaagtgcttgcctacatattggttttttgtgctgtactgggttccgcagtcatagctaaaggaactcttctatttattacgtcacaactgaagaaagacagacaaattactcactgcaatagacgacttgctttagaccaacagttcataacggtacacagtttagaagaaagaataacatggctatgggcagcacttattgtattcggtgtgccggagttaggggtgtttttgagatccgtcaggatatgcttcttcaaaactgccaagaaaccaaccaaaacacagtttattattgctttcataacagagacactacaagcaataggaatagcagcacttgtattaataattctaccagaattagacgctgtgaaaggagccatgttgatgaacgccacgtgcgctatccctgcattgctaaacattttcacgagagaccgaatggattctaagttttctataaaattgatattggatgtattggcgatatcggcacaagccacggcgtttgttgtttggcctcttatggaaagaacgccagttctatggaccataccagttgcatgtgtgttagtgtctctaggcttctgggagaattttgttgacacctacaataaaagttatgtttttacggtgctgcaggaactacgcgacaacctcaagaggactcggtactacactcagcgggtgctatctgtttggaagattatagtgtttatggcatgcattttaatatcgctgcatatgcaaaatgacaatccgtttacctttttcactcacgccagcaaagcctttggagagagacagtatgtcgttaacgaggttctaatagtagtccgagatgacgaaaccataggctatgacgtcaccggaggtatattcgaattggacgcgatatggacctcagcattgtgggtcgcattaattcaagtgggagcagcctacttctgtttcggaagtggcaagtttgcttgcaaaattcttatacaaaattttagtttcactttagcattgactctcgtcgggcccgtggcaatcaacctccttattgctttctgcggaatgagaaatgcagacccttgcgctttccatagaactatacctgacaatttgttttacgagataccacctgtg

SEQ ID NO: 118 - DNA, 878 bp, Manduca sexta
  source 1..878: mol_type = genomic DNA
ttgctgtgcgttttccttggtagtgtatcgtcatgttgcgttaatgggcggtactacccgaggtaccatttgtcgccaccgcatggctggatgaacgaccccaacggattctgctacttcaaaggtgaataccatatgttttaccagtacaatcccatgtcaagtttggaggctggcatagctcattggggtcatgcgaaaagtaaagatttgtgccattggaaacacttagacccgccatctatcctgatcagtggtacgatcaaacgggagtattttctggaagtgcgctagtagagaatgacgtcatgtacctttattatactggaaatgtaaatcttactgatgaaatgccatttgagggacaattccaagctcttggtatcagtactgacggtgtccacgtagaaaagtataaagacaatccaataatgtacacgccaaaccatcaacctcacatccgagacccaaaagtttgggaacacgacggctcttattatatggtcttaggaaacgcatatgatgattatacaaagggccaaatagttatgtacgaatcatcagacaagatcaactggcaagaagtaactatactatataaatcaaatggatctttcggttacatgtgggagtgtccagatttattcgaaatagacggcaagtttgtacttctgttctctcctcaaggcgtgaagtctgtgggcgatatgtaccagaatctgtatcaagcaggatacatcgtcggagaattcgattacgatactcattcattcacaatactaaccgaattcagagaattggatcacggtcatgatttttacgctacacaaacaatgaaagatcctagtggaagaagaatagtcgttgcttgggcaagt

SEQ ID NO: 119 - DNA, 1333 bp, Manduca sexta
  source 1..1333: mol_type = genomic DNA
atgagggaaatcgtgcacatccaggctggccaatgcggcaaccagatcggagctaagttctgggagatcatctctgacgagcatggcatcgaccccaccggcgcttaccatggcgactcggacctgcagctggagcgcatcaacgtgtactacaatgaggcctccggcggcaagtacgtgccgcgcgccatcctcgtggacctcgagcccggcaccatggactctgtccgctccggacctttcggacagatcttccgcccggacaacttcgtcttcggacagtccggcgccggtaacaactgggccaagggacactacacagagggcgccgagcttgtcgactcggtcttagacgtcgtacgtaaggaagcagaatcatgcgactgcctccagggattccaactcacacactcgctcggcggcggtaccggttccggaatgggcaccctccttatctccaaaatcagggaagaataccccgacagaattatgaacacatattcagttgtaccatcacccaaagtgtctgatacagtagtagaaccttacaatgcaacactgtcagtccaccaactcgtagaaaacaccgacgaaacctactgtatcgacaatgaggctctctatgacatctgcttccgcacgctcaaactttccacacccacatatggcgaccttaaccacctggtgtcgctcacaatgtccggcgtgaccacctgcctcaggttccccggtcagctgaatgcggatctccgcaagctggcggtgaacatggtgcccttcccgcgtctgcacttcttcatgccgggcttcgctccgctcacgtcgcgcggcagccagcagtaccgcgccctcaccgtgcccgaactcacccagcagatgttcgacgctaagaacatgatggcggcgtgcgacccgcgtcacggccgctacctcaccgtcgccgccatcttccgtggtcgcatgtccatgaaggaggtcgacgagcagatgctcaacatccagaacaagaactcgtcgtacttcgttgaatggatccccaacaacgtgaagaccgccgtgtgcgacatcccgccccgtggtctcaagatgtcggccactttcatcggcaactccaccgctatccaggagctgttcaagcgcatctctgaacagttcaccgctatgttcaggcgcaaggctttcttgcattggtacaccggcgagggcatggacgagatggagttcaccgaggccgagagcaacatgaacgacctggtgtccgagtaccaacagtaccaggaggccaccgccgacgaggacgccgagttcgacgaggagcaagagcaggagatcg

SEQ ID NO: 120 - DNA, 439 bp, Plutella xylostella
  source 1..439: mol_type = genomic DNA
cccgatacagttggagtacctgccccggcccctggggctggtggtggtccagcacaccgccacccccgcgtgtgacactgacgccgcgtgtgtggagctggtgcagaacatacagaccaatcatatggatgtgctgaagttttgggatattggaccgaacttcctgattggtgggaacggcaaggtgtacgagggccctggttggctgcacgtcggcgcccacacttacggctacaacaggaagtctatcgggatctctttcattaggaattttaatgctaagaccccaacaaaagcagcgttgaatgcggctgaagcattgctgaagtgtggagtgagagaaggacacctgtctcactcatacgcagtggtcggccatagacaactgatcgcaacagagagcccaggcaggaaactgtaccaaatcatcaggcgctgg

SEQ ID NO: 121 - DNA, 971 bp, Plutella xylostella
  source 1..971: mol_type = genomic DNA
tcccgaagccacctagagaaccggtagagactacagagacatcacaaaataataccgaaaatcaaccttacaatgtcgaagaagaggaaatacccgaaccagaaaagcctaagaaagaaaaaaagaatcccaagcctaccaaaaaaactttctttaatcgtgacaaaactaacaaacacgacgatacccgcaaacatacaaaatccggaaaggaccagacatcaattaatactcaaggtaacttgaaaattatacttcctgtaacttaaacggcccgttgaccccagtttacctttcgcctttcttgatatatttttgtaatccagccttactttggtaatacatacttgccccacttgtatttagttaatggtggcactagctagatagtaatgttaaatgatgataagcagtagtgattcatcattcaaatgtatcattgtcctttaatgttaagcgcaaatagattttcattgttctcccatgtgcttcatgttttatgtatttataggtaggtacttaatgttttataaatatttttttgttaattgggaatccccagtccccattgtctggaccagtttatatataattgaactaacaagagtgtgctttaaatactattctctgcaattatgataattaaacaacatgaatttctcttcacttcccttctcttatttaaataatattgtaggaaactgtaataactaatacaagattataaatttcattctagcaactggtgatgtaatccatgtggtaaattccaaagatgtgcaggtcggccatcagtatgtgtacaacatgggaactcccggagctaactcacagaagaataacccatttgatgatgaagaaacagtagaaaagacaaatctaataactctggtcatggaagcaaaaattatggtaataacacatttttaactaggcataaggtcataatttagccagaatcatcagcttgt

SEQ ID NO: 122 - DNA, 298 bp, Plutella xylostella
  source 1..298: mol_type = genomic DNA
caatgctgcggagggtctatgcgggtggacacctctacacgtagcggcggcgcgaggcgacgtcgacacggctcgctacttgctcgagaagtgcgctggcgtcgatccctctgccctggactacgccggtcgtacggccaggaaactggcgttgaagaataaagcggccgccctgtttgacggcagtgagggcagcgaggaggaggatagtgacagtgaggatgagatgcttctggaaagcgaccagagtctgttcgaccggatccgtgacggtatgaacgccatcaacgtcgcctga

SEQ ID NO: 123 - DNA, 229 bp, Plutella xylostella
  source 1..229: mol_type = genomic DNA
taacctgtgccagcaaccaatggctcctatggcgcagcagctgatggacccgtcccccagcgacccaccctccatcacggggctgctgatggatcgcccggaccagccctactccggggagctgtctggactctccgccctgctggctgaggcagcccccgcagagatgctcagcgatagcctcaacagactgtctacgggggacttgttgagacaagttgatatgtga

SEQ ID NO: 124 - DNA, 608 bp, Plutella xylostella
  source 1..608: mol_type = genomic DNA
gaaggattgaagtgagcgccagaatgccgcgcggtgattggttgattccagatattctgctggagccgaaagaaaacctttacggagtacgcaattacgcgtcaggtctactcagcatagcctcagtcagaggaaacactgcttactcgaagaccctcaaaggaggccccatactgtgtgacaaggaaccgcagagaagtgccaagttgagcgaaaaagttggatatgaccattggaataaagccttccataactacaccatgatttgggcaccaagtggcatcaccatgctggtggacggcgagcagtacggggacatccgtcccggcgacggcttcagccaggacccggcggtgagcagcgtggtggccgcgccgcagtggctgaagggcaccagcatggcgccctttgatgttatgttctacatatcccttggtctccgcgtgggcggagtgaacgacttccccgacactcctgagaagccgtggaagaacaaggccactaaagccatgctgaatttctggaacgcccgggaacagtggcagagcagctggtttgaggacaccactgcactcctcatagactatgtcagggtttatgcgctgtga

SEQ ID NO: 125 - DNA, 929 bp, Plutella xylostella
  source 1..929: mol_type = genomic DNA
cgtatctcgcacgacctgaaagagctgcgaaactcatccgtcttttccttctttatgatcaatgccctctttgttctcatcgtattcttgctgcaactgaacaaggacaacctccacataaagtggcccttcggagtcaaaactaacattacgtatgatgaggtgacgcaagaggtgctgatctccaaggatacctgcaactagagcctattggtctggtgttcgtgttctttttcgcattgattttagtcatccagttcactgccatgttgttccatcgattcggaactttgtcgcatatattatcgtctacggaactgaactggttctgcaataagaaggcggaagacttatctcaagacgcactgctagataagaatgcgatagcaatagtgaaggatctccagaaactaaacgggctcgatgacgggtatgacaatgactcggggtcgggcccgcacaatgtgggaaggagaaagacgatacacaacctggagaaagcgagacagaagaagaggaacataggaacgctcgacgtcgctttcaagaagcgattcttcaacatgaacgctaatgaaggaccaggaacaccagttctgaaccgcaagatgacgttgcgaagagagacgttgaaggcgttggaaacgaggaggaattctgtgatggccgaacgaaggaagtcgcaaatgcaaacacttggagctaacaacgaatatggagtcactggaatcttaaacaacaacccagcggtgatgccgcgccaccggccgtcgacagccaacatttcggtcaaggacgtcttcgcggaacccaacgggggacaagtgaaccgagggtacgagaccacgcacggcgacgagggagacggcaactccatcagactgcagccgagaaccaaccaggtctccttccaggggagataccaataa

SEQ ID NO: 126 - DNA, 364 bp, Plutella xylostella
  source 1..364: mol_type = genomic DNA
gaaggaggtcgacgagcaaatgttgaacatccagaacaagaacagcagctacttcgtcgaatggatcccgaacaacgtcaaaacggccgtgtgcgacataccgcctcgtggactgaagatgtctgccacctttatcgggaacacgacagcaatccaagagctcttcaagaggatttctgagcagttcactgctatgttcaggagggaagcgttcctccactggtatactggtgaaggcatggacgagatggagttcacagaggcggagagcaacatgaacgacctggtctccgagtaccagcagtaccaggacgccacggctgaagacgagggagaattcgacgaggatattgaagacgagtga

SEQ ID NO: 127 - DNA, 590 bp, Spodoptera frugiperda
  source 1..590: mol_type = genomic DNA
cctgattggtggaaacgggagagtttatgaaggagccggctggcatcacgttggggcccatactttgggatacaatgcaagatctgtggggatctccttcattggcgattttagaacaaaattaccaacacccgaagcactgaaagccttcaacagtctcctggaatgtggagtcacgaacaattatctgtcaaaggactatcacctggtggcccatagtcagctctctatgactgacagtccyggagacatgytgaggaagcaggtggaatcgtggcctcmttggctggataatgccaaagacatacttaagtagaaraagactaaacgccgtactttgagccatttaatggttacttaacccagtccttagcaatttgatacaaggccaatgtctctaagggcggcagtaaaggtcaaaacacatttaatgagtgtgtttaagattttgctagtgaaaattgttttgaagtacgtatttgatgtaagtgatgatatcagtacccttagtatgagtttgctttacgttccacgagatggaaacgagagcgcgttcggcgctctgattggttcgttcattcatgccggcc

SEQ ID NO: 128 - DNA, 828 bp, Spodoptera frugiperda
  source 1..828: mol_type = genomic DNA
gccagacatctaccacaggaccactcaacgtacgaccaagtacaactcctcgggttcgacgaagatggacgaccagtgtttgagcacgaagacttactcccagaactagaggagtcctaccagccagagcacctggcgaggactcgcagacaggcgcagggcagcgtcaccctcaactccgacggcggcataggcctgggcgctaagatcccgctcgcacacaacgacaagaatgtggtgagcgccatcggctccatggacttcaacaacaagttgcagcctgcttccaagggcttcggtctggctctggacaacgtcaacgggcacggactgacggtgatgaaggaaagtatccccgggttcggggacaggctgtcgggcgctggcaagctgaacgtgttccacaacgacaaccacaacgtggccgtgaccggctctctcgccaggaacatgcccagcatcccgaacgtgcccaacttcaacacgtacggcgggggcgtcgactacatgtacaagaacaaggtgggagcgtctctgggcatggccagtactccgttcttggaccgcaaggactactccgcgatgggcaacctgaacctgttccgcagcccgaccactaccgtggacttcagcggcggctttaagaagttcgaatctcccttcatgagcagcggctggaagcctaacttcggccttactttcggcagatctttctagatatattttgtaatctaaatttaactttaactttgttgtataatattttgtcgaattaagatcagtattgttcatactaatattatattatcagtgtttcttataaattaa

SEQ ID NO: 129 - DNA, 508 bp, Spodoptera frugiperda
  source 1..508: mol_type = genomic DNA
caaggctcgtaccttgcagttgaaccgacaatctattcctaaagcctttttaaggtcaggaaaaatagttcctacatctaaatgcagtagaatttgcgaaacgaatttaaataaaaatggcgtcgattgtgtttgtgattttgtgtgttaccgtcgctgcggtgaaaagcgcgattttaaacccgtggagtaaagttgaggccaacaaatgtggtgtagaagccagtactaacttggtccatcacaatccatggttggtctacatcgagtattggcgtggaaactcagatactgagatccgatgcgccggtactttaatcgacagcaaacatgtcgtcacagctgcccactgcgttaggactctgaagtttagtcatttgatcgcccgtcttggcgaatacgacgtaaattctaaggaggactgcgttcagggcgtgtgtgccgatcccatcgtcagaatcaaggtggctgagatcatcgtgcatcctaactacagcaaccgggaaca

SEQ ID NO: 130 - DNA, 708 bp, Spodoptera frugiperda
  source 1..708: mol_type = genomic DNA
agcaacaaaatgcgtgtcctcgcttgcttggcccttctcttagctgtggtagcagccgtcccctccaatccccagaggattgtgggtggttcggtcaccaccattgaccggtaccccaccattgcatccctgctgtactcgtggaacttgagttcctactggcaggcgtgcggtggttccatcttgaacaaccgtgccatccttactgctgcccactgcacagttggtgacgccgccaacagatggagaatccgtcttggctccacctgggccaacagcggtggtgtcgttcacaacgtcaacactaacatcgtccacccctcatacaactctgcaactttgaacaacgacatcgctatcctccgctccgccaccaccttctccttcaacaacaatgttcaggctgcctccattgctggtgccaactacttgcccggtgacaacaccgccgcctgggccgctggatggggaactacctccgctggtggctctagctctgagcagctccatcacgttgagctgcgcatcatcaaccaggctacttgcaaaaacaattacgctacccgcggtatcaccatcacctacaacatgttgtgctctggctggcccaccggtggtcgcgaccagtgccagggtgactctggtggtcctctctaccacaacggcatcgttgttggtgtctgctctttcggtattggctgtgctcag

SEQ ID NO: 131 - DNA, 1003 bp, Spodoptera frugiperda
  source 1..1003: mol_type = genomic DNA
aacagttttctattggcagtcaaagacttcagtcgaaaaataatcctcatcagagtcgtgaagcaaggtgcccaaaatataatgtaacctagatacctattaataaattatttgtcaaccaaaacgttacgttcaaagtccttaaaatcaaaatatcttatgattagttttgatttaaaaatagaggttcgaaatcgccaacccaaataggtttagtttacgattcaggaaaaatcctaacgtagggaaacattattttacaagacttttggcttaaaaactttgagaaccaatgtcaaatttgataataactaatgaggtataaaagcttgatcctattaggacttattttcataacaccatcgagtttgtatttaatrragacgtgkgttaactaacaacatgaagaccggtgtaaaatattctgttmtttggatattctctctattytgctatatagaggcaacatttcgttgtgactacacgtacagcaaggaagcgaagggctggttcaaacatgtggtgataccagctacttgggctgacgcacgwctgcactgcacgttggaaggtgcaacgctggcttctccactcaaccaggctatmagtaatgagatgcagtccmtcctggcraacctctcggcgctgcaatcagaagtcttcactggaattcacrcgactktttcacgrmrcaacttatatcatacyatygaaggtatacctcttagtaaaattccattagattgggcaacaaatgagccaaatggtgggagagatgaaaactgtatcacgtttaactccgatggccaagcggcagacagatcctgtaargagactcgaccttacatctgctaccgacacacwactaaagtgactgtgkccaatgaatgtgggactgtagatcctgaatacaatttggataaaagaacgggckcytgctataagttccacacrgtacctcgcacgttcgagcgtgccaacttcgcgtgttctgctgaaggtg

SEQ ID NO: 132 - DNA, 391 bp, Spodoptera frugiperda; identical to SEQ ID NO: 98
  source 1..391: mol_type = genomic DNA

SEQ ID NO: 133 - DNA, 500 bp, Spodoptera frugiperda
  source 1..500: mol_type = genomic DNA
gcaacaatcgcaccaactctttcgtgcgcagtggaagtttgttcatccgtccctctctaacatcagacgagttcggagaagctttcctctcatctggacactggaacgtcgagggtggtgctcctgctgatagatgcacaaatccacaatggtggggttgcgagagaacaggcacgccgaccaacattttgaacccaatcaagagtgctcgtgtccgtaccgtcaattccttcagcttccgttacggacgcctcgaagtccgcgctaaaatgcccgccggagattggatttggccagctatctggttgatgcctgcgtacaacacttacggtacttggcccgcatcaggagagattgacttagttgagtcccgaggcaaccgtaacatgttccacaatggtgtccatatcggtacacaggaagcaggctcgaccttgcactacggaccttacccagcgatgaacggttgggagcgcgcccattgggtcagaaggaaccct

SEQ ID NO: 134 - DNA, 504 bp, Spodoptera frugiperda
  source 1..504: mol_type = genomic DNA
tcgttctcctcgtcgctttcttggggacctcatggtttacgggagatgtttctgcgagtccgcggccgcaagagccgcgtgtggatcaaaatccgaatcaggtgtcaccttatggagggtccgggtaccacgcacctccgcagtaccaaccgcagtaccaaccacagccgtactacccacagccgcagtactacccacagccgtactacccacctcctcagtattacccaccgcagccacaaacacctgagaatgctccactcataaacacatggaatggtttccacgactgggctcagaatatcgttcaaagtgctttggggcagaaattcccgaaaggtagacagtaactttttaattgtcaattgaagataaggcccatttcaccaactgctgtttaattttaaggagctcctaaactaacataggtgacacttagcgatattctggattttttgtgaacgtataaataatatccaaatgtaaaagataagaggccaagaa

SEQ ID NO: 135 - DNA, 949 bp, Spodoptera frugiperda
  source 1..949: mol_type = genomic DNA
gaatttaggagcagcgtgcgggcgcatccatcctgtgggctcaggcttcatggcatggtatcaaatgttcgagtacgctattggtcattggctgcaaaaggcgactgaacacatgattggctgtgtactctgtagccctggatgcttctccctcttcagaggaaaggctttgatggacgacaacgttatgaagaaatataccttaacttcccacgaggcacgacactatgtgcaatacgatcaaggcgaggaccgttggtgcacgctactgctgcagcgcgggtaccgcgtggagtacagcgcggtgtcggacgcgtacacgcactgccccgagcacttcgacgagttcttcaaccagcgccgccgctgggtgccctccacgctcgccaacatcttcgacctgctcggcagcgccaagctcaccgtcaagtccaacgacaacatctccaccctctatatagtctatcagttcatgttgatagtgggtacggtgttgggtcccggcacgatcttcctgatgatggggggagccatgaacgccatcattcagatcagcaacgcgtacgcgatgatgttgaacctcgtaccactcgtcatcttccttatagtctgtatgacttgtcagtcaaagacgcagctcttcctcgctaacctcataacatgcgcatacgcaatggtgatgatgatcgtgatagtggggatagttctgcagatagtggaggatggatggctggctccgtccagtatgttcacagctttaatattcggtacattcttcgtcaccgcggcactacacccgcaagagatcaaatgtttgttgttcatagcagtgtactatgtaaccatccctagtatgtacatgttgttgatcatatactccatctgtaatctcaacaacgtatcctggggtaccagggagacaccgcagaagaaaactgctaaggaaatg

SEQ ID NO: 136 - DNA, 623 bp, Tribolium castaneum
  source 1..623: mol_type = genomic DNA
atgagtggcagtgaccctttaacaaatacccaacaatccgatcaagattattaccatccactctgttattcaattcaagtggacgacgaaaatgaacaatcagctctcctgcccgcatttcatcaaaggaaaagtttgcgagttcaggataaaatctttattgtatttttattttcaattctaattaccggactagccattggcctctatctccttgcaactgagggacacgaatggaaagctgcaggagtctataatattacagttcgggaacagtggcaagctcacgtcccttcatcaacaatgccaaagttggaacttcccgtaagaagagttttatttcttcctgcaaataccactagctgcggcagcaaatcccactgtgccaaagtcctccaggaactacaattacagcatatgctgcagtggaaagaacctgacatctcctacaatttcataatgactgcagatggcagaattttcgaggggagaggatgggactttgaaacttctgttcaaaattgtacggttaatgatactgtgacagttgcttttttggacgaattagatgcgaaagcaccgacgtttagacaagctgaagcggcaaaaatgttcctggaagt

SEQ ID NO: 137 - DNA, 718 bp, Tribolium castaneum
  source 1..718: mol_type = genomic DNA
acattacaacgttccgcgaccctcaatccaagcattcaggccccgtggcttcaaagtgagcatccctcatactgaaggcatccaattattcgcctttcatggaaatattaataaacccctgcacggtctcgaggccggacaattttcccaagacgtcctccaaagggagggagacgagtgggtgttccaagactccagtgccaaattaaacgtgggagataaaatctattattggctctttatcattaaagaagacctgggctacagatacgatcacggcgagtacgaagtgaaagttttagccacccgtgacttcgattctcctcaaacaacctctgtgacgccgaatcttgcccccaatctcggcatttgcgaaaaggtgatggtaaatctcacgcggaagctgctcgatttgcagcaagaaattgagtcccttagggagacgaacgatattttggaggatatggttcagaagcacactgatacggcgactactctcactttggacggcttgatgataaaggatgacgacgaacttgtttcggtgattcaagcaattattaaagataaacttggactaaaaagcaggattcaaaatgtgacgaggcaggagaatggaatggtcaagtttcaagtggcgagtttgagagaaaagttggaggttgtcaaagcggccaaaagaaaactcaagtcgtcgagctttacgatcacgtattaa

SEQ ID NO: 138 - DNA, 661 bp, Tribolium castaneum
  source 1..661: mol_type = genomic DNA
ccgttgaagaggatgcccagtgaagaaatcagtatcagtgatctgcctagcgaaatgaaagaagttttactagaaattagcccgaactttgatgaaaatctgaaacgggctttcaggaacgaaggagtgaggctgcagagagtgcagaacaatggacgatttattcatcagctggacgacgttctcttatccatagacgataccaaaatcgagttacgcaacctgaaattcccctggatccccgacttccgcatcgtggacttgtccagcgacctgcccatgtcatgcctcgacctaaacctaaacctgggcaatttgcgaattgagggcgagtacgaagccaacaacaccacactcaggcgatggctcccggtatctcacattggtcgaatcgtgatcggttttaacaacgtccgagcgaacggaaaagtcggactcgtgctcgagcaggattctttcgttccgcagaattatgatattagatatgagccgacggatgttgttattagggttagctatcacgtggatggcgagaatgaggtgcaaaatgagattagcaattcagatattgaggccacgctaggcaagaccgtgtgggtgcagttgactgagatattgtccaacctgttgcataggcaattgggcgaggttgtagtggagt

SEQ ID NO: 139 - DNA, 767 bp, Tribolium castaneum
  source 1..767: mol_type = genomic DNA
atggcggcgcgtcatcgctttgccacagggagccctgaggaaacagagcccctgtattcgtcgacgcaaatgcccgaaaaagtccgggaaaaatggaacgtcttcgacgaccccccaagagagcccacttcggttccgaagtcaaaagaacctacatcgagtggggggtgaagtttttgaaagttgtgacaatcataactgtgttttttgtggtccttggtgctgcagtggtttcgaaagggacaaccttgttcatgacgtcacaaataaaaaagaatgtgacaagggcttattgcaacaaaaagatagaccgcaacctccaattcgtcgtctccctccccgaagtggagcgcgtgcaatggatctggctcctcattttcgcttacttgatccccgaagtgggtacctggatccgcgccgtccgcaaatgcctctacaagctctggaaaatgccctccctctccgaattcctctccctcttcggcacggaaacgtgccccgccatcggaagcgcaattttgatattcgtcgtcctccccgagctggacgtcgtcaaaggggcgatgctcacaaacgcggtttgtttcgtgcccggagttgtggcaatgttctcgcgcaaaccgtgctccataaacgagaacctgaaaatggggctggacatcgccagcataactgcacaggcgtcaagcttcgtggtgtggcccttggttgaaaataacccgaccttgtacctaatccccgtttccgtgattttgatttcggtgggt